(19) United States
(12) Patent Application Publication
(10) Pub. No.: US 2025/0154190 A1
(43) Pub. Date: May 15, 2025

(54) STEREOSELECTIVE TECHNOLOGIES FOR CHIRAL COMPOUNDS

(71) Applicant: WAVE LIFE SCIENCES LTD., Singapore (SG)

(72) Inventors: Pachamuthu Kandasamy, Lexington, MA (US); Subramanian Marappan, Acton, MA (US); Mamoru Shimizu, Arlington, MA (US); Chandra Vargeese, Schwenksville, PA (US); Jayakanthan Kumarasamy, Arlington, MA (US); Ik-Hyeon Paik, Needham, MA (US); Marissa Sauter, Tewksbury, MA (US); Arindom Chatterjee, Burlington, MA (US)

(21) Appl. No.: 18/836,993

(22) PCT Filed: Feb. 13, 2023
(86) PCT No.: PCT/US2023/012949

§ 371 Date: Aug. 8, 2024

Related U.S. Application Data
(60) Provisional application No. 63/309,467, filed on Feb. 11, 2022.

(51) Int. Cl. C07H 19/067; C07D 207/08; C07D 207/09; C07H 19/16; G01N 33/53

(52) U.S. Cl. CPC C07H 19/067; C07D 207/08; C07D 207/09; C07H 19/16; G01N 33/5308

(57) ABSTRACT

Among other things, the present disclosure provides technologies for stereoselective preparation chiral compounds. In some embodiments, prepared chiral compounds are useful for chirally controlled preparation of oligonucleotides. In some embodiments, such oligonucleotides target transcripts associate with various conditions, disorders or diseases.

### CROSS-REFERENCE TO RELATED APPLICATION

This application claims priority to U.S. Provisional Application No. 63/309,467, filed Feb. 11, 2022, the entirety of which is incorporated herein by reference.

### BACKGROUND

Chiral compounds are useful for many purposes including in stereoselective synthesis. For example, chiral compounds among other things can be used as chiral auxiliaries in stereoselective synthesis of oligonucleotides. These compounds are also useful, among other things, as biologically active agents including in many cases therapeutic agents.

### SUMMARY

Among other things, stereopure and stereo-enriched chiral compounds are important reagents for stereoselective oligonucleotide synthesis. In some embodiments, the present disclosure provides technologies (e.g., compounds, methods, etc.) for stereoselective preparation of chiral compounds. In some embodiments, provided technologies are particular useful as they provide higher selectivity, shorter synthetic routes, higher overall yields, milder reaction conditions, lower manufacture costs, and/or are easier to scale up comparing to reference technologies, e.g., those reported existing ones. For example, in some embodiments, provided technologies utilize more stable intermediates compared to existing technologies (e.g., more stable ketones compared to aldehydes).

In some embodiments, the present disclosure provides technologies for preparing chiral compounds, e.g., chiral phosphoramidites or salts thereof. In some embodiments, the present disclosure provides technologies for preparing cis cyclic phosphoramidites (e.g., relative to -L-R¹) which phosphorus atom is chiral and is a ring atom. In some embodiments, provided technologies can deliver increased cis cyclic phosphoramidite levels relative to corresponding P epimers. In some embodiments, the present disclosure provides technologies for epimerization of P chiral centers. For example, in some embodiments, the present disclosure provides technologies for epimerization of cis cyclic phosphoramidites at chiral phosphorus atoms.

In some embodiments, the present disclosure provides technologies for preparing oligonucleotides comprising PN linkages. In some embodiments, oligonucleotides comprise sulfonyl PN linkages. In some embodiments, provided technologies utilize reduced amounts and/or equivalents of azide agents. In some embodiments, provided technologies reduce cost and/or improve safety.

In some embodiments, the present disclosure provides a method for preparing a compound of formula P:

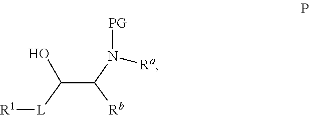

or a salt thereof,
comprising reducing a compound of formula INT-1:

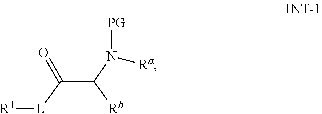

or a salt thereof to provide a compound of formula P or a salt thereof,
   wherein each variable is independently as described herein

In some embodiments, the present disclosure provides a method for preparing a compound of formula P-a:

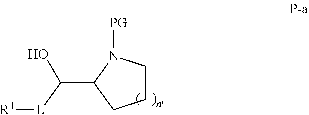

or a salt thereof,
comprising reducing a compound of formula INT-1-a:

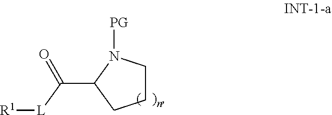

or a salt thereof to provide a compound of formula P-a or a salt thereof,
   wherein each variable is independently as described herein.

In some embodiments, provided technologies do not require cryogenic conditions and can be performed at larger scale with easier operational conditions. In some embodiments, provided technologies provided chiral compounds with higher stereoselectivity. In some embodiments, provided technologies provided chiral compounds with higher stereopurity. In some embodiments, provided technologies provided chiral compounds with higher chemical purity.

### BRIEF DESCRIPTION OF THE DRAWINGS

FIG. 1 NMR spectra showing isomerization of OMeU-L-DPSE cis-isomer 8-3 to trans-isomer 8-4.

FIG. 2 NMR spectra showing isomerization of OMeU-L-PSM cis-isomer 8-11 to trans-isomer 8-12.

### DETAILED DESCRIPTION OF CERTAIN EMBODIMENTS

### 1. Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, etc.) is from 5′ to 3′. Unless otherwise specified, oligonucleotides described herein may be provided and/or utilized in a salt form, particularly a pharmaceutically acceptable salt form. As those skilled in the art will appreciate, oligonucleotides may be in various forms, e.g., acid, base or salt forms. In some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation, or combinations thereof. Unless otherwise specified, aliphatic groups contain 1-100 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkenyl: As used herein, the term “alkenyl” refers to an alkyl group, as defined herein, having one or more double bonds.

Alkynyl: As used herein, the term “alkynyl” refers to an alkyl group, as defined herein, having one or more triple bonds.

Aryl: The term “aryl” used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. In some embodiments, an aryl group has a radical or point of attachment on an aromatic ring.

Chiral control: As used herein, “chiral control” refers to an ability to control the stereochemical designation of a chiral linkage phosphorus in a chiral internucleotidic linkage within an oligonucleotide. In some embodiments, a control is achieved through a chiral element that is absent from the sugar and base moieties of an oligonucleotide, for example, in some embodiments, a control is achieved through use of one or more chiral auxiliaries during oligonucleotide preparation as exemplified in the present disclosure. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in a chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share 1) a common base sequence, 2) a common pattern of backbone linkages, and 3) a common pattern of backbone phosphorus modifications, wherein the plurality of oligonucleotides share the same stereochemistry at one or more chiral internucleotidic linkages (chirally controlled internucleotidic linkages), and the level of the plurality of oligonucleotides in the composition is pre-determined. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. In some embodiments, oligonucleotides of a plurality share the same constitution and may be optionally in various forms (e.g., acid, basic, salt, etc.).

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule.

Halogen: The term “halogen” means F, Cl, Br, or I.

Heteroaliphatic: The term “heteroaliphatic” is given its ordinary meaning in the art and refers to aliphatic groups as described herein in which one or more carbon atoms is replaced with a heteroatom (e.g., oxygen, nitrogen, sulfur, silicon, phosphorus, and the like).

Heteroalkyl: The term “heteroalkyl” is given its ordinary meaning in the art and refers to alkyl groups as described herein in which one or more carbon atoms is replaced with a heteroatom (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon or hydrogen. In some embodiments, a heteroatom is oxygen, sulfur, nitrogen, phosphorus, or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.).

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, etc.), pattern of backbone chiral centers (i.e. pattern of linkage phosphorus stereochemistry (Rp/Sp)), and pattern of backbone phosphorus modifications (e.g., pattern of “-XLR¹” groups in formula I). In some embodiments, oligonucleotides of a common designated “type” are structurally identical to one another.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds (e.g., phosphate linkage groups of oligonucleotides, phosphorothioate linkage groups of oligonucleotides, etc.) with bases. Representative alkali or alkaline earth metal salts include salts of sodium, lithium, potassium, calcium, magnesium, and the like. In some embodiments, pharmaceutically acceptable salts are ammonium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Predetermined: By predetermined (or pre-determined) is meant deliberately selected, for example as opposed to randomly occurring or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that cannot be controlled to intentionally generate the particular chemistry and/or stereochemistry features is not a “predetermined” composition. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled.

Protecting Group: The phrase “protecting group,” as used herein, refers to temporary substituents which protect a potentially reactive functional group from undesired chemical transformations. Examples of such protecting groups include esters of carboxylic acids, silyl ethers of alcohols, and acetals and ketals of aldehydes and ketones, respectively. A “Si protecting group” is a protecting group comprising a Si atom, such as Si-trialkyl (e.g., trimethylsilyl, tributylsilyl, t-butyldimethylsilyl), Si-triaryl, Si-alkyl-diphenyl (e.g., t-butyldiphenylsilyl), or Si-aryl-dialkyl (e.g., Si-phenyldialkyl). Generally, a Si protecting group is attached to an oxygen atom. The field of protecting group chemistry has been reviewed (Greene, T. W.; Wuts, P. G. M. Protective Groups in Organic Synthesis, 2nd ed.; Wiley: New York, 1991). Such protecting groups (and associated protected moieties) are described in detail below.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates, allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benznylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include triphenylmethylamino (—NH—CPh₃), t-butyloxycarbonylamino (—NHBOC), ethyloxycarbonylamino, methyloxycarbonylamino, trichloroethyloxycarbonylamino, allyloxycarbonylamino (-NHAlloc), benzyloxocarbonylamino (—NHCBZ), allylamino, benzylamino (-NHBn), fluorenylmethylcarbonyl (-NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂ C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{∘} include=O and ═S.

Suitable divalent substituents include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Unsaturated: The term “unsaturated” as used herein, means that a moiety has one or more units of unsaturation.

### 2. Detailed Description of Certain Embodiments

Chiral compounds have a variety of applications. For example, chiral compounds comprising —OH and —NH— groups are widely used chiral auxiliaries. In some embodiments, chiral compounds described herein are chiral auxiliaries comprising —OH and —NH— groups. In some embodiments, compounds disclosed herein are used for preparing phosphoramidites. In some embodiments, phosphoramidites of present disclosure are used as monomers for oligonucleotide synthesis.

Among other things, the present disclosure provides technologies (e.g., compounds, methods, etc.) for stereoselective preparation of chiral compounds. In some embodiments, provided technologies are particular useful as they provide higher selectivity, shorter synthetic routes, higher overall yield, milder reaction conditions, lower manufacture cost, and/or are easier to scale up compared to existing ones (e.g., those reported in U.S. Pat. No. 9,598,458). For example, in some embodiments, provided technologies utilize more stable intermediates compared to existing technologies (e.g., more stable ketones compared to aldehydes). In some embodiments, provided technologies do not require cryogenic conditions and can be performed at larger scale with easier operational conditions. In some embodiments, provided technologies provided chiral compounds with higher stereopurity. In some embodiments, provided technologies provided chiral compounds with higher chemical purity.

In some embodiments, the present disclosure provides stereoselective methods for preparing chiral compounds. In some embodiments, the present disclosure provides methods for preparing chiral compounds that are useful for various purposes such as chiral auxiliaries, synthetic materials, biological agents, etc.

In some embodiments, the present disclosure provides a method for preparing a compound of formula P:

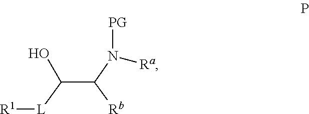

or a salt thereof,
comprising reducing a compound of formula INT-1:

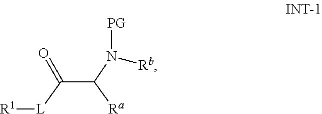

or a salt thereof to provide a compound of formula P or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or

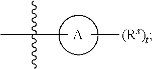

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a compound of formula P has the structure of formula P-a. In some embodiments, a compound of formula INT-1 has the structure of formula INT-1-a.

In some embodiments, the present disclosure provides a method for preparing a compound of formula P-a:

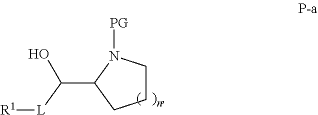

or a salt thereof,
comprising reducing a compound of formula INT-1-a:

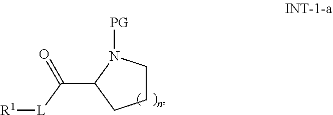

or a salt thereof to provide a compound of formula P or a salt thereof, wherein:
the reduction of a compound of formula INT-1-a or a salt thereof is carried out in the presence of a reducing agent;
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

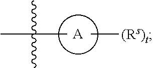

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a compound of formula P or P-a has the structure of formula P-b, wherein each variable is independently as described herein.

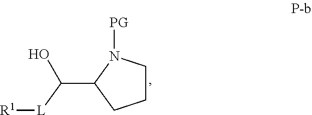

In some embodiments, a compound of formula INT-1 or INT-1-a has the structure of formula INT-1-b. In some embodiments, the present disclosure provides a method for preparing a compound of formula P-b:

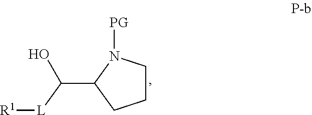

or a salt thereof,
comprising reducing a compound of formula INT-1-b:

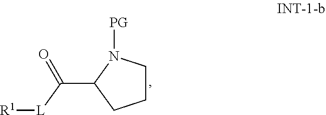

or a salt thereof to provide a compound of formula P or a salt thereof, wherein:
the reduction of a compound of formula INT-1-b or a salt thereof is carried out in the presence of a reducing agent; and
each variable is independently as described herein.

In some embodiments, a compound of formula P has the structure of P-1, P-2, P-3 or P-4, wherein each variable is independently as described herein. In some embodiments, a compound of formula P-a has the structure of P-a-1, P-a-2, P-a-3 or P-a-4, wherein each variable is independently as described herein. In some embodiments, a compound of formula P-b has the structure of P-b-1, P-b-2, P-b-3 or P-b-4, wherein each variable is independently as described herein.

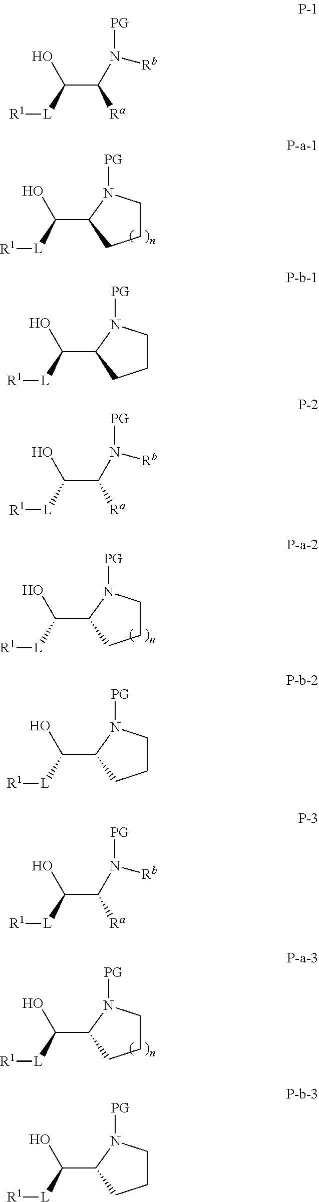

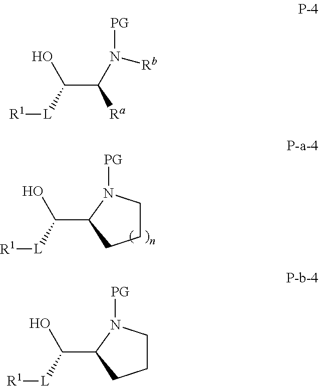

In some embodiments, a compound of formula INT-1 has the structure of INT-1-1 or INT-1-2, wherein each variable is independently as described herein. In some embodiments, a compound of formula INT-1-a has the structure of INT-1-a-1 or INT-1-a-2, wherein each variable is independently as described herein. In some embodiments, a compound of formula INT-1 has the structure of INT-1-b-1 or INT-1-b-2, wherein each variable is independently as described herein.

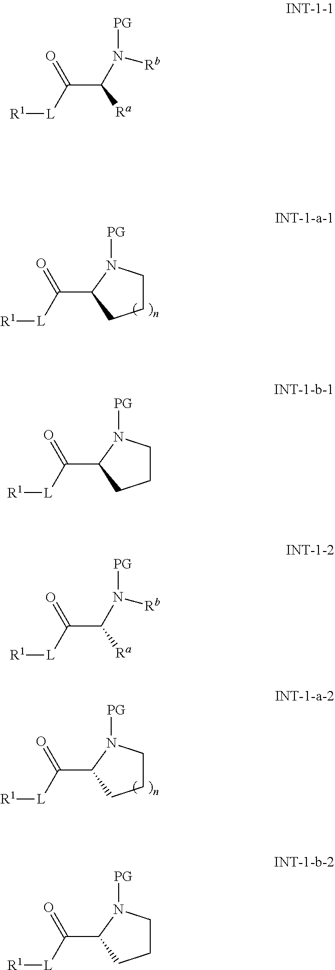

In some embodiments, in a provided method a compound of formula P or a salt thereof is a compound of formula P-1, P-a-1, P-b-1, or a salt thereof, and a compound of INT-1 is a compound of INT-1-1, INT-1-a-1, or INT-1-b-1, respectively, or salt thereof. In some embodiments, in a provided method a compound of formula P or a salt thereof is a compound of formula P-2, P-a-2, P-b-2, or a salt thereof, and a compound of INT-1 is a compound of INT-1-2, INT-1-a-2, or INT-1-b-2, respectively, or salt thereof. In some embodiments, in a provided method a compound of formula P or a salt thereof is a compound of formula P-3, P-a-3, P-b-3, or a salt thereof, and a compound of INT-1 is a compound of INT-1-2, INT-1-a-2, or INT-1-b-2, respectively, or salt thereof. In some embodiments, in a provided method a compound of formula P or a salt thereof is a compound of formula P-4, P-a-4, P-b-4, or a salt thereof, and a compound of INT-1 is a compound of INT-1-1, INT-1-a-1, or INT-1-b-1, respectively, or salt thereof. In some embodiments, a mixture of compounds of INT-1-1 and INT-1-2, or of INT-1-a-1 and INT-1-a-2, or of INT-1-b-1 and INT-1-b-2, or salts thereof, is utilized. In some embodiments, compounds of INT-1-1, INT-1-a-1 or INT-1-b-1, or salts thereof are selectively reduced. In some embodiments, compounds of INT-1-2, INT-1-a-2 or INT-1-b-2, or salts thereof are selectively reduced. In some embodiments, products are formed stereoselectively as described herein.

In some embodiments, a compound of formula P-a or a salt thereof has the structure of

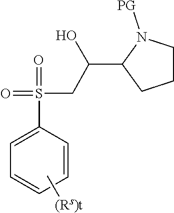

or a salt thereof. In some embodiments, it has the structure of

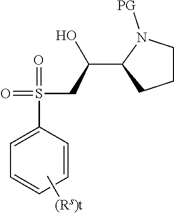

or a salt thereof. In some embodiments, it has the structure of

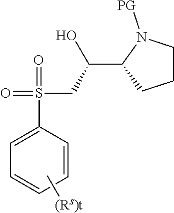

or a salt thereof. In some embodiments, it has the structure of

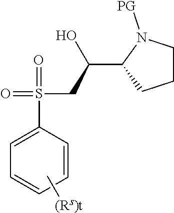

or a salt thereof. In some embodiments, it has the structure of

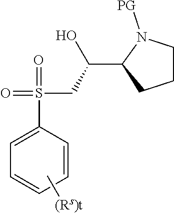

or a salt thereof. In some embodiments, it has the structure of

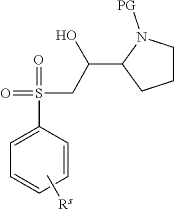

or a salt thereof. In some embodiments, it has the structure of

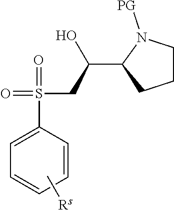

or a salt thereof. In some embodiments, it has the structure of

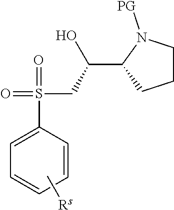

or a salt thereof. In some embodiments, it has the structure of

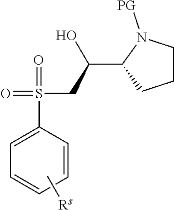

or a salt thereof. In some embodiments, it has the structure of

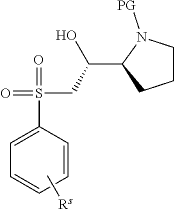

or a salt thereof. In some embodiments, R^{s} is R as described herein. In some embodiments, R^{s} is H, halogen, CN, COOR, OR, N(R)₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H. In some embodiments, R^{s} is halogen. In some embodiments, R^{s} is F. In some embodiments, R^{s} is Cl. In some embodiments, R^{s} is Br. In some embodiments, R^{s} is CN. In some embodiments, R^{s} is COOR. In some embodiments, R^{s} is COOR wherein R is not H. In some embodiments, R^{s} is OR. In some embodiments, R^{s} is OR wherein R is not H. In some embodiments, R^{s} is N(R)₂. In some embodiments, R^{s} is R as described herein.

In some embodiments, a compound of formula P-a or a salt thereof has the structure of

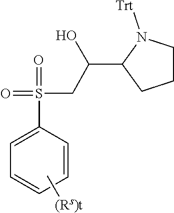

or a salt thereof. In some embodiments, it has the structure of

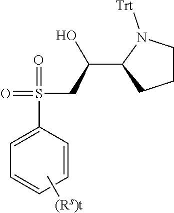

or a salt thereof. In some embodiments, it has the structure of

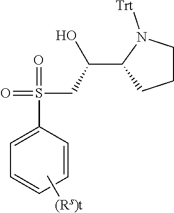

or a salt thereof. In some embodiments, it has the structure of

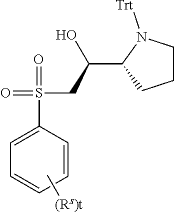

or a salt thereof. In some embodiments, it has the structure of

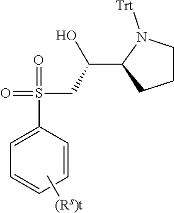

or a salt thereof. In some embodiments, it has the structure of

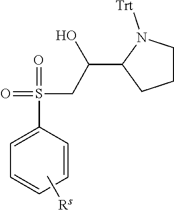

or a salt thereof. In some embodiments, it has the structure of

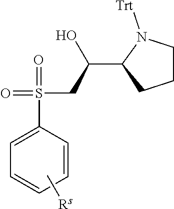

or a salt thereof. In some embodiments, it has the structure of

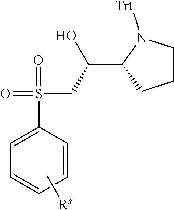

or a salt thereof. In some embodiments, it has the structure of

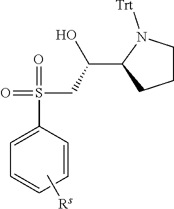

or a salt thereof. In some embodiments, it has the structure of

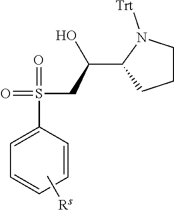

or a salt thereof. In some embodiments, R^{s} is R as described herein. In some embodiments, R^{s} is H, halogen, CN, COOR, OR, N(R)₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H. In some embodiments, R^{s} is halogen. In some embodiments, R^{s} is F. In some embodiments, R^{s} is Cl. In some embodiments, R^{s} is Br. In some embodiments, R^{s} is CN. In some embodiments, R^{s} is COOR. In some embodiments, R^{s} is COOR wherein R is not H. In some embodiments, R^{s} is OR. In some embodiments, R^{s} is OR wherein R is not H. In some embodiments, R^{s} is N(R)₂. In some embodiments, R^{s} is R as described herein.

In some embodiments, R^{s} is —H. In some embodiments, R^{s} is halogen. In some embodiments, R^{s} is optionally substituted C₁₋₆ aliphatic. In some embodiments, R^{s} is optionally substituted C₁₋₆ alkyl. In some embodiments, a compound of formula P-a is

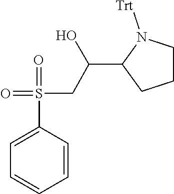

or a salt thereof. In some embodiments, a compound of formula P-a is

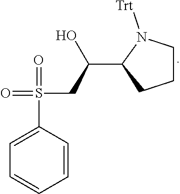

In some embodiments, a compound of formula P-a is

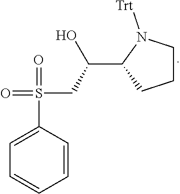

In some embodiments, a compound of formula P-a has the structure of

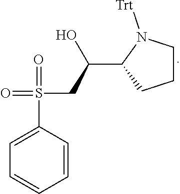

In some embodiments, a compound of formula P-a has the structure of

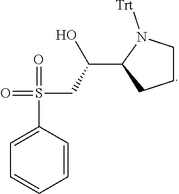

In some embodiments, a compound of formula P-a has the structure of:

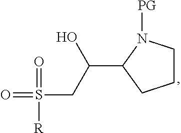

or a salt thereof, wherein:
PG is an amino protection group; and
R is independently —H, or an optionally substituted group selected from C₁₋₃₀ aliphatic.

In some embodiments, R is optionally substituted C₁₋₆ aliphatic. In some embodiments, it has the structure of

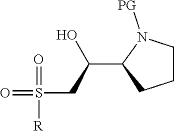

or a salt thereof. In some embodiments, it has the structure of

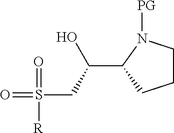

or a salt thereof. In some embodiments, it has the structure of

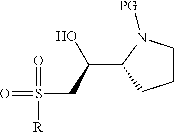

or a salt thereof. In some embodiments, it has the structure of

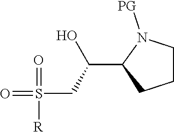

or a salt thereof. In some embodiments, R is optionally substituted C₁₋₁₀ aliphatic.

In some embodiments, a compound of formula P-a has the structure of:

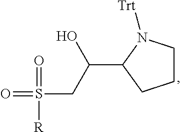

or a salt thereof, wherein:
R is independently —H, or an optionally substituted group selected from C₁₋₃₀ aliphatic. In some embodiments, R is optionally substituted C₁₋₆ aliphatic.

In some embodiments, a compound of formula P-a has the structure of:

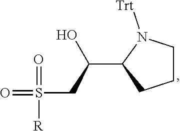

or a salt thereof, wherein:
R is independently —H, or an optionally substituted group selected from C₁₋₃₀ aliphatic. In some embodiments, R is optionally substituted C₁₋₆ aliphatic.

In some embodiments, a compound of formula P-a has the structure of:

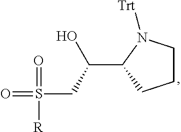

or a salt thereof, wherein:
R is independently —H, or an optionally substituted group selected from C₁₋₃₀ aliphatic. In some embodiments, R is optionally substituted C₁₋₆ aliphatic.

In some embodiments, a compound of formula P-a has the structure of:

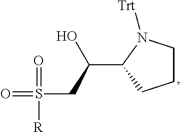

or a salt thereof, wherein:
R is independently —H, or an optionally substituted group selected from C₁₋₃₀ aliphatic. In some embodiments, R is optionally substituted C₁₋₆ aliphatic.

In some embodiments, a compound of formula P-a has the structure of:

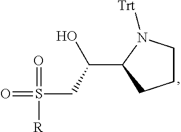

or a salt thereof, wherein:
R is independently —H, or an optionally substituted group selected from C₁₋₃₀ aliphatic. In some embodiments, R is optionally substituted C₁₋₆ aliphatic.

In some embodiments, a compound of formula P-a or a salt thereof has the structure of:

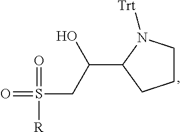

or a salt thereof, wherein R is an optionally substituted group selected from methyl, ethyl, propyl, isopropyl, n-butyl, tert-butyl, cyclobutyl, cyclopentyl, and cyclohexyl. In some embodiments, R is selected from methyl, ethyl, propyl, isopropyl, n-butyl, tert-butyl, cyclobutyl, cyclopentyl, and cyclohexyl. In some embodiments, it has the structure of

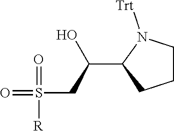

or a salt thereof. In some embodiments, it has the structure of

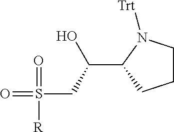

or a salt thereof. In some embodiments, it has the structure of

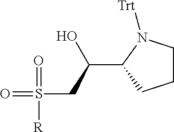

or a salt thereof. In some embodiments, it has the structure of

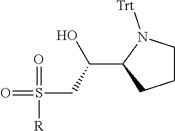

or a salt thereof.

In some embodiments, a compound of formula P-a or a salt thereof has the structure of:

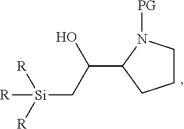

or a salt thereof, wherein:
PG is an amino protecting group; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, it has the structure of

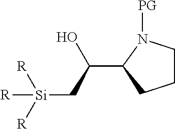

or a salt thereof. In some embodiments, it has the structure of

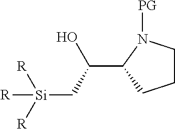

or a salt thereof. In some embodiments, it has the structure of

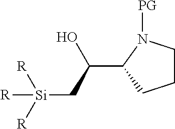

or a salt thereof. In some embodiments, it has the structure of

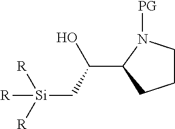

or a salt thereof.

In some embodiments, a compound of formula P-a or a salt thereof has the structure of:

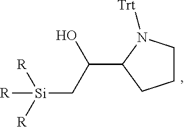

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula P-a or a salt thereof has the structure of:

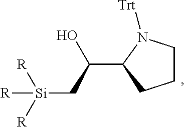

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula P-a or a salt thereof has the structure of:

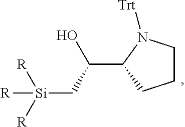

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula P-a or a salt thereof has the structure of:

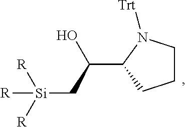

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula P-a or a salt thereof has the structure of:

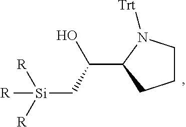

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

As described herein, in some embodiments, each R is independently —H, or an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₀ aryl, C₆₋₂₀ arylaliphatic, C₆₋₂₀ arylheteroaliphatic having 1-5 heteroatoms independently selected from oxygen, nitrogen, sulfur, phosphorus and silicon, 5-10 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon; or:
two R groups are optionally and independently taken together to form a covalent bond; or:
two R groups are optionally and independently taken together with the atom to form an optionally substituted, 3-15 membered, monocyclic, bicyclic or polycyclic ring having, in addition to the atom, 0-5 heteroatoms independently selected from oxygen, nitrogen, sulfur, phosphorus and silicon; or
two or more R groups are optionally and independently taken together with their intervening atoms to form an optionally substituted, 3-15 membered, monocyclic, bicyclic or polycyclic ring having, in addition to the intervening atoms, 0-5 heteroatoms independently selected from oxygen, nitrogen, sulfur, phosphorus and silicon.

In some embodiments, each R is independently an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₀ aryl, C₆₋₂₀ arylaliphatic, C₆₋₂₀ arylheteroaliphatic having 1-5 heteroatoms independently selected from oxygen, nitrogen, sulfur, phosphorus and silicon, 5-10 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon; or:
two R groups are optionally and independently taken together to form a covalent bond; or:
two R groups are optionally and independently taken together with the atom to form an optionally substituted, 3-15 membered, monocyclic, bicyclic or polycyclic ring having, in addition to the atom, 0-5 heteroatoms independently selected from oxygen, nitrogen, sulfur, phosphorus and silicon; or
two or more R groups are optionally and independently taken together with their intervening atoms to form an optionally substituted, 3-15 membered, monocyclic, bicyclic or polycyclic ring having, in addition to the intervening atoms, 0-5 heteroatoms independently selected from oxygen, nitrogen, sulfur, phosphorus and silicon.

In some embodiments, e.g., as in —Si(R)₃, each R is independently an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₀ aryl, C₆₋₂₀ arylaliphatic, C₆₋₂₀ arylheteroaliphatic having 1-5 heteroatoms independently selected from oxygen, nitrogen, sulfur, phosphorus and silicon, 5-10 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon.

In some embodiments, a compound of formula P-a is

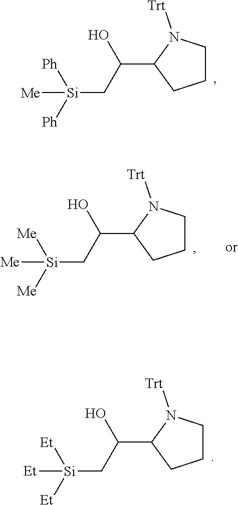

In some embodiments, it is

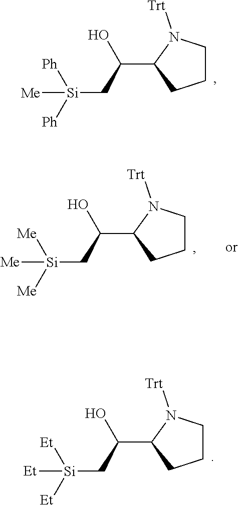

In some embodiments, it is

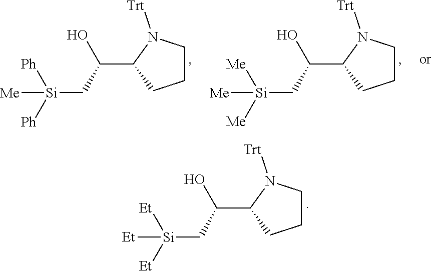

In some embodiments, it is

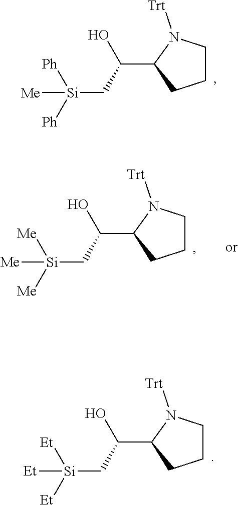

In some embodiments, it is

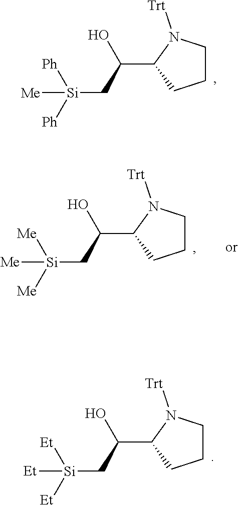

In some embodiments, a compound of formula INT-1 or a salt thereof has the structure of

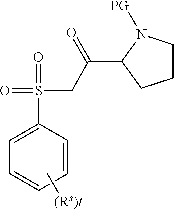

or a salt thereof, wherein each variable is independently as described herein. In some embodiments, it is

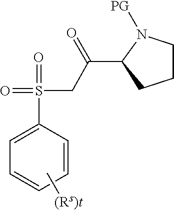

or a salt thereof. In some embodiments, it is

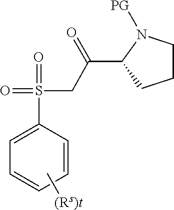

or a salt thereof. In some embodiments, t is 1 and it is

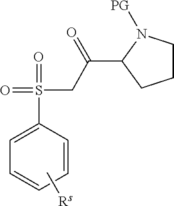

or a salt thereof. In some embodiments, it is

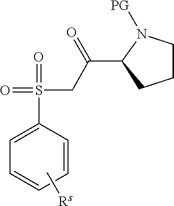

or a salt thereof. In some embodiments, it is

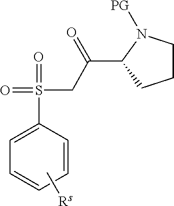

or a salt thereof. In some embodiments, R^{s} is R as described herein. In some embodiments, R^{s} is H, halogen, CN, COOR, OR, N(R)₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H. In some embodiments, R^{s} is halogen. In some embodiments, R^{s} is F. In some embodiments, R^{s} is Cl. In some embodiments, R^{s} is Br. In some embodiments, R^{s} is CN. In some embodiments, R^{s} is COOR. In some embodiments, R^{s} is COOR wherein R is not H. In some embodiments, R^{s} is OR. In some embodiments, R^{s} is OR wherein R is not H. In some embodiments, R^{s} is N(R)₂. In some embodiments, R^{s} is R as described herein.

In some embodiments, a compound of formula INT-1 or a salt thereof has the structure of

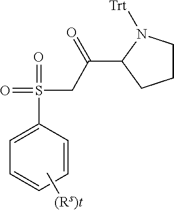

or a salt thereof, wherein each variable is independently as described herein. In some embodiments, it is

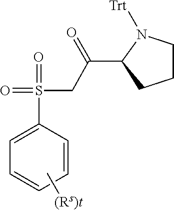

or a salt thereof. In some embodiments, it is

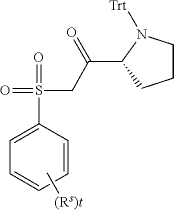

or a salt thereof. In some embodiments, R^{s} is R as described herein. In some embodiments, R^{s} is H, halogen, CN, COOR, OR, N(R)₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H. In some embodiments, R^{s} is halogen. In some embodiments, R^{s} is F. In some embodiments, R^{s} is Cl. In some embodiments, R^{s} is Br. In some embodiments, R^{s} is CN. In some embodiments, R^{s} is COOR. In some embodiments, R^{s} is COOR wherein R is not H. In some embodiments, R^{s} is OR. In some embodiments, R^{s} is OR wherein R is not H. In some embodiments, R^{s} is N(R)₂. In some embodiments, R^{s} is R as described herein.

In some embodiments, a compound of formula INT-1 or a salt thereof has the structure of

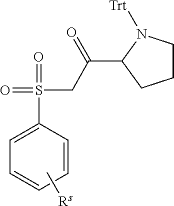

or a salt thereof, wherein each variable is independently as described herein. In some embodiments, it is

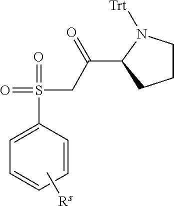

or a salt thereof. In some embodiments, it is

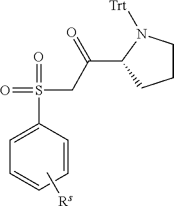

or a salt thereof. In some embodiments, R^{s} is R as described herein. In some embodiments, R^{s} is H, halogen, CN, COOR, OR, N(R)₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon. In some embodiments, R^{s} is H. In some embodiments, R^{s} is halogen. In some embodiments, R^{s} is F. In some embodiments, R^{s} is Cl. In some embodiments, R^{s} is Br. In some embodiments, R^{s} is CN. In some embodiments, R^{s} is COOR. In some embodiments, R^{s} is COOR wherein R is not H. In some embodiments, R^{s} is OR. In some embodiments, R^{s} is OR wherein R is not H. In some embodiments, R^{s} is N(R)₂. In some embodiments, R^{s} is R as described herein.

In some embodiments, R^{s} is —H. In some embodiments, R^{s} is halogen. In some embodiments, R^{s} is optionally substituted C₁₋₆ aliphatic. In some embodiments, R^{s} is optionally substituted C₁₋₆ alkyl. In some embodiments, a compound of formula INT-1 or a salt thereof is

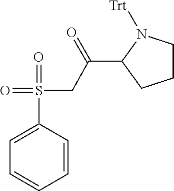

or a salt thereof. In some embodiments, it is

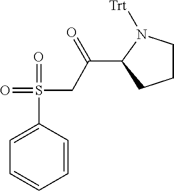

or a salt thereof. In some embodiments, it is

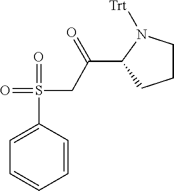

or a salt thereof.

In some embodiments, a compound of formula INT-1 has the structure of

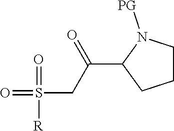

or a salt thereof, wherein PG is an amino protection group, and R is independently —H, or an optionally substituted group selected from C₁₋₃₀ aliphatic. In some embodiments, it has the structure of

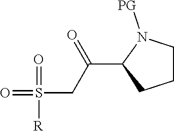

or a salt thereof. In some embodiments, it has the structure of

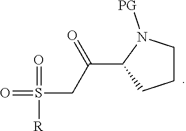

or a salt thereof. In some embodiments, R is optionally substituted C₁₋₆ aliphatic.

In some embodiments, a compound of formula INT-1 or a salt thereof has the structure of:

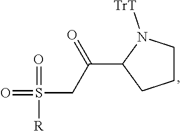

or a salt thereof, wherein R is independently —H, or an optionally substituted group selected from C₁₋₃₀ aliphatic. In some embodiments, it has the structure of

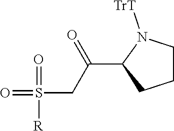

or a salt thereof. In some embodiments, it has the structure of

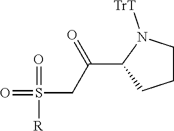

or a salt thereof. In some embodiments, R is optionally substituted C₁₋₆ aliphatic.

In some embodiments, a compound of formula INT-1 or a salt thereof has the structure of:

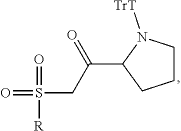

or a salt thereof, wherein R is an optionally substituted group selected from methyl, ethyl, propyl, isopropyl, n-butyl, tert-butyl, cyclobutyl, cyclopentyl, and cyclohexyl. In some embodiments, R is selected from methyl, ethyl, propyl, isopropyl, n-butyl, tert-butyl, cyclobutyl, cyclopentyl, and cyclohexyl. In some embodiments, it has the structure of

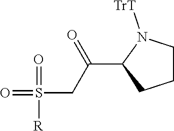

or a salt thereof. In some embodiments, it has the structure of

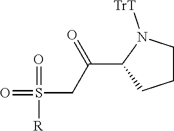

or a salt thereof.

In some embodiments, a compound of formula INT-1 or a salt thereof has the structure of:

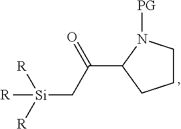

or a salt thereof, wherein:
PG is an amino protecting group; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, it has the structure of

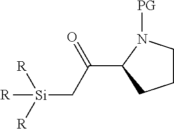

or a salt thereof. In some embodiments, it has the structure of

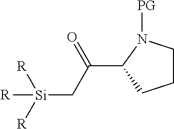

or a salt thereof.

In some embodiments, a compound of formula INT-1 or a salt thereof has the structure of:

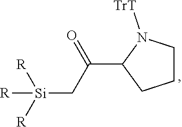

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula P-a or a salt thereof has the structure of:

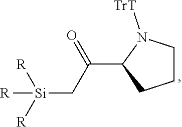

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula P-a or a salt thereof has the structure of:

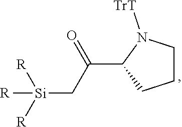

or a salt thereof, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, a compound of formula INT-1 has the structure of:

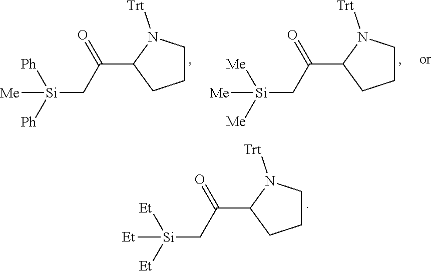

In some embodiments, a compound of formula INT-1, INT-1-a or NT-1-b is

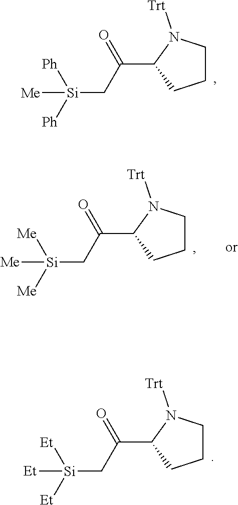

In some embodiments, it is

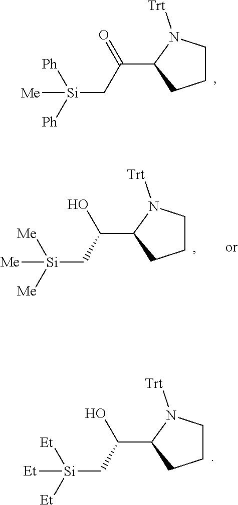

In some embodiments, a compound of formula INT-1, INT-1-a or INT-1-b is

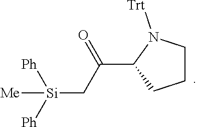

In some embodiments, it is

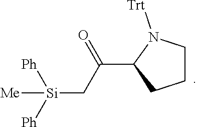

In some embodiments, it is

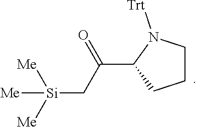

In some embodiments, it is

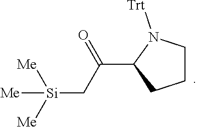

In some embodiments, it is

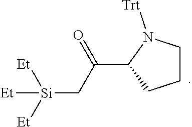

In some embodiments, it is

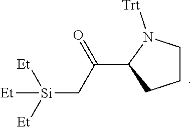

In some embodiments, a reduction is performed in the presence of a reducing agent which is HCOOH or a salt thereof and a metal complex, e.g., a Ru complex as described herein. In some embodiments, a reducing agent is HCOONa. In some embodiments, reduction is performed is the presence of water. In some embodiments, reduction is performed in a suitable solvent system, e.g., which is or comprises EtOAc, in accordance with the present disclosure.

Various technologies can be utilized to prepare a compound of formula INT-1 or a salt thereof in accordance with the present disclosure.

In some embodiments, the present disclosure provides a method for preparing a compound of formula INT-1:

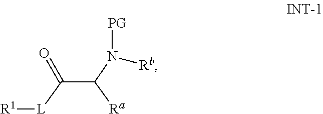

or a salt thereof,
comprising reacting a compound of formula INT-2:

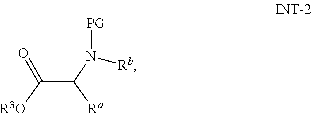

or a salt thereof with a compound of formula INT-3:

R¹-L-H, INT-3

or a salt thereof to provide the compound of formula INT-1 or a salt thereof,
wherein R³ is R, and each other viable is independently as described herein.

In some embodiments, a compound of formula INT-1 has the structure of formula INT-1-a. In some embodiments, a compound of formula INT-2 has the structure of formula INT-2-a. In some embodiments, a compound of formula INT-2-a has the structure of formula INT-2-b.

In some embodiments, the present disclosure provides a method for preparing a compound formula INT-1-a:

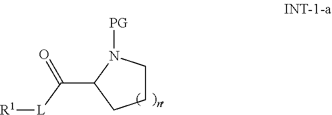

or a salt thereof,
comprising reacting a compound of formula INT-2-a:

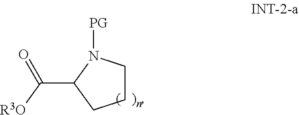

or a salt thereof with a compound of formula INT-3:

R¹-L-H, INT-3

or a salt thereof to provide the compound of formula INT-1-a or a salt thereof, wherein R³ is R, and each other viable is independently as described herein.

In some embodiments, the present disclosure provides a method for preparing a compound formula INT-1-b:

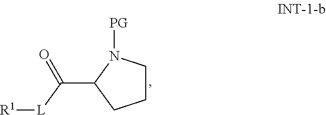

or a salt thereof,
comprising reacting a compound of formula INT-2-b:

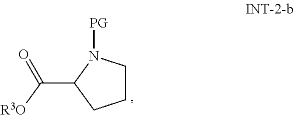

or a salt thereof with a compound of formula INT-3:

R¹-L-H, INT-3

or a salt thereof to provide the compound of formula INT-1-b or a salt thereof, wherein R³ is R, and each other viable is independently as described herein.

In some embodiments, a compound of formula INT-2 has the structure of INT-2-1 or INT-2-2, wherein each variable is independently as described herein. In some embodiments, a compound of formula INT-2-a has the structure of INT-2-a-1 or INT-2-a-2, wherein each variable is independently as described herein. In some embodiments, a compound of formula INT-2 has the structure of INT-2-b-1 or INT-2-b-2, wherein each variable is independently as described herein.

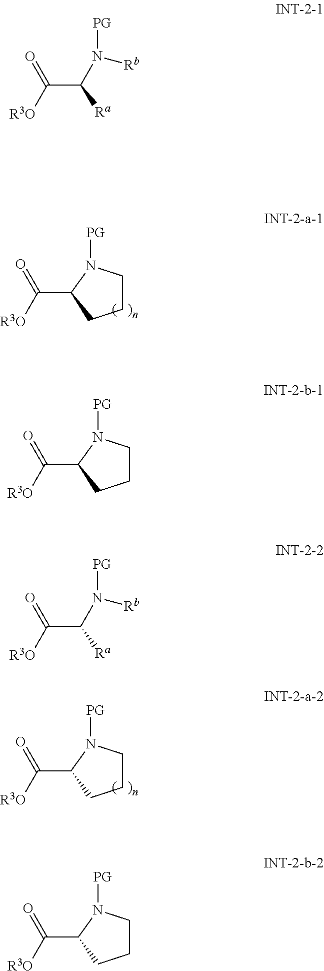

In some embodiments, in a provided method a compound of formula INT-1 or a salt thereof is a compound of formula INT-1-1, INT-1-a-1, INT-1-b-1, or a salt thereof, and a compound of INT-2 is a compound of INT-2-1, INT-2-a-1, or INT-2-b-1, respectively, or salt thereof. In some embodiments, in a provided method a compound of formula INT-1 or a salt thereof is a compound of formula INT-1-2, INT-1-a-2, INT-1-b-2, or a salt thereof, and a compound of INT-2 is a compound of INT-2-2, INT-2-a-2, or INT-2-b-2, respectively, or salt thereof. In some embodiments, products are formed stereoselectively as described herein.

In some embodiments, a compound of formula INT-3 or a salt thereof is a salt. In some embodiments, it is a Li⁺ salt.

In some embodiments, a reaction with a compound of formula INT-3 or a salt thereof is performed in the presence of a base. In some embodiments, a base is a lithium salt. In some embodiments, a base is LiHMDS. In some embodiments, a useful solvent system is or comprises THF.

Various technologies can be utilized to prepare a compound of formula INT-2 or a salt thereof in accordance with the present disclosure. In some embodiments, the present disclosure provides a method for preparing a compound formula INT-2 or a salt thereof, comprising:
providing a compound of formula INT-4:

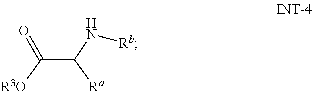

or a salt thereof, and
reacting a compound of formula INT-4 or a salt thereof with an amino protecting agent to provide a compound of formula INT-2 or a salt thereof, wherein each variable is independently as described herein.

In some embodiments, a compound of formula INT-4 has the structure of formula INT-4-a. In some embodiments, the present disclosure provides a method for preparing a compound formula INT-2-a or a salt thereof, comprising:
providing a compound of formula INT-4-a:

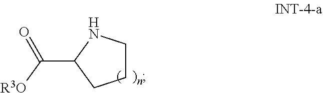

or a salt thereof, and
reacting a compound of formula INT-4-a or a salt thereof with an amino protecting agent to provide a compound of formula INT-2-a or a salt thereof, wherein each variable is independently as described herein.

In some embodiments, a compound of formula INT-4 or INT-4-a has the structure of formula INT-4-b. In some embodiments, the present disclosure provides a method for preparing a compound of formula INT-2-b or a salt thereof, comprising:
providing a compound of formula INT-4-b:

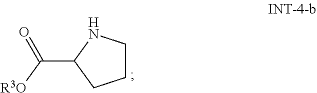

or a salt thereof, and
reacting a compound of formula INT-4-b or a salt thereof with an amino protecting agent to provide a compound of formula INT-2-b or a salt thereof, wherein each variable is independently as described herein.

In some embodiments, a compound of formula INT-4 has the structure of INT-4-1 or INT-4-2, wherein each variable is independently as described herein. In some embodiments, a compound of formula INT-4-a has the structure of INT-4-a-1 or INT-4-a-2, wherein each variable is independently as described herein. In some embodiments, a compound of formula INT-4 has the structure of INT-4-b-1 or INT-4-b-2, wherein each variable is independently as described herein.

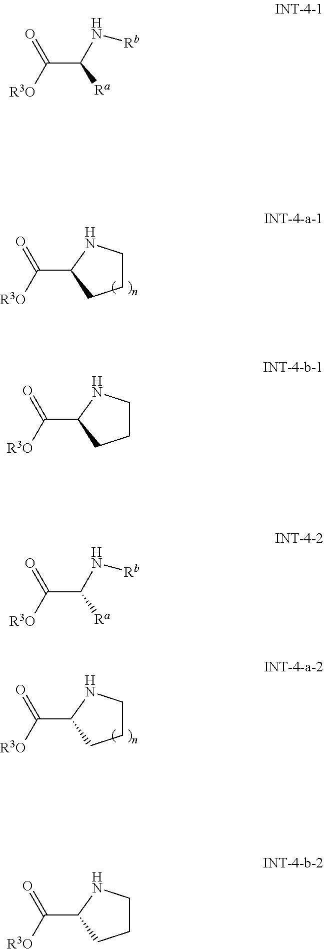

In some embodiments, in a provided method a compound of formula INT-2 or a salt thereof is a compound of formula INT-2-1, INT-2-a-1, INT-2-b-1, or a salt thereof, and a compound of INT-4 is a compound of INT-4-1, INT-4-a-1, or INT-4-b-1, respectively, or salt thereof. In some embodiments, in a provided method a compound of formula INT-2 or a salt thereof is a compound of formula INT-2-2, INT-2-a-2, INT-2-b-2, or a salt thereof, and a compound of INT-4 is a compound of INT-4-2, INT-4-a-2, or INT-4-b-2, respectively, or salt thereof. In some embodiments, products are formed stereoselectively as described herein.

Suitable technologies, e.g., amino protecting agents, methods, etc. for protecting amino groups are widely known and can be utilized in accordance with the present disclosure. In some embodiments, an amino protecting agent has the structure of PG-LG, wherein LG is a leaving group and PG is as described herein. In some embodiments, LG is —Cl. In some embodiments, LG is —OH. In some embodiments, an amino protecting agent is TrtCl

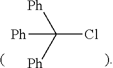 [SMILES: ClC(c1ccccc1)(c1ccccc1)c1ccccc1]

In some embodiments, a protecting reaction is performed in the presence of abase. In some embodiments, a base is N(R)₃. In some embodiments, each R is independently C₁₋₆ alkyl. In some embodiments, a base is TEA. In some embodiments, a useful solvent system is or comprises DCM.

Various technologies can be utilized to prepare a compound of formula INT-4-a or a salt thereof in accordance with the present disclosure. Certain useful methods are described below. Those skilled in the art will appreciate that other suitable technologies, e.g., various esterification technologies, may be utilized in accordance with the present disclosure. In some embodiments, the present disclosure provides a method for preparing a compound of formula INT-4:

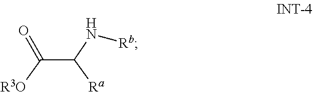

or a salt thereof, comprising:
providing a compound of formula INT-5:

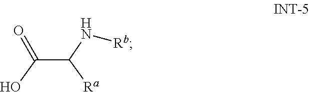

or a salt thereof; and
reacting a compound of formula INT-5 or a salt thereof with a compound having the structure of R³OH or a salt thereof to provide a compound of formula INT-4 or a salt thereof, wherein each variable is independently as described herein.

In some embodiments, a compound of formula INT-5 has the structure of formula INT-5-a. In some embodiments, the present disclosure provides a method for preparing a compound of formula INT-4-a:

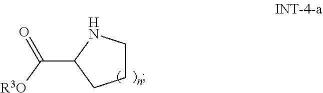

or a salt thereof, comprising:
providing a compound of formula INT-5-a:

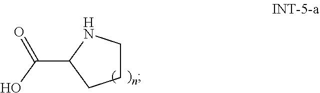

or a salt thereof; and
reacting a compound of formula INT-5-a or a salt thereof with a compound having the structure of a R³OH or a salt thereof to provide a compound of formula INT-4-a or a salt thereof, wherein each variable is independently as described herein.

In some embodiments, a compound of formula INT-5 or INT-5-a has the structure of formula INT-5-b. In some embodiments, the present disclosure provides a method for preparing a compound of formula INT-4-b:

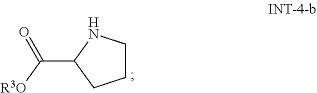

or a salt thereof, comprising:
providing a compound of formula INT-5-b:

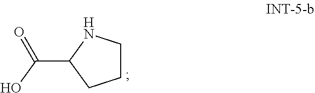

or a salt thereof; and
reacting a compound of formula INT-5-b or a salt thereof with a compound having the structure of a R³OH or a salt thereof to provide a compound of formula INT-4-b or a salt thereof, wherein each variable is independently as described herein.

In some embodiments, R³ is R as described herein. In some embodiments, R³ is optionally substituted C₁₋₁₀ aliphatic. In some embodiments, R^{s} is C₁₋₆ aliphatic. In some embodiments, R³ is methyl. In some embodiments, R³ is ethyl. In some embodiments, R³ is propyl. In some embodiments, R³ is isopropyl. In some embodiments, R is butyl.

In some embodiments, a compound of formula INT-5 has the structure of INT-5-1 or INT-5-2, wherein each variable is independently as described herein. In some embodiments, a compound of formula INT-5-a has the structure of INT-5-a-1 or INT-5-a-2, wherein each variable is independently as described herein. In some embodiments, a compound of formula INT-5 has the structure of INT-5-b-1 or INT-5-b-2, wherein each variable is independently as described herein.

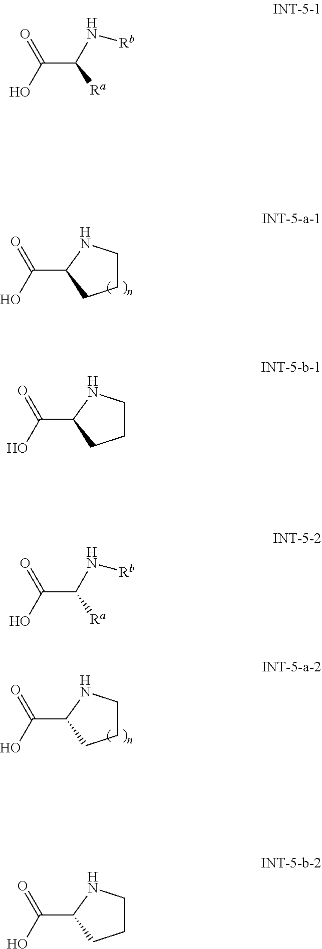

In some embodiments, in a provided method a compound of formula INT-4 or a salt thereof is a compound of formula INT-4-1, INT-4-a-1, INT-4-b-1, or a salt thereof, and a compound of INT-5 is a compound of INT-5-1, INT-5-a-1, or INT-5-b-1, respectively, or salt thereof. In some embodiments, in a provided method a compound of formula INT-4 or a salt thereof is a compound of formula INT-4-2, INT-4-a-2, INT-4-b-2, or a salt thereof, and a compound of INT-5 is a compound of INT-5-2, INT-5-a-2, or INT-5-b-2, respectively, or salt thereof. In some embodiments, products are formed stereoselectively as described herein.

In some embodiments, PG of a compound, e.g., of a compound having the structure of formula P, P-1, P-2, P-3, P-4, P-a, P-a-1, P-a-2, P-a-3, P-a-4, P-b, P-b-1, P-b-2, P-b-3, or P-b-4, or a salt thereof, can be removed. Various suitable deprotection technologies are available in the art and can be utilized in accordance with the present disclosure. In some embodiments, a method comprises removing a protecting group. In some embodiments, a method comprises removing a protecting group in a compound having the structure of formula P, P-1, P-2, P-3, P-4, P-a, P-a-1, P-a-2, P-a-3, P-a-4, P-b, P-b-1, P-b-2, P-b-3, or P-b-4, or a salt thereof, to provide a compound having the structure of formula DP, DP-1, DP-2, DP-3, DP-4, DP-a, DP-a-1, DP-a-2, DP-a-3, DP-a-4, DP-b, DP-b-1, DP-b-2, DP-b-3, or DP-b-4, respectively, or a salt thereof, wherein each variable is independently as described herein.

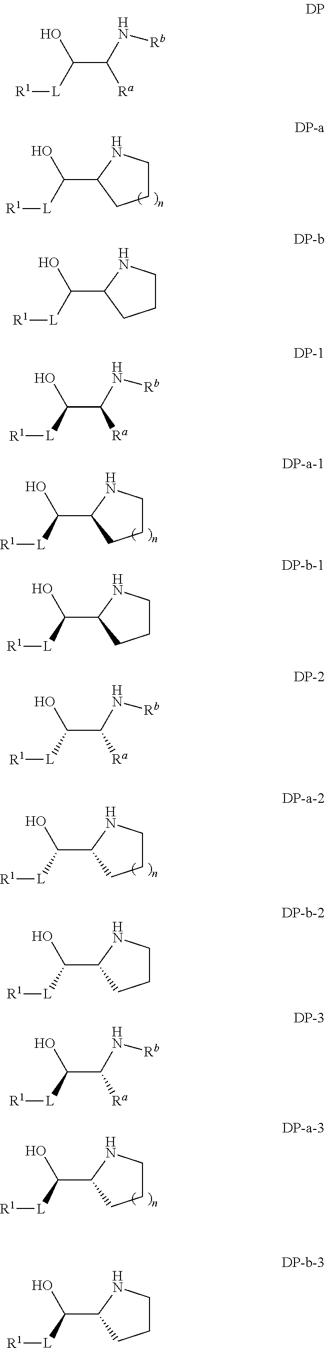

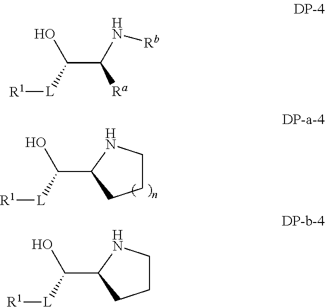

Various protecting/de-protecting technologies are available to those skilled in the art and may be utilized in accordance with the present disclosure. For example, in some embodiments, a Trt protecting group

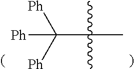 [SMILES: *C(c1ccccc1)(c1ccccc1)c1ccccc1]

can be removed under acidic conditions, e.g., using HCl.

Those skilled in the art that various preparation methods may be combined to provide multistep processes. For example, in some embodiments, the present disclosure provides a method for preparing a compound of formula DP:

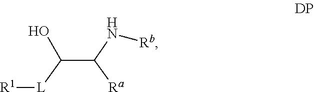

or a salt thereof, comprising:
(a) providing a compound having the a compound of formula INT-2:

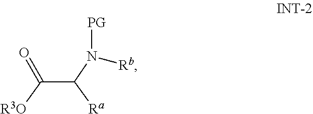

or a salt thereof, and
(b) reacting the compound of formula INT-2 or a salt thereof with a compound of formula INT-3:

R¹-L-H, INT-3

or a salt thereof to provide a compound of formula a compound formula INT-1:

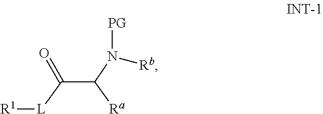

or a salt thereof, and
(c) reducing the compound of formula INT-1 or a salt thereof to provide a compound of formula P:

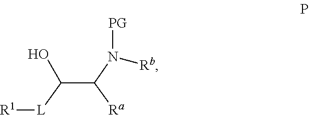

or a salt thereof, wherein the reduction of the compound of formula INT-1 or a salt thereof is carried out in the presence of a reducing agent; and
(d) deprotect the compound of formula P to provide a compound of formula DP or a salt thereof, wherein each variable is independently as described herein.

In some embodiments, the present disclosure provides a method for preparing a compound of formula DP-a:

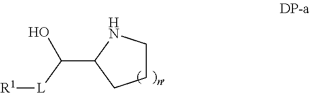

or a salt thereof, comprising:
(a) providing a compound having the a compound of formula INT-2-a:

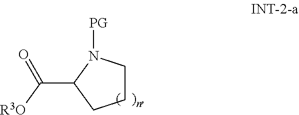

or a salt thereof, and
(b) reacting the compound of formula INT-2-a or a salt thereof with a compound of formula INT-3:

R¹-L-H, INT-3

or a salt thereof to provide a compound of formula a compound formula INT-1-a:

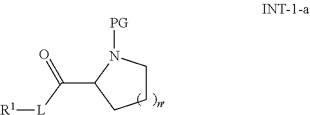

or a salt thereof, and
(c) reducing the compound of formula INT-1-a or a salt thereof to provide a compound of formula P-a:

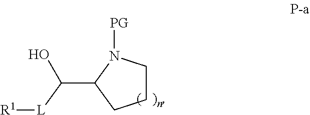

or a salt thereof, wherein the reduction of the compound of formula INT-1-a or a salt thereof is carried out in the presence of a reducing agent; and
(d) deprotect the compound of formula P-a to provide a compound of formula DP-a or a salt thereof;
   wherein:
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

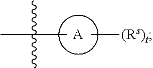

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a method for preparing a compound of formula I:

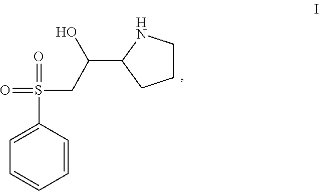 [SMILES: O=S(=O)(CC(O)C1CCCN1)c1ccccc1]

or a salt thereof, comprising:
(a) providing a compound having the structure of formula INT-2-b:

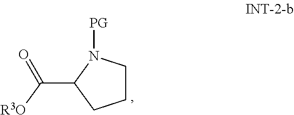

wherein:
PG is an amino protecting group;
R³ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon;
(b) reacting a compound of formula INT-2-b or a salt thereof with

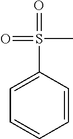

or a salt thereof to provide a compound of formula INT-1-c:

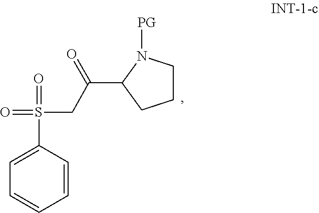

or a salt thereof, and
(c) reducing a compound of formula INT-1-c to provide a compound of formula P-I:

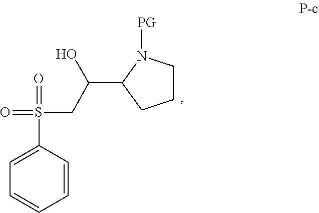

or a salt thereof, wherein the reduction of a compound of formula INT-1-c or a salt thereof is carried out in the presence of a reducing agent; and
(d) deprotect a compound of formula P-c or a salt thereof to provide said compound of formula I or a salt thereof.

Those skilled in the art reading the present disclosure will appreciate that various technologies, e.g., reagents, conditions, etc., can be utilized to perform various reaction in accordance with the present disclosure. Certain useful technologies are described herein as examples.

### Reduction

Various reduction technologies are available to those skilled in the art to conduct reduction reactions in accordance with the present disclosure. Among other things, various reducing agents and related conditions may be utilized to convert a compound of formula INT-1 or a salt thereof into a compound of formula P or a salt thereof.

In some embodiments, a reducing agent is a hydride compound. In some embodiments, a reducing agent comprises BH. In some embodiments, a reducing agent is a borohydride. In some embodiments, as demonstrated herein, a reducing agent is NaBH₄. In some embodiments, as demonstrated herein, a reducing agent is LiBH₄. In some embodiments, a reducing agent is NaBH₃CN. In some embodiments, a reducing agent is LiAlH₄. In some embodiments, a borohydride reducing agent provides a trans amino alcohol compound, e.g., after reduction and/or deprotection (e.g., a compound of formula P-3, P-a-3, P-b-3, P-4, P-a-4, P-b-4, DP-3, DP-a-3, DP-b-3, DP-4, DP-a-4, or DP-b-4, or a salt thereof).

In some embodiments, reduction, e.g., of a compound of formula INT-1 or a salt thereof, is carried out in the presence of HCOOH or a salt thereof. In some embodiments, a reduction is carried out in the presence of HCOONa. In some embodiments, a reduction is carried out in the presence of HCOOK. In some embodiments, a reduction is carried out in the presence of HCOOLi. In some embodiments, a reduction is carried out in the presence of HCOONH₄. In some embodiments, a reducing agent is hydrogen.

In some embodiments, a reduction, e.g., of a compound of formula INT-1 or a salt thereof is carried out in the presence of H₂. In some embodiments, reduction of a compound of formula INT-1 or a salt thereof is carried out in the presence of an agent that produces H₂ in situ. In some embodiments, another agent, e.g., an agent that promotes, accelerates, or catalyzes reduction by H₂, is utilized in the presence of H₂. In some embodiments, such an agent is or comprises a metal. In some embodiments, such an agent is or comprises a metal complex. In some embodiments, a metal is Ru. In some embodiments, a reduction is carried out in in the presence of an agent comprising a metal and one or more ligands. In some embodiments, a reduction is carried out in in the presence of an agent comprising a metal and one or more chiral ligands. In some embodiments, a chiral ligand comprises phosphorus. In some embodiments, a chiral ligand comprises nitrogen. In some embodiments, a reduction is stereoselective, e.g., in the presence of an agent comprising a metal, e.g., Ru, and one or more chiral ligands. Certain useful metal complexes are described herein.

In some embodiments, a reducing agent is or comprises HCOOH or a salt thereof, and is utilized in the presence of a metal complex as described herein. In some embodiments, such a reducing technology provides a cis amino alcohol compound, e.g., after reduction and/or deprotection (e.g., a compound of formula P-1, P-a-1, P-b-1, P-2, P-a-2, P-b-2, DP-1, DP-a-1, DP-b-1, DP-2, DP-a-2, or DP-b-2, or a salt thereof).

### Metal Complexes

In some embodiments, a reduction is carried out in the presence of an agent comprising a metal. In some embodiments, such an agent is a metal complex comprising a suitable metal and one or more suitable ligands. In some embodiments, a metal is a transition metal. In some embodiments, a metal is Ru. In some embodiments, a metal is Rh. In some embodiments, a metal is Pd. In some embodiments, a metal is Fe. In some embodiments, a metal is Co. In some embodiments, a metal is Ni. In some embodiments, a metal is Os. In some embodiments, a metal is Ir. In some embodiments, a metal is Pt.

In some embodiments, a metal complex comprises one or more nitrogen ligand. In some embodiments, a ligand is NHR^{M1}—CH₂—CH₂—N(—)S(O)₂R^{M2}, wherein each of R^{M1} and R^{M2} is independently R as described herein and each —CH₂— is independently as described herein. In some embodiments, a ligand is NHR^{M1}—C(R^{M3})₂—C(R^{M4})₂—N(—)S(O)₂R^{M2}, wherein each of R^{M1}, R′, R^{M3} and R^{M4} is independently R as described herein and each —CH₂— is independently as described herein. In some embodiments, a ligand is NHR^{M1}—CHR^{M3}—CHR^{M4}—N(—)S(O)₂R^{M2}, wherein each of R^{M1}, R^{M2}, R^{M3} and R^{M4} is independently R as described herein and each —CH₂— is independently as described herein. In some embodiments, a metal complex comprises or has the structure of Ru[NHR^{M1}—CH₂—CH₂—N(—)S(O)₂R^{M2}](R^{M5})(R^{M6}—R^{M7}) or a salt thereof, wherein each —CH₂— is independently optionally substituted and each variable is independently as described herein. In some embodiments, a metal complex comprises or has the structure of Ru[NHR^{M1}—C(R^{M3})₂—C(R^{M4})₂—N(—)S(O)₂R^{M2}](R^{M5})(R^{M6}—R^{M7}) or a salt thereof, wherein each variable is independently as described herein. In some embodiments, a metal complex comprises or has the structure of Ru[NHR^{M1}—CHR^{M3}—CHR^{M4}—N(—)S(O)₂R^{M2}](R^{M5})(R^{M6}—R^{M7}) or a salt thereof, wherein each variable is independently as described herein.

In some embodiments, R^{M1} is —H. In some embodiments, R^{M1} is not —H.

In some embodiments, R^{M2} is —H. In some embodiments, R^{M2} is not —H. In some embodiments, R^{M2} is optionally substituted phenyl. In some embodiments, R^{M2} is p-methylphenyl. In some embodiments, R^{M2} is pentafluorophenyl.

In some embodiments, R^{M3} is not —H. In some embodiments, R^{M3} is optionally substituted phenyl. In some embodiments, R^{M3} is phenyl. In some embodiments, R^{M4} is not —H. In some embodiments, R^{M4} is optionally substituted phenyl. In some embodiments, R^{M4} is phenyl. In some embodiments, —NHR^{M1} and —N(—)S(O)₂R^{M2} are trans. In some embodiments, an agent is enriched for a stereoisomer. In some embodiments, an agent is stereopure.

In some embodiments, a metal complex comprises a ligand R^{M5} which is halogen. In some embodiments, R^{M5} is —C₁.

In some embodiments, a metal complex comprises a ligand R^{M6}—H wherein R^{M6} is R, wherein R is optionally substituted aryl or heteroaryl as described herein. In some embodiments, a metal complex comprises a ligand R^{M6}_R^{M7} wherein R^{M6} is R, wherein R is optionally substituted aryl or heteroaryl as described herein, and R^{M7} is R as described herein. In some embodiments, R^{M7} is —H. In some embodiments, R^{M7} is optionally substituted C₁₋₆ alkyl. In some embodiments, R^{M7} is methyl. In some embodiments, R^{M7} is isopropyl. In some embodiments, R^{M6} is optionally substituted phenyl. In some embodiments, R^{M6}—H is p-cymene. In some embodiments, R^{M6}—H is mesitylene. In some embodiments, R^{M7} and R^{M1} are taken together to form a linker, e.g., an optionally substituted bivalent C₁₋₆ linear or branched aliphatic or heteroaliphatic group having 1-3 heteroatoms. In some embodiments, R^{M7} and R^{M1} are taken together to form an optionally substituted bivalent C₁₋₆ linear or branched aliphatic or heteroaliphatic group having 1-3 heteroatoms independently selected from nitrogen, oxygen and sulfur. In some embodiments, a linker is optionally substituted —(CH₂)m- wherein m is 1-6. In some embodiments, a linker is —(CH₂)m- wherein n is 6. In some embodiments, m is 1. In some embodiments, m is 2. In some embodiments, m is 3. In some embodiments, m is 4. In some embodiments, m is 5. In some embodiments, m is 6.

In some embodiments, an agent is N-[(1S,2S)-2-amino-1,2-diphenyl-ethyl]-4-methyl-benzenesulfonamide;chlororuthenium;1-isopropyl-4-methyl-benzene

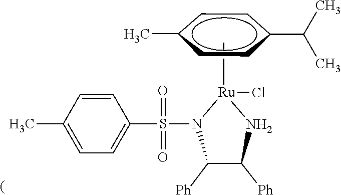

Ru—[(S, S)-Ts-DPEN] or RuCl(p-cymene)[(S, S)-Ts-DPEN], CAS #: 192139-90-5).

In some embodiments, an agent is N-[(1R,2R)-2-amino-1,2-diphenyl-ethyl]-4-methyl-benzenesulfonamide;chlororuthenium;1-isopropyl-4-methyl-benzene

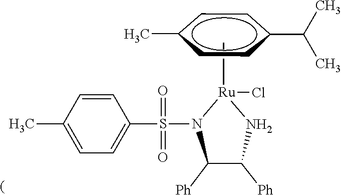

Ru—[(R, R)-Ts-DPEN] or RuCl(p-cymene)[(R, R)-Ts-DPEN], CAS #: 192139-92-7).

In some embodiments, an agent is [N-[(1S,2S)-2-(Amino-κN)-1,2-diphenylethy]-2,3,4,5,6-pentafluorobenzenesulfonamidato-κN]chloro[(1,2,3,4,5,6-η)-1-methyl-4-(1-methylethyl)benzene]-ruthenium (

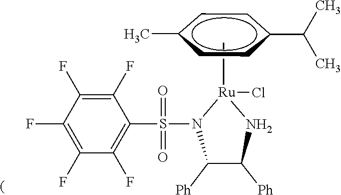

or RuCl(p-cymene)[(S, S)-Fsdpen] or RuCl[(S, S)-FsDPEN](p-cymene), CAS #: 1026995-72-1).

In some embodiments, an agent is [N-[(1R, 2R)-2-(Amino-κN)-1,2-diphenylethyl]-2,3,4,5,6-pentafluorobenzenesulfonamidato-κN]chloro[(1,2,3,4,5,6-η)-1-methyl-4-(1-methylethyl)benzene]-ruthenium

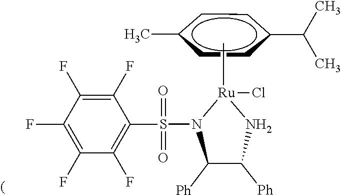

or RuCl(p-cymene)[(R, R)-Fsdpen] or RuCl[(R, R)-FsDPEN](p-cymene), CAS #: 1026995-71-0).

In some embodiments, an agent is RuCl[(S, S)-TsDPEN](mesitylene). In some embodiments, an agent is RuCl[(R, R)-TsDPEN](mesitylene).

In some embodiments, an agent is [(R, R)-Teth-TsDpen RuCl]. In some embodiments, an agent is [(S, S)-Teth-TsDpen RuCl].

In some embodiments, an agent, e.g., an agent comprising a metal, is utilized in an amount of about or no more than about 1, 0.9, 0.8, 0.7, 0.6, 0.5, 0.4, 0.3, 0.2, 0.1, 0.05, 0.04, 0.03, 0.025, 0.02, 0.01, or 0.005 equivalent of a compound to be reduced. In some embodiments, an agent is utilized in an amount of about or no more than about 0.05 equivalent. In some embodiments, an agent is utilized in an amount of about or no more than about 0.025 equivalent. In some embodiments, an agent is utilized in an amount of about or no more than about 0.01 equivalent.

In some embodiments, RuCl(p-cymene)[(S, S)-Ts-DPEN] is utilized in an amount of about or no more than about 0.05 equivalent. In some embodiments, RuCl(p-cymene)[(S, S)-Ts-DPEN] is utilized in an amount of about or no more than about 0.025 equivalent. In some embodiments, RuCl(p-cymene)[(S, S)-Ts-DPEN] is utilized in an amount of about or no more than about 0.01 equivalent.

In some embodiments, RuCl(p-cymene)[(R, R)-Ts-DPEN] is utilized in an amount of about or no more than about 0.05 equivalent. In some embodiments, RuCl(p-cymene)[(R, R)-Ts-DPEN] is utilized in an amount of about or no more than about 0.025 equivalent. In some embodiments, RuCl(p-cymene)[(R, R)-Ts-DPEN] is utilized in an amount of about or no more than about 0.01 equivalent.

In some embodiments, RuCl(p-cymene)[(S, S)-Fsdpen] is utilized in an amount of about or no more than about 0.05 equivalent. In some embodiments, RuCl(p-cymene)[(S, S)-Fsdpen] is utilized in an amount of about or no more than about 0.025 equivalent. In some embodiments, RuCl(p-cymene)[(S, S)-Fsdpen] is utilized in an amount of about or no more than about 0.01 equivalent.

In some embodiments, RuCl(p-cymene)[(R, R)-Fsdpen] is utilized in an amount of about or no more than about 0.05 equivalent. In some embodiments, RuCl(p-cymene)[(R, R)-Fsdpen] is utilized in an amount of about or no more than about 0.025 equivalent. In some embodiments, RuCl(p-cymene)[(R, R)-Fsdpen] is utilized in an amount of about or no more than about 0.01 equivalent.

### Selectivity

In some embodiments, provided technologies provide high selectivity. For example, in various embodiments, products are formed with high selectivity. In some embodiments, a chiral element, e.g., a chiral center, is formed with high stereoselectivity. In some embodiments, stereoselectivity is or comprises diastereoselectivity. In some embodiments, selectivity is or comprises enantioselectivity. In some embodiments, selectivity is or comprises selective transformation of a certain stereoisomer (e.g., a diastereomer, an enantiomer, etc.). In some embodiments, selectivity is or comprises selective transformation of an enantiomer. In some embodiments, selectivity is or comprises selective transformation of a diastereomer. In some embodiments, selectivity is or comprises selective production of a certain stereoisomer. In some embodiments, selectivity is or comprises selective production of a certain diastereomer. In some embodiments, selectivity is or comprises selective production of a certain enantiomer. In some embodiments, reaction conditions of the present disclosure does not cause epimerization of chiral centers (e.g., in some embodiments, less than about 5%, 4%, 3%, 2%, 1%, 0.5%, 0.2%, or 0.1%; in some embodiments, no detectable epimerization).

In some embodiments, selectivity is presented as ratios, e.g., ratios of two potential configurations of a chiral center (e.g., R or S) or to two forms of a compound (e.g., trans or cis). In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more. In some embodiments, selectivity is presented as diastereomeric excess (de) and/or enantiomeric excess (ee) In some embodiments, de is |D1-D2|, wherein D1 and D2 are the mole fractions of two diastereoisomers in a composition (D1+D2=1). In some embodiments, de is |D1-D21, wherein D1 and D2 are the mole fraction yields of two diastereomers formed in a reaction (D1+D2=1). In some embodiments, ee is |F1-F2|, wherein F1 and F2 are the mole fractions of two enantiomers in a composition (F1+F2=1). In some embodiments, ee is |F1-F2|, wherein F1 and F2 are the mole fraction yields of two enantiomers formed in a reaction (F1+F2=1). In some embodiments, provided technologies can provide de and/or ee at or above certain levels. In some embodiments, selectivity is presented as product purity. In some embodiments, a product has a purity of or above certain levels. In some embodiments, a product has certain diastereomeric purity at or above certain levels. In some embodiments, a product has certain enantiomeric purity at or above certain levels. In some embodiments, a product has certain stereopurity at or above certain levels. In some embodiments, a level is about or at least about 60%, 65%, 70%, 75%, 80%, 85%, 90%, 91%, 92%, 93%, 94%, 95%, 96%, 97%, 98%, 99%, or 99.5%. In some embodiments, a level is about or at least about 80%. In some embodiments, a level is about or at least about 85%. In some embodiments, a level is about or at least about 90%. In some embodiments, a level is about or at least about 95%. In some embodiments, a level is about or at least about 97%. In some embodiments, a level is about or at least about 99%.

In some embodiments, —OH and —N(PG)- are cis in a reduction product. In some embodiments, —OH and —N(PG)- are cis in a reduction product, and a cis product is formed with a selectivity of about 80%, 85%, 90%, 91%, 92%, 93%, 94%, 95%, 96%, 97%, 98%, 99% or more. In some embodiments, the selectivity is about 90% or more. In some embodiments, the selectivity is about 94% or more. In some embodiments, the selectivity is about 95% or more. In some embodiments, the selectivity is about 96% or more.

In some embodiments, —OH and —N(PG)- are trans in a reduction product. In some embodiments, —OH and —N(PG)- are trans in a reduction product, and a trans product is formed with a selectivity of about 80%, 85%, 90%, 91%, 92%, 93%, 94%, 95%, 96%, 97%, 98%, 99% or more. In some embodiments, the selectivity is about 90% or more. In some embodiments, the selectivity is about 94% or more. In some embodiments, the selectivity is about 95% or more. In some embodiments, the selectivity is about 96% or more.

In some embodiments, purity of a compound is or greater than about 85%, 90%, 91%, 92%, 93%, 94%, 95%, 96%, 97%, 98%, 99%, 99.7%, or 99.9%. In some embodiments, purity of a compound is or greater than about 85%. In some embodiments, purity of a compound is or greater than about 85%. In some embodiments, purity of a compound is or greater than about 90%. In some embodiments, purity of a compound is or greater than about 95%. In some embodiments, purity of a compound is or greater than about 96%. In some embodiments, purity of a compound is or greater than about 97%. In some embodiments, purity of a compound is or greater than about 98%. In some embodiments, purity of a compound is or greater than about 99%. In some embodiments, purity of a compound is or greater than about 99.7%. In some embodiments, purity of a compound is or greater than about 99.9%.

Various chemical reactions are typically performed in the presence of a solvent system. In some embodiments, a solvent system is a single solvent. In some embodiments, a solvent system is or comprises a mixture of several solvents. In some embodiments, a solvent is polar. In some embodiments, a solvent is non-polar. In some embodiments, a solvent is protic. In some embodiments, a solvent is non-protic. In some embodiments, a solvent is polar but is not protic. Suitable solvent systems for various reactions are available to those skilled in the art and can be utilized in accordance with the present disclosure. For example, in some embodiments, reduction, e.g., of a compound of formula INT-1 or a salt thereof is carried out in the presence of a protic solvent. In some embodiments, reduction, e.g., of a compound of formula INT-1 or a salt thereof is carried out in the presence of a combination of two or more protic solvents. In some embodiments, a protic solvent is methanol. In some embodiments, a protic solvent is ethanol. In some embodiments, a solvent system is or comprises methanol. In some embodiments, a solvent system is or comprises ethanol.

In some embodiments, reactions are performed, or are performed for periods of time, at temperatures that are higher or lower than or about a standard ambient temperature (25° C.). In some embodiments, a reaction temperature is lower than a standard ambient temperature. In some embodiments, a temperature is about or no more than about −78, −60, −50, −40, −30, −20, −10, 0 or 10° C. In some embodiments, a temperature is about or no more than about 10° C. In some embodiments, a temperature is about or no more than about 15° C. In some embodiments, a temperature is about or no more than about 20° C. In some embodiments, a reaction temperature is about a standard ambient temperature. In some embodiments, a reaction temperature is higher than a standard ambient temperature. In some embodiments, a reaction temperature is about or at least about 35, 40, 50, 60, 70, 80, 90, 100, or 100° C. In some embodiments, a reaction comprises refluxing in a boiling solvent system, e.g., in ether, toluene, etc. In some embodiments, temperature changes during a reaction process, e.g., increasing from a lower temperature to a higher temperature, decreasing from a higher temperature to a lower temperature, or both.

Certain embodiments for various variables in various formulae are described herein as examples. Those skilled in the art reading the present disclosure will be able to select an embodiment for each variable and combine them; such combinations are within the scope the present disclosure.

### PG

Suitable protecting groups are widely known by those skilled in the art and can be utilized as described herein. In some embodiments, as described herein, amino groups are protected so that various reactions can proceed as described. In some embodiments, protection of a group, e.g., an amino group, reduces or prevents interference of a reaction by such a group, and/or reduces or prevents reactions at such a group. In some embodiments, a protecting group, e.g., an amino protecting group, is —C(O)R′, an amino protecting group, is —C(O)R wherein R is as described herein. In some embodiments, it is —C(O)OR, e.g., Boc. In some embodiments, it is —S(O)₂R wherein R is as described herein. In some embodiments, it is R wherein R is not hydrogen. In some embodiments, it is optionally substituted C₁₋₆ aliphatic. In some embodiments, it is optionally substituted methyl, wherein one or more substituent is an aromatic group. In some embodiments, it is optionally substituted benzyl. In some embodiments, it is —CH₂—R, wherein the —CH₂— is optionally substituted and R as described herein and is not —H. In some embodiments, it is —CH₂—R, wherein the —CH₂— is optionally substituted and R is an optionally substituted group selected from C₆₋₁₀ aryl and 5-10 membered heteroaryl having 1-5 heteroatoms independently selected from nitrogen, oxygen and sulfur. In some embodiments, it is —CH₂—R, wherein the —CH₂— is optionally substituted and R is an optionally substituted group selected from phenyl and 5-6 membered heteroaryl having 1-5 heteroatoms independently selected from nitrogen, oxygen and sulfur. In some embodiments, it is —CH₂—R, wherein the —CH₂— is optionally substituted and R is optionally substituted phenyl. In some embodiments, it is —CH(R)₂ wherein each R is independently as described herein and is not —H. In some embodiments, it is —CH(R)₂ wherein each R is independently an optionally substituted group selected from C₆₋₁₀ aryl and 5-10 membered heteroaryl having 1-5 heteroatoms independently selected from nitrogen, oxygen and sulfur. In some embodiments, it is —CH(R)₂ wherein each R is independently an optionally substituted group selected from phenyl and 5-6 membered heteroaryl having 1-5 heteroatoms independently selected from nitrogen, oxygen and sulfur. In some embodiments, it is —CH(R)₂ wherein each R is independently an optionally substituted phenyl. In some embodiments, it is —C(R)₃ wherein each R is independently as described herein and is not —H. In some embodiments, it is —C(R)₃ wherein each R is independently an optionally substituted group selected from C₆₋₁₀ aryl and 5-10 membered heteroaryl having 1-5 heteroatoms independently selected from nitrogen, oxygen and sulfur. In some embodiments, it is —C(R)₃ wherein each R is independently an optionally substituted group selected from phenyl and 5-6 membered heteroaryl having 1-5 heteroatoms independently selected from nitrogen, oxygen and sulfur. In some embodiments, it is —C(R)₃ wherein each R is independently an optionally substituted phenyl. In some embodiments, it is -Trt

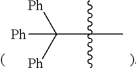 [SMILES: *C(c1ccccc1)(c1ccccc1)c1ccccc1]

Technologies for incorporating and removing protecting groups are widely known and may be utilized in accordance with the present disclosure. For example, in some embodiments, protecting groups such as Boc, Trt, etc. can be removed by utilizing an acid.

### L

In some embodiments, L is —CH₂—. In some embodiments, L is substituted —CH₂—. In some embodiments, L is —CH₂— substituted with one or two suitable substituents. In some embodiments, L is mono-substituted. In some embodiments, L is di-substituted. In some embodiments, L is —CH(CN)—.

### R¹

In some embodiments, R¹ is R as described herein. In some embodiments, R¹ is —H. In some embodiments, R¹ is not —H.

In some embodiments, R¹ is —P(O)(R²)₂ wherein each R² is independently as described herein. In some embodiments, at least one R² is not —H. In some embodiments, each R² is not —H. In some embodiments, at least one R² is —OR. In some embodiments, at least one R² is —OR wherein R is as described herein and is not —H. In some embodiments, each R² is independently —OR. In some embodiments, each R² is independently —OR wherein R is as described herein and is not —H. In some embodiments, at least one R² is independently —N(R′)₂, wherein each R′ is independently as described herein. In some embodiments, at least one R² is independently —N(R)₂, wherein each R is independently as described herein. In some embodiments, each R² is independently —N(R′)₂, wherein each R′ is independently as described herein. In some embodiments, each R² is independently —N(R)₂, wherein each R is independently as described herein. In some embodiments, at least one R² is

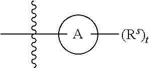

as described herein. In some embodiments, each R² is independently

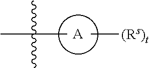

as described herein.

In some embodiments, R¹ is —S(O)₂R². In some embodiments, R² is R as described herein. In some embodiments, R² is R as described herein and is not —H. In some embodiments, R² is optionally substituted C₁₋₁₀ aliphatic. In some embodiments, R² is C₁₋₆ aliphatic. In some embodiments, R² is C₁₋₆ alkyl. In some embodiments, R² is methyl. In some embodiments, R² is ethyl. In some embodiments, R² is n-propyl. In some embodiments, R² is isopropyl. In some embodiments, R² is n-butyl. In some embodiments, R² is cyclobutyl. In some embodiments, R² is cyclopentyl. In some embodiments, R² is cyclopropyl. In some embodiments, R² is cyclohexyl. In some embodiments, R² is optionally substituted phenyl. In some embodiments, R² is phenyl. In some embodiments, R² is —OR. In some embodiments, R² is —OR wherein R is not —H. In some embodiments, R² is —N(R′)₂ wherein each R′ is independently as described herein. In some embodiments, R² is —N(R)₂ wherein each R is independently as described herein. In some embodiments, R² is —NMe₂. In some embodiments, R² is

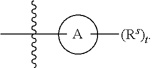

In some embodiments, Ring A is an optionally substituted phenyl ring (as appreciated by those skilled in the art, in addition to —S(O)₂— and R^{s} group(s)). Various useful embodiments of R^{s} and t are described herein as examples. In some embodiments, R¹ is —S(O)₂R² wherein R² is optionally substituted phenyl. In some embodiments, R¹ is —S(O)₂R² wherein R² is phenyl.

In some embodiments, R¹ is —Si(R)₃ wherein each R is independently described therein. In some embodiments, each R is not —H. In some embodiments, R¹ is —Si(R)₃, wherein each R is independently an optionally substituted C₁₋₃₀ aliphatic group. In some embodiments, R¹ is —Si(R)₃, wherein each R is independently an optionally substituted C₁₋₁₀ aliphatic group. In some embodiments, R¹ is —Si(R)₃, wherein each R is independently selected from the group of methyl, ethyl, propyl, isopropyl, n-butyl, tert-butyl, cyclobutyl, cyclopentyl, and cyclohexyl. In some embodiments, R¹ is —Si(R)₃, wherein each R is independently an optionally substituted C₁₋₄ aliphatic group. In some embodiments, R¹ is —Si(R)₃, wherein each R is independently methyl. In some embodiments, R¹ is —Si(R)₃, wherein each R is independently ethyl. In some embodiments, R¹ is —Si(R)₃, wherein each R is independently propyl. In some embodiments, R¹ is —Si(R)₃, wherein each R is independently isopropyl. In some embodiments, R¹ is —Si(R)₃, wherein each R is independently n-butyl. In some embodiments, R¹ is —Si(R)₃, wherein each R is independently tert-butyl.

In some embodiments, R¹ is —Si(R)₃, wherein each R is independently an optionally substituted group selected from C₁₋₃₀ aliphatic and C₆₋₃₀ aryl. In some embodiments, R¹ is —Si(R)₃, wherein each R is independently an optionally substituted group selected from C₁₋₁₀ aliphatic and phenyl. In some embodiments, R¹ is —Si(R)₃, wherein each R is independently an optionally substituted group selected from C₁₋₄ aliphatic and phenyl. In some embodiments, R¹ is —Si(R)₃, wherein each R is independently an optionally substituted group selected from C₁₋₄ aliphatic and phenyl, wherein the substituent is halogen, —CN, —C(O)OR′, —OR′, or —N(R′)₂, wherein R′ is described therein. In some embodiments, R¹ is —Si(R)₃, wherein each R is independently C₁₋₄ aliphatic or optionally substituted phenyl, wherein the substituent is halogen, —CN, —C(O)OR′, —OR′, or —N(R′)₂, wherein R¹ is described therein. In some embodiments, R¹ is —Si(R)₃, wherein each R is independently C₁₋₄ aliphatic or phenyl. In some embodiments, R¹ is —Si(R)₃ wherein one R group is optionally substituted C₁₋₆ aliphatic and the other two are independently optionally substituted phenyl. In some embodiments, R¹ is —Si(Ph)₂Me.

### R²

In some embodiments, R² is R as described herein. In some embodiments, R² is —H. In some embodiments, R² is not —H. In some embodiments, R² is optionally substituted C₁₋₁₀ aliphatic. In some embodiments, R² is optionally substituted C₁₋₁₀ alkyl. In some embodiments, R² is C₁₋₁₀ alkyl. In some embodiments, R² is methyl. In some embodiments, R² is ethyl. In some embodiments, R² is isopropyl. In some embodiments, R² is n-butyl. In some embodiments, R² is cyclobutyl. In some embodiments, R² is cyclopentyl. In some embodiments, R² is cyclohexyl. In some embodiments, R² is optionally substituted phenyl. In some embodiments, R² is phenyl.

In some embodiments, R² is —OR. In some embodiments, R² is —OH. In some embodiments, R² is —OR wherein R is not —H. In some embodiments, R is optionally substituted C₁₋₆ aliphatic.

In some embodiments, R² is —N(R′)₂ wherein each R′ is independently as described herein. In some embodiments, R² is —NHR′ wherein R′ is as described herein. In some embodiments, R² is —N(R)₂ wherein each R is independently as described herein. In some embodiments, R² is —NHR wherein R is as described herein. In some embodiments, R² is —NH₂. In some embodiments, R² is —N(R)₂ wherein each R is independently C₁₋₆ aliphatic. In some embodiments, R² is —NMe₂. In some embodiments, R² is —N(Et)₂. In some embodiments, R² is —N(Me)Et.

In some embodiments, R² is

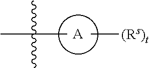

as described herein.

In some embodiments, one occurrence of R² is

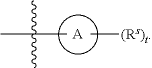

In some embodiments, t is 1 and Ring A is optionally substituted

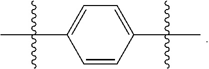 [SMILES: *c1ccc(*)cc1]

In some embodiments, R² is optionally substituted

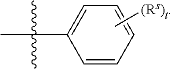

In some embodiments, one occurrence of R² is

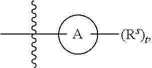

the other occurrence is —OR. In some embodiments, one occurrence of R² is

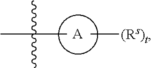

the other occurrence is —R.

### Ring A

As described herein, Ring A is optionally substituted (in addition to the group

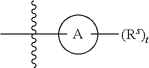

is bonded to and the R^{s} groups). In some embodiments, Ring A is substituted. In some embodiments, Ring A is unsubstituted.

In some embodiments, Ring A is an optionally substituted 5-10 membered aromatic ring having 0-5 heteroatoms independently selected from oxygen, nitrogen, and sulfur. In some embodiments, Ring A is an optionally substituted 5-6 membered aromatic ring having 0-5 heteroatoms independently selected from oxygen, nitrogen, and sulfur. In some embodiments, Ring A is an optionally substituted phenyl ring. In some embodiments, Ring A is a phenyl ring. In some embodiments, Ring A is an optionally substituted 10-membered bicyclic aryl ring. In some embodiments, Ring A is an optionally substituted 5-9 membered heteroaryl ring having 1-4 heteroatoms independently selected from nitrogen, oxygen and sulfur. In some embodiments, Ring A is an optionally substituted 5-membered heteroaryl ring having 1-4 heteroatoms independently selected from nitrogen, oxygen and sulfur. In some embodiments, Ring A is an optionally substituted 6-membered heteroaryl ring having 1-4 heteroatoms independently selected from nitrogen, oxygen and sulfur. In some embodiments, Ring A is an optionally substituted 9-membered bicyclic heteroaryl ring having 1-4 heteroatoms independently selected from nitrogen, oxygen and sulfur. In some embodiments, a heteroatom is nitrogen. In some embodiments, Ring A is protected.

### R^{s}

In some embodiments, an occurrence of R^{s} is R′ as described herein. In some embodiments, an occurrence of R^{s} is R as described herein. In some embodiments, an occurrence of R^{s} is —H. In some embodiments, an occurrence of R^{s} is not —H. In some embodiments, each occurrence of R^{s} is not —H.

In some embodiments, an occurrence of R^{s} is R as described herein and is not —H. For example, in some embodiments, it is optionally substituted C₆₋₁₀ aryl. In some embodiments, it is optionally substituted phenyl. In some embodiments, it is optionally substituted heteroaryl, e.g., 5-6 membered heteroaryl having 1-4 heteroatoms independently selected from nitrogen, oxygen and sulfur.

In some embodiments, an occurrence of R^{s} is halogen. In some embodiments, an occurrence of R^{s} is F. In some embodiments, an occurrence of R^{s} is Cl. In some embodiments, an occurrence of R^{s} is Br. In some embodiments, an occurrence of R^{s} is I. In some embodiments, an occurrence of R^{s} is —CN.

In some embodiments, an occurrence of R^{s} is C(O)OR′, wherein R′ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, an occurrence of R^{s} is C(O)OR, wherein R is as described herein. In some embodiments, an occurrence of R^{s} is C(O)OR, wherein R is as described herein and is not —H. In some embodiments, an occurrence of R^{s} is —C(O)OMe. In some embodiments, an occurrence of R^{s} is —C(O)OEt.

In some embodiments, an occurrence of R^{s} is —OR′, wherein R′ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R′ is optionally substituted C₁₋₆ aliphatic. In some embodiments, an occurrence of R^{s} is —OH. In some embodiments, an occurrence of R^{s} is —OR wherein R is as described herein and is not —H. In some embodiments, an occurrence of R^{s} is —OMe. In some embodiments, an occurrence of R^{s} is -OEt. In some embodiments, an occurrence of R^{s} is —O-propyl. In some embodiments, an occurrence of R^{s} is —O-isopropyl. In some embodiments, an occurrence of R^{s} is —O— butyl. In some embodiments, an occurrence of R^{s} is —O-tert-butyl. In some embodiments, an occurrence of R^{s} is —O—CH₂-Ph. In some embodiments, an occurrence of R^{s} is —O-Ph.

In some embodiments, an occurrence of R^{s} is —N(R′)₂, wherein R′ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, each R′ is independently H or optionally substituted C₁₋₆ aliphatic. In some embodiments, an occurrence of R^{s} is —NH₂. In some embodiments, an occurrence of R^{s} is —N(R)₂, wherein each variable is independent as described herein. In some embodiments, an occurrence of R^{s} is —NHMe. In some embodiments, an occurrence of R^{s} is —NMe₂. In some embodiments, an occurrence of R^{s} is -NHEt. In some embodiments, an occurrence of R^{s} is —N(Et)₂.

In some embodiments, an occurrence of R^{s} is optionally substituted C₆₋₁₀ aryl. In some embodiments, it is optionally substituted phenyl. In some embodiments, it is phenyl. In some embodiments, an occurrence of R^{s} is 5-20 membered heteroaryl having 1-3 heteroatoms independently selected from oxygen, nitrogen, sulfur, phosphorus and silicon. In some embodiments, an occurrence of R^{s} is

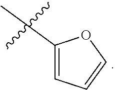 [SMILES: *c1ccco1]

In some embodiments, an occurrence of R^{s} is

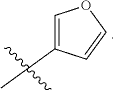 [SMILES: *c1ccoc1]

In some embodiments, an occurrence of R^{s} is

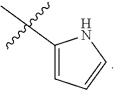 [SMILES: *c1ccc[nH]1]

In some embodiments, an occurrence of R^{s} is

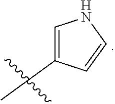 [SMILES: *c1cc[nH]c1]

In some embodiments, an occurrence of R^{s} is

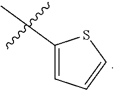 [SMILES: *c1cccs1]

In some embodiments, an occurrence of R^{s} is

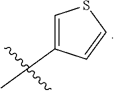 [SMILES: *c1ccsc1]

In some embodiments, an occurrence of R^{s} is

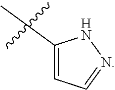 [SMILES: *c1ccn[nH]1]

In some embodiments, an occurrence of R^{s} is

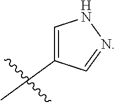 [SMILES: *c1cn[nH]c1]

In some embodiments, an occurrence of R^{s} is

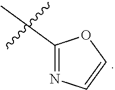 [SMILES: *c1ncco1]

In some embodiments, an occurrence of R² is

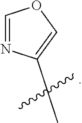 [SMILES: *c1cocn1]

In some embodiments, an occurrence of R^{s} is

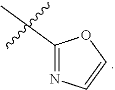 [SMILES: *c1ncco1]

In some embodiments, an occurrence of R^{s} is

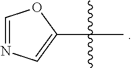 [SMILES: *c1cnco1]

t

In some embodiments, t is 0. In some embodiments, t is 1-5. In some embodiments, t is 1. In some embodiments, t is 2. In some embodiments, t is 3. In some embodiments, t is 4. In some embodiments, t is 5.

n

In some embodiments, n is 0. In some embodiments, n is 1. In some embodiments, n is 2. In some embodiments, n is 3.

### Ring B

As described herein, R^{a} and R^{b} are taken together with their intervening atoms to form Ring B. In some embodiments, Ring B is optionally substituted (in addition to the groups that are bonded to the nitrogen atom to which R^{a} is bonded and the carbon atom to which R is bonded). In some embodiments, Ring B is substituted. In some embodiments, Ring B is unsubstituted.

In some embodiments, Ring B is 4-15, 4-12, 4-10, or 4-7 membered. In some embodiments, Ring B is 4-membered. In some embodiments, Ring B is 5-membered. In some embodiments, Ring B is 6-membered. In some embodiments, Ring B is 7-membered. In some embodiments, Ring B is 8-membered. In some embodiments, Ring B is 9-membered. In some embodiments, Ring B is 10-membered. In some embodiments, Ring B is 11-membered. In some embodiments, Ring B is 12-membered. In some embodiments, Ring B is 13-membered. In some embodiments, Ring B is 14-membered. In some embodiments, Ring B is 15-membered.

In some embodiments, Ring B is saturated. In some embodiments, Ring B is partially unsaturated. In some embodiments, the carbon to which R^{a} is bonded is sp³.

In some embodiments, Ring B is monocyclic. In some embodiments, Ring B is bicyclic. In some embodiments, Ring B is polycyclic. In some embodiments, each monocyclic unit is independently a 3-10 (e.g., 3-7, 4-7, 3-6, 3, 4, 5, 6, 7, 8, 9, or 10) membered saturated, partially unsaturated or aromatic ring having 0-5 heteroatoms. In some embodiments, each monocyclic unit is independently a 3-7 membered saturated, partially unsaturated or aromatic ring having 0-4 heteroatoms independently selected from nitrogen, oxygen and sulfur. In some embodiments, each monocyclic unit is independently a 4-7 membered saturated, partially unsaturated or aromatic ring having 0-4 heteroatoms independently selected from nitrogen, oxygen and sulfur. In some embodiments, each monocyclic unit is independently a 5-7 membered saturated, partially unsaturated or aromatic ring having 0-4 heteroatoms independently selected from nitrogen, oxygen and sulfur. In some embodiments, each monocyclic unit is independently saturated or partially unsaturated. In some embodiments, each monocyclic unit is independently saturated.

In some embodiments, Ring B has 0 heteroatoms in addition to the nitrogen atom to which R is attached. In some embodiments, Ring B has 1-4 additional heteroatoms. In some embodiments, Ring B has 1 additional heteroatom. In some embodiments, Ring B has 2 additional heteroatoms. In some embodiments, Ring B has 3 additional heteroatoms. In some embodiments, Ring B has 4 additional heteroatoms. In some embodiments, each additional heteroatom is independently selected from nitrogen, oxygen and sulfur.

In some embodiments, Ring B is an optionally substituted azetidine ring. In some embodiments, Ring B is an optionally substituted pyrrolidine ring. In some embodiments, Ring B is an optionally substituted piperidine ring.

### R′

In some embodiments, R′ is R as described herein. In some embodiments, R′ is —H. In some embodiments, R′ is not —H.

In some embodiments, R′ is —C(O)R wherein R is as described herein. In some embodiments, R′ is —C(O)OR wherein R is as described herein. In some embodiments, R′ is —C(O)N(R)₂ wherein each R is independently as described herein. In some embodiments, the two R groups are together with the nitrogen to which they are attached to form a ring as described herein. In some embodiments, R′ is —S(O)₂R wherein R is as described herein. In some embodiments, R′ is —S(O)₂R wherein R is as described herein and is not —H.

### R

In some embodiments, each R is independently —H, or an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₀ aryl, C₆₋₂₀ arylaliphatic, C₆₋₂₀ arylheteroaliphatic having 1-5 heteroatoms independently selected from oxygen, nitrogen, sulfur, phosphorus and silicon, 5-10 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon; or:
two R groups are optionally and independently taken together to form a covalent bond; or:
two R groups are optionally and independently taken together with the atom to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the atom, 0-5 (e.g., 0, 1, 2, 3, 4, or 5) heteroatoms independently selected from oxygen, nitrogen, sulfur, phosphorus and silicon; or
two or more R groups are optionally and independently taken together with their intervening atoms to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the intervening atoms, 0-5 (e.g., 0, 1, 2, 3, 4, or 5) heteroatoms independently selected from oxygen, nitrogen, sulfur, phosphorus and silicon.

In some embodiments, each R is independently an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₀ aryl, C₆₋₂₀ arylaliphatic, C₆₋₂₀ arylheteroaliphatic having 1-5 heteroatoms independently selected from oxygen, nitrogen, sulfur, phosphorus and silicon, 5-10 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon; or:
two R groups are optionally and independently taken together to form a covalent bond; or:
two R groups are optionally and independently taken together with the atom to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the atom, 0-5 (e.g., 0, 1, 2, 3, 4, or 5) heteroatoms independently selected from oxygen, nitrogen, sulfur, phosphorus and silicon; or
two or more R groups are optionally and independently taken together with their intervening atoms to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the intervening atoms, 0-5 (e.g., 0, 1, 2, 3, 4, or 5) heteroatoms independently selected from oxygen, nitrogen, sulfur, phosphorus and silicon.

In some embodiments, each R is independently an optionally substituted group selected from C₁₋₁₀ aliphatic, C₁₋₁₀ heteroaliphatic having 1-5 heteroatoms independently selected from nitrogen, oxygen and sulfur, C₆₋₁₀ aryl, C₆₋₂₀ arylaliphatic, C₆₋₂₀ arylheteroaliphatic having 1-5 heteroatoms independently selected from nitrogen, oxygen and sulfur, 5-10 membered heteroaryl having 1-5 heteroatoms independently selected from nitrogen, oxygen and sulfur, and 3-10 membered heterocyclyl having 1-5 heteroatoms independently selected from nitrogen, oxygen and sulfur; or:
two R groups are optionally and independently taken together to form a covalent bond; or:
two R groups are optionally and independently taken together with the atom to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the atom, 0-5 (e.g., 0, 1, 2, 3, 4, or 5) heteroatoms independently selected from nitrogen, oxygen and sulfur; or
two or more R groups are optionally and independently taken together with their intervening atoms to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the intervening atoms, 0-5 (e.g., 0, 1, 2, 3, 4, or 5) heteroatoms independently selected from nitrogen, oxygen and sulfur.

In some embodiments, each R is independently an optionally substituted group selected from C₁₋₁₀ aliphatic, C₁₋₁₀ heteroaliphatic having 1-5 heteroatoms independently selected from nitrogen, oxygen and sulfur, C₆₋₁₀ aryl, C₆₋₂₀ arylaliphatic, C₆₋₂₀ arylheteroaliphatic having 1-5 heteroatoms independently selected from nitrogen, oxygen and sulfur, 5-10 membered heteroaryl having 1-5 heteroatoms independently selected from nitrogen, oxygen and sulfur, and 3-10 membered heterocyclyl having 1-5 heteroatoms independently selected from nitrogen, oxygen and sulfur.

In some embodiments, R is —H. In some embodiments, R is not —H.

In some embodiments, R is optionally substituted C₁₋₃₀ (e.g., C₁₋₂₅, C₁₋₂₀, C₁₋₁₅, etc.) aliphatic. In some embodiments, R is optionally substituted C₁₋₁₀ aliphatic. In some embodiments, an aliphatic group is an alkyl group. In some embodiments, R is C₁₋₆ aliphatic. In some embodiments, R is C₁₋₆ alkyl. In some embodiments, R is optionally substituted methyl. In some embodiments, R is optionally substituted ethyl. In some embodiments, R is optionally substituted n-propyl. In some embodiments, R is optionally substituted isopropyl. In some embodiments, R is n-butyl. In some embodiments, R is t-butyl. In some embodiments, R is pentyl. In some embodiments, R is hexyl.

In some embodiments, an aliphatic group is or comprises a cycloaliphatic ring. In some embodiments, R is optionally substituted C₃₋₃₀ (e.g., C₃₋₂₅, C₃₋₂₀, C₃₋₁₅, C₄₋₁₀, C₃₋₉, C₃₋₇, or 3, 4, 5, 6, 7, 8, 9, 10, 11, 12, 13, 14, 15, 16, 17, 18, 19, 20, 21, 22, 23, 24, 25, 26, 27, 28, 29 or 30 etc.) cycloaliphatic. In some embodiments, R is optionally substituted C₃₋₁₀ cycloaliphatic. In some embodiments, an aliphatic group is a cycloalkyl group. In some embodiments, a cycloaliphatic group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered cycloaliphatic ring. In some embodiments, a cycloaliphatic group is saturated. In some embodiments, it is partially unsaturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted cycloheptyl.

In some embodiments, R is optionally substituted C₁₋₃₀ (e.g., C₁₋₂₅, C₁₋₂₀, C₁₋₁₅, etc.) heteroaliphatic having 1-10 (e.g., 1, 2, 3, 4, 5, 6, 7, 8, 9, or 10, etc.) heteroatoms independently selected from oxygen, nitrogen, sulfur, phosphorus and silicon. In some embodiments, R is optionally substituted C₁₋₃₀ (e.g., C₁₋₂₅, C₁₋₂₀, C₁₋₁₅, etc.) heteroaliphatic having 1-10 (e.g., 1, 2, 3, 4, 5, 6, 7, 8, 9, or 10, etc.) heteroatoms independently selected from nitrogen, oxygen and sulfur. In some embodiments, R is C₁₋₁₅ heteroaliphatic having 1-5 (e.g., 1, 2, 3, 4, or 5, etc.) heteroatoms independently selected from nitrogen, oxygen and sulfur. In some embodiments, R is C₁₋₁₀ heteroaliphatic having 1-5 (e.g., 1, 2, 3, 4, or 5, etc.) heteroatoms independently selected from nitrogen, oxygen and sulfur. In some embodiments, R is C₁₋₁₀ heteroaliphatic having 1-2 (e.g., 1, 2, 3, 4, or 5, etc.) heteroatoms independently selected from nitrogen, oxygen and sulfur. In some embodiments, R is C₁₋₁₀ heteroaliphatic having one heteroatom selected from nitrogen, oxygen and sulfur. In some embodiments, a heteroatom is nitrogen. In some embodiments, a heteroatom is oxygen. In some embodiments, a heteroatom is sulfur.

In some embodiments, R is optionally substituted C₆₋₃₀ (e.g., C₆₋₃₀, C₆₋₂₀, C₆₋₁₀, etc.) aryl. In some embodiments, R is optionally substituted C₁₋₁₀ aryl. In some embodiments, an aryl ring is monocyclic. In some embodiments, an aryl ring is bicyclic. In some embodiments, an aryl ring is polycyclic. In some embodiments, each monocyclic unit is independently a 6-membered aromatic ring. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is optionally substituted C₆₋₃₀ (e.g., C₇₋₃₀, C₇₋₂₀, C₇₋₁₅, etc.) arylaliphatic. In some embodiments, R is optionally substituted C₆₋₁₀ aryl-C₁₋₂₀ aliphatic. In some embodiments, R is optionally substituted C₆₋₁₀ aryl-C₁₋₁₅ aliphatic. In some embodiments, R is optionally substituted C₆₋₁₀ aryl-C₁₋₁₀ aliphatic. In some embodiments, R is optionally substituted C₆₋₁₀ aryl-C₁₋₁₀ alkyl. In some embodiments, R is optionally substituted phenyl-C₁₋₁₅ aliphatic. Suitable aryl and aliphatic groups include those described above.

In some embodiments, R is C₆₋₃₀ (e.g., C₇₋₃₀, C₇₋₂₀, C₇₋₁₅, etc.) arylheteroaliphatic having 1-10 (e.g., 1, 2, 3, 4, 5, 6, 7, 8, 9, or 10, etc.) heteroatoms independently selected from oxygen, nitrogen, sulfur, phosphorus and silicon. In some embodiments, R is optionally substituted C₆₋₁₀ aryl-C₁₋₂₀ heteroaliphatic having 1-10 (e.g., 1, 2, 3, 4, 5, 6, 7, 8, 9, or 10, etc.) heteroatoms independently selected from oxygen, nitrogen, sulfur, phosphorus and silicon. In some embodiments, R is optionally substituted C₆₋₁₀ aryl-C₁₋₂₀ heteroaliphatic having 1-10 (e.g., 1, 2, 3, 4, 5, 6, 7, 8, 9, or 10, etc.) heteroatoms independently selected from nitrogen, oxygen and sulfur. In some embodiments, R is optionally substituted C₆₋₁₀ aryl-C₁₋₁₅ heteroaliphatic having 1-10 (e.g., 1, 2, 3, 4, 5, 6, 7, 8, 9, or 10, etc.) heteroatoms independently selected from nitrogen, oxygen and sulfur. In some embodiments, R is optionally substituted C₆₋₁₀ aryl-C₁₋₁₀ heteroaliphatic having 1-5 (e.g., 1, 2, 3, 4, or 5, etc.) heteroatoms independently selected from nitrogen, oxygen and sulfur. In some embodiments, aryl is phenyl. Suitable aryl and heteroaliphatic groups include those described above.

In some embodiments, R is 5-30 (e.g., 5-25, 5-20, 5-15, 5-10, 5-9, or 5, 6, 7, 8, 9, 10, 11, 12, 13, 14, 15, 16, 17, 18, 19, 20, 21, 22, 23, 24, 25, 26, 27, 28, 29 or 30, etc.) membered heteroaryl having 1-10 (e.g., 1-5, 1-4, or 1, 2, 3, 4, 5, 6, 7, 8, 9, or 10, etc.) heteroatoms independently selected from oxygen, nitrogen, sulfur, phosphorus and silicon. In some embodiments, R is 5-10 (e.g., 5-9, or 5 or 6, etc.) membered heteroaryl having 1-4 (e.g., 1, 2, 3, or 4, etc.) heteroatoms independently selected from nitrogen, oxygen and sulfur. In some embodiments, a heteroaryl ring is monocyclic. In some embodiments, a heteroaryl ring is bicyclic. In some embodiments, a heteroaryl ring is polycyclic. In some embodiments, each monocyclic unit is independently a 5- or 6-membered aromatic ring having 0-4 heteroatoms, e.g., independently selected from nitrogen, oxygen and sulfur, wherein at least one monocyclic unit contains 1-4 heteroatoms. In some embodiments, R is optionally substituted 5-membered monocyclic heteroaryl having 1-4 heteroatoms independently selected from nitrogen, oxygen and sulfur. In some embodiments, R is optionally substituted 6-membered monocyclic heteroaryl having 1-4 heteroatoms independently selected from nitrogen, oxygen and sulfur. In some embodiments, R is optionally substituted 9-membered bicyclic heteroaryl having 1-4 heteroatoms independently selected from nitrogen, oxygen and sulfur. In some embodiments, R is optionally substituted 10-membered bicyclic heteroaryl having 1-4 heteroatoms independently selected from nitrogen, oxygen and sulfur. In some embodiments, a heteroaryl ring has one heteroatom. In some embodiments, a heteroaryl ring has two or more heteroatoms. In some embodiments, a heteroaryl ring has three or more heteroatoms. In some embodiments, a heteroaryl ring has four or more heteroatoms. In some embodiments, a heteroatom is nitrogen. In some embodiments, a heteroatom is oxygen. In some embodiments, a heteroatom is sulfur.

In some embodiments, R is 3-30 (e.g., 3-25, 3-20, 3-15, 3-10, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered heterocyclyl having 1-10 (e.g., 1, 2, 3, 4, 5, 6, 7, 8, 9, or 10, etc.) heteroatoms independently selected from oxygen, nitrogen, sulfur, phosphorus and silicon. In some embodiments, R is 3-30 (e.g., 3-25, 3-20, 3-15, 3-10, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered heterocyclyl having 1-10 (e.g., 1, 2, 3, 4, 5, 6, 7, 8, 9, or 10, etc.) heteroatoms independently selected from nitrogen, oxygen and sulfur. In some embodiments, R is 3-20 (e.g., 3-20, 3-15, 3-10, or 3, 4, 5, 6, 7, 8, 9, 10, 11, 12, 13, 14, 15, 16, 17, 18, 19, or 20, etc.) membered heterocyclyl having 1-5 (e.g., 1, 2, 3, 4, or 5, etc.) heteroatoms independently selected from nitrogen, oxygen and sulfur. In some embodiments, R is 3-10 (e.g., 3, 4, 5, 6, 7, 8, 9, or 10, etc.) membered heterocyclyl having 1-5 (e.g., 1, 2, 3, 4, or 5, etc.) heteroatoms independently selected from nitrogen, oxygen and sulfur. In some embodiments, a heterocyclyl group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered heterocyclyl ring having 1-5 (e.g., 1, 2, 3, 4, or 5, etc.) heteroatoms independently selected from nitrogen, oxygen and sulfur. In some embodiments, a heterocyclyl group is saturated. In some embodiments, it is partially unsaturated. In some embodiments, a heterocyclyl ring has one heteroatom. In some embodiments, a heterocyclyl ring has two or more heteroatoms. In some embodiments, a heterocyclyl ring has three or more heteroatoms. In some embodiments, a heterocyclyl ring has four or more heteroatoms. In some embodiments, a heteroatom is nitrogen. In some embodiments, a heteroatom is oxygen. In some embodiments, a heteroatom is sulfur.

In some embodiments, two R groups are optionally and independently taken together to form a covalent bond. In some embodiments, two R groups attached to neighboring atoms are optionally and independently taken together to form a covalent bond.

In some embodiments, two R groups are optionally and independently taken together with the atom to form an optionally substituted, 3-30 (e.g., 3-25, 3-20, 3-15, 3-10, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered, monocyclic, bicyclic or polycyclic ring having, in addition to the atom, 0-10 (e.g., 0, 1, 2, 3, 4, 5, 6, 7, 8, 9, or 10, etc.) heteroatoms independently selected from oxygen, nitrogen, sulfur, phosphorus and silicon. In some embodiments, two or more R groups are optionally and independently taken together with their intervening atoms to form an optionally substituted, 3-30 (e.g., 3-25, 3-20, 3-15, 3-10, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered, monocyclic, bicyclic or polycyclic ring having, in addition to the intervening atoms, 0-10 (e.g., 0, 1, 2, 3, 4, 5, 6, 7, 8, 9, or 10, etc.) heteroatoms independently selected from oxygen, nitrogen, sulfur, phosphorus and silicon.

As described herein, in various instances, two or more R groups, or two or more groups that are or can be R (e.g., R^{s}, R′, etc.,), can be together with their intervening atom(s) to form an optionally substituted ring as described herein. In some embodiments, a formed ring is substituted (in addition to groups attached to the intervening atom(s). In some embodiments, a formed ring is unsubstituted. In some embodiments, a formed ring is 3-30, 3-25, 3-20, 3-15, 3-10, 3-8, 3-6, 5-6, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is 11-membered. In some embodiments, a formed ring is 12-membered. In some embodiments, a formed ring is saturated. In some embodiments, a formed ring is partially unsaturated. In some embodiments, a formed ring is aromatic. In some embodiments, a formed ring is monocyclic. In some embodiments, it is bicyclic. In some embodiments, it is polycyclic. In some embodiments, each monocyclic unit is independently a 3-15 (e.g., 3-15, 3-10, 3-8, 3-6, 5-6, or 3, 4, 5, 6, 7, 8, 9, 10, 11, 12, 13, 14, or 15, etc.) membered ring which is independently saturated, partially unsaturated or aromatic and has 0-4 heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a formed ring has, in addition to the intervening atom(s), 0-10 (e.g., 0, 1, 2, 3, 4, 5, 6, 7, 8, 9, or 10, etc.) heteroatoms independently selected from oxygen, nitrogen, sulfur, phosphorus and silicon. In some embodiments, a formed ring has, in addition to the intervening atom(s), 0-5 (e.g., 0, 1, 2, 3, 4, or 5, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, reduction is carried out in the present of Ru—[(S, S)-Ts-DPEN], and a product is

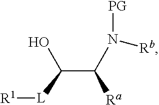

wherein each variable is independently as described herein. In some embodiments, reduction is carried out in the present of Ru—[(S, S)-Ts-DPEN], and a product is

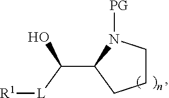

wherein R¹, L, PG, and n are independently as described herein. In some embodiments, a product is

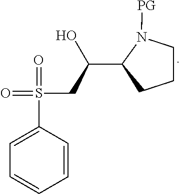

In some embodiments, a product is

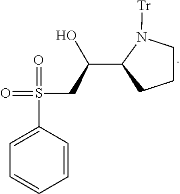

In some embodiments, such a product is formed with selectivity as described herein.

In some embodiments, reduction is carried out in the present of Ru—[(R, R)-Ts-DPEN], and a product is

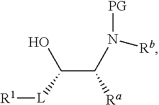

wherein each variable is independently as described herein. In some embodiments, reduction is carried out in the present of Ru—[(R, R)-Ts-DPEN], and a product is

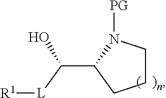

wherein R¹, L, PG, and n are independently as described herein. In some embodiments, a product is

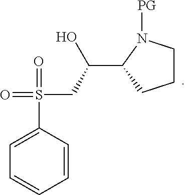

In some embodiments, a product is

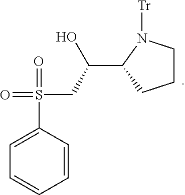

In some embodiments, such a product is formed with selectivity as described herein.

In some embodiments, reduction is carried out in the present of an agent that delivers hydride, and a product is

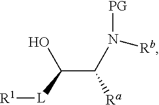

wherein each variable is independently as described herein. In some embodiments, reduction is carried out in the present of an agent that delivers hydride, and a product is

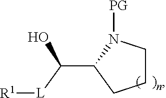

wherein R¹, L, PG, and n are independently as described herein. In some embodiments, an agent is NaBH₄. In some embodiments, an agent is LiBH₄. In some embodiments, a product is

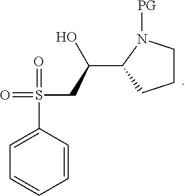

In some embodiments, a product is

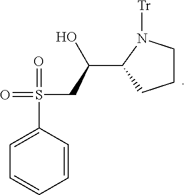

In some embodiments, such a product is formed with selectivity as described herein.

In some embodiments, reduction is carried out in the present of an agent that delivers hydride, and a product is

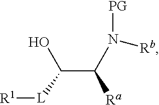

wherein each variable is independently as described herein. In some embodiments, reduction is carried out in the present of an agent that delivers hydride, and a product is

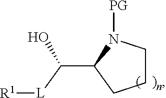

wherein R¹, L, PG, and n are independently as described herein. In some embodiments an agent is NaBH₄. In some embodiments, an agent is LiBH₄. In some embodiments, a product is

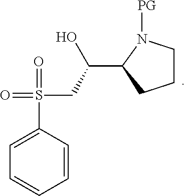

In some embodiments, a product is

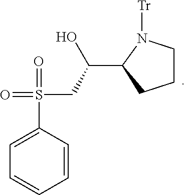

In some embodiments, such a product is formed with selectivity as described herein.

In some embodiments, the present disclosure provides a method of preparing a compound of formula P-a-1

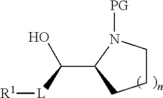

or a salt thereof comprising a step of reducing a compound of formula INT-1-a-1

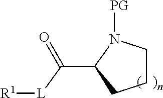

or a salt thereof. In some embodiments, reduction is carried out in the presence of Ru—[(S, S)-Ts-DPEN].

In some embodiments, the present disclosure provides a method of preparing a compound of formula P-a-2

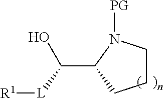

or a salt thereof comprising a step of reducing a compound of formula INT-1-a-2

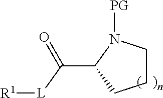

or a salt thereof. In some embodiments, reduction is carried out in the presence of Ru—[(R, R)-Ts-DPEN].

In some embodiments, the present disclosure provides a method of preparing a compound of formula P-a-3:

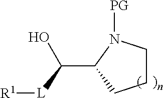

or a salt thereof comprising a step of reducing a compound of formula INT-1-a-2

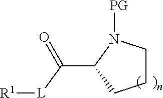

or a salt thereof. In some embodiments, reduction is carried out in the presence of NaBH₄. In some embodiments, an agent is LiBH₄.

In some embodiments, the present disclosure provides a method of preparing a compound of formula P-a-4

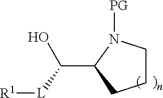

or a salt thereof comprising a step of reducing a compound of formula INT-1-a-1

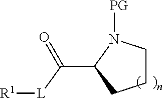

or a salt thereof. In some embodiments, reduction is carried out in the presence of NaBH₄. In some embodiments, an agent is LiBH₄.

### Certain Compounds and Compositions

In some embodiments, the present disclosure provides various compounds and compositions that have purity as described herein and/or are produced with selectivity as described herein.

In some embodiments, the present disclosure provides a compound having the structure of formula INT-1:

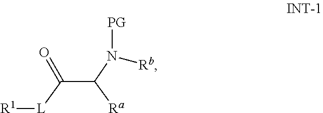

or a salt thereof, wherein:
PG is an amino protecting group;
L is optionally substituted —CH₂—;
R^{t} is R, —P(O)(R²)₂, —S(O)₂R², or —Si(R)₃;
R² is R, —OR, —N(R′)₂, or

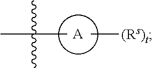

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a compound of formula INT-1-1 or a salt thereof. In some embodiments, the present disclosure provides a compound of formula INT-1-2 or a salt thereof. In some embodiments, a compound of formula INT-1 has the structure of INT-1-a. In some embodiments, a compound of formula INT-1 has the structure of INT-1-b.

In some embodiments, the present disclosure provides a compound having the structure of formula INT-1-a:

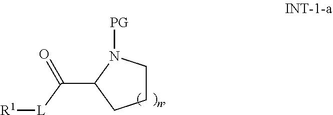

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

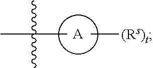

Ring A is an optionally substituted 5-10 membered aromatic ring having 0-5 heteroatoms independently selected from oxygen, nitrogen, sulfur, phosphorus and silicon;
each R is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a compound of formula INT-1-a-1 or a salt thereof. In some embodiments, the present disclosure provides a compound of formula INT-1-a-2 or a salt thereof. In some embodiments, the present disclosure provides a compound of formula INT-1-b-1 or a salt thereof. In some embodiments, the present disclosure provides a compound of formula INT-1-b-2 or a salt thereof.

Certain embodiments for variables are described above, and those skilled in the art reading the present disclosure will be able to select and combine them.

In some embodiments, the present disclosure provides a compound having the structure of

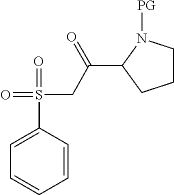

or a salt thereof. In some embodiments, PG is an amino protecting group other than Boc. In some embodiments, PG is -Trt. In some embodiments, the present disclosure provides a compound having the structure of

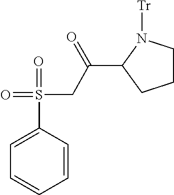

or a salt thereof. In some embodiments, the present disclosure provides a compound having the structure of

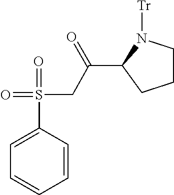

or a salt thereof. In some embodiments, the present disclosure provides a compound having the structure of

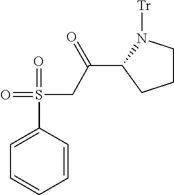

or a salt thereof.

In some embodiments, the present disclosure provides a compound having the structure of

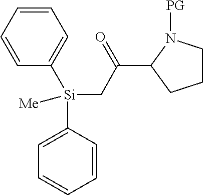

or a salt thereof. In some embodiments, PG is an amino protecting group other than Boc. In some embodiments, PG is -Trt. In some embodiments, the present disclosure provides a compound having the structure of

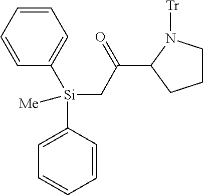

or a salt thereof. In some embodiments, the present disclosure provides a compound having the structure of

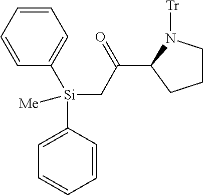

or a salt thereof. In some embodiments, the present disclosure provides a compound having the structure of

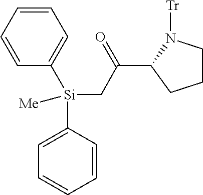

or a salt thereof.

In some embodiments, the present disclosure provides a composition comprising:
(1) a compound of formula INT-1

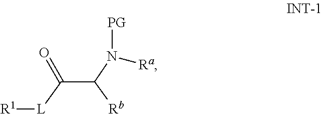

or a salt thereof, and
(2) a compound of formula P:

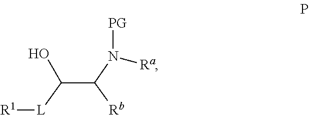

or a salt thereof; wherein
PG is an amino protecting group;
L is optionally substituted —CH₂—;
R¹ is R, —P(O)(R²)₂, —S(O)₂R², or —Si(R)₃;
R² is R, —OR, —N(R′)₂, or;

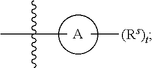

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a composition comprising:
(1) a compound of formula INT-1-a:

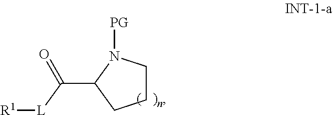

or a salt thereof, and
(2) a compound of formula P-a:

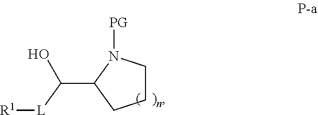

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

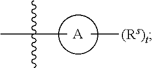

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the composition comprises

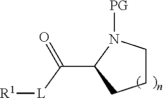

or a salt thereof and

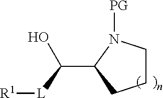

or a salt thereof. In some embodiments, the composition comprises

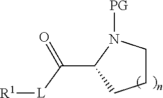

or a salt thereof and

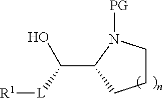

or a salt thereof. In some embodiments, the composition comprises

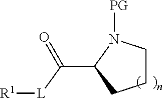

or a salt thereof and

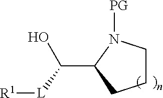

or a salt thereof. In some embodiments, the composition comprises

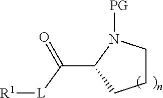

or a salt thereof and

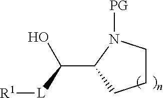

or a salt thereof.

In some embodiments, a composition further comprises a metal complex as described herein. In some embodiments, a composition further comprises a reducing agent as described herein.

In some embodiments, a compound of formula P is a compound of formula P-1 or P-4, and a compound of formula INT-1 is a compound of formula INT-1-1.

In some embodiments, a compound of formula P is a compound of formula P-1, and a compound of formula INT-1 is a compound of formula INT-1-1. In some embodiments, a compound of formula P is a compound of formula P-a-1 or P-b-1, and a compound of formula INT-1 is a compound of formula INT-1-a-1 or INT-1-b-1, respectively. In some embodiments, such a composition further comprises a metal complex, e.g., a Ru complex as described herein (e.g., Ru—[(S, S)-Ts-DPEN]). In some embodiments, such a composition further comprises a reducing agent such as HCOOH or a salt thereof. In some embodiments, a compound of formula P-1, P-a-1 or P-b-1 or a salt thereof is enriched over compound(s) of formula P-2, P-a-2, P-b-2, P-3, P-a-3, P-b-3, P-4, P-a-4, and/or P-b-4, or salt(s) thereof. In some embodiments, a compound of formula P-1, P-a-1 or P-b-1 or a salt has a purity, diastereomeric purity, and/or enantiomeric purity independently as described herein.

In some embodiments, a compound of formula P is a compound of formula P-2, and a compound of formula INT-1 is a compound of formula INT-1-2. In some embodiments, a compound of formula P is a compound of formula P-a-2 or P-b-2, and a compound of formula INT-1 is a compound of formula INT-1-a-2 or INT-1-b-2, respectively. In some embodiments, such a composition further comprises a metal complex, e.g., a Ru complex as described herein (e.g., Ru—[(R, R)-Ts-DPEN]). In some embodiments, such a composition further comprises a reducing agent such as HCOOH or a salt thereof. In some embodiments, a compound of formula P-2, P-a-2 or P-b-2 or a salt thereof is enriched over compound(s) of formula P-1, P-a-1, P-b-1, P-3, P-a-3, P-b-3, P-4, P-a-4, and/or P-b-4, or salt(s) thereof. In some embodiments, a compound of formula P-1, P-a-1 or P-b-1 or a salt has a purity, diastereomeric purity, and/or enantiomeric purity independently as described herein.

In some embodiments, a compound of formula P is a compound of formula P-3, and a compound of formula INT-1 is a compound of formula INT-1-2. In some embodiments, a compound of formula P is a compound of formula P-a-3 or P-b-3, and a compound of formula INT-1 is a compound of formula INT-1-a-2 or INT-1-b-2, respectively. In some embodiments, such a composition does not contain a transition metal complex, e.g., a Ru complex as described herein (e.g., Ru—[(R, R)-Ts-DPEN] or Ru—[(S, S)-Ts-DPEN]). In some embodiments, such a composition further comprises a reducing agent such as a borohydride (e.g., LiBH₄, NaBH₄, etc.). In some embodiments, a compound of formula P-3, P-a-3 or P-b-3 or a salt thereof is enriched over compound(s) of formula P-1, P-a-1, P-b-1, P-2, P-a-2, P-b-2, P-4, P-a-4, and/or P-b-4, or salt(s) thereof. In some embodiments, a compound of formula P-1, P-a-1 or P-b-1 or a salt has a purity, diastereomeric purity, and/or enantiomeric purity independently as described herein.

In some embodiments, a compound of formula P is a compound of formula P-4, and a compound of formula INT-1 is a compound of formula INT-1-1. In some embodiments, a compound of formula P is a compound of formula P-a-4 or P-b-4, and a compound of formula INT-1 is a compound of formula INT-1-a-1 or INT-1-b-1, respectively. In some embodiments, such a composition does not contain a transition metal complex, e.g., a Ru complex as described herein (e.g., Ru—[(R, R)-Ts-DPEN] or Ru—[(S, S)-Ts-DPEN]). In some embodiments, such a composition further comprises a reducing agent such as a borohydride (e.g., LiBH₄, NaBH₄, etc.). In some embodiments, a compound of formula P-4, P-a-4 or P-b-4 or a salt thereof is enriched over compound(s) of formula P-1, P-a-1, P-b-1, P-2, P-a-2, P-b-2, P-3, P-a-3, and/or P-b-3, or salt(s) thereof. In some embodiments, a compound of formula P-1, P-a-1 or P-b-1 or a salt has a purity, diastereomeric purity, and/or enantiomeric purity independently as described herein.

In some embodiments, the present disclosure provides a composition comprising:
(1) a compound of formula INT-1:

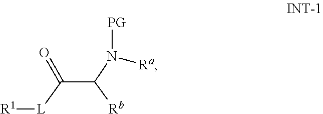

or a salt thereof, and
(2) a compound of formula INT-3:

R¹-L-H, INT-3

or a salt thereof; wherein
PG is an amino protecting group;
L is optionally substituted —CH₂—;
R¹ is R, —P(O)(R²)₂, —S(O)₂R², or —Si(R)₃;
R² is R, —OR, —N(R′)₂, or

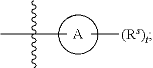

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a composition comprising:
(1) a compound of formula INT-1-a:

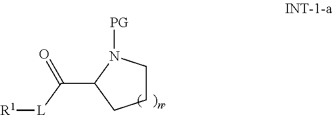

or a salt thereof, and
(2) a compound of formula INT-3:

R¹-L-H, INT-3

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

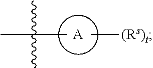

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a compound of formula INT-1 is a compound of formula INT-1-a. In some embodiments, a compound of formula INT-1 is a compound of formula INT-1-b. In some embodiments, a compound of INT-1 is a compound of formula INT-1-1. In some embodiments, a compound of INT-1 is a compound of formula INT-1-2. In some embodiments, a compound of INT-1-a is a compound of formula INT-1-a-1. In some embodiments, a compound of INT-1 is a compound of formula INT-1-a-2. In some embodiments, a compound of INT-1 is a compound of formula INT-1-1. In some embodiments, a compound of INT-1 is a compound of formula INT-1-2.

In some embodiments, the present disclosure provides a composition comprising:
(1) a compound of formula INT-1:

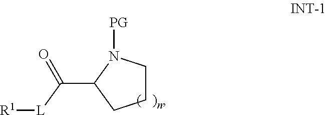

or a salt thereof, and
(2) a compound of formula INT-2:

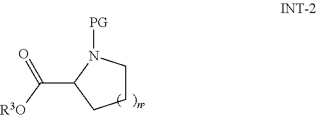

or a salt thereof; wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or

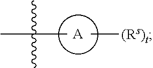

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a composition comprising:
(1) a compound of formula INT-1-a:

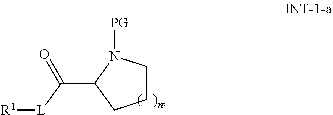

or a salt thereof, and
(2) a compound of formula INT-2-a:

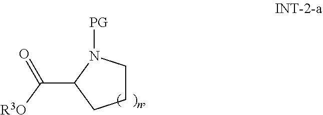

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

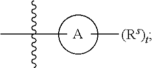

R³ is R;
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a compound of formula INT-1 is a compound of formula INT-1-a. In some embodiments, a compound of formula INT-1 is a compound of formula INT-1-b. In some embodiments, a compound of INT-1 is a compound of formula INT-1-1. In some embodiments, a compound of INT-1 is a compound of formula INT-1-2. In some embodiments, a compound of INT-1-a is a compound of formula INT-1-a-1. In some embodiments, a compound of INT-1 is a compound of formula INT-1-a-2. In some embodiments, a compound of INT-1 is a compound of formula INT-1-1. In some embodiments, a compound of INT-1 is a compound of formula INT-1-2. In some embodiments, a compound of formula INT-2 is a compound of formula INT-2-a. In some embodiments, a compound of formula INT-2 is a compound of formula INT-2-b. In some embodiments, a compound of INT-2 is a compound of formula INT-2-1. In some embodiments, a compound of INT-2 is a compound of formula INT-2-2. In some embodiments, a compound of INT-2-a is a compound of formula INT-2-a-1. In some embodiments, a compound of INT-2 is a compound of formula INT-2-a-2. In some embodiments, a compound of INT-2 is a compound of formula INT-2-1. In some embodiments, a compound of INT-2 is a compound of formula INT-2-2.

In some embodiments, a composition comprises a compound of formula INT-1-1, INT-1-a-1, or INT-1-b-1, or a salt thereof, and a compound of formula INT-2-1, INT-2-a-1, or INT-2-b-1, or a salt thereof. In some embodiments, each compound of formula INT-1-1, INT-1-a-1, INT-1-b-1, INT-2-1, INT-2-a-1, or INT-2-b-1, or a salt thereof independently has a purity, diastereomeric purity and/or enantiomeric purity as described herein.

In some embodiments, a composition comprises a compound of formula INT-1-2, INT-1-a-2, or INT-1-b-2, or a salt thereof, and a compound of formula INT-2-2, INT-2-a-2, or INT-2-b-2, or a salt thereof. In some embodiments, each compound of formula INT-1-2, INT-1-a-2, INT-1-b-2, INT-2-2, INT-2-a-2, or INT-2-b-2, or a salt thereof independently has a purity, diastereomeric purity and/or enantiomeric purity as described herein.

In some embodiments, a compound comprising a compound of formula INT-1 or a salt thereof and a compound of formula INT-2 or a salt thereof further comprises a compound of formula INT-3 or a salt thereof. In some embodiments, a compound has the structure of formula INT-3 or a salt thereof, e.g., a Li⁺ salt.

In some embodiments, a composition comprises

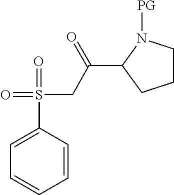

or a salt thereof and

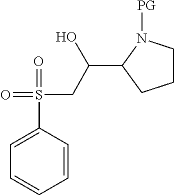

or a salt thereof, wherein PG is an amino protecting group as described herein. In some embodiments, the composition comprises

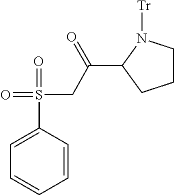

or a salt thereof and

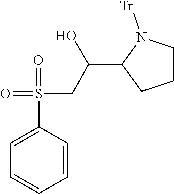

or a salt thereof.

In some embodiments, a composition comprises

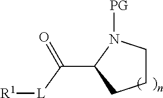

or a salt thereof and R¹-L-H or a salt thereof. In some embodiments, the composition comprises

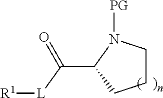

or a salt thereof and R¹-L-H or a salt thereof.

In some embodiments, the composition comprises

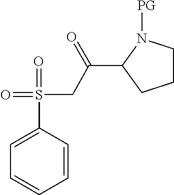

or a salt thereof and

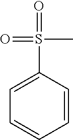

or a salt thereof, wherein PG is an amino protecting group. In some embodiments, the composition comprises

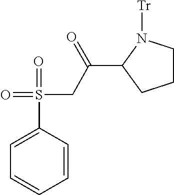

or a salt thereof and

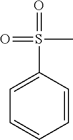

or a salt thereof.

In some embodiments, a composition comprises

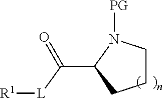

or a salt thereof and

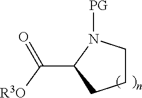

or a salt thereof. In some embodiments, a composition comprises

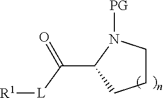

or a salt thereof and

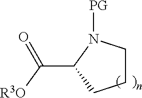

or a salt thereof.

In some embodiments, the composition comprises

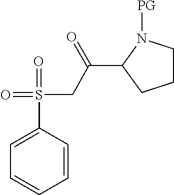

or a salt thereof and

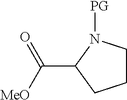

or a salt thereof, wherein PG is an amino protecting group as described herein. In some embodiments, the composition comprises

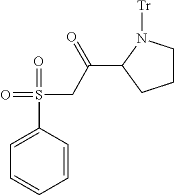

or a salt thereof and

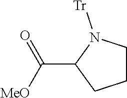

or a salt thereof.

### Certain Application of Compounds and Compositions

As appreciated by those skilled in the art, compounds of the present disclosure, e.g., compounds of formula P, DP, etc. or salts thereof are useful for many purposes, e.g., as pharmaceuticals, chiral auxiliaries, etc. or agents useful for their preparation.

In some embodiments, provided compounds, e.g., compounds of formula P, DP, or salts thereof, are useful as chiral agents for stereoselective synthesis. For example, in some embodiments, they are useful for chirally controlled preparation of oligonucleotides. Certain uses are described in, e.g., WO2019/055951, WO2020/191252, etc. and are incorporated herein by reference.

### Certain Phosphoramidite Technologies

In some embodiments, the present disclosure provides a compound of formula PMT:

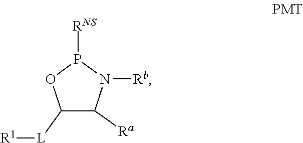

or a salt thereof, wherein:
R^{NS} is a nucleoside;
L is optionally substituted —CH₂—;
R¹ is R, —P(O)(R²)₂, —S(O)₂R², or —Si(R)₃;
R² is R, —OR, —N(R′)₂, or

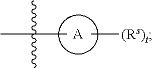

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

As will be appreciated by those skilled in the art, a compound of formula PMT can exist as various diastereomers. In some embodiments, the present disclosure provides cis isomers of phosphoramidites having the structure of formula PMT or salts thereof. In some embodiments, cis isomers comprises R^{NS}, -L-R¹ and R^{a} pointing to the same direction of a plane defined by ring structure

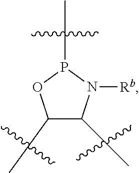

e.g., both above the plane or both below the plane. In some embodiments, a cis isomer of a phosphoramidite of formula PMT or a salt thereof has a structure of formula PMT-A:

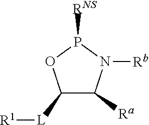

or a salt thereof. In some embodiments, a cis isomer of a phosphoramidite of formula PMT or a salt thereof has a structure of formula PMT-B

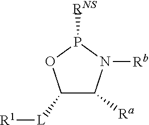

or a salt thereof.

In some embodiments, the present disclosure provides trans isomers of phosphoramidites having the structure of formula PMT or salts thereof. In some embodiments, trans isomers comprises R^{NS} and -L-R¹ pointing to opposite directions of a plane defined by ring structure

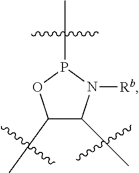

e.g., one above the plane and one below the plane. In some embodiments, trans isomers comprises R^{NS} and R^{a} pointing to opposite directions of a plane defined by ring structure

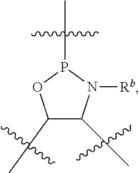

e.g., one above the plane and one below the plane. In some embodiments, trans isomer of a phosphoramidite of formula PMT or a salt thereof has a structure of formula PMT-A′:

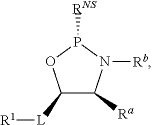

or a salt thereof. In some embodiments, cis isomer of a phosphoramidite of formula PMT or a salt thereof has a structure of formula PMT-B′:

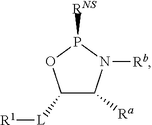

or a salt thereof.

In some embodiments, -L-R¹ and R^{a} are cis.

In some embodiments, the present disclosure provides a method of preparing a compound or composition as described herein, comprising reacting a compound having the structure of formula CA:

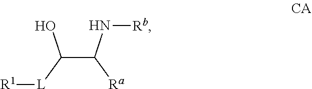

or a salt thereof with a nucleoside, wherein each of the variable groups is independently as described herein.

In some embodiments, a compound of formula CA has a structure of formula CA-A:

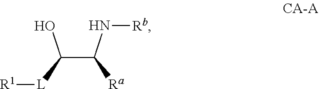

or a salt thereof wherein each of the variable groups is independently as described herein.

In some embodiments, a compound of formula CA has a structure of formula CA-B:

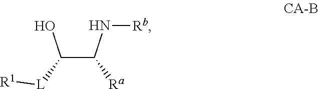

or a salt thereof wherein each of the variable groups is independently as described herein.

### R^{NS}

In some embodiments, R^{NS} is a nucleoside comprising a protecting group. In some embodiments, R^{NS} is a nucleoside suitably protected for oligonucleotide synthesis.

In some embodiments, R^{NS} is —SU-BA wherein each SU and BA is independently as described herein. In some embodiments, R^{NS} is —O—SU-BA wherein each SU and BA is independently as described herein. In some embodiments, SU is a sugar as described herein. In some embodiments, BA is a nucleobase as described herein.

In some embodiments, R^{NS} is —O—SU-BA wherein BA is an optionally substituted group selected from C₁₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety; —O—SU— is —O-L^{s}- or

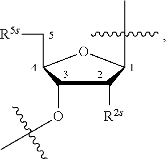

wherein —O—SU— is connected to the phosphorus atom in formula PMT-A or PMT-B through the oxygen atom; L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R^{5s} is R′ or —OR′; R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and -Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon.

### SU

In some embodiments, SU is a sugar as described herein. For example, in some embodiments, SU is optionally substituted

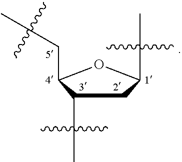

In some embodiments, SU is a sugar having the structure of

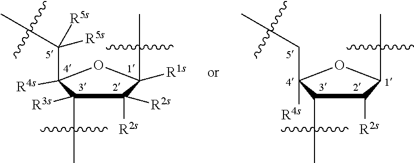

as described herein.

In some embodiments, —O—SU— is.

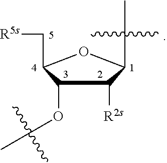

In some embodiments, —O—SU— is —O-L^{s}-. In some embodiments, L^{s} is -Cy-. In some embodiments, L^{s} is optionally substituted 3-30 membered carbocyclylene. In some embodiments, L^{s} is optionally substituted 6-30 membered arylene. In some embodiments, L^{s} is optionally substituted 5-30 membered heteroarylene having 1-10 heteroatoms independently selected from oxygen, nitrogen and sulfur. In some embodiments, L^{s} is optionally substituted 5-30 membered heteroarylene having 1-5 heteroatoms independently selected from oxygen, nitrogen and sulfur. In some embodiments, L^{s} is optionally substituted 3-30 membered heterocyclylene having 1-10 heteroatoms independently selected from oxygen, nitrogen, sulfur, phosphorus and silicon. In some embodiments, L^{s} is optionally substituted 3-30 membered heterocyclylene having 1-5 heteroatoms independently selected from oxygen, nitrogen, sulfur, phosphorus and silicon. In some embodiments, L^{s} is optionally substituted 5-30 membered heterocyclylene having 1-10 heteroatoms independently selected from oxygen, nitrogen, sulfur, phosphorus and silicon. In some embodiments, L^{s} is optionally substituted 5-30 membered heterocyclylene having 1-5 heteroatoms independently selected from oxygen, nitrogen, sulfur, phosphorus and silicon. In some embodiments, L^{s} is optionally substituted 5-10 membered heterocyclylene having one oxygen atom. In some embodiments, L^{s} is optionally substituted 5-membered heterocyclylene having one oxygen atom. In some embodiments, L^{s} is optionally substituted 6-membered heterocyclylene having one oxygen atom. In some embodiments, L^{s} is optionally substituted 5-10 membered bicyclic heterocyclylene having one or two oxygen atoms. In some embodiments, L^{s} is optionally substituted 7-10 membered bicyclic heterocyclylene having one or two oxygen atoms. In some embodiments, L^{s} is optionally substituted 7-10 membered bicyclic heterocyclylene having two oxygen atoms. In some embodiments, L^{s} is optionally substituted 7-membered bicyclic heterocyclylene having two oxygen atoms.

In some embodiments, SU is a sugar moiety used in oligonucleotide synthesis. In some embodiments, SU is an optionally substituted saturated monocyclic, bicyclic or polycyclic saturated aliphatic ring wherein one or more methylene units are replaced with —O—. In some embodiments, SU is a ribose or deoxyribose moiety found in natural DNA or RNA molecules.

In some embodiments, R^{NS} is —SU-BA, wherein SU is a sugar moiety as described herein. In some embodiments, a sugar has a structure of

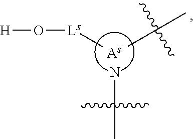

wherein Ring A^{s} is an optionally substituted 3-30 membered, monocyclic, bicyclic or polycyclic ring having, in addition to the nitrogen, 0-10 heteroatoms, Ring A^{s} is connected to the phosphorus atom in formula PMT-A or PMT-B through the nitrogen atom and L^{s} is as described herein. In some embodiments, R^{NS} is

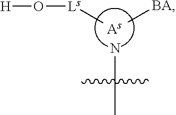

In some embodiments,

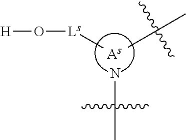

is

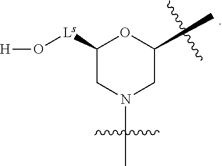

In some embodiments, R^{NS} is

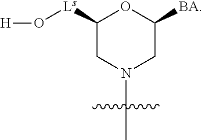

In some embodiments, —OH is optionally substituted or protected, e.g., as -ODMTr.

In some embodiments, the present disclosure provides a compound having a structure of PMT-A1:

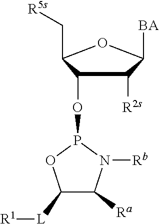

or a salt thereof, wherein each of the variable groups is independently as described herein. In some embodiments, the present disclosure provides a compound having a structure of PMT-B1:

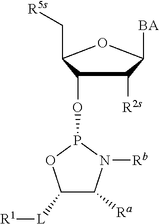

or a salt thereof, wherein each of the variable groups is independently as described herein.

### R^{5s}

In some embodiments, R^{5s} is R′. In some embodiments, R^{5s} is —OR′. In some embodiments, R^{5s} is a protected hydroxyl group suitable for oligonucleotide synthesis. In some embodiments, R^{5s} is —OR′, wherein R′ is optionally substituted C₁₋₆ aliphatic. In some embodiments, R^{5s} is DMTrO-. Example protecting groups are widely known in the art for use in accordance with the present disclosure. For additional examples, see Greene, T. W.; Wuts, P. G. M. Protective Groups in Organic Synthesis, 2nd ed.; Wiley: New York, 1991, and WO/2011/005761, WO/2013/012758, WO/2014/012081, WO/2015/107425, WO/2010/064146, WO/2014/010250, WO/2011/108682, WO/2012/039448, and WO/2012/073857, the protecting groups of each of which are hereby incorporated by reference.

### R^{2s}

In some embodiments, R^{2s} is —H. In some embodiments, R^{2s} is —F. In some embodiments, R^{2s} is —CN. In some embodiments, R^{2s} is —N₃. In some embodiments, R^{2s} is —NO. In some embodiments, R^{2s} is —NO₂. In some embodiments, R^{2s} is —R′. In some embodiments, R^{2s} is —OR′. In some embodiments, R^{2s} is —OR′, wherein R′ is optionally substituted C₁₋₆ aliphatic. In some embodiments, R^{2s} is —OMe. In some embodiments, R^{2s} is —SR′. In some embodiments, R^{2s} is —N(R′)₂. In some embodiments, R^{2s} is —O-L-OR′. In some embodiments, R^{2s} is —O-L-OR′, wherein L is optionally substituted C₁₋₆ alkylene, and R′ is optionally substituted C₁₋₆ aliphatic. In some embodiments, R^{2s} is —O-(optionally substituted C₁₋₆ alkylene)-OR′. In some embodiments, R^{2s} is —O-(optionally substituted C₁₋₆ alkylene)-OR′, wherein R′ is optionally substituted C₁₋₆ alkyl. In some embodiments, R^{2s} is —OCH₂CH₂OMe. In some embodiments, R^{2s} is —O-L-SR′. In some embodiments, R^{2s} is —O-L-N(R′)₂. In some embodiments, R^{2s} is L connecting C2 with C1, C2, C3, C4 or C5. In some embodiments, R^{2s} is L connecting C2 with C1. In some embodiments, R^{2s} is L connecting C2 with C2. In some embodiments, R^{2s} is L connecting C2 with C3. In some embodiments, R^{2s} is L connecting C2 with C4. In some embodiments, R^{2s} is L connecting C2 with C5. In some embodiments, R^{2s} is (C2)-O-(optionally substituted methylene)-(C4). In some embodiments, R^{2s} is (C2)-O-(methylene)-(C4). In some embodiments, R^{2s} is (C2)-O-(methylmethylene)-(C4). In some embodiments, R^{2s} is (C2)-O—((R)-methylmethylene)-(C4). In some embodiments, R^{2s} is (C2)-O—((S)-methylmethylene)-(C4). In some embodiments, R^{2s} is (C2)-O-(ethylmethylene)-(C4). In some embodiments, R^{2s} is (C2)-O—((R)-ethylmethylene)-(C4). In some embodiments, R^{2s} is (C2)-O—((S)-ethylmethylene)-(C4). In some embodiments, R^{2s} comprises a chiral carbon in R configuration. In some embodiments, R^{2s} comprises a chiral carbon in S configuration.

### BA Nucleobase

In some embodiments, in various formulae, BA is a nucleobase as described herein. In some embodiments, BA is an optionally substituted group selected from C₃₋₃₀ cycloaliphatic, C₆₋₃₀ aryl, C₅₋₃₀ heteroaryl having 1-10 heteroatoms, C₃₋₃₀ heterocyclyl having 1-10 heteroatoms, a natural nucleobase moiety, and a modified nucleobase moiety. In some embodiments, BA is an optionally substituted, saturated, partially unsaturated or aromatic C₃₋₃₀ (e.g., C₃₋₂₅, C₃₋₂₀, C₃₋₁₅, C₅₋₃₀, C₅₋₂₀, C₅₋₁₅, C₅₋₁₀, 3, 4, 5, 6, 7, 8, 9, 10, 11, 12, 13, 14, 15, 16, 17, 18, 19, 20, 21, 22, 23, 24, 25, 26, 27, 28, 29, 30, etc.) monocyclic, bicyclic or polycyclic ring having 0-10 (e.g., 0, 1-10, 1, 2, 3, 4, 5, 6, 7, 8, 9, 10, etc.) heteroatoms. In some embodiments, BA is optionally substituted C₆₋₃₀ (e.g., C₆₋₂₅, C₆₋₂₀, C₆₋₁₄, 6, 10, 14, etc.) aryl. In some embodiments, BA is optionally substituted 6-14 membered aryl. In some embodiments, BA is optionally substituted C₅₋₃₀ (e.g., C₅₋₂₅, C₅₋₂₀, C₅₋₁₅, C₅₋₁₄, 5, 6, 9, 10, 12, 13, 14, etc.) heteroaryl having 1-5 (e.g., 1-3, 1, 2, 3, 4, 5, etc.) heteroatoms. In some embodiments, a heteroatom is nitrogen. In some embodiments, each monocyclic wring in BA is optionally substituted 3-10 (e.g., 3-7, 5-10, 3, 4, 5, 6, 7, 8, 9, 10, etc.) membered saturated, partially unsaturated or aromatic ring having 1-5 (e.g., 1-3, 1, 2, 3, 4, 5, etc.) heteroatoms. In some embodiments, one or more ring heteroatom is nitrogen. In some embodiments, BA comprises one or more partially unsaturated monocyclic rings. In some embodiments, BA comprises one or more aromatic rings. In some embodiments, BA comprises one or more heteroaryl rings. In some embodiments, BA comprises one or more heteroaryl rings, one or more of which independently comprise a nitrogen atom. In some embodiments, BA comprises one or more heterocyclyl rings, one or more of which independently comprise a nitrogen atom. In some embodiments, a ring, e.g., a monocyclic ring unit in BA, or BA, is 5-membered. In some embodiments, a monocyclic ring unit in BA, or BA, is 6-membered. In some embodiments, a bicyclic ring unit in BA, or BA, is 8-10-membered. In some embodiments, it is 8-membered. In some embodiments, it is 9-membered. In some embodiments, it is 10-membered. Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)]. In some embodiments, a nucleobase is or comprise an optionally substituted ring having at least one nitrogen atom. In some embodiments, a nucleobase comprise Ring BA as described herein, wherein at least one monocyclic ring of Ring BA comprise a nitrogen ring atom.

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytidine, 5-hydroxymethylcytidine, 5-formylcytosine, or 5-carboxylcytosine. In some embodiments, an oligonucleotide comprises one or more 5-methylcytidine. In some embodiments, each nucleobase in an oligonucleotide is selected from the group consisting of optionally substituted A, T, C, G and U, and optionally substituted tautomers of A, T, C, G and U. In some embodiments, each nucleobase in an oligonucleotide is optionally protected A, T, C, G and U. In some embodiments, each nucleobase in an oligonucleotide is optionally substituted A, T, C, G or U. In some embodiments, each nucleobase in an oligonucleotide is selected from the group consisting of A, T, C, G, U, and 5mC.

In some embodiments, a nucleobase, e.g., BA, comprises at least one optionally substituted ring which comprises a heteroatom ring atom. In some embodiments, a nucleobase comprises at least one optionally substituted ring which comprises a nitrogen ring atom. In some embodiments, such a ring is aromatic. In some embodiments, a nucleobase is bonded to a sugar through a heteroatom. In some embodiments, a nucleobase is bonded to a sugar through a nitrogen atom. In some embodiments, a nucleobase is bonded to a sugar through a ring nitrogen atom.

In some embodiments, a nucleobase, e.g., BA, is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the nucleobases of each of which are incorporated herein by reference.

In some embodiments, BA is optionally substituted or protected U, or is an optionally substituted or protected tautomer of U, or is optionally substituted or protected C, or is an optionally substituted or protected tautomer of C, or is optionally substituted or protected A, or is an optionally substituted or protected tautomer of A, or is optionally substituted or protected nucleobase of pseudoisocytosine, or is an optionally substituted or protected tautomer of the nucleobase of pseudoisocytosine.

In some embodiments, a nucleobase, e.g., BA, is an optionally substituted purine base residue. In some embodiments, a nucleobase is a protected purine base residue. In some embodiments, a nucleobase is an optionally substituted adenine residue. In some embodiments, a nucleobase is a protected adenine residue. In some embodiments, a nucleobase is an optionally substituted guanine residue. In some embodiments, a nucleobase is a protected guanine residue. In some embodiments, a nucleobase is an optionally substituted cytosine residue. In some embodiments, a nucleobase is a protected cytosine residue. In some embodiments, a nucleobase is an optionally substituted thymine residue. In some embodiments, a nucleobase is a protected thymine residue. In some embodiments, a nucleobase is an optionally substituted uracil residue. In some embodiments, a nucleobase is a protected uracil residue. In some embodiments, a nucleobase is an optionally substituted 5-methylcytosine residue. In some embodiments, a nucleobase is a protected 5-methylcytosine residue.

In some embodiments, a nucleobase, e.g., BA, is an optionally substituted group, which group is formed by removing a —H from

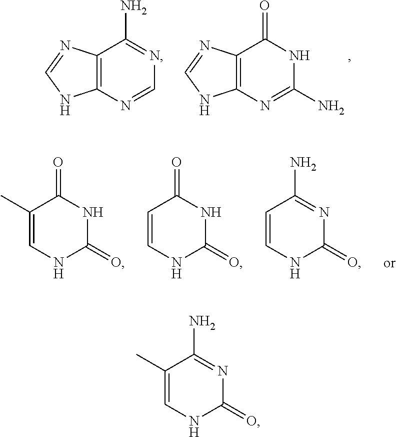

or a tautomer thereof. In some embodiments, a nucleobase, e.g., BA, is an optionally substituted group, which group is formed by removing a —H from

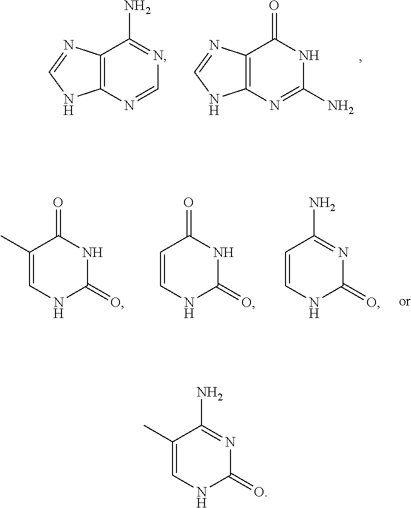

In some embodiments, a nucleobase, e.g., BA, is an optionally substituted group which group is selected from

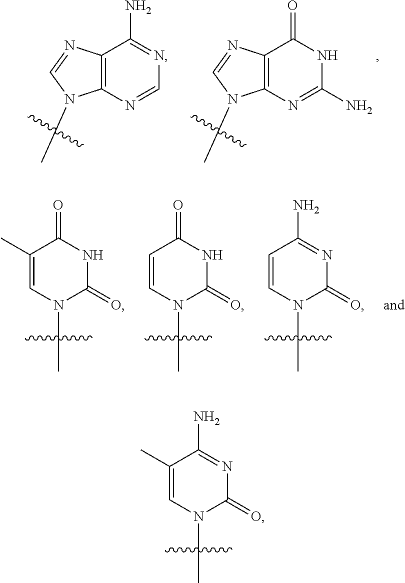

and tautomeric forms thereof. In some embodiments, a nucleobase, e.g., BA, is an optionally substituted group which group is selected from

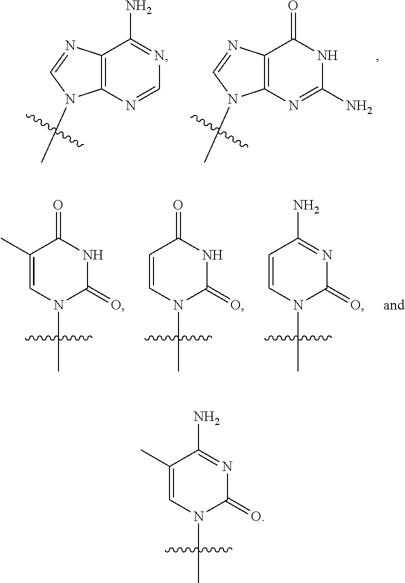

In some embodiments, a nucleobase, e.g., BA, is an optionally substituted group, which group is formed by removing a —H from

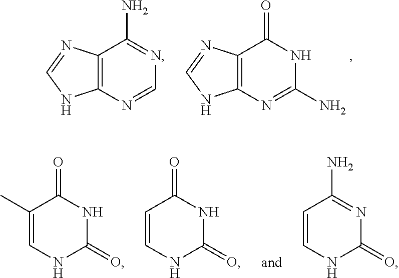

and tautomers thereof. In some embodiments, a nucleobase, e.g., BA, is an optionally substituted group, which group is formed by removing a —H from

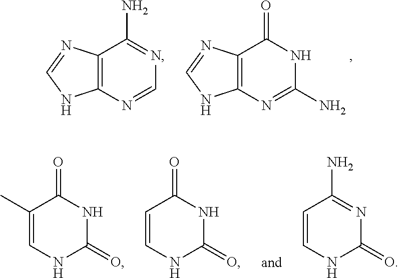

In some embodiments, a nucleobase, e.g., BA, is an optionally substituted group which group is selected from

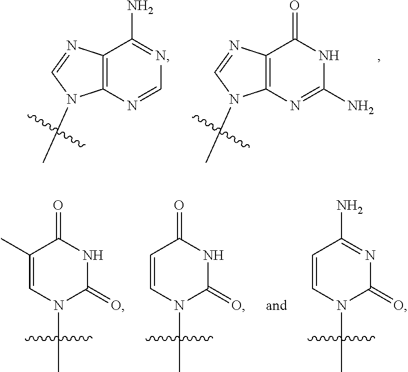

and tautomeric forms thereof. In some embodiments, a nucleobase, e.g., BA, is an optionally substituted group which group is selected from

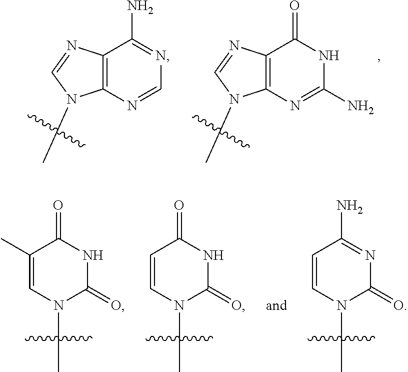

In some embodiments, a nucleobase, e.g., BA is optionally substituted

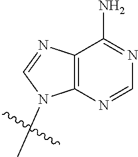 [SMILES: *n1cnc2c(N)ncnc21]

or a tautomeric form thereof. In some embodiments, a nucleobase, e.g., BA is optionally substituted

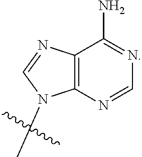 [SMILES: *n1cnc2c(N)ncnc21]

In some embodiments, a nucleobase, e.g., BA is optionally substituted

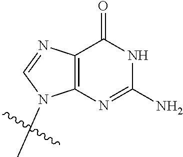 [SMILES: *n1cnc2c(=O)[nH]c(N)nc21]

or a tautomeric form thereof. In some embodiments, a nucleobase, e.g., BA is optionally substituted

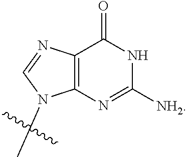 [SMILES: *n1cnc2c(=O)[nH]c(N)nc21]

In some embodiments, a nucleobase, e.g., BA is optionally substituted

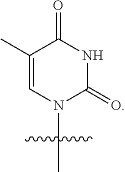 [SMILES: *n1cc(C)c(=O)[nH]c1=O]

or a tautomeric form thereof. In some embodiments, a nucleobase, e.g., BA is optionally substituted

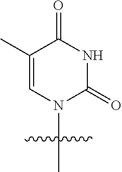 [SMILES: *n1cc(C)c(=O)[nH]c1=O]

In some embodiments, a nucleobase, e.g., BA is optionally substituted

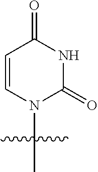 [SMILES: *n1ccc(=O)[nH]c1=O]

or a tautomeric form thereof. In some embodiments, a nucleobase, e.g., BA is optionally substituted

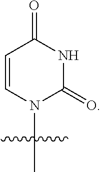 [SMILES: *n1ccc(=O)[nH]c1=O]

In some embodiments, a nucleobase, e.g., BA is optionally substituted

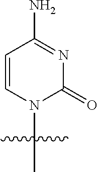 [SMILES: *n1ccc(N)nc1=O]

or a tautomeric form thereof. In some embodiments, a nucleobase, e.g., BA is optionally substituted

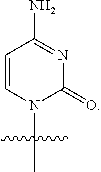 [SMILES: *n1ccc(N)nc1=O]

In some embodiments, a nucleobase, e.g., BA is

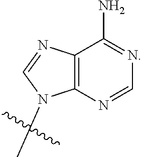 [SMILES: *n1cnc2c(N)ncnc21]

In some embodiments, a nucleobase, e.g., BA is

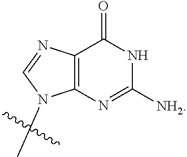 [SMILES: *n1cnc2c(=O)[nH]c(N)nc21]

In some embodiments, a nucleobase, e.g., BA is

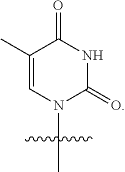 [SMILES: *n1cc(C)c(=O)[nH]c1=O]

In some embodiments, a nucleobase, e.g., BA is

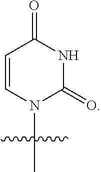 [SMILES: *n1ccc(=O)[nH]c1=O]

In some embodiments, a nucleobase, e.g., BA is

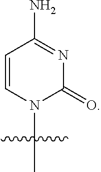 [SMILES: *n1ccc(N)nc1=O]

In some embodiments, a nucleobase, e.g., BA, is

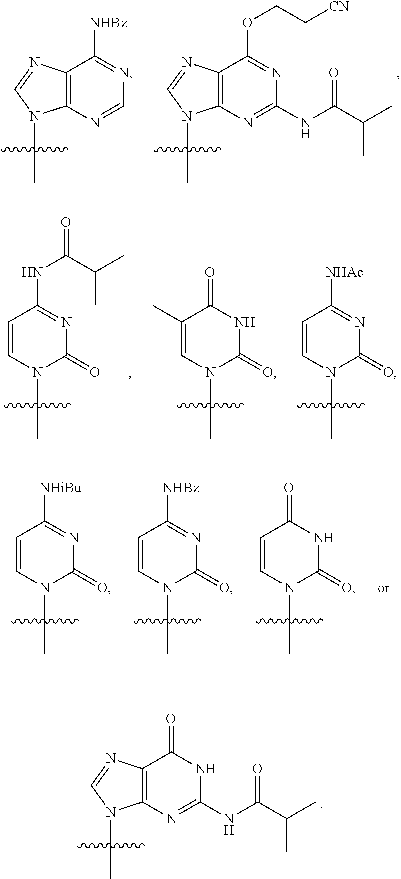

In some embodiments, a nucleobase, e.g., BA, is

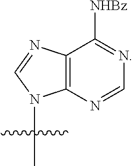

In some embodiments, a nucleobase, e.g., BA, is

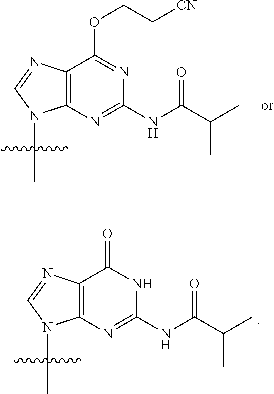 [SMILES: *n1cnc2c(OCCC#N)nc(NC(=O)C(C)C)nc21 ; *n1cnc2c(=O)[nH]c(NC(=O)C(C)C)nc21]

In some embodiments, a nucleobase, e.g., BA, is

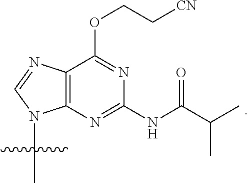 [SMILES: *n1cnc2c(OCCC#N)nc(NC(=O)C(C)C)nc21]

In some embodiments, a nucleobase, e.g., BA, is

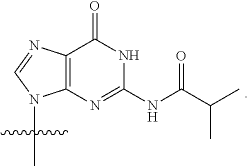 [SMILES: *n1cnc2c(=O)[nH]c(NC(=O)C(C)C)nc21]

In some embodiments, a nucleobase, e.g., BA, is

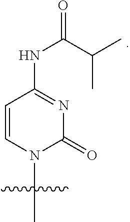 [SMILES: *n1ccc(NC(=O)C(C)C)nc1=O]

In some embodiments, a nucleobase, e.g., BA, is

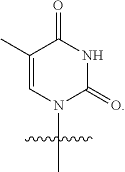 [SMILES: *n1cc(C)c(=O)[nH]c1=O]

In some embodiments, a nucleobase, e.g., BA, is

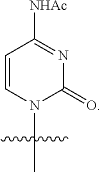 [SMILES: *n1ccc(NC(C)=O)nc1=O]

In some embodiments, a nucleobase, e.g., BA, is

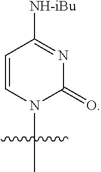

In some embodiments, a nucleobase, e.g., BA, is

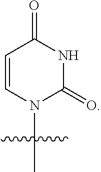 [SMILES: *n1ccc(=O)[nH]c1=O]

In some embodiments, a protection group is —Ac. In some embodiments, a protection group is -Bz. In some embodiments, a protection group is -iBu for nucleobase.

In some embodiments, a nucleobase, e.g., BA, is optionally substituted hypoxanthine or a tautomer thereof.

In some embodiments, a nucleobase, e.g., BA, is an optionally substituted purine base residue. In some embodiments, a nucleobase is a protected purine base residue. In some embodiments, a nucleobase is an optionally substituted adenine residue. In some embodiments, a nucleobase is a protected adenine residue. In some embodiments, a nucleobase is an optionally substituted guanine residue. In some embodiments, a nucleobase is a protected guanine residue. In some embodiments, a nucleobase is an optionally substituted cytosine residue. In some embodiments, a nucleobase is a protected cytosine residue. In some embodiments, a nucleobase is an optionally substituted thymine residue. In some embodiments, a nucleobase is a protected thymine residue. In some embodiments, a nucleobase is an optionally substituted uracil residue. In some embodiments, a nucleobase is a protected uracil residue. In some embodiments, a nucleobase is an optionally substituted 5-methylcytosine residue. In some embodiments, a nucleobase is a protected 5-methylcytosine residue.

In some embodiments, a nucleobase is a nucleobase illustrated in US 2011/0294124, US 2015/0211006, US 2015/0197540, WO 2015/107425, WO 2017/192679, WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the nucleobases of each of which are independently incorporated herein by reference. In some embodiments, BA is such a nucleobase.

### R¹

In some embodiments, R¹ is R as described herein. In some embodiments, R¹ is —H. In some embodiments, R¹ is not —H.

In some embodiments, R¹ is —P(O)(R²)₂ wherein each R² is independently as described herein. In some embodiments, at least one R² is not —H. In some embodiments, each R² is not —H. In some embodiments, at least one R² is —OR. In some embodiments, at least one R² is —OR wherein R is as described herein and is not —H. In some embodiments, each R² is independently —OR. In some embodiments, each R² is independently —OR wherein R is as described herein and is not —H. In some embodiments, at least one R² is independently —N(R′)₂, wherein each R′ is independently as described herein. In some embodiments, at least one R² is independently —N(R)₂, wherein each R is independently as described herein. In some embodiments, each R² is independently —N(R′)₂, wherein each R′ is independently as described herein. In some embodiments, each R² is independently —N(R)₂, wherein each R is independently as described herein. In some embodiments, at least one R² is

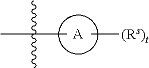

as described herein. In some embodiments, each R² is independently

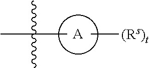

as described herein.

In some embodiments, R¹ is —S(O)₂R². In some embodiments, R² is R as described herein. In some embodiments, R² is R as described herein and is not —H. In some embodiments, R² is optionally substituted C₁₋₁₀ aliphatic. In some embodiments, R² is C₁₋₆ aliphatic. In some embodiments, R² is C₁₋₆ alkyl. In some embodiments, R² is methyl. In some embodiments, R² is ethyl. In some embodiments, R² is n-propyl. In some embodiments, R² is isopropyl. In some embodiments, R² is n-butyl. In some embodiments, R² is cyclobutyl. In some embodiments, R² is cyclopentyl. In some embodiments, R² is cyclopropyl. In some embodiments, R² is cyclohexyl. In some embodiments, R² is optionally substituted phenyl. In some embodiments, R² is phenyl. In some embodiments, R² is —OR. In some embodiments, R² is —OR wherein R is not —H. In some embodiments, R² is —N(R′)₂ wherein each R′ is independently as described herein. In some embodiments, R² is —N(R)₂ wherein each R is independently as described herein. In some embodiments, R² is —NMe₂. In some embodiments, R² is

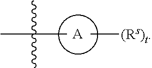

In some embodiments, Ring A is an optionally substituted phenyl ring (as appreciated by those skilled in the art, in addition to —S(O)₂— and R group(s)). Various useful embodiments of R and t are described herein as examples. In some embodiments, R¹ is —S(O)₂R² wherein R² is optionally substituted phenyl. In some embodiments, R¹ is —S(O)₂R² wherein R² is phenyl.

In some embodiments, R¹ is —Si(R)₃ wherein each R is independently described therein. In some embodiments, each R is not —H. In some embodiments, R¹ is —Si(R)₃, wherein each R is independently an optionally substituted C₁₋₃₀ aliphatic group. In some embodiments, R¹ is —Si(R)₃, wherein each R is independently an optionally substituted C₁₋₁₀ aliphatic group. In some embodiments, R¹ is —Si(R)₃, wherein each R is independently selected from the group of methyl, ethyl, propyl, isopropyl, n-butyl, tert-butyl, cyclobutyl, cyclopentyl, and cyclohexyl. In some embodiments, R¹ is —Si(R)₃, wherein each R is independently an optionally substituted C₁₋₄ aliphatic group. In some embodiments, R¹ is —Si(R)₃, wherein each R is independently methyl. In some embodiments, R¹ is —Si(R)₃, wherein each R is independently ethyl. In some embodiments, R¹ is —Si(R)₃, wherein each R is independently propyl. In some embodiments, R¹ is —Si(R)₃, wherein each R is independently isopropyl. In some embodiments, R¹ is —Si(R)₃, wherein each R is independently n-butyl. In some embodiments, R¹ is —Si(R)₃, wherein each R is independently tert-butyl.

In some embodiments, R¹ is —Si(R)₃, wherein each R is independently an optionally substituted group selected from C₁₋₃₀ aliphatic and C₆₋₃₀ aryl. In some embodiments, R¹ is —Si(R)₃, wherein each R is independently an optionally substituted group selected from C₁₋₁₀ aliphatic and phenyl. In some embodiments, R¹ is —Si(R)₃, wherein each R is independently an optionally substituted group selected from C₁₋₄ aliphatic and phenyl. In some embodiments, R¹ is —Si(R)₃, wherein each R is independently an optionally substituted group selected from C₁₋₄ aliphatic and phenyl, wherein the substituent is halogen, —CN, —C(O)OR′, —OR′, or —N(R′)₂, wherein R′ is described therein. In some embodiments, R¹ is —Si(R)₃, wherein each R is independently C₁₋₄ aliphatic or optionally substituted phenyl, wherein the substituent is halogen, —CN, —C(O)OR′, —OR′, or —N(R′)₂, wherein R′ is described therein. In some embodiments, R¹ is —Si(R)₃, wherein each R is independently C₁₋₄ aliphatic or phenyl. In some embodiments, R¹ is —Si(R)₃ wherein one R group is optionally substituted C₁₋₆ aliphatic and the other two are independently optionally substituted phenyl. In some embodiments, R¹ is —Si(Ph)₂Me.

### R²

In some embodiments, R² is R′ as described herein. In some embodiments, R² is R as described herein. In some embodiments, R² is —H. In some embodiments, R² is not —H. In some embodiments, R² is optionally substituted C₁₋₁₀ aliphatic. In some embodiments, R² is optionally substituted C₁₋₁₀ alkyl. In some embodiments, R² is C₁₋₁₀ alkyl. In some embodiments, R² is methyl. In some embodiments, R² is ethyl. In some embodiments, R² is isopropyl. In some embodiments, R² is n-butyl. In some embodiments, R² is cyclobutyl. In some embodiments, R² is cyclopentyl. In some embodiments, R² is cyclohexyl. In some embodiments, R² is optionally substituted phenyl. In some embodiments, R² is phenyl.

In some embodiments, R² is —OR. In some embodiments, R² is —OH. In some embodiments, R² is —OR wherein R is not —H. In some embodiments, R is optionally substituted C₁₋₆ aliphatic.

In some embodiments, R² is —N(R′)₂ wherein each R′ is independently as described herein. In some embodiments, R² is —NHR′ wherein R′ is as described herein. In some embodiments, R² is —N(R)₂ wherein each R is independently as described herein. In some embodiments, R² is —NHR wherein R is as described herein. In some embodiments, R² is —NH₂. In some embodiments, R² is —N(R)₂ wherein each R is independently C₁₋₆ aliphatic. In some embodiments, R² is —NMe₂. In some embodiments, R² is —N(Et)₂. In some embodiments, R² is —N(Me)Et.

In some embodiments, R² is

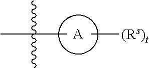

as described herein.

In some embodiments, one occurrence of R² is

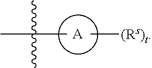

In some embodiments, t is 1 and Ring A is optionally substituted

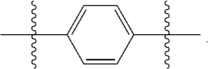 [SMILES: *c1ccc(*)cc1]

In some embodiments, R² is optionally substituted

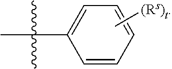

In some embodiments, one occurrence of R² is

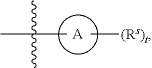

the other occurrence is —OR.
In some embodiments, one occurrence of R² is

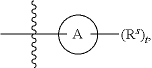

the other occurrence is —R.

### Ring A

As described herein, Ring A is optionally substituted (in addition to the group

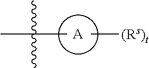

is bonded to and the R^{s} groups). In some embodiments, Ring A is substituted. In some embodiments, Ring A is unsubstituted.

In some embodiments, Ring A is an optionally substituted 5-10 membered aromatic ring having 0-5 heteroatoms independently selected from oxygen, nitrogen, and sulfur. In some embodiments, Ring A is an optionally substituted 5-6 membered aromatic ring having 0-5 heteroatoms independently selected from oxygen, nitrogen, and sulfur. In some embodiments, Ring A is an optionally substituted phenyl ring. In some embodiments, Ring A is a phenyl ring. In some embodiments, Ring A is an optionally substituted 10-membered bicyclic aryl ring. In some embodiments, Ring A is an optionally substituted 5-9 membered heteroaryl ring having 1-4 heteroatoms independently selected from nitrogen, oxygen and sulfur. In some embodiments, Ring A is an optionally substituted 5-membered heteroaryl ring having 1-4 heteroatoms independently selected from nitrogen, oxygen and sulfur. In some embodiments, Ring A is an optionally substituted 6-membered heteroaryl ring having 1-4 heteroatoms independently selected from nitrogen, oxygen and sulfur. In some embodiments, Ring A is an optionally substituted 9-membered bicyclic heteroaryl ring having 1-4 heteroatoms independently selected from nitrogen, oxygen and sulfur. In some embodiments, a heteroatom is nitrogen. In some embodiments, Ring A is protected.

### R^{s}

In some embodiments, an occurrence of R^{s} is R′ as described herein. In some embodiments, an occurrence of R^{s} is R as described herein. In some embodiments, an occurrence of R^{s} is —H. In some embodiments, an occurrence of R^{s} is not —H. In some embodiments, each occurrence of R^{s} is not —H.

In some embodiments, an occurrence of R^{s} is R as described herein and is not —H. For example, in some embodiments, it is optionally substituted C₆₋₁₀ aryl. In some embodiments, it is optionally substituted phenyl. In some embodiments, it is optionally substituted heteroaryl, e.g., 5-6 membered heteroaryl having 1-4 heteroatoms independently selected from nitrogen, oxygen and sulfur.

In some embodiments, an occurrence of R^{s} is halogen. In some embodiments, an occurrence of R^{s} is F. In some embodiments, an occurrence of R^{s} is Cl. In some embodiments, an occurrence of R^{s} is Br. In some embodiments, an occurrence of R^{s} is I. In some embodiments, an occurrence of R^{s} is —CN.

In some embodiments, an occurrence of R^{s} is C(O)OR′, wherein R′ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, an occurrence of R^{s} is C(O)OR, wherein R is as described herein. In some embodiments, an occurrence of R^{s} is C(O)OR, wherein R is as described herein and is not —H. In some embodiments, an occurrence of R^{s} is —C(O)OMe. In some embodiments, an occurrence of R^{s} is —C(O)OEt.

In some embodiments, an occurrence of R^{s} is —OR′, wherein R′ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R′ is optionally substituted C₁₋₆ aliphatic. In some embodiments, an occurrence of R^{s} is —OH. In some embodiments, an occurrence of R^{s} is —OR wherein R is as described herein and is not —H. In some embodiments, an occurrence of R^{s} is —OMe. In some embodiments, an occurrence of R^{s} is -OEt. In some embodiments, an occurrence of R^{s} is —O-propyl. In some embodiments, an occurrence of R^{s} is —O-isopropyl. In some embodiments, an occurrence of R^{s} is —O— butyl. In some embodiments, an occurrence of R^{s} is —O-tert-butyl. In some embodiments, an occurrence of R^{s} is —O—CH₂-Ph. In some embodiments, an occurrence of R^{s} is —O-Ph.

In some embodiments, an occurrence of R^{s} is —N(R′)₂, wherein R′ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, each R′ is independently H or optionally substituted C₁₋₆ aliphatic. In some embodiments, an occurrence of R^{s} is —NH₂. In some embodiments, an occurrence of R^{s} is —N(R)₂, wherein each variable is independent as described herein. In some embodiments, an occurrence of R^{s} is —NHMe. In some embodiments, an occurrence of R^{s} is —NMe₂. In some embodiments, an occurrence of R^{s} is -NHEt. In some embodiments, an occurrence of R^{s} is —N(Et)₂.

In some embodiments, an occurrence of R^{s} is optionally substituted C₆₋₁₀ aryl. In some embodiments, it is optionally substituted phenyl. In some embodiments, it is phenyl. In some embodiments, an occurrence of R^{s} is 5-20 membered heteroaryl having 1-3 heteroatoms independently selected from oxygen, nitrogen, sulfur, phosphorus and silicon. In some embodiments, an occurrence of R^{s} is

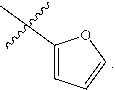 [SMILES: *c1ccco1]

In some embodiments, an occurrence of R^{s} is

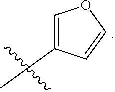 [SMILES: *c1ccoc1]

In some embodiments, an occurrence of R^{s} is

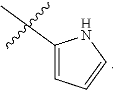 [SMILES: *c1ccc[nH]1]

In some embodiments, an occurrence of R^{s} is

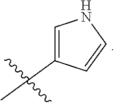 [SMILES: *c1cc[nH]c1]

In some embodiments, an occurrence of R^{s} is

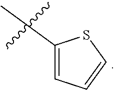 [SMILES: *c1cccs1]

In some embodiments, an occurrence of R^{s} is

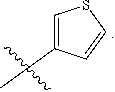 [SMILES: *c1ccsc1]

In some embodiments, an occurrence of R^{s} is

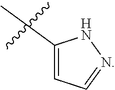 [SMILES: *c1ccn[nH]1]

In some embodiments, an occurrence of R^{s} is

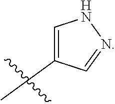 [SMILES: *c1cn[nH]c1]

In some embodiments, an occurrence of R^{s} is

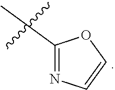 [SMILES: *c1ncco1]

In some embodiments, an occurrence of R² is

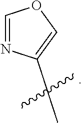 [SMILES: *c1cocn1]

In some embodiments, an occurrence of R^{s} is

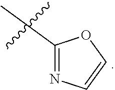 [SMILES: *c1ncco1]

In some embodiments, an occurrence of R^{s} is

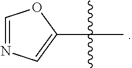 [SMILES: *c1cnco1]

t

In some embodiments, t is 0. In some embodiments, t is 1-5. In some embodiments, t is 1. In some embodiments, t is 2. In some embodiments, t is 3. In some embodiments, t is 4. In some embodiments, t is 5.

### L

In some embodiments, L is —CH₂—. In some embodiments, L is substituted —CH₂—. In some embodiments, L is —CH₂— substituted with one or two suitable substituents. In some embodiments, L is mono-substituted. In some embodiments, L is di-substituted. In some embodiments, L is —CH(CN)—.

### Ring B

As described herein, R^{a} and R^{b} are taken together with their intervening atoms to form an optionally substituted Ring B. In some embodiments, Ring B is optionally substituted (in addition to the groups that are bonded to the nitrogen atom to which R^{a} is bonded and the carbon atom to which R is bonded). In some embodiments, Ring B is substituted. In some embodiments, Ring B is unsubstituted.

In some embodiments, Ring B is 4-15, 4-12, 4-10, or 4-7 membered. In some embodiments, Ring B is 4-membered. In some embodiments, Ring B is 5-membered. In some embodiments, Ring B is 6-membered. In some embodiments, Ring B is 7-membered. In some embodiments, Ring B is 8-membered. In some embodiments, Ring B is 9-membered. In some embodiments, Ring B is 10-membered. In some embodiments, Ring B is 11-membered. In some embodiments, Ring B is 12-membered. In some embodiments, Ring B is 13-membered. In some embodiments, Ring B is 14-membered. In some embodiments, Ring B is 15-membered.

In some embodiments, Ring B is saturated. In some embodiments, Ring B is partially unsaturated. In some embodiments, the carbon to which R^{a} is bonded is sp³.

In some embodiments, Ring B is monocyclic. In some embodiments, Ring B is bicyclic. In some embodiments, Ring B is polycyclic. In some embodiments, each monocyclic unit is independently a 3-10 (e.g., 3-7, 4-7, 3-6, 3, 4, 5, 6, 7, 8, 9, or 10) membered saturated, partially unsaturated or aromatic ring having 0-5 (e.g., 0, 1-5, 1, 2, 3, 4, 5, etc.) heteroatoms. In some embodiments, each monocyclic unit is independently a 3-7 (e.g., 3, 4, 5, 6, 7, etc.) membered saturated, partially unsaturated or aromatic ring having 0-4 heteroatoms independently selected from nitrogen, oxygen and sulfur. In some embodiments, each monocyclic unit is independently a 4-7 membered saturated, partially unsaturated or aromatic ring having 0-4 heteroatoms independently selected from nitrogen, oxygen and sulfur. In some embodiments, each monocyclic unit is independently a 5-7 membered saturated, partially unsaturated or aromatic ring having 0-4 heteroatoms independently selected from nitrogen, oxygen and sulfur. In some embodiments, each monocyclic unit is independently saturated or partially unsaturated. In some embodiments, each monocyclic unit is independently saturated.

In some embodiments, Ring B has 0 heteroatoms in addition to the nitrogen atom to which R is attached. In some embodiments, Ring B has 1-4 additional heteroatoms. In some embodiments, Ring B has 1 additional heteroatom. In some embodiments, Ring B has 2 additional heteroatoms. In some embodiments, Ring B has 3 additional heteroatoms. In some embodiments, Ring B has 4 additional heteroatoms. In some embodiments, each additional heteroatom is independently selected from nitrogen, oxygen and sulfur.

In some embodiments, Ring B is an optionally substituted azetidine ring. In some embodiments, Ring B is an optionally substituted pyrrolidine ring. In some embodiments, Ring B is an optionally substituted piperidine ring.

In some embodiments, Ring B is optionally substituted

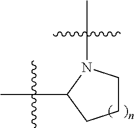

and n is 0, 1, 2, or 3. In some embodiments, Ring B is optionally substituted

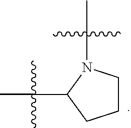 [SMILES: *C1CCCN1*]

In some embodiments, Ring B is

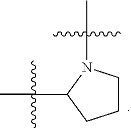 [SMILES: *C1CCCN1*]

n

In some embodiments, n is 0. In some embodiments, n is 1. In some embodiments, n is 2. In some embodiments, n is 3.

In some embodiments, the present disclosure provides a compound as described herein having a diastereomeric purity of about or at least about 10%, 15%, 20%, 25%, 30%, 35%, 40%, 45%, 50%, 55%, 60%, 65%, 70%, 75%, 80%, 85%, 90%, or 100%. In some embodiments, a compound as described herein having a diastereomeric purity of about or at least about 10%. In some embodiments, a compound as described herein having a diastereomeric purity of about or at least about 15%. In some embodiments, a compound as described herein having a diastereomeric purity of about or at least about 20%. In some embodiments, a compound as described herein having a diastereomeric purity of about or at least about 25%. In some embodiments, a compound as described herein having a diastereomeric purity of about or at least about 30%. In some embodiments, a compound as described herein having a diastereomeric purity of about or at least about 35%. In some embodiments, a compound as described herein having a diastereomeric purity of about or at least about 40%. In some embodiments, a compound as described herein having a diastereomeric purity of about or at least about 45%. In some embodiments, a compound as described herein having a diastereomeric purity of about or at least about 50%. In some embodiments, a compound as described herein having a diastereomeric purity of about or at least about 55%. In some embodiments, a compound as described herein having a diastereomeric purity of about or at least about 60%. In some embodiments, a compound as described herein having a diastereomeric purity of about or at least about 65%. In some embodiments, a compound as described herein having a diastereomeric purity of about or at least about 70%. In some embodiments, a compound as described herein having a diastereomeric purity of about or at least about 75%. In some embodiments, a compound as described herein having a diastereomeric purity of about or at least about 80%. In some embodiments, a compound as described herein having a diastereomeric purity of about or at least about 85%. In some embodiments, a compound as described herein having a diastereomeric purity of about or at least about 90%. In some embodiments, a compound as described herein having a diastereomeric purity of about or at least about 100%.

In some embodiments, the present disclosure provides a compound as described herein having a purity of about or at least about 10%, 15%, 20%, 25%, 30%, 35%, 40%, 45%, 50%, 55%, 60%, 65%, 70%, 75%, 80%, 85%, 90%, or 100%. In some embodiments, a compound as described herein having a purity of about or at least about 10%. In some embodiments, a compound as described herein having a purity of about or at least about 15%. In some embodiments, a compound as described herein having a purity of about or at least about 20%. In some embodiments, a compound as described herein having a purity of about or at least about 25%. In some embodiments, a compound as described herein having a purity of about or at least about 30%. In some embodiments, a compound as described herein having a purity of about or at least about 35%. In some embodiments, a compound as described herein having a purity of about or at least about 40%. In some embodiments, a compound as described herein having a purity of about or at least about 45%. In some embodiments, a compound as described herein having a purity of about or at least about 50%. In some embodiments, a compound as described herein having a purity of about or at least about 55%. In some embodiments, a compound as described herein having a purity of about or at least about 60%. In some embodiments, a compound as described herein having a purity of about or at least about 65%. In some embodiments, a compound as described herein having a purity of about or at least about 70%. In some embodiments, a compound as described herein having a purity of about or at least about 75%. In some embodiments, a compound as described herein having a purity of about or at least about 80%. In some embodiments, a compound as described herein having a purity of about or at least about 85%. In some embodiments, a compound as described herein having a purity of about or at least about 90%. In some embodiments, a compound as described herein having a purity of about or at least about 100%.

In some embodiments, the ratio of the compound as described herein and its diastereomer with respect to the chiral phosphorus is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%. In some embodiments, the ratio of the compound as described herein and its diastereomer with respect to the chiral phosphorus is about or at least about 50%:50%. In some embodiments, the ratio of the compound as described herein and its diastereomer with respect to the chiral phosphorus is about or at least about 60%:40%. In some embodiments, the ratio of the compound as described herein and its diastereomer with respect to the chiral phosphorus is about or at least about 70%:30%. In some embodiments, the ratio of the compound as described herein and its diastereomer with respect to the chiral phosphorus is about or at least about 80%:20%. In some embodiments, the ratio of the compound as described herein and its diastereomer with respect to the chiral phosphorus is about or at least about 90%:10%. In some embodiments, the ratio of the compound as described herein and its diastereomer with respect to the chiral phosphorus is about or at least about 91%:9%. In some embodiments, the ratio of the compound as described herein and its diastereomer with respect to the chiral phosphorus is about or at least about 92%:8%. In some embodiments, the ratio of the compound as described herein and its diastereomer with respect to the chiral phosphorus is about or at least about 93%:7%. In some embodiments, the ratio of the compound as described herein and its diastereomer with respect to the chiral phosphorus is about or at least about 94%:6%. In some embodiments, the ratio of the compound as described herein and its diastereomer with respect to the chiral phosphorus is about or at least about 95%:5%. In some embodiments, the ratio of the compound as described herein and its diastereomer with respect to the chiral phosphorus is about or at least about 96%:4%. In some embodiments, the ratio of the compound as described herein and its diastereomer with respect to the chiral phosphorus is about or at least about 97%:3%. In some embodiments, the ratio of the compound as described herein and its diastereomer with respect to the chiral phosphorus is about or at least about 98%:2%. In some embodiments, the ratio of the compound as described herein and its diastereomer with respect to the chiral phosphorus is about or at least about 99%:1%. In some embodiments, the ratio of the compound as described herein and its diastereomer with respect to the chiral phosphorus is about or at least about 99.5%:0.5%. In some embodiments, the ratio of the compound as described herein and its diastereomer with respect to the chiral phosphorus is about or at least about 99.9%:0.1%.

In some embodiments, the ratio of cis isomer:trans isomer with respect to the chiral phosphorus of the compound as described herein is about or at least about 50%:50%, about 60%:40%, about 70%:30%, about 80%:20%, about 90%:10%, about 91%:9%, about 92%:8%, about 93%:7%, about 94%:6%, about 950%:50, about 96%:4%, about 97%:3%, about 98%:2%, about 99%:1%, about 99.5%:0.5%, or about 99.9%:0.1%. In some embodiments, the ratio of cis isomer:trans isomer with respect to the chiral phosphorus is about or at least about 50%:50%. In some embodiments, the ratio of cis isomer:trans isomer with respect to the chiral phosphorus is about or at least about 60%:40%. In some embodiments, the ratio of cis isomer trans isomer with respect to the chiral phosphorus is about or at least about 70%:30%. In some embodiments, the ratio of cis isomer:trans isomer with respect to the chiral phosphorus is about or at least about 80%:20%. In some embodiments, the ratio of cis isomer:trans isomer with respect to the chiral phosphorus is about or at least about 90%:10%. In some embodiments, the ratio of cis isomer:trans isomer with respect to the chiral phosphorus is about or at least about 91%:9%. In some embodiments, the ratio of cis isomer:trans isomer with respect to the chiral phosphorus is about or at least about 92%:8%. In some embodiments, the ratio of cis isomer:trans isomer with respect to the chiral phosphorus is about or at least about 93%:7%. In some embodiments, the ratio of cis isomer:trans isomer with respect to the chiral phosphorus is about or at least about 94%:6%. In some embodiments, the ratio of cis isomer:trans isomer with respect to the chiral phosphorus is about or at least about 95%:5%. In some embodiments, the ratio of cis isomer:trans isomer with respect to the chiral phosphorus is about or at least about 96%:4%. In some embodiments, the ratio of cis isomer:trans isomer with respect to the chiral phosphorus is about or at least about 97%:3%. In some embodiments, the ratio of cis isomer:trans isomer with respect to the chiral phosphorus is about or at least about 98%:2%. In some embodiments, the ratio of cis isomer:trans isomer with respect to the chiral phosphorus is about or at least about 99%:1%. In some embodiments, the ratio of cis isomer:trans isomer with respect to the chiral phosphorus is about or at least about 99.5%:0.5%. In some embodiments, the ratio of cis isomer:trans isomer with respect to the chiral phosphorus is about or at least about 99.9%:0.1%.

In some embodiments, the ratio of trans isomer:cis isomer with respect to the chiral phosphorus of the compound as described herein is about or at least about 50%:50%, about 60%:40%, about 70%:30%, about 80%:20%, about 90%:10%, about 91%:9%, about 92%:8%, about 93%:7%, about 94%:6%, about 95%:0:5%, about 96%:4%, about 97%:3%, about 98%:2%, about 99%:1%, about 99.5%:0.5%, or about 99.9%:0.1%. In some embodiments, the ratio of trans isomer:cis isomer with respect to the chiral phosphorus is about or at least about 50%:50%. In some embodiments, the ratio of trans isomer:cis isomer with respect to the chiral phosphorus is about or at least about 60%:40%. In some embodiments, the ratio of trans isomer:cis isomer with respect to the chiral phosphorus is about or at least about 70%:30%. In some embodiments, the ratio of trans isomer:cis isomer with respect to the chiral phosphorus is about or at least about 80%:20%. In some embodiments, the ratio of trans isomer:cis isomer with respect to the chiral phosphorus is about or at least about 90%:10%. In some embodiments, the ratio of trans isomer:cis isomer with respect to the chiral phosphorus is about or at least about 91%:9%. In some embodiments, the ratio of trans isomer:cis isomer with respect to the chiral phosphorus is about or at least about 92%:8%. In some embodiments, the ratio of trans isomer:cis isomer with respect to the chiral phosphorus is about or at least about 93%:7%. In some embodiments, the ratio of trans isomer:cis isomer with respect to the chiral phosphorus is about or at least about 94%:6%. In some embodiments, the ratio of trans isomer:cis isomer with respect to the chiral phosphorus is about or at least about 95%:5%. In some embodiments, the ratio of trans isomer:cis isomer with respect to the chiral phosphorus is about or at least about 96%:4%. In some embodiments, the ratio of trans isomer:cis isomer with respect to the chiral phosphorus is about or at least about 97%:3%. In some embodiments, the ratio of trans isomer:cis isomer with respect to the chiral phosphorus is about or at least about 98%:2%. In some embodiments, the ratio of trans isomer:cis isomer with respect to the chiral phosphorus is about or at least about 99%:10%. In some embodiments, the ratio of trans isomer:cis isomer with respect to the chiral phosphorus is about or at least about 99.5%:0.5%. In some embodiments, the ratio of trans isomer:cis isomer with respect to the chiral phosphorus is about or at least about 99.9%:0.10%.

In some embodiments, the present disclosure provides a method of preparing a compound of

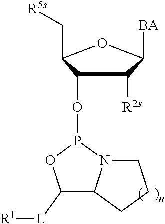

or a salt thereof, and the ratio of trans isomer:cis isomer with respect to the chiral phosphorus is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%. In some embodiments, the ratio of cis isomer:trans isomer with respect to the chiral phosphorus is about or at least about 50%:50%. In some embodiments, the ratio of cis isomer:trans isomer with respect to the chiral phosphorus is about or at least about 60%:40%. In some embodiments, the ratio of cis isomer:trans isomer with respect to the chiral phosphorus is about or at least about 70%:30%. In some embodiments, the ratio of cis isomer:trans isomer with respect to the chiral phosphorus is about or at least about 80%:20%. In some embodiments, the ratio of cis isomer:trans isomer with respect to the chiral phosphorus is about or at least about 90%:10%. In some embodiments, the ratio of cis isomer:trans isomer with respect to the chiral phosphorus is about or at least about 91%:9%. In some embodiments, the ratio of cis isomer:trans isomer with respect to the chiral phosphorus is about or at least about 92%:8%. In some embodiments, the ratio of cis isomer:trans isomer with respect to the chiral phosphorus is about or at least about 93%:7%. In some embodiments, the ratio of cis isomer:trans isomer with respect to the chiral phosphorus is about or at least about 94%:6%. In some embodiments, the ratio of cis isomer:trans isomer with respect to the chiral phosphorus is about or at least about 95%:5%. In some embodiments, the ratio of cis isomer:trans isomer with respect to the chiral phosphorus is about or at least about 96%:4%. In some embodiments, the ratio of cis isomer:trans isomer with respect to the chiral phosphorus is about or at least about 97%:3%. In some embodiments, the ratio of cis isomer:trans isomer with respect to the chiral phosphorus is about or at least about 98%:2%. In some embodiments, the ratio of cis isomer:trans isomer with respect to the chiral phosphorus is about or at least about 99%:1%. In some embodiments, the ratio of cis isomer:trans isomer with respect to the chiral phosphorus is about or at least about 99.5%:0.5%. In some embodiments, the ratio of cis isomer:trans isomer with respect to the chiral phosphorus is about or at least about 99.9%:0.1%.

In some embodiments, the present disclosure provides a method of preparing a compound of

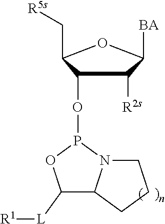

or a salt thereof, and the ratio of trans isomer:cis isomer with respect to the chiral phosphorus is about or less than about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%. In some embodiments, the ratio of cis isomer:trans isomer with respect to the chiral phosphorus is about or less about 50%:50%. In some embodiments, the ratio of cis isomer:trans isomer with respect to the chiral phosphorus is about or less about 60%:40%. In some embodiments, the ratio of cis isomer:trans isomer with respect to the chiral phosphorus is about or less about 70%:30%. In some embodiments, the ratio of cis isomer:trans isomer with respect to the chiral phosphorus is about or less about 80%:20%. In some embodiments, the ratio of cis isomer:trans isomer with respect to the chiral phosphorus is about or less about 90%:10%. In some embodiments, the ratio of cis isomer:trans isomer with respect to the chiral phosphorus is about or less about 91%:9%. In some embodiments, the ratio of cis isomer:trans isomer with respect to the chiral phosphorus is about or less about 92%:8%. In some embodiments, the ratio of cis isomer:trans isomer with respect to the chiral phosphorus is about or less about 93%:7%. In some embodiments, the ratio of cis isomer:trans isomer with respect to the chiral phosphorus is about or less about 94%:6%. In some embodiments, the ratio of cis isomer:trans isomer with respect to the chiral phosphorus is about or less about 95%:5%. In some embodiments, the ratio of cis isomer:trans isomer with respect to the chiral phosphorus is about or less about 96%:4%. In some embodiments, the ratio of cis isomer:trans isomer with respect to the chiral phosphorus is about or less about 97%:3%. In some embodiments, the ratio of cis isomer:trans isomer with respect to the chiral phosphorus is about or less about 98%:2%. In some embodiments, the ratio of cis isomer:trans isomer with respect to the chiral phosphorus is about or less about 99%:1%. In some embodiments, the ratio of cis isomer:trans isomer with respect to the chiral phosphorus is about or less about 99.5%:0.5%. In some embodiments, the ratio of cis isomer:trans isomer with respect to the chiral phosphorus is about or less about 99.9%:0.1%.

In some embodiments, a pair of trans isomer and cis isomer are

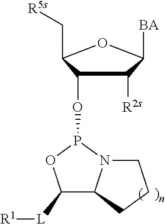

or a salt thereof and

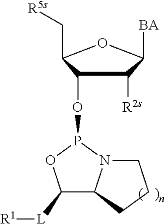

or a salt thereof, respectively. In some embodiments, a pair of trans isomer and cis isomer are

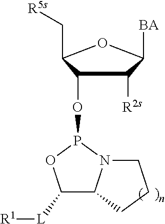

or a salt thereof and

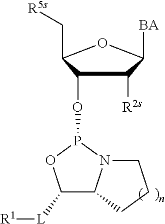

or a salt thereof, respectively.

In some embodiments, a method provided herein is performed in the presence of base. In some embodiments, a base is a sterically hindered base (compared to triethyl amine). In some embodiments, a base is of low nucleophilicity (compared to triethyl amine). In some embodiments, a base is a tertiary amine that has the structure of N(R)₃ wherein the three R groups are taken together with nitrogen to form an optionally substituted 8-20 (e.g., 8-10, 8, 9, 10, 11, 12, 13, 14, 15, 16, 17, 18, 19, 20, etc.) membered bicyclic or polycyclic ring having 0-3 (e.g., 0, 1-3, 1, 2, 3, etc.) heteroatoms in addition to the nitrogen atom. In some embodiments, a base is a tertiary amine that has the structure of N(R)₃ wherein the three R groups are taken together with nitrogen to form an optionally substituted 8-20 (e.g., 8-10, 8, 9, 10, 11, 12, 13, 14, 15, 16, 17, 18, 19, 20, etc.) membered bicyclic or polycyclic ring having 0-3 (e.g., 0, 1-3, 1, 2, 3, etc.) nitrogen atoms in addition to the nitrogen atom. In some embodiments, a base is DBU. In some embodiments, a base is DBN. In some embodiments, a base is DABCO. In some embodiments, a base is N-methylmorpholine (NMM). In some embodiments, a base is N,N-diisopropylethylamine (DIPEA). In some embodiments, a base is dibutyl aniline. In some embodiments, a base or a mixture of bases comprising a base provides higher levels of cis phosphoramidites compared to a reference base, e.g., TEA.

In some embodiments, equivalent of a base is about or at least about 1, 1.1, 1.2, 1.3, 1.4, 1.5, 1.6, 1.7, 1.8, 1.9, 2, 2.5, 3, 3.5, 4, 4.5, 5, 6, 7, 8, 9, or 10 relative to a nucleoside. In some embodiments, equivalent of a base is about or at least about 1 relative to a nucleoside. In some embodiments, equivalent of a base is about or at least about 1.1 relative to a nucleoside. In some embodiments, equivalent of a base is about or at least about 1.2 relative to a nucleoside. In some embodiments, equivalent of a base is about or at least about 1.3 relative to a nucleoside. In some embodiments, equivalent of a base is about or at least about 1.4 relative to a nucleoside. In some embodiments, equivalent of a base is about or at least about 1.5 relative to a nucleoside. In some embodiments, equivalent of a base is about or at least about 1.6 relative to a nucleoside. In some embodiments, equivalent of a base is about or at least about 1.7 relative to a nucleoside. In some embodiments, equivalent of a base is about or at least about 1.8 relative to a nucleoside. In some embodiments, equivalent of a base is about or at least about 1.9 relative to a nucleoside. In some embodiments, equivalent of a base is about or at least about 2 relative to a nucleoside. In some embodiments, equivalent of a base is about or at least about 2.5 relative to a nucleoside. In some embodiments, equivalent of a base is about or at least about 3 relative to a nucleoside. In some embodiments, equivalent of a base is about or at least about 3.5 relative to a nucleoside. In some embodiments, equivalent of a base is about or at least about 4 relative to a nucleoside. In some embodiments, equivalent of a base is about or at least about 4.5 relative to a nucleoside. In some embodiments, equivalent of a base is about or at least about 5 relative to a nucleoside. In some embodiments, equivalent of a base is about or at least about 6 relative to a nucleoside. In some embodiments, equivalent of a base is about or at least about 7 relative to a nucleoside. In some embodiments, equivalent of a base is about or at least about 8 relative to a nucleoside. In some embodiments, equivalent of a base is about or at least about 9 relative to a nucleoside. In some embodiments, equivalent of a base is about or at least about 10 relative to a nucleoside.

In some embodiments, a method provided herein is performed in the presence of another base. In some embodiments, the ratio of a first base and another base is about or at least about 5:1. In some embodiments, the ratio of a first base and another base is about or at least about 4:1. In some embodiments, the ratio of a first base and another base is about or at least about 3:1. In some embodiments, the ratio of a first base and another base is about or at least about 2:1. In some embodiments, the ratio of a first base and another base is about or at least about 1:1. In some embodiments, an another base is triethylamine (TEA). In some embodiments, an another base is N-methylmorpholine (NMM). In some embodiments, a first base is DBU. In some embodiments, a first base is DBN. In some embodiments, equivalent of a first base is about or at least about 1, 1.1, 1.2, 1.3, 1.4, 1.5, 1.6, 1.7, 1.8, 1.9, 2, 2.5, 3, 3.5, 4, 4.5, 5, 6, 7, 8, 9, or 10 relative to a nucleoside as described herein. In some embodiments, a mixture is 1:1 NMM: DBu. In some embodiments, a mixture is 1:1 NMM: DBN. In some embodiments, a mixture is 1:1 DBU: TEA. In some embodiments, a mixture is 1:1 DBN: TEA. In some embodiments, a mixture is 2:1 DBN: TEA. In some embodiments, a mixture is 3:1 DBN: TEA.

In some embodiments, an acid, e.g., a mildly acidic compound, provides increased levels of cis cyclic phosphoramidites. In some embodiments, an acid is pentafluorophenol.

In some embodiments, a method provided herein is performed at a reduced temperature. In some embodiments, a provided method comprises the reaction temperature from a reduced temperature to an ambient temperature (about 25° C.). In some embodiments, a reduced temperature is about −78° C. In some embodiments, a reduced temperature is about −20° C. In some embodiments, a reduced temperature is about 0° C.

In some embodiments, the present disclosure provides a method for isomerizing a compound as described herein with respect to its chiral phosphorus, comprising contacting the compound with a phosphoramidite activator for oligonucleotide synthesis.

In some embodiments, the present disclosure provides a method for isomerizing a compound as described herein with respect to its chiral phosphorus, comprising contacting the compound with an acid for oligonucleotide synthesis.

In some embodiments, the present disclosure provides a method for preparing an oligonucleotide, comprising steps of:
1) contacting a phosphoramidite composition comprising a compound as described herein with an acid to provide an isomerized phosphoramidite composition, wherein the isomerized phosphoramidite composition comprises a lower level of the compound compared to the phosphoramidite composition; and
2) contacting the isomerized phosphoramidite composition with a coupling partner.

In some embodiments, the present disclosure provides a method for preparing an oligonucleotide, comprising steps of:
1) contacting a phosphoramidite composition comprising a compound as described herein with a phosphoramidite activator to provide an isomerized phosphoramidite composition, wherein the isomerized phosphoramidite composition comprises a lower level of the compound compared to the phosphoramidite composition; and
2) contacting the isomerized phosphoramidite composition with a coupling partner.

In some embodiments, a coupling partner comprises —OH. In some embodiments, a coupling partner is or comprise a nucleoside as described herein. In some embodiments, a coupling partner is an oligonucleotide. In some embodiments, a coupling partner is linked to a solid support. In some embodiments, a coupling partner is linked to a solid support through a linker. In some embodiments, provided technologies provide epimerization of P chiral centers, e.g., in phosphoramidites as described herein.

Provided technologies can be utilized with various phosphoramidites, e.g., U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the phosphoramidites of each of which are independently incorporated herein by reference.

In some embodiments, an activator is an acid. In some embodiments, an activator is a mildly acidic compound. In some embodiments, an acid is a mild acid. In some embodiments, a method for isomerizing a compound as described herein with respect to its chiral phosphorus, comprising contacting the compound with a mildly acidic compound. In some embodiments, a mildly acidic compound is a salt of a base with an acid. In some embodiments, a base has the structure of N(R)₃, wherein two R groups are taken together with the nitrogen to form an optionally substituted 5-10 membered ring having 0-3 (e.g., 0, 1-3, 1, 2, 3, etc.) heteroatoms in addition to the nitrogen atom. In some embodiments, a mildly acidic compound is a salt of a heteroaryl base comprising a sp2 nitrogen atom. In some embodiments, a mildly acidic compound is a salt of a heteroaryl base comprising a sp3 nitrogen atom.

In some embodiments, an activator is a mildly acidic compound. In some embodiments, a method for isomerizing a compound as described herein with respect to its chiral phosphorus, comprising contacting the compound with a salt of a base. In some embodiments, a salt is a triflate.

In some embodiments, an activator is CMPT. In some embodiments, an activator is CMIMT. In some embodiments, an activator is 4-nitrophenol.

In some embodiments, pKa of a compound, e.g., an acid, an activator, etc., is about or at least about 1, 2, 3, 4, 5, 6, 7, 8, 9, 10, 11, 12, or 13. In some embodiments, it is about or at least 4. In some embodiments, it is about or at least 5. In some embodiments, it is about or at least 6. In some embodiments, it is about or at least 7. In some embodiments, it is about or at least 8. In some embodiments, it is about or at least 9. In some embodiments, it is about or at least 10. In some embodiments, pKa is for a solvent (e.g., water) at a specific temperature (e.g., about 25° C.).

Certain activators are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the activators of each of which are independently incorporated herein by reference.

As shown in the Figures, in some embodiments, provided technologies can provide rapid epimerization of chiral phosphorus in phosphoramidites. In some embodiments, about or at least about 10%, 20%, 30%, 40%, 50%, 60%, 70%, 80%, 90%, 95%, 99%, or about 100% epimerization is achieved. In some embodiments, such levels of epimerization is achieved within, e.g., within about or no more than about 1, 2, 3, 4, 5, 6, 7, 8, 9, 10, 11, 12, 13, 14, 15, 16, 17, 18, 19, or 20 minutes.

In some embodiments, the present disclosure provides a method for assessing level of a compound in a composition, comprising using a compound or composition as described herein as a reference.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

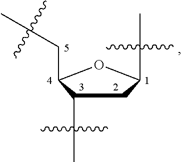

wherein a nucleobase is attached to the 1′ position, and the 3′ and 5′ positions are connected to internucleotidic linkages (as appreciated by those skilled in the art, if at the 5′-end of oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

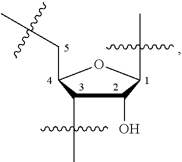

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target nucleic acid recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In some embodiments, as most commonly in natural nucleic acids, an internucleotidic linkage connects with one sugar at the 5′ position and another sugar at the 3′ position unless otherwise indicated.

In some embodiments, a sugar is an optionally substituted natural DNA or RNA sugar. In some embodiments, a sugar is optionally substituted

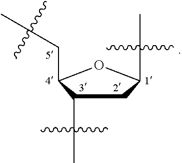

In some embodiments, the 2′ position is optionally substituted. In some embodiments, a sugar is

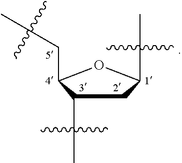

In some embodiments, a sugar has the structure of

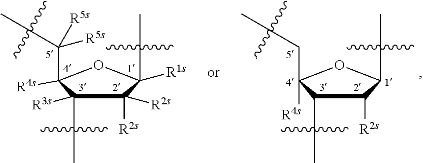

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the substituents, sugar modifications, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —F, —Cl, —Br, —I, —CN, —N₃, —NO, —NO₂, -L^{s}-R′, -L^{s}-OR′, -L^{s}-SR′, -L^{s}-N(R′)₂, —O-L^{s}-OR′, —O-L^{s}-SR′, or —O-L^{s}-N(R′)₂, wherein each R′ is independently as described herein, and each L^{s} is independently a covalent bond or optionally substituted bivalent C₁₋₆ aliphatic or heteroaliphatic having 1-4 heteroatoms; or two R^{s} are taken together to form a bridge -L^{s}-. In some embodiments, R′ is optionally substituted C₁₋₁₀ aliphatic. In some embodiments, a sugar has the structure of

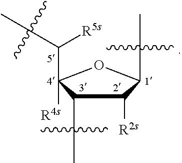

In some embodiments, a sugar has the structure of

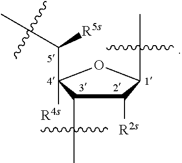

In some embodiments, a sugar has the structure of

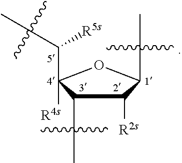

In some embodiments, a sugar has the structure of

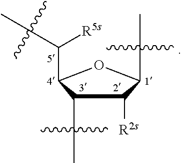

In some embodiments, a sugar has the structure of

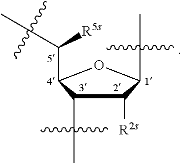

In some embodiments, a sugar has the structure of

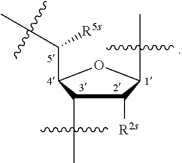

In some embodiments, a sugar has the structure of

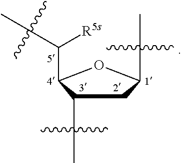

In some embodiments, a sugar has the structure of

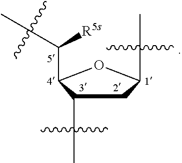

In some embodiments, a sugar has the structure of

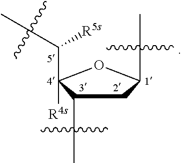

In some embodiments, R^{5s} is optionally substituted C₁₋₆ aliphatic. In some embodiments, R^{5s} is optionally substituted C₁₋₆ alkyl. In some embodiments, R^{5s} is optionally substituted methyl. In some embodiments, R^{5s} is methyl. In some embodiments, a sugar has the structure of

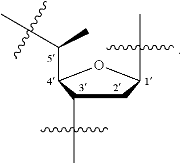

In some embodiments, a sugar has the structure of

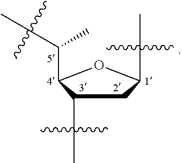

In some embodiments, a sugar has the structure of

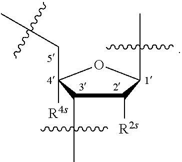

Various such sugars are utilized in Table 1. In some embodiments, a sugar has the structure of

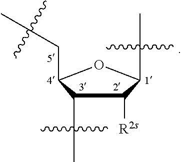

In some embodiments, a 2′-modified sugar has the structure of

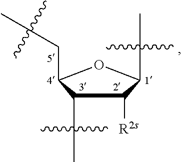

wherein R^{2s} is a 2′-modification. In some embodiments, a sugar has the structure of

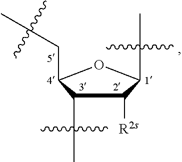

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, a modified nucleoside is mA, mT, mC, m5mC, mG, mU, etc., in which R^{2s} is —OMe. In some embodiments, R^{2s} is —OCH₂CH₂OMe. In some embodiments, a modified nucleoside is Aeo, Teo, Ceo, m5Ceo, Geo, Ueo, etc., in which R^{2s} is —OCH₂CH₂OMe. In some embodiments, R^{2s} is —OCH₂CH₂OH. In some embodiments, an oligonucleotide comprises a 2′-F modified sugar having the structure of

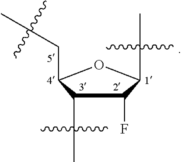

In some embodiments, an oligonucleotide comprises a 2′-OMe modified sugar having the structure of

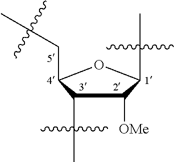

(e.g., as in m(U), m(A), etc.). In some embodiments, an oligonucleotide comprises a 2′-MOE modified sugar having the structure of

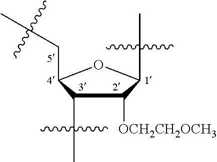

(e.g., as in [moe](G), [moe]([m5C]), etc.).

In some embodiments, a sugar has the structure of

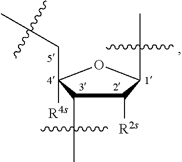

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a sugar has the structure of

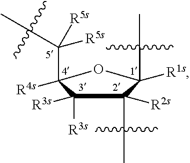

wherein each variable is independently as described herein. In some embodiments, a sugar has the structure of

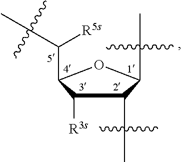

wherein each variable is independently as described herein. In some embodiments, R^{5s} is —H. In some embodiments, a sugar has the structure of

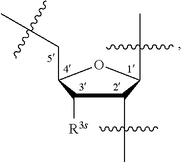

wherein each variable is independently as described herein. In some embodiments, R^{3S} is —OH. In some embodiments, R^{3S} is —H. In some embodiments, a sugar is

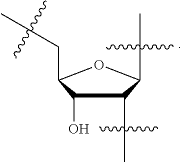

In some embodiments, a sugar is

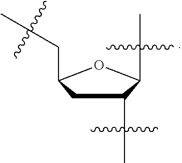 [SMILES: *C[C@@H]1C[C@H](*)[C@H](*)O1]

In some embodiments, a sugar is optionally substituted

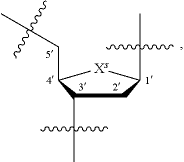

wherein X^{s} is —S—, —Se—, or optionally substituted —CH₂—. In some embodiments, the 2′ position is optionally substituted. In some embodiments, a sugar is

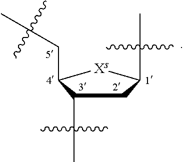

In some embodiments, a sugar has the structure of

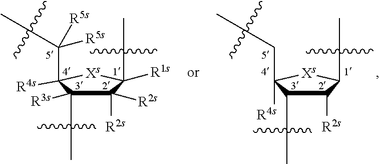

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the substituents, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —F, —Cl, —Br, —I, —CN, —N₃, —NO, —NO₂, -L^{s}-R′, -L^{s}-OR′, -L^{s}-SR′, -L^{s}-N(R′)₂, —O-L^{s}-OR′, —O-L^{s}-SR′, or —O-L^{s}-N(R′)₂, wherein each R′ is independently as described herein, and each L^{s} is independently a covalent bond or optionally substituted bivalent C₁₋₆ aliphatic or heteroaliphatic having 1-4 heteroatoms; or two R^{s} are taken together to form a bridge -L^{s}-. In some embodiments, R′ is optionally substituted C₁₋₁₀ aliphatic. In some embodiments, a sugar has the structure of R^{2s}

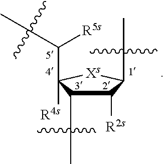

In some embodiments, a sugar has the structure of

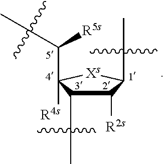

In some embodiments, a sugar has the structure of

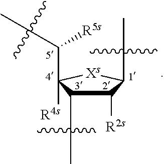

In some embodiments, a sugar has the structure of

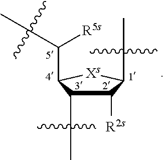

In some embodiments, a sugar has the structure of

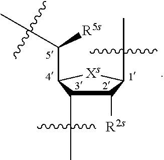

In some embodiments, a sugar has the structure of

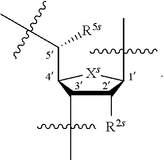

In some embodiments, a sugar has the structure of

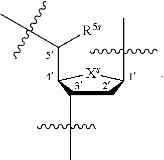

In some embodiments, a sugar has the structure of

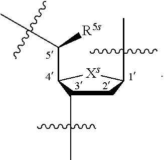

In some embodiments, a sugar has the structure of

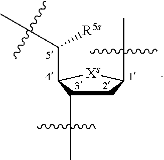

In some embodiments, R^{5s} is optionally substituted C₁₋₆ aliphatic. In some embodiments, R^{5s} is optionally substituted C₁₋₆ alkyl. In some embodiments, R^{5s} is optionally substituted methyl. In some embodiments, R^{5s} is methyl. In some embodiments, a sugar has the structure of

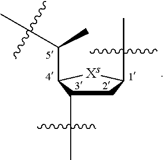

In some embodiments, a sugar has the structure of

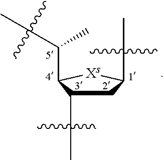

In some embodiments a sugar has the structure of

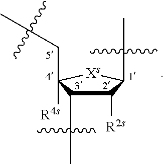

Various such sugars are utilized in Table 1. In some embodiments, a sugar has the structure of

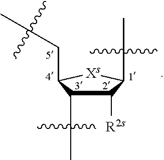

In some embodiments, a 2′-modified sugar has the structure of

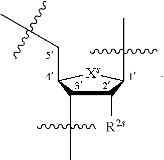

wherein R^{2s} is a 2′-modification. In some embodiments, a sugar has the structure of

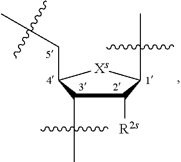

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, R^{2s} is —OCH₂CH₂OMe. In some embodiments, R^{2s}is —OCH₂CH₂OH. In some embodiments, a modified sugar has the structure of

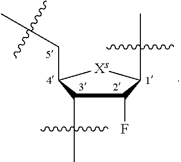

In some embodiments, a modified sugar has the structure of

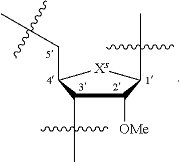

In some embodiments, a modified sugar having the structure of

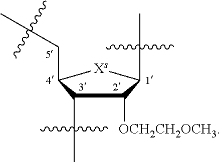

In some embodiments, a modified sugar having the structure of

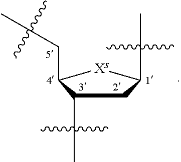

In some embodiments, X^{s} is —S—. In some embodiments, X^{s} is optionally substituted —CH₂—. In some embodiments, X^{s} is —CH₂—. In some embodiments, a modified sugar having the structure of

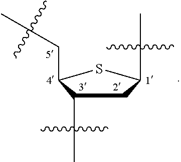

In some embodiments, a modified sugar having the structure of

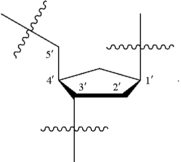

In some embodiments, a sugar has the structure of

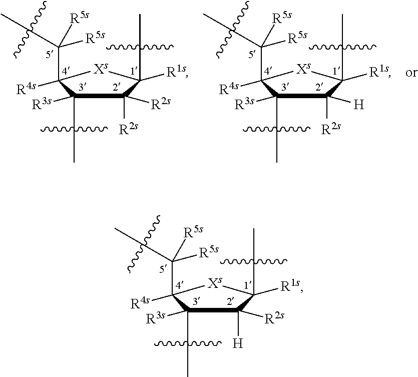

wherein each R^{2s} is independently —H, —F, —OH or —OR^{ak}, wherein R^{ak} is optionally substituted C₁₋₆ aliphatic, and each of the other variables is independently as described herein. In some embodiments, each of R^{1s}, R^{3s}, R^{4s}, and R^{5s} is independently —H. In some embodiments, each of R^{1s}, R^{3S} and R^{4s}, and one of R^{5s}, are independently —H, and the other R^{5s} is independently C₁₋₆ aliphatic. In some embodiments, an occurrence of R^{5s} is C₁₋₆ aliphatic, e.g., methyl. In some embodiments, R^{2s} is —H. In some embodiments, R^{2s} is —F. In some embodiments, R^{2s} is —OR^{ak}. In some embodiments, R^{2s} is —OMe. In some embodiments, R^{2s} is —OCH₂CH₂CH₃. In some embodiments, at least one occurrence of R^{2s} is —H. In some embodiments, at least one occurrence of R^{2s} is not —H. In some embodiments, X^{s} is —O—. In some embodiments, X^{s} is —S—. In some embodiments, X^{s} is optionally substituted —CH₂—. In some embodiments, X^{s} is —CH₂—.

In some embodiments, a sugar has the structure of

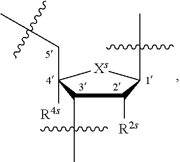

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4. In some embodiments, X^{s} is —S—. In some embodiments, X^{s} is optionally substituted —CH₂—. In some embodiments, X is —CH₂—. In some embodiments, X is —Se—.

In some embodiments, a sugar has the structure of

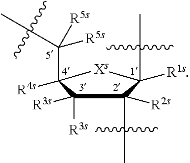

wherein each variable is independently as described herein. In some embodiments, a sugar has the structure of

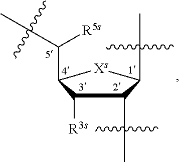

wherein each variable is independently as described herein. In some embodiments, R^{5s} is —H. In some embodiments, a sugar has the structure of

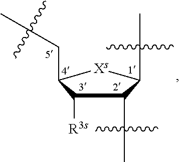

wherein each variable is independently as described herein. In some embodiments, R^{3S} is —OH. In some embodiments, R^{3S} is —H. In some embodiments, X^{s} is —S—. In some embodiments, X^{s} is optionally substituted —CH₂—. In some embodiments, X^{s} is —CH₂—.

In some embodiments, a nucleoside comprising a modified sugar has the structure of

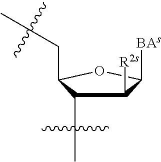

or a salt form thereof, wherein BA^{s} is —H or an optionally substituted or protected nucleobase (e.g., BA), and R^{2s} is as described herein. In some embodiments, R^{2s} is —OH, halogen, or optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, R^{2s} is —F. In some embodiments, a nucleoside comprising a modified sugar has the structure of

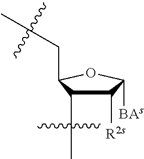

or a salt form thereof, wherein each variable is independently as described herein. In some embodiments, R^{2s} is —H, —OH, halogen, or optionally substituted C₁-C₆ alkoxy. In some embodiments, R^{2s} is —H. In some embodiments, R^{2s} is —F. In some embodiments, a nucleoside comprising a modified sugar has the structure of

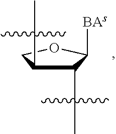

wherein each variable is as described herein. In some embodiments, a nucleoside comprising a modified sugar has the structure of

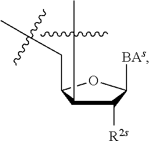

or a salt form thereof, wherein each variable is independently as described herein. In some embodiments, R^{2s} is —H, —OH, halogen, or optionally substituted C₁-C₆ alkoxy. In some embodiments, R^{2s} is —H. In some embodiments, R^{2s} is —F. In some embodiments, a nucleoside comprising a modified sugar has the structure of

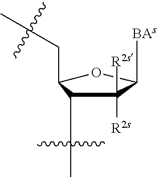

or a salt form thereof, wherein R^{2s′} is R^{s}, and each of R^{s}, R^{2s} and BA^{s} is independently as described herein. In some embodiments, each of R^{2s} and R^{2s′} is independently —H, —OH, halogen, or optionally substituted C₁-C₆ alkoxy. In some embodiments, R^{2s} is —H. In some embodiments, R^{2s} is —OH. In some embodiments, R^{2s} is halogen. In some embodiments, R^{2s} is —F. In some embodiments, R^{2s} is optionally substituted C₁-C₆ alkoxy. In some embodiments, R^{2s′} is —H. In some embodiments, R^{2s′} is —OH. In some embodiments, R^{2s′} is halogen. In some embodiments, R^{2s′} is —F. In some embodiments, R^{2s′} is optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. Certain such nucleosides including sugars and nucleobases and uses thereof are described in WO 2020/154342. In some embodiments, an oligonucleotide comprises arabinoside, 2′-deoxy-2′-fluoro-arabinoside, 2′-OR arabinoside, a deoxycytidine, DNA-abasic, RNA-abasic, or 2′-OR abasic, wherein R is not hydrogen (e.g., optionally substituted C₁₋₆ aliphatic). In some embodiments, 2′-OR is 2′-OMe. In some embodiments, 2′-OR is 2′-MOE. In some embodiments, an oligonucleotide comprises 2′-O-methyl-arabinocytidine (amC). In some embodiments, oligonucleotides comprise such nucleosides. In some embodiments, monomers comprise such nucleosides. In some embodiments, phosphoramidites comprise such nucleosides (in some embodiments, one connecting site (e.g., a —CH₂— connecting site) is bonded to an optionally substituted —OH, e.g., (-ODMTr), and one connecting site (e.g., a ring connecting site) is bonded to O which is also bonded to P of a phosphoramidite). In some embodiments, one or more or each of a 5′ immediate nucleoside (e.g., N₁), an opposite nucleoside (N₀) and a 3′ immediate nucleoside (e.g., N₋₁) is independently such a nucleoside. In some embodiments, 5′-N₁N₀N₋₁-3′ is amCCA. In some embodiments, a sugar has the structure of

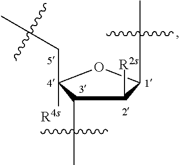

wherein each variable is as described herein and Cl′ is bonded to a nucleobase. In some embodiments, a sugar is an arabinose. In some embodiments, a sugar has the structure of

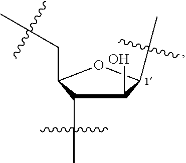

wherein Cl′ is bonded to a nucleobase.

In some embodiments, a sugar is optionally substituted

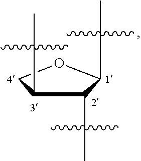

wherein a nucleobase is bonded at position 1′. In some embodiments, a sugar is

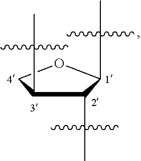

wherein a nucleobase is bonded at position 1′.

In some embodiments, a sugar is optionally substitute

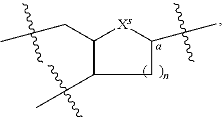

wherein position a is bonded to a nucleobase, X is —O—, —S—, —Se— or optionally substituted —CH₂—. In some embodiments, a sugar is

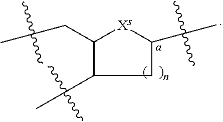

In some embodiments, a sugar is optionally substituted

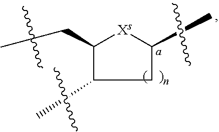

wherein position a is bonded to a nucleobase, X^{s} is —O—, —S—, —Se— or optionally substituted —CH₂—. In some embodiments, a sugar is

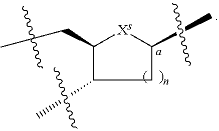

In some embodiments, X^{s} is —O—. In some embodiments, X^{s} is —S—. In some embodiments, X is —Se—. In some embodiments, X^{s} is optionally substituted —CH₂—. In some embodiments, X is —CH₂—. In some embodiments, n is 0. In some embodiments, n is 1. In some embodiments, n is 2. In some embodiments, n is 3.

In some embodiments, a modified sugar comprises an optionally substituted 6-membered ring having 0-1 oxygen atom. In some embodiments, a modified sugar comprises an optionally substituted 6-membered ring having an oxygen atom. For example, in some embodiments, a modified sugar has the structure of optionally substituted

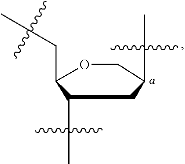

wherein position a is bonded to a nucleobase. In some embodiments, a modified sugar has the structure of

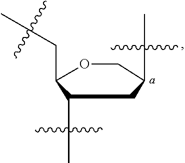

wherein position a is bonded to a nucleobase. In some embodiments, a modified sugar has the structure of optionally substituted

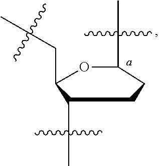

wherein position a is bonded to a nucleobase. In some embodiments, a modified sugar has the structure of

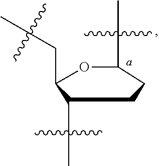

wherein position a is bonded to a nucleobase. In some embodiments, a modified sugar has the structure of

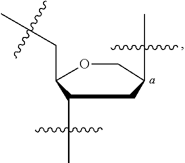

wherein position a is bonded to a nucleobase. In some embodiments, a modified sugar has the structure of optionally substituted

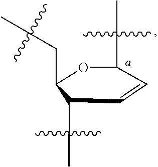

wherein position a is bonded to a nucleobase. In some embodiments, a modified sugar has the structure of

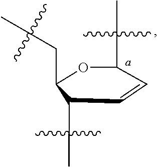

wherein position a is bonded to a nucleobase. In some embodiments, a modified sugar has the structure of optionally substituted

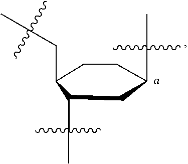

wherein position a is bonded to a nucleobase. In some embodiments, a modified sugar has the structure of

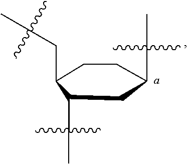

wherein position a is bonded to a nucleobase. In some embodiments, a modified sugar has the structure of optionally substituted

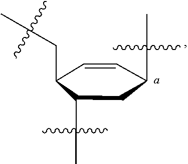

wherein position a is bonded to a nucleobase. In some embodiments, a modified sugar has the structure of

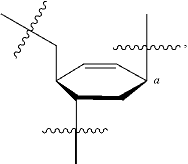

wherein position a is bonded to a nucleobase.

In some embodiments, a nucleoside comprising a modified sugar has the structure of

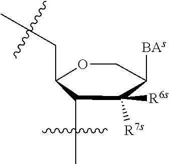

or a salt form thereof, wherein each of R^{6s} and R^{7s} is independently R^{s}, BA^{s} is —H or an optionally substituted or protected nucleobase (e.g., BA), and R^{s} is independently as described herein. In some embodiments, R^{6s} is —H, —OH or halogen, and R^{7s} is —H, —OH, halogen or optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, a nucleoside comprising a modified sugar has the structure of

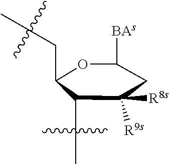

or a salt form thereof, wherein each of R^{8s} and R^{9s} is independently R^{s}, and each of R^{s} and BA^{s} is independently as described herein. In some embodiments, R^{8s} is —H or halogen, and R^{9s} is —H, —OH, halogen, or optionally substituted C₁-C₆ alkoxy. In some embodiments, a nucleoside comprising a modified sugar has the structure of

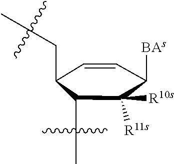

or a salt form thereof, wherein each of R^{10s} and R^{11s} is independently R^{s}, and each of R^{s} and BA^{s} is independently as described herein. In some embodiments, R^{10s} is —H or halogen, and R^{11s} is —H, —OH, halogen, or optionally substituted C₁-C₆ alkoxy. In some embodiments, a nucleoside comprising a modified sugar has the structure of

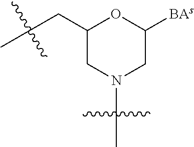

or a salt form thereof, wherein BA^{s} is as described herein. In some embodiments, a nucleoside comprising a modified sugar has the structure of

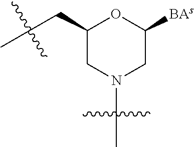

or a salt form thereof, wherein BA^{s} is as described herein. Those skilled in the art appreciate that in some embodiments, the nitrogen may be directly bonded to linkage phosphorus. In some embodiments, a halogen is —F. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. In some embodiments, an oligonucleotide comprises alpha-homo-DNA, beta-homo-DNA moieties. Certain such nucleosides including sugars and nucleobases and uses thereof are described in WO 2020/154343. In some embodiments, oligonucleotides comprise such nucleosides. In some embodiments, monomers comprise such nucleosides. In some embodiments, phosphoramidites comprise such nucleosides (in some embodiments, one connecting site (e.g., a —CH₂— connecting site) is bonded to an optionally substituted —OH, e.g., -ODMTr, and one connecting site (e.g., a ring connecting site) is bonded to P of a phosphoramidite (e.g., when the connecting ring atom is N) or to O which is also bonded to P of a phosphoramidite(e.g., when the connecting ring atom is C)). In some embodiments, one or more or each of a 5′ immediate nucleoside (e.g., N₁), an opposite nucleoside (N₀) and a 3′ immediate nucleoside (e.g., N₋₁) is independently such a nucleoside.

In some embodiments, a modified sugar has the structure of

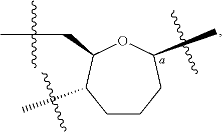

wherein position a is bonded to a nucleobase. In some embodiments, a modified sugar has the structure of

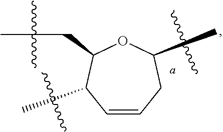

wherein position a is bonded to a nucleobase. In some embodiments, a modified sugar has the structure of

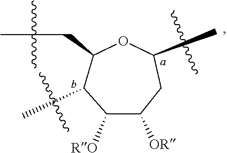

wherein position a is bonded to a nucleobase, position b is bonded to an internucleoside linkage and R″ is —H or optionally substituted C₁₋₆ aliphatic. In some embodiments, a modified sugar has the structure of

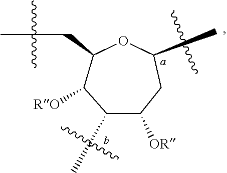

wherein position a is bonded to a nucleobase, position b is bonded to an internucleoside linkage and R″ is —H or C₁₋₆ aliphatic. In some embodiments, a modified sugar has the structure of

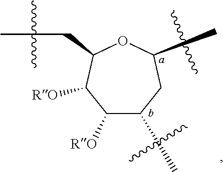

wherein position a is bonded to a nucleobase, position b is bonded to an internucleoside linkage and R″ is —H or C₁₋₆ aliphatic. In some embodiments, R″ is methyl.

In some embodiments, a nucleoside comprising a modified sugar has the structure of

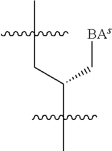

or a salt form thereof, wherein each variable is as described herein. In some embodiments, a nucleoside comprising a modified sugar has the structure of

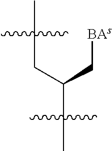

or a salt form thereof, wherein each variable is as described herein. In some embodiments, a nucleoside comprising a modified sugar has the structure of

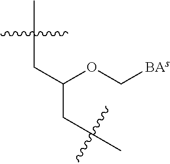

or a salt form thereof, wherein each variable is as described herein. In some embodiments, a nucleoside comprising a modified sugar has the structure of

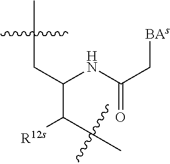

or a salt form thereof, wherein R^{12s} is R^{s}, and each of R^{s} and BA^{s} is independently as described herein. In some embodiments, R^{12s} is —H, —OH, halogen, optionally substituted C₁₋₆ alkyl, optionally substituted C₁₋₆ heteroalkyl, or optionally substituted C₁₋₆ alkoxy. In some embodiments, a halogen is —F. In some embodiments, a nucleoside comprising a modified sugar has the structure of

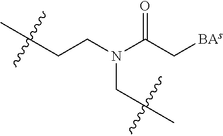

or a salt form thereof, wherein each variable is as described herein. In some embodiments, a nucleotide comprising a modified sugar has the structure of

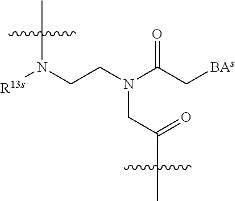

or a salt form thereof, wherein R^{13s} is R^{s}, and each of R^{s} and BA^{s} is independently as described herein. In some embodiments, R^{13s}, is —H or optionally substituted C₁-C₆ alkyl. In some embodiments, a nucleoside comprising a modified sugar has the structure of

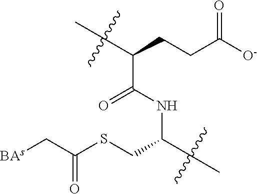

or a salt form thereof, wherein each variable is as described herein. In some embodiments, a nucleotide comprising a modified sugar has the structure of

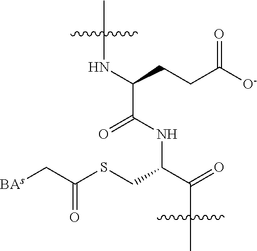

or a salt form thereof, wherein each variable is as described herein. In some embodiments, a linkage is an amide linkage. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. Certain such nucleosides and nucleotides including sugars and nucleobases and uses thereof are described in WO 2020/154344. In some embodiments, oligonucleotides comprise such nucleosides. In some embodiments, oligonucleotides comprise such nucleosides (in some embodiments, one connecting site (e.g., a —CH₂— connecting site) is bonded to an optionally substituted —OH, e.g., (-ODMTr), and one connecting site (e.g., a ring connecting site) is bonded to O which is also bonded to P of a phosphoramidite. In some embodiments, one or more or each of a 5′ immediate nucleoside (e.g., N₁), an opposite nucleoside (N₀) and a 3′ immediate nucleoside (e.g., N₋₁) is independently such a nucleoside.

In some embodiments, a sugar is an acyclic sugar, e.g. a UNA sugar. In some embodiments, a sugar is optionally substituted

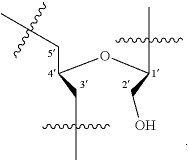

In some embodiments, the 2′ position is optionally substituted. In some embodiments, a sugar is

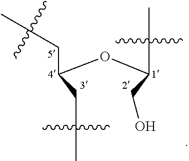

In some embodiments, a sugar has the structure of

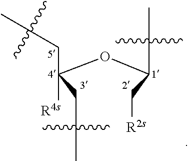

In some embodiments, R^{2s} is —OH. In some embodiments, a sugar is

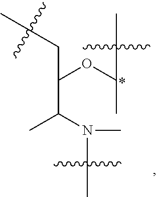 [SMILES: *CC(OC(*)C)C(C)N(*)C]

wherein “*” indicates the carbon atom bonded to a nucleobase. In some embodiments, a sugar is

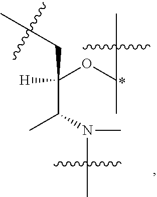 [SMILES: *C[C@@H](OC(*)C)[C@@H](C)N(*)C]

wherein “*” indicates the carbon atom bonded to a nucleobase. In some embodiments, the carbon atom bonded to a nitrogen atom of a nucleobase and is of R configuration (e.g., sm18). In some embodiments, an oligonucleotide comprises a sugar described herein.

In some embodiments, a sugar is optionally substituted

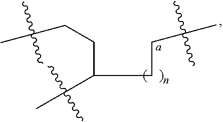

wherein position a is bonded to a nucleobase, X^{s} is —O—, —S—, —Se— or optionally substituted —CH₂—. In some embodiments, a sugar is

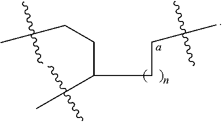

In some embodiments, X^{s} is —O—. In some embodiments, X^{s} is —S—. In some embodiments, X^{s} is —Se—. In some embodiments, X^{s} is optionally substituted —CH₂—. In some embodiments, X^{s} is —CH₂—. In some embodiments, n is 0. In some embodiments, n is 1. In some embodiments, n is 2. In some embodiments, n is 3.

In some embodiments, a sugar is connected not through 5′ and 3′ positions. Those skilled in the art appreciate that for such sugars, 5′ can refer to the side/direction toward 5′-end of an oligonucleotide, and 3′ can refer to the side/direction toward to 3′-end of an oligonucleotide.

In some embodiments, each of R^{1s}, R^{2s}, R^{3s}, R^{4s}, and R^{5s} is independently R^{s}, wherein R^{s} is independently —H, halogen, —CN, —N₃, —NO, —NO₂, -L^{s}-R′, -L^{s}-Si(R′)₃, -L^{s}-OR′, -L^{s}-SR′, -L^{s}-N(R′)₂, —O-L^{s}-R′, —O-L^{s}S—Si(R)₃, —O-L^{s}-OR′, —O-L^{s}-SR′, or —O-L^{s}-N(R′)₂; wherein L^{s} is L^{B} as described herein, and each other variable is independently as described herein. In some embodiments, each of R^{1s} and R^{2s} is independently R^{s}. In some embodiments, R^{s} is —H. In some embodiments, R^{s} is not —H. In some embodiments, L^{s} is a covalent bond. In some embodiments, each of R^{2s} and R^{4s} are independently —H, —F, —OR, —N(R)₂. In some embodiments, R^{2s} is —H, —F, —OR, —N(R)₂. In some embodiments, R^{4s} is —H. In some embodiments, R^{2s} and R^{4s} form 2′-O-L^{s}-, wherein L^{s} is optionally substituted C₁₋₆ alkylene. In some embodiments, L^{s} is optionally substituted —CH₂—. In some embodiments, L^{s} is optionally substituted —CH₂—.

In some embodiments, R is hydrogen. In some embodiments, R is not hydrogen. In some embodiments, R is an optionally substituted group selected from C₁₋₁₀ aliphatic, C₁₋₁₀ heteroaliphatic having 1-10 heteroatoms independently selected from oxygen, nitrogen, sulfur, phosphorus and silicon, C₆₋₂₀ aryl, a 5-20 membered heteroaryl ring having 1-10 heteroatoms independently selected from oxygen, nitrogen, sulfur, phosphorus and silicon, and a 3-20 membered heterocyclic ring having 1-10 heteroatoms independently selected from oxygen, nitrogen, sulfur, phosphorus and silicon.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂OCH₃.

In some embodiments, R is optionally substituted phenyl. In some embodiments, R is phenyl.

In some embodiments, R^{2s} is a 2′-modification as described in the present disclosure, and R^{4s} is —H. In some embodiments, R^{2s} is —OR, wherein R is not hydrogen. In some embodiments, R^{2s} is —F. In some embodiments, R^{2s} is —OMe. In some embodiments, R^{2s} is —OCH₂CH₂CH₃, e.g., in various Xₑₒ utilized in Table 1 (X being m5C, T, G, A, etc.). In some embodiments, R^{2s} is selected from —H, —F, and —OR, wherein R is optionally substituted C₁₋₆ alkl. In some embodiments, R^{2s} is selected from —H, —F, and —OMe.

In some embodiments, a sugar is a bicyclic sugar, e.g., sugars wherein R^{2s} and R^{4s} are taken to form an optionally substituted ring as described in the present disclosure. In some embodiments, a sugar is selected from LNA sugars, BNA sugars, cEt sugars, etc. In some embodiments, a bridge is between the 2′ and 4′-carbon atoms (corresponding to R^{2s} and R^{4s} taken together with their intervening atoms to form an optionally substituted ring as described herein). In some embodiments, a bridge is 2′-L^{a}-L^{b}-4′, wherein La is —O—, —S— or N(R), and L^{b} is an optionally substituted C₁₋₄ bivalent aliphatic chain, e.g., methylene.

In some embodiments, a sugar is a 2′-OMe, 2′-MOE, 2′-F, a LNA (locked nucleic acid) sugar, an ENA (ethylene bridged nucleic acid) sugar, a BNA(NMe) (Methylamino bridged nucleic acid) sugar, 2′-F ANA (2′-F arabinose), alpha-DNA (alpha-D-ribose), 2′/5′ ODN (e.g., 2′/5′ linked oligonucleotide), Inv (inverted sugar, e.g., inverted desoxyribose), AmR (Amino-Ribose), ThioR (Thio-ribose), HNA (hexose nucleic acid), CeNA (cyclohexene nucleic acid), or MOR (Morpholino) sugar.

Those skilled in the art after reading the present disclosure will appreciate that various types of sugar modifications are known and can be utilized in accordance with the present disclosure. In some embodiments, a sugar modification is a 2′-modification (e.g., R^{2s}). In some embodiments, a 2′-modification is 2′-F. In some embodiments, a 2′-modification is 2′-OR, wherein R is not hydrogen. In some embodiments, a 2′-modification is 2′-OR, wherein R is optionally substituted C₁₋₆ aliphatic. In some embodiments, a 2′-modification is 2′-OR, wherein R is optionally substituted C₁₋₆ alkyl. In some embodiments, a 2′-modification is 2′-OMe. In some embodiments, a 2′-modification is 2′-MOE. In some embodiments, a 2′-modification is —O-L^{b}- or -L^{b}-L^{b}- which connects the 2′-carbon of a sugar moiety to another carbon of a sugar moiety. In some embodiments, a 2′-modification is 2′-O-L^{b}-4′ or 2′-L^{b}-L^{b}-4′ which connects the 2′-carbon of a sugar moiety to the 4′-carbon of a sugar moiety. In some embodiments, a 2′-modification is S-cEt. In some embodiments, a modified sugar is an LNA sugar. In some embodiments, -L^{b}- is —C(R)₂—. In some embodiments, a 2′-modification is (C2-O—C(R)₂—C4), wherein each R is independently as described in the present disclosure. In some embodiments, a 2′-modification is a LNA sugar modification (C2—O—CH₂—C4). In some embodiments, a 2′-modification is (C2-O—CHR—C4), wherein R is as described in the present disclosure. In some embodiments, a 2′-modification is (C2-O—(R)—CHR—C4), wherein R is as described in the present disclosure and is not hydrogen. In some embodiments, a 2′-modification is (C2-O—(S)—CHR—C4), wherein R is as described in the present disclosure and is not hydrogen. In some embodiments, R is optionally substituted C₁₋₆ aliphatic. In some embodiments, R is optionally substituted C₁₋₆ alkyl. In some embodiments, R is unsubstituted C₁₋₆ alkyl. In some embodiments, R is methyl. In some embodiments, R is ethyl. In some embodiments, a 2′-modification is (C2-O—CHR—C4), wherein R is optionally substituted C₁₋₆ aliphatic. In some embodiments, a 2′-modification is (C2-O—CHR—C4), wherein R is optionally substituted C₁₋₆ alkyl. In some embodiments, a 2′-modification is (C2-O—CHR—C4), wherein R is methyl. In some embodiments, a 2′-modification is (C2-O—CHR—C4), wherein R is ethyl. In some embodiments, a 2′-modification is (C2-O—(R)—CHR—C4), wherein R is optionally substituted C₁₋₆ aliphatic. In some embodiments, a 2′-modification is (C2-O—(R)—CHR—C4), wherein R is optionally substituted C₁₋₆ alkyl. In some embodiments, a 2′-modification is (C2-O—(R)—CHR—C4), wherein R is methyl. In some embodiments, a 2′-modification is (C2-O—(R)—CHR—C4), wherein R is ethyl. In some embodiments, a 2′-modification is (C2-O—(S)—CHR—C4), wherein R is optionally substituted C₁₋₆ aliphatic. In some embodiments, a 2′-modification is (C2-O—(S)—CHR—C4), wherein R is optionally substituted C₁₋₆ alkyl. In some embodiments, a 2′-modification is (C2-O—(S)—CHR—C4), wherein R is methyl. In some embodiments, a 2′-modification is (C2-O—(S)—CHR—C4), wherein R is ethyl. In some embodiments, a 2′-modification is C2-O—(R)—CH(CH₂CH₃)—C4. In some embodiments, a 2′-modification is C2-O—(S)—CH(CH₂CH₃)—C4. In some embodiments, a sugar is a natural DNA sugar. In some embodiments, a sugar is a natural RNA sugar. In some embodiments, a sugar is an optionally substituted natural DNA sugar. In some embodiments, a sugar is a natural DNA sugar optionally substituted at 2′. In some embodiments, a sugar is a natural DNA sugar substituted at 2′ (2′-modification). In some embodiments, a sugar is a natural DNA sugar modified at 2′ (2′-modification).

In some embodiments, a sugar is an optionally substituted ribose or deoxyribose. In some embodiments, a sugar is an optionally modified ribose or deoxyribose, wherein one or more hydroxyl groups of the ribose or deoxyribose moiety is optionally and independently replaced by halogen, R′, —N(R′)₂, —OR′, or —SR′, wherein each R′ is as described herein. In some embodiments, a sugar is an optionally substituted deoxyribose, wherein the 2′ position of the deoxyribose is optionally substituted. In some embodiments, a sugar is an optionally substituted deoxyribose, wherein the 2′ position of the deoxyribose is optionally substituted with halogen, R′, —N(R′)₂, —OR′, or —SR′, wherein each R′ is independently described in the present disclosure. In some embodiments, a sugar is an optionally substituted deoxyribose, wherein the 2′ position of the deoxyribose is optionally substituted with halogen. In some embodiments, a sugar is an optionally substituted deoxyribose, wherein the 2′ position of the deoxyribose is optionally substituted with one or more —F. In some embodiments, a sugar is an optionally substituted deoxyribose, wherein the 2′ position of the deoxyribose is optionally substituted with —OR′, wherein each R′ is independently described in the present disclosure. In some embodiments, a sugar is an optionally substituted deoxyribose, wherein the 2′ position of the deoxyribose is optionally substituted with —OR′, wherein each R′ is independently optionally substituted C₁-C₆ aliphatic. In some embodiments, a sugar is an optionally substituted deoxyribose, wherein the 2′ position of the deoxyribose is optionally substituted with —OR′, wherein each R′ is independently an optionally substituted C₁-C₆ alkyl. In some embodiments, a sugar is an optionally substituted deoxyribose, wherein the 2′ position of the deoxyribose is optionally substituted with —OMe. In some embodiments, a sugar is an optionally substituted deoxyribose, wherein the 2′ position of the deoxyribose is optionally substituted with —O-methoxyethyl.

In some embodiments, provided oligonucleotides comprise one or more modified sugars. In some embodiments, provided oligonucleotides comprise one or more modified sugars and one or more natural sugars.

Examples of bicyclic sugars include sugars of alpha-L-methyleneoxy (4′-CH₂—O-2′) LNA, beta-D-methyleneoxy (4′-CH₂—O-2′) LNA, ethyleneoxy (4′-(CH₂)₂—O-2′) LNA, aminooxy (4′-CH₂—O—N(R)-2′) LNA, and oxyamino (4′-CH₂—N(R)—O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose.

In some embodiments, a bicyclic sugar may be further defined by isomeric configuration. For example, a sugar comprising a 4′-(CH₂)—O-2′ bridge may be in the alpha-L configuration or in the beta-D configuration. In some embodiments, a 4′ to 2′ bridge is a -L-4′-(CH₂)—O-2′, b-D-4′-CH₂—O-2′, 4′-(CH₂)₂—O-2′, 4′-CH₂—O—N(R′)-2′, 4′-CH₂—N(R′)—O-2′, 4′-CH(R′)—O-2′, 4′-CH(CH₃)—O-2′, 4′-CH₂—S-2′, 4′-CH₂—N(R′)-2′, 4′-CH₂—CH(R′)-2′, 4′-CH₂—CH(CH₃)-2′, and 4′-(CH₂)₃-2′, wherein each R′ is as described in the present disclosure. In some embodiments, R′ is —H, a protecting group or optionally substituted C₁-C₁₂ alkyl. In some embodiments, R′ is —H or optionally substituted C₁-C₁₂ alkyl.

In some embodiments, a bicyclic sugar is a sugar of alpha-L-methyleneoxy (4′-CH₂—O-2′) BNA, beta-D-methyleneoxy (4′-CH₂—O-2′) BNA, ethyleneoxy (4′-(CH₂)₂—O-2′) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA (also referred to as constrained ethyl or cEt), methylene-thio (4′-CH₂—S-2′) BNA, methylene-amino (4′-CH₂—N(R)-2′) BNA, methyl carbocyclic (4′-CH₂—CH(CH₃)-2′) BNA, propylene carbocyclic (4′-(CH₂)₃-2′) BNA, or vinyl BNA.

In some embodiments, a sugar modification is a modification described in U.S. Pat. No. 9,006,198. In some embodiments, a modified sugar is described in U.S. Pat. No. 9,006,198. In some embodiments, a sugar modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the sugar modifications and modified sugars of each of which are independently incorporated herein by reference.

In some embodiments a modified sugar is one described in U.S. Pat. Nos. 5,658,873, 5,118,800, 5,393,878, 5,514,785, 5,627,053, 7,034,133; 7084125, U.S. Pat. Nos. 7,399,845, 5,319,080, 5,591,722, 5,597,909, 5,466,786, 6,268,490, 6,525,191, 5,519,134, 5,576,427, 6,794,499, 6,998,484, 7,053,207, 4,981,957, 5,359,044, 6,770,748, 7,427,672, 5,446,137, 6,670,461, 7,569,686, 7,741,457, 8,022,193, 8,030,467, 8,278,425, 5,610,300, 5,646,265, 8,278,426, 5,567,811, 5,700,920, 8,278,283, 5,639,873, 5,670,633, 8,314,227, US 2008/0039618, US 2009/0012281, WO 2021/030778, WO 2020/154344, WO 2020/154343, WO 2020/154342, WO 2020/165077, WO 2020/201406, WO 2020/216637, or WO 2020/252376.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, an oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof. In some embodiments, a sugar is as in flexible nucleic acids or serinol nucleic acids. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc. In some embodiments, a sugar is a (R)-GNA sugar. In some embodiments, a sugar is a (S)-GNA sugar. In some embodiments, a nucleoside having a GNA sugar is utilized as N₋₁, N₀ and/or N₁. In some embodiments, N₀ is a nucleoside having a GNA sugar. In some embodiments, a sugar is bicyclic sugar. In some embodiments, a sugar is a LNA sugar. In some embodiments, a sugar is an acyclic sugar. In some embodiments, a sugar is a UNA sugar. In some embodiments, a nucleoside having a UNA sugar is utilized as N₋₁, N₀ and/or N₁. In some embodiments, N₀ is a nucleoside having a UNA sugar. In some embodiments, a nucleoside is abasic. In some embodiments, an abasic sugar is utilized as N₋₁, N₀ and/or N₁. In some embodiments, N₀ is a nucleoside having an abasic sugar.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂)ₙOCH₃, —O(CH₂)ₙNH₂, MOE, DMAOE, or DMAEOE, wherein n is from 1 to about 10. In some embodiments, a modified sugar is one described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. Commonly used 2′-modifications include but are not limited to 2′-OR, wherein R is not hydrogen and is as described in the present disclosure. In some embodiments, a modification is 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA. In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, one or more of the sugars of an oligonucleotide are modified. In some embodiments, a modified sugar comprises a 2′-modification. In some embodiments, each modified sugar independently comprises a 2′-modification. In some embodiments, a 2′-modification is 2′-OR. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR or 2′-F. In some embodiments, each sugar modification is independently 2′-OR or 2′-F, wherein R is optionally substituted C₁₋₆ alkyl. In some embodiments, each sugar modification is independently 2′-OR or 2′-F, wherein at least one is 2′-F. In some embodiments, each sugar modification is independently 2′-OR or 2′-F, wherein R is optionally substituted C₁₋₆ alkyl, and wherein at least one is 2′-OR. In some embodiments, each sugar modification is independently 2′-OR or 2′-F, wherein at least one is 2′-F, and at least one is 2′-OR. In some embodiments, each sugar modification is independently 2′-OR or 2′-F, wherein R is optionally substituted C₁₋₆ alkyl, and wherein at least one is 2′-F, and at least one is 2′-OR. In some embodiments, each sugar modification is independently 2′-OR. In some embodiments, each sugar modification is independently 2′-OR, wherein R is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

Modified sugars include cyclobutyl or cyclopentyl moieties in place of a pentofuranosyl sugar. Representative examples of such modified sugars include those described in U.S. Pat. Nos. 4,981,957, 5,118,800, 5,319,080, or U.S. Pat. No. 5,359,044. In some embodiments, the oxygen atom within the ribose ring is replaced by nitrogen, sulfur, selenium, or carbon. In some embodiments, —O— is replaced with —N(R′)—, —S—, —Se— or —C(R′)₂—. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

A non-limiting example of modified sugars is glycerol, which is part of glycerol nucleic acids (GNAs), e.g., as described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603.

A flexible nucleic acid (FNA) is based on a mixed acetal aminal of formyl glycerol, e.g., as described in Joyce G F et al., PNAS, 1987, 84, 4398-4402 and Heuberger B D and Switzer C, J. Am. Chem. Soc., 2008, 130, 412-413.

In some embodiments, an oligonucleotide, and/or a modified nucleoside thereof, comprises a sugar or modified sugar described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the sugars and modified sugars of each of which are independently incorporated herein by reference.

In some embodiments, one or more hydroxyl group in a sugar is optionally and independently replaced with halogen, R′ —N(R′)₂, —OR′, or —SR′, wherein each R′ is independently described in the present disclosure.

In some embodiments, a modified nucleoside is any modified nucleoside described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the modified nucleosides of each of which are independently incorporated herein by reference.

In some embodiments, a sugar modification is 5′-vinyl (R or S), 5′-methyl (R or S), 2′-SH, 2′-F, 2′-OCH₃, 2′-OCH₂CH₃, 2′-OCH₂CH₂F or 2′-O(CH₂)₂₀CH₃. In some embodiments, a substituent at the 2′ position, e.g., a 2′-modification, is allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), wherein each allyl, amino and alkyl is optionally substituted, and each of R₁, Rₘ and Rₙ is independently R′ as described in the present disclosure. In some embodiments, each of R₁, Rₘ and Rₙ is independently —H or optionally substituted C₁-C₁₀ alkyl.

In some embodiments, bicyclic sugars comprise a bridge, e.g., -L^{b}-L^{b}-, -L-, etc. between two sugar carbons, e.g., between the 4′ and the 2′ ribosyl ring carbon atoms. In some embodiments, a bridge is 4′-(CH₂)—O-2′ (e.g., LNA sugars), 4′-(CH₂)—S-2′, 4′-(CH₂)₂—O-2′ (e.g., ENA sugars), 4′-CH(R′)—O-2′ (e.g., 4′-CH(CH₃)—O-2′, 4′-CH(CH₂OCH₃)—O-2′, and examples in U.S. Pat. No. 7,399,845, etc.), 4′-CH(R′)₂—O-2′ (e.g., 4′-C(CH₃)(CH₃)—O-2′ and examples in WO 2009006478, etc.), 4′-CH₂—N(OR′)-2′ (e.g., 4′-CH₂—N(OCH₃)-2′, examples in WO 2008150729, etc.), 4′-CH₂—O—N(R′)-2′ (e.g., 4′-CH₂—O—N(CH₃)-2′, examples in US 20040171570, etc.), 4′-CH₂—N(R′)—O-2′ [e.g., wherein R is —H, C₁-C₁₂ alkyl, or a protecting group (e.g., see U.S. Pat. No. 7,427,672)], 4′-C(R′)₂—C(H)(R′)-2′ (e.g., 4′-CH₂—C(H)(CH₃)-2′, examples in Chattopadhyaya et al., J. Org. Chem., 2009, 74, 118-134, etc.), or 4′-C(R′)₂—C(═C(R′)₂)-2′ (e.g., 4′-CH₂—C(═CH₂)-2′, examples in WO 2008154401, etc.).

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (F-HNA).

In some embodiments, sugars comprise rings having more than 5 atoms and/or more than one heteroatom, e.g., morpholino sugars which are described in e.g., Braasch et al., Biochemistry, 2002, 41, 4503-4510; U.S. Pat. Nos. 5,698,685; 5,166,315; 5,185,444; 5,034,506; etc.).

As those skilled in the art will appreciate, modifications of sugars, nucleobases, internucleotidic linkages, etc. can and are often utilized in combination in oligonucleotides, e.g., see various oligonucleotides in Table 1.

In some embodiments, a nucleoside has a six-membered cyclohexenyl in place of the pentofuranosyl residue in naturally occurring nucleosides. Example cyclohexenyl nucleosides and preparation and uses thereof are described in, e.g., WO 2010036696; Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; WO 2006047842; WO 2001049687; etc.

Many monocyclic, bicyclic and tricyclic ring systems are suitable as sugar surrogates (modified sugars) and may be utilized in accordance with the present disclosure. See, e.g., Leumann, Christian J. Bioorg. & Med. Chem., 2002, 10, 841-854. Such ring systems can undergo various additional substitutions to further enhance their properties and/or activities.

In some embodiments, a 2′-modified sugar is a furanosyl sugar modified at the 2′ position. In some embodiments, a 2′-modification is halogen, —R′ (wherein R′ is not —H), —OR′ (wherein R′ is not —H), —SR′, —N(R′)₂, optionally substituted —CH₂—CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)ₙCH₃, —O(CH₂)ₙF, —O(CH₂)ₙONH₂, —OCH₂C(═O)N(H)CH₃, and —O(CH₂)ₙON[(CH₂)ₙCH₃]2, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted —O-alkaryl, optionally substituted —O-aralkyl, —SH, —SCH₃, —OCN, —C₁, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification (e.g., see Baker et al., J. Biol. Chem., 1997, 272, 11944-12000). In some cases, a 2′-MOE modification has been reported as having improved binding affinity compared to unmodified sugars and to some other modified nucleosides, such as 2′-O-methyl, 2′-O-propyl, and 2′-O-aminopropyl. Oligonucleotides having the 2′-MOE modification have also been reported to be capable of inhibiting gene expression with promising features for in vivo use (see, e.g., Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926; etc.).

In some embodiments, a 2′-modified or 2′-substituted sugar or nucleoside is a sugar or nucleoside comprising a substituent at the 2′ position of the sugar which is other than —H (typically not considered a substituent) or —OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′-O(CH₂)₂SCH₃, —O(CH₂)₂ON(Rₘ)(Rₙ), or —OCH₂C(═O)N(Rₘ)(Rₙ), where each Rₘ and Rₙ is independently —H or optionally substituted C₁-C₁₀ alkyl.

Certain modified sugars, their preparation and uses are described in U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,670,633, 5,700,920, 5,792,847, 6,600,032 and WO 2005121371.

In some embodiments, a sugar is the sugar of N-methanocarba, LNA, cMOE BNA, cEt BNA, α-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun. 1; 42(10): 6542-6551), formacetal, thioformacetal, MMI [e.g., methylene(methylimino), Peoc'h et al. 2006 Nucleosides and Nucleotides 16 (7-9)], a PMO (phosphorodiamidate linked morpholino) linkage (which connects two sugars), or a PNA (peptide nucleic acid) linkage. In some embodiments, examples of internucleotidic linkages and/or sugars are described in Allerson et al. 2005 J. Med. Chem. 48: 901-4; BMCL 2011 21: 1122; BMCL 2011 21: 588; BMCL 2012 22: 296; Chattopadhyaya et al. 2007 J. Am. Chem. Soc. 129: 8362; Chem. Bio. Chem. 2013 14: 58; Curr. Prot. Nucl. Acids Chem. 2011 1.24.1; Egli et al. 2011 J. Am. Chem. Soc. 133: 16642; Hendrix et al. 1997 Chem. Eur. J. 3: 110; Hyrup et al. 1996 Bioorg. Med. Chem. 4: 5; Imanishi 1997 Tet. Lett. 38: 8735; J. Am. Chem. Soc. 1994, 116, 3143; J. Med. Chem. 2009 52: 10; J. Org. Chem. 2010 75: 1589; Jepsen et al. 2004 Oligo. 14: 130-146; Jones et al. J. Org. Chem. 1993, 58, 2983; Jung et al. 2014 ACIEE 53: 9893; Kodama et al. 2014 AGDS; Koizumi 2003 BMC 11: 2211; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Lima et al. 2012 Cell 150: 883-894; Mesmaeker et al. Angew. Chem., Int. Ed. Engl. 1994, 33, 226; Migawa et al. 2013 Org. Lett. 15: 4316; Mol. Ther. Nucl. Acids 2012 1: e47; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Murray et al. 2012 Nucl. Acids Res. 40: 6135; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Obika et al. 2008 J. Am. Chem. Soc. 130: 4886; Obika et al. 2011 Org. Lett. 13: 6050; Oestergaard et al. 2014 JOC 79: 8877; Pallan et al. 2012 Biochem. 51: 7; Pallan et al. 2012 Chem. Comm. 48: 8195-8197; Petersen et al. 2003 TRENDS Biotech. 21: 74-81; Prakash et al. 2010 J. Med. Chem. 53: 1636; Prakash et al. 2015 Nucl. Acids Res. 43: 2993-3011; Prakash et al. 2016 Bioorg. Med. Chem. Lett. 26: 2817-2820; Rajwanshi et al. 1999 Chem. Commun. 1395-1396; Schultz et al. 1996 Nucleic Acids Res. 24: 2966; Seth et al. 2008 Nucl. Acid Sym. Ser. 52: 553; Seth et al. 2009 J. Med. Chem. 52: 10-13; Seth et al. 2010 J. Am. Chem. Soc. 132: 14942; Seth et al. 2010 J. Med. Chem. 53: 8309-8318; Seth et al. 2010 J. Org. Chem. 75: 1569-1581; Seth et al. 2011 BMCL 21: 4690; Seth et al. 2012 Bioo. Med. Chem. Lett. 22: 296-299; Seth et al. 2012 Mol. Ther-Nuc. Acids. 1, e47; Seth et al., Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Starrup et al. 2010 Nucl. Acids Res. 38: 7100; Swayze et al. 2007 Nucl. Acids Res. 35: 687; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 2007090071; WO 2016079181; U.S. Pat. Nos. 6,326,199; 6,066,500; or U.S. Pat. No. 6,440,739.

### Internucleotidic Linkages

Among other things, the present disclosure provides various internucleotidic linkages, including various modified internucleotidic linkages, that may be utilized together with other structural elements, e.g., various sugars as described herein, to provide oligonucleotides and compositions thereof.

Various internucleotidic linkages may be utilized in oligonucleotides in accordance with the present disclosure. In some embodiments, an oligonucleotide comprises one or more types of internucleotidic linkage. In some embodiments, an oligonucleotide comprises two or more types of internucleotidic linkage. In some embodiments, an oligonucleotide comprises at least three types of internucleotidic linkages. In some embodiments, a linkage contains a linkage phosphorus atom bonded to an oxygen atom which oxygen atom is not bonded to or is not part of a backbone sugar (“a PO linkage”, e.g., a natural phosphate linkage). In some embodiments, a linkage contains a linkage phosphorus atom bonded to a sulfur atom which sulfur atom is not bonded to or is not part of a backbone sugar (“a PS linkage”, e.g., a phosphorothioate internucleotidic linkage). In some embodiments, a linkage contains a linkage phosphorus atom bonded to a nitrogen atom which nitrogen atom is not bonded to or is not part of a backbone sugar (“a PN linkage”, e.g., n001). In some embodiments, an oligonucleotide comprises one or more PS linkages. In some embodiments, an oligonucleotide comprises one or more PO linkages. In some embodiments, an oligonucleotide comprises one or more PN linkages. In some embodiments, an oligonucleotide comprises one or more PS and one or more PO linkages. In some embodiments, an oligonucleotide comprises one or more PS and one or more PN linkages. In some embodiments, an oligonucleotide comprises one or more PS, one or more PN and one or more PO linkages.

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, provided oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S⁻)O—.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate. In some embodiments, a modified internucleotidic linkage is a PN linkage. In some embodiments, a modified internucleotidic linkage is a PS linkage. In some embodiments, a modified internucleotidic linkage is a PO linkage (e.g., other than a natural phosphate linkage). In some embodiments, each modified internucleotidic linkage is independently a PN internucleotidic linkage or a PS internucleotidic linkage. In some embodiments, an oligonucleotide comprises one or more PN internucleotidic linkages, one or more PS internucleotidic linkages, and one or more PO internucleotidic linkages. In some embodiments, one or more PN internucleotidic linkages are independently phosphoryl guanidine internucleotidic linkages. In some embodiments, one or more PN internucleotidic linkages are independently n001. In some embodiments, one or more PS internucleotidic linkages are independently phosphorothioate internucleotidic linkages. In some embodiments, each PS internucleotidic linkage is independently a phosphorothioate internucleotidic linkage. In some embodiments, one or more PO internucleotidic linkages are independently natural phosphate linkages. In some embodiments, each PO internucleotidic linkage is independently a natural phosphate linkage.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In some embodiments, an internucleotidic linkage comprises a P-modification, wherein a P-modification is a modification at a linkage phosphorus. In some embodiments, a modified internucleotidic linkage is a moiety which does not comprise a phosphorus but serves to link two sugars or two moieties that each independently comprises a nucleobase, e.g., as in peptide nucleic acid (PNA).

In some embodiments, an oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the internucleotidic linkages (e.g., those of Formula I, I-a, I-b, I-c, etc.) of each of which are independently incorporated herein by reference. In some embodiments, a modified internucleotidic linkage is a chiral internucleotidic linkage. In some embodiments, a modified internucleotidic linkage is a phosphorothioate internucleotidic linkage.

In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage has the structure of Formula I-n-1, I-n-2, I-n-3, I-n-4, II, II-a-1, II-a-2, II-b-1, II-b-2, II-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the non-negatively charged internucleotidic linkages (e.g., those of Formula I-n-1, I-n-2, I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

In some embodiments, an internucleotidic linkage is a PN linkage. In some embodiments, a PN linkage comprises a phosphoryl guanidine linkage.

In some embodiments, an internucleotidic linkage has the structure of —OP(O)(NHSO₂R^{s})O— or a salt form thereof, wherein R^{s} is as described herein. In some embodiments, an internucleotidic linkage has the structure of —P(O)(NHSO₂R^{s})O— or a salt form thereof, wherein R^{s} is as described herein. In some embodiments, an internucleotidic linkage has the structure of —P(O)(NHSO₂R^{s})— or a salt form thereof, wherein R^{s} is as described herein. In some embodiments, R^{s} is R′ as described herein. In some embodiments, R^{s} is —N(R′)₂ wherein each R′ is independently as described herein. In some embodiments, R^{s} is —NHR′ wherein R′ is as described herein. In some embodiments, R′ is R as described herein. For example, in some embodiments, R is optionally substituted C₁₋₁₅ (e.g., C₁₋₁₄, C₁₋₁₃, C₁₋₁₂, C₁₋₁₀, C₁₋₆, etc.) aliphatic. In some embodiments, R is optionally substituted C₁₋₁₂ aliphatic. In some embodiments, R is optionally substituted C₁₋₁₅ (e.g., C₁₋₁₄, C₁₋₁₃, C₁₋₁₂, C₁₋₁₀, C₁₋₆, etc.) alkyl. In some embodiments, R is optionally substituted C₂₋₁₅ (e.g., C₂₋₁₄, C₂₋₁₃, C₂₋₁₂, C₂₋₁₀, C₂₋₆, etc.) alkynyl. In some embodiments, R is optionally substituted C₁₋₁₂ alkyl. In some embodiments, R is optionally substituted C₂₋₁₂ alkynyl. In some embodiments, R is optionally substituted C₂₋₁₀ alkynyl. In some embodiments, R is optionally substituted C₂₋₆ alkynyl. In some embodiments, R comprises an optionally substituted terminal alkynyl group. In some embodiments, R is optionally substituted HC≡C(CH₂)n-, wherein n is 1, 2, 3, 4, 5, 6, 7, 8, 9, 10, 11, 12, 13, 14, 15, 16, 17, 18, 19, or 20. In some embodiments, R′ is optionally substituted phenyl. In some embodiments, R is optionally substituted heteroaryl. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is optionally substituted methyl. In some embodiments, R is methyl. In some embodiments, R is —CF₃. In some embodiments, R is methyl. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted propyl. In some embodiments, R is optionally substituted butyl. In some embodiments, R is n-butyl. In some embodiments, R is —(CH₂)₆NH₂. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted phenyl. In some embodiments, R is phenyl. In some embodiments, R is p-methylphenyl. In some embodiments, R is 4-dimethylaminophenyl. In some embodiments, R is 3-pyridinyl. In some embodiments, R is

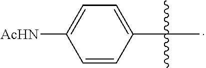 [SMILES: *c1ccc(NC(C)=O)cc1]

In some embodiments, R is n-C₁₂H₂₅—. In some embodiments, R is n-C₆H₁₃—. In some embodiments, —NHSO₂R^{s} is selected from:

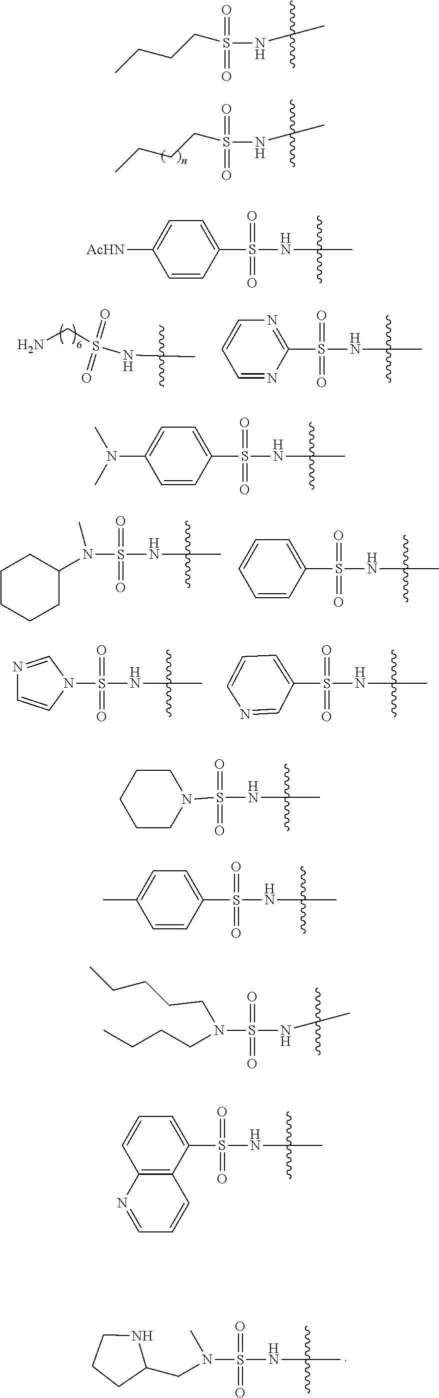

In some embodiments, an internucleotidic linkage is described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the internucleotidic linkages of each of which are independently incorporated herein by reference.

### Certain Technologies for Preparing Oligonucleotides

Various technologies can be utilized to prepare oligonucleotides in accordance with the present disclosure, e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the oligonucleotide preparation technologies of each of which are independently incorporated herein by reference.

In some embodiments, the present disclosure provides technologies that can reduce amounts and/or equivalents of azides utilized for constructing PN linkages. In some embodiments, PN linkages are sulfonyl PN linkages. As those skilled in the art appreciate, utilization of azides particularly when at large scales (e.g., commercial manufacturing scales) may present safety challenges. In some embodiments, provided technologies reduce cost and/or improve safety.

In some embodiments, the present disclosure provides a method comprising:
1) contacting a P(III) agent with an azide to provide a composition for coupling;
2) contacting the composition for coupling with a coupling partner.

In some embodiments, a P(III) agent is contacted with an azide in the absence of the coupling partner. In some embodiments, a P(III) agent, e.g. a phosphoramidite and an azide are contacted with each other to provide a composition for coupling. In some embodiments, a composition for coupling is formed as a single composition and then is added to a nucleoside or an oligonucleotide for coupling.

In some embodiments, the ratio of an azide to a coupling partner is about or less than about 2, 1.9, 1.8, 1.7, 1.6, 1.5, 1.4, 1.3, 1.2, or 1.1. In some embodiments, the ratio of an azide to a coupling partner is about or less than about 2. In some embodiments, the ratio of an azide to a coupling partner is about or less than about 1.9. In some embodiments, the ratio of an azide to a coupling partner is about or less than about 1.8. In some embodiments, the ratio of an azide to a coupling partner is about or less than about 1.7. In some embodiments, the ratio of an azide to a coupling partner is about or less than about 1.6. In some embodiments, the ratio of an azide to a coupling partner is about or less than about 1.5. In some embodiments, the ratio of an azide to a coupling partner is about or less than about 1.4. In some embodiments, the ratio of an azide to a coupling partner is about or less than about 1.3. In some embodiments, the ratio of an azide to a coupling partner is about or less than about 1.2. In some embodiments, the ratio of an azide to a coupling partner is about or less than about 1.1.

In some embodiments, the ratio of an azide to a coupling partner is about 1.5-1.

In some embodiments, the ratio of an azide to a P(III) agent is about or less than about 2, 1.9, 1.8, 1.7, 1.6, 1.5, 1.4, 1.3, 1.2, or 1.1. In some embodiments, the ratio of an azide to a P(III) agent is about or less than about 2. In some embodiments, the ratio of an azide to a P(III) agent is about or less than about 1.9. In some embodiments, the ratio of an azide to a P(III) agent is about or less than about 1.8. In some embodiments, the ratio of an azide to a P(III) agent is about or less than about 1.7. In some embodiments, the ratio of an azide to a P(III) agent is about or less than about 1.6. In some embodiments, the ratio of an azide to a P(III) agent is about or less than about 1.5. In some embodiments, the ratio of an azide to a P(III) agent is about or less than about 1.4. In some embodiments, the ratio of an azide to a P(III) agent is about or less than about 1.3. In some embodiments, the ratio of an azide to a P(III) agent is about or less than about 1.2. In some embodiments, the ratio of an azide to a P(III) agent is about or less than about 1.1.

In some embodiments, the ratio of an azide to a P(III) agent is about 1.5-1

In some embodiments, a P(III) agent has a structure of

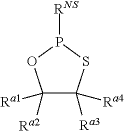

or a salt thereof, wherein R^{NS} is a nucleoside as described herein and each of R^{a1}, R^{a2}, R^{a3} and R^{a4} is independently R as described herein. In some embodiments, R^{a2} is —H. In some embodiments, R^{a2} is optionally substituted C₁₋₆ aliphatic. In some embodiments, R^{a2} is optionally substituted C₁₋₆ alkyl. In some embodiments, R^{a4} is —H. In some embodiments, R^{a4} is optionally substituted C₁₋₆ aliphatic. In some embodiments, R^{a4} is optionally substituted C₁₋₆ alkyl. In some embodiments, R^{a4} is methyl. In some embodiments, R^{a1} is optionally substituted C₁₋₆ alkyl. In some embodiments, R^{a1} is methyl. In some embodiments, R^{a3} is optionally substituted C₁₋₆ alkyl. In some embodiments, R^{a3} is methyl. In some embodiments, a P(III) agent has a structure of

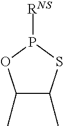

or a salt thereof.

In some embodiments, R^{a1} and R^{a3} are taken together to form an optionally substitute 3-10 (e.g., 3-6, 4-10, 5-6, 3, 4, 5, 6, 7, 8, 9, or 10, etc.) membered ring having 0-5 (e.g., 0, 1-5, 1, 2, 3, 4, 5, etc.) heteroatoms. In some embodiments, R^{a1} and R^{a3} are taken together to form an optionally substitute 3-6 membered ring. In some embodiments, R^{a1} and R^{a3} are taken together to form an optionally substitute 5-6 membered ring. In some embodiments, R^{a1} and R^{a3} are taken together to form an optionally substitute 5-membered ring. In some embodiments, R^{a1} and R^{a3} are taken together to form an optionally substitute 6-membered ring. In some embodiments, a formed ring has no heteroatom.

In some embodiments, a P(III) agent has a structure of

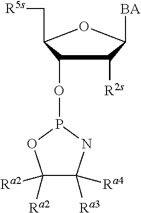

or a salt thereof, wherein each of the variable groups is independently as described herein. In some embodiments, a P(III) agent has a structure of

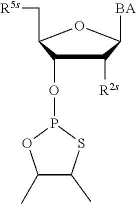

or a salt thereof, wherein each of the variable groups is independently as described herein. In some embodiments, a P(III) agent has a structure of

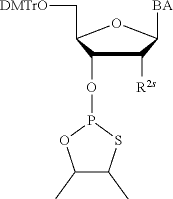

or a salt thereof, wherein each of the variable groups is independently as described herein. In some embodiments, a P(III) agent has a structure of

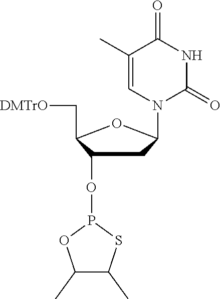

or a salt thereof.

Among other things, the present disclosure provides the following Embodiments as examples:

1. A method for preparing a compound of formula P:

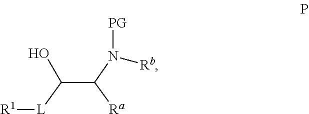

or a salt thereof,
comprising reducing a compound of formula INT-1:

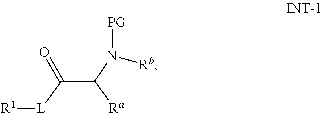

or a salt thereof to provide a compound of formula P or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or

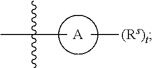

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   2. A method for preparing a compound of formula P-a:

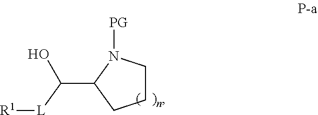

or a salt thereof,
comprising reducing a compound of formula INT-1-a:

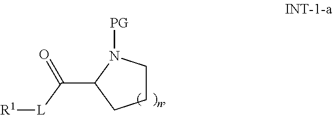

or a salt thereof to provide a compound of formula P-a or a salt thereof, wherein:
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

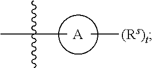

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   3. The method of any one of the preceding Embodiments, wherein the reducing is carried out in the presence of HCOOH or a salt thereof.
   4. The method of any one of the preceding Embodiments, wherein the reducing is carried out in the present of an agent comprising a metal.
   5. The method of any one of the preceding Embodiments, wherein the reduction is carried out in the present of an agent comprising Ru.
   6. The method of Embodiment 5, wherein the agent is a Ru complex comprising one or more ligands.
   7. The method of Embodiment 5, wherein the agent comprises a ligand having the structure of NHR^{M1}—CH₂—CH₂—N(—)S(O)₂R^{M2}, NHR^{M1}—C(R^{M3})₂—C(R^{M4})₂—N(—)S(O)₂R^{M2} or NHR^{M1}—CHR^{M3}—CHR^{M4}—N(—)S(O)₂R^{M2}, wherein each of R^{M1}, R^{M2}, R^{M3} and R^{M4} is independently R.
   8. The method of Embodiment 5, wherein the agent comprises a ligand having the structure of R^{M6}_R^{M7} wherein R^{M6} is R wherein R is optionally substituted aryl or heteroaryl, R^{M7} is R, or is taken together with R^{M1} to form a linker if a ligand comprising R^{M1} is present.
   9. The method of Embodiment 5, wherein the agent has the structure of Ru[NHR^{M1}—CH₂—CH₂—N(—)S(O)₂R^{M2}](R^{M5})(R^{M6}_R^{M7}) or a salt thereof, wherein:
each —CH₂— is independently optionally substituted;
each of R^{M1} and R^{M2} is independently R;
R^{M5} is halogen;
R^{M6} is R wherein R is optionally substituted aryl or heteroaryl; and
R^{M7} is R, or is taken together with R^{M1} to form a linker.
   10. The method of Embodiment 5, wherein the agent has the structure of Ru[NHR^{M1}—C(R^{M3})₂—C(R^{M4})₂—N(—)S(O)₂R^{M2}](R^{M5})(R^{M6}_R^{M7}) or a salt thereof, wherein:
each —CH₂— is independently optionally substituted;
each of R^{M1}, R^{M2}, R^{M3} and R^{M4} is independently R;
R^{M5} is halogen;
R^{M6} is R wherein R is optionally substituted aryl or heteroaryl; and
R^{M7} is R, or is taken together with R^{M1} to form a linker.
   11. The method of Embodiment 5, wherein the agent has the structure of Ru[NHR^{M1}—CHR^{M3}—CHR^{M4}—N(—)S(O)₂R^{M2}](R^{M5})(R^{M6}_R^{M7}) or a salt thereof, wherein:
each —CH₂— is independently optionally substituted;
each of R^{M1}, R^{M2}, R^{M3} and R^{M4} is independently R;
R^{M5} is halogen;
R^{M6} is R wherein R is optionally substituted aryl or heteroaryl; and
R^{M7} is R, or is taken together with R^{M1} to form a linker.
   12. The method of any one of the preceding Embodiments, wherein R^{M1} is —H.
   13. The method of any one of the preceding Embodiments, wherein R^{M2} is optionally substituted methyl.
   14. The method of Embodiment 13, wherein R^{M2} is p-methylphenyl.
   15. The method of Embodiment 13, wherein R^{M2} is pentafluorophenyl.
   16. The method of any one of the preceding Embodiments, wherein —NHR^{M1} and —N(—)S(O)₂R^{M2} are trans.
   17. The method of any one of the preceding Embodiments, wherein R^{M3} is —H.
   18. The method of any one of the preceding Embodiments, wherein R^{M4} is —H.
   19. The method of any one of the preceding Embodiments, wherein R^{M5} is —Cl.
   20. The method of any one of the preceding Embodiments, wherein R^{M6} is optionally substituted phenyl.
   21. The method of any one of the preceding Embodiments, wherein R^{M7} is —H or optionally substituted C₁₋₆ alkyl.
   22. The method of any one of the preceding Embodiments, wherein R^{M6}_R^{M7} is p-cymene.
   23. The method of any one of the preceding Embodiments, wherein R^{M6}_R^{M7} is mesitylene.
   24. The method of any one of Embodiments 8-11 and 13-20, wherein R^{M1} and R^{M7} are taken together to form a linker.
   25. The method of Embodiment 24, wherein a linker is an optionally substituted bivalent C₁₋₆ linear or branched aliphatic group.
   26. The method of Embodiment 24, wherein a linker is an optionally substituted bivalent C₁₋₆ linear or branched heteroaliphatic group having 1-3 heteroatoms each independently selected from nitrogen, oxygen and sulfur.
   27. The method of Embodiment 24, wherein a linker is optionally substituted —(CH₂)m- wherein m is 1-6.
   28. The method of Embodiment 27, wherein m is 3.
   29. The method of any one of the preceding Embodiments, wherein a compound of formula P or a salt thereof is a compound of formula P-1, P-a-1, or P-b-1 or a salt thereof.
   30. The method of any one of the preceding Embodiments, wherein a compound of formula INT-1 or a salt thereof is a compound of formula INT-1-1, INT-1-a-1 or INT-1-b-1, or a salt thereof.
   31. The method of any one of Embodiments 1-28, wherein a compound of formula P or a salt thereof is a compound of formula P-2, P-a-2, or P-b-2, or a salt thereof.
   32. The method of Embodiment 31, wherein a compound of formula INT-1 or a salt thereof is a compound of formula INT-1-2, INT-1-a-2 or INT-1-b-2, or a salt thereof.
   33. The method of any one of Embodiments 29-30, wherein the reduction is carried out in the present of RuCl(p-cymene)[(S, S)-Ts-DPEN].
   34. The method of any one of Embodiments 29-30, wherein the reduction is carried out in the present of RuCl(p-cymene)[(S, S)-Fsdpen].
   35. The method of any one of Embodiments 29-30, wherein the reduction is carried out in the present of RuCl[(S, S)-TsDPEN](mesitylene).
   36. The method of any one of Embodiments 31-32, wherein the reduction is carried out in the present of RuCl(p-cymene)[(R, R)-Ts-DPEN].
   37. The method of any one of Embodiments 31-32, wherein the reduction is carried out in the present of RuCl(p-cymene)[(R, R)-Fsdpen].
   38. The method of any one of Embodiments 31-32, wherein the reduction is carried out in the present of RuCl[(R, R)-TsDPEN](mesitylene).
   39. The method of Embodiment of any one of Embodiments 1-2, wherein the reducing is carried out in the presence of H₂.
   40. The method of Embodiment of any one of Embodiments 1-2, wherein the reducing is carried out in the presence of an agent that delivers hydride.
   41. The method of Embodiment 40, wherein the agent that delivers hydride comprises or is NaBH₄.
   42. The method of Embodiment 40, wherein the agent that delivers hydride comprises or is LiBH₄.
   43. The method of any one of Embodiments 40-42, wherein a compound of formula P or a salt thereof is a compound of formula P-3, P-a-3, or P-b-3, or a salt thereof.
   44. The method of Embodiment 43, wherein a compound of formula INT-1 or a salt thereof is a compound of formula INT-1-2, INT-1-a-2 or INT-1-b-2, or a salt thereof.
   45. The method of any one of Embodiments 40-42, wherein a compound of formula P or a salt thereof is a compound of formula P-4, P-a-4, or P-b-4, or a salt thereof.
   46. The method of Embodiment 45, wherein a compound of formula INT-1 or a salt thereof is a compound of formula INT-1-1, INT-1-a-1 or INT-1-b-1, or a salt thereof.
   47. The method of any one of the preceding Embodiments, wherein the reducing is carried out in the presence of a protic solvent.
   48. The method of Embodiment 47, wherein the protic solvent is methanol.
   49. The method of Embodiment 47, wherein the protic solvent is ethanol.
   50. The method of any one of the preceding Embodiments, wherein L is —CH₂—.
   51. The method of any one of Embodiments 1-50, wherein L is —CH(CN)—.
   52. The method of any one of the preceding Embodiments, wherein R¹ is —S(O)₂R².
   53. The method of Embodiment 52, wherein R² is

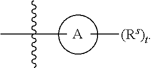

54. The method of Embodiment 53, wherein Ring A is an optionally substituted phenyl ring.
55. The method of any one of Embodiments 52-54, wherein t is 1.
56. The method of any one of the preceding Embodiments, wherein an occurrence of R^{s} is halogen.
57. The method of any one of the preceding Embodiments, wherein an occurrence of R^{s} is Cl.
58. The method of any one of the preceding Embodiments, wherein an occurrence of R^{s} is Br.
59. The method of any one of the preceding Embodiments, wherein an occurrence of R^{s} is CN.
60. The method of any one of the preceding Embodiments, wherein an occurrence of R^{s} is COOR′, wherein R′ is optionally substituted C₁₋₆ aliphatic.
61. The method of any one of the preceding Embodiments, wherein an occurrence of R^{s} is —COOMe.
62. The method of any one of the preceding Embodiments, wherein an occurrence of R^{s} is —COOEt.
63. The method of any one of the preceding Embodiments, wherein an occurrence of R^{s} is —OR′.
64. The method of any one of the preceding Embodiments, wherein an occurrence of R^{s} is —OR′, wherein R is optionally substituted C₁₋₆ aliphatic.
65. The method of Embodiment 63, wherein an occurrence of R^{s} is —OMe.
66. The method of any one of the preceding Embodiments, wherein an occurrence of R^{s} is N(R′)₂.
67. The method of Embodiment 66, wherein an occurrence of R^{s} is NMe₂.
68. The method of any one of the preceding Embodiments, wherein an occurrence of R^{s} is optionally substituted C₆₋₁₀ aryl.
69. The method of Embodiment 68, wherein an occurrence of R^{s} is phenyl.
70. The method of any one of the preceding Embodiments, wherein an occurrence of R^{s} is 5-20 membered heteroaryl having 1-4 heteroatoms independently selected from oxygen, nitrogen, sulfur, phosphorus and silicon.
71. The method of any one of the preceding Embodiments, wherein an occurrence of R^{s} is 5-10 membered heteroaryl having 1-4 heteroatoms independently selected from oxygen, nitrogen, sulfur, phosphorus and silicon.
72. The method of any one of Embodiments 1-52, wherein R² is optionally substituted phenyl.
73. The method of any one of Embodiments 1-52, wherein R² is phenyl.
74. The method of any one of Embodiments 1-52, wherein R² is optionally substituted C₁₋₆ aliphatic.
75. The method of Embodiment 74, wherein R² is optionally substituted C₁₋₆ alkyl.
76. The method of Embodiment 75, wherein R² is methyl.
77. The method of Embodiment 75, wherein R² is ethyl.
78. The method of Embodiment 75, wherein R² is propyl.
79. The method of Embodiment 75, wherein R² is isopropyl.
80. The method of Embodiment 75, wherein R² is n-butyl.
81. The method of Embodiment 75, wherein R² is cyclopentyl.
82. The method of Embodiment 75, wherein R² is cyclohexyl.
83. The method of Embodiment 75, wherein R² is cyclobutyl.
84. The method of any one of Embodiments 1-51, wherein R² is —N(R′)₂.
85. The method of Embodiment 84, wherein R² is —NMe₂.
86. The method of any one of Embodiments 1-51, wherein R¹ is —Si(R)₃.
87. The method of Embodiment 86, wherein each R is not —H.
88. The method of Embodiment 86, wherein each R is independently an optionally substituted C₁₋₃₀ aliphatic group.
89. The method of Embodiment 87, wherein each R is independently a C₁₋₁₀ aliphatic group.
90. The method of Embodiment 89, wherein each R is independently methyl.
91. The method of Embodiment 89, wherein each R is independently ethyl.
92. The method of Embodiment 86, wherein each R is independently an optionally substituted group selected from C₁₋₃₀ aliphatic and C₆₋₃₀ aryl.
93. The method of Embodiment 92, wherein each R is independently an optionally substituted group selected from C₁₋₁₀ aliphatic and phenyl.
94. The method of Embodiment 86 wherein R¹ is —Si(Ph)₂Me.
95. The method of any one of Embodiments 1-51, wherein R¹ is —P(O)(R²)₂.
96. The method of Embodiment 95, wherein an occurrence of R² is

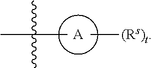

97. The method of any one of Embodiments 95-96, wherein Ring A is an optionally substituted phenyl ring.
98. The method of any one of Embodiments 95-97, wherein an occurrence of R² is optionally substituted

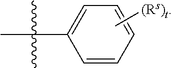

99. The method of any one of any one of Embodiments 95-98, wherein t is 1.
100. The method of any one of any one of Embodiments 95-99, wherein an occurrence of R^{s} is halogen.
101. The method of any one of any one of Embodiments 95-100, wherein an occurrence of R^{s} is Cl.
102. The method of any one of Embodiments 95-100, wherein an occurrence of R^{s} is Br.
103. The method of any one of Embodiments 95-99, wherein an occurrence of R^{s} is CN.
104. The method of any one of Embodiments 95-99, wherein an occurrence of R^{s} is COOR′, wherein R′ is optionally substituted C₁₋₆ aliphatic.
105. The method of Embodiment 104, wherein an occurrence of R^{s} is —COOMe.
106. The method of Embodiment 104, wherein an occurrence of R^{s} is —COOEt.
107. The method of any one of Embodiments 95-99, wherein an occurrence of R^{s} is —OR′.
108. The method of Embodiment 107, wherein an occurrence of R^{s} is —OMe.
109. The method of any one of Embodiments 95-99, wherein an occurrence of R^{s} is N(R′)₂.
110. The method of Embodiment 109, wherein an occurrence of R^{s} is NMe₂.
111. The method of any one of Embodiments 95-99, wherein an occurrence of R^{s} is optionally substituted C₆₋₁₀ aryl.
112. The method of Embodiment 111, wherein an occurrence of R^{s} is phenyl.
113. The method of any one of Embodiments 95-99, wherein an occurrence of R^{s} is 5-20 membered heteroaryl having 1-3 heteroatoms independently selected from oxygen, nitrogen, sulfur, phosphorus and silicon.
114. The method of any one of Embodiments 95-99, wherein an occurrence of R² is

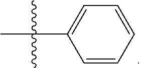 [SMILES: *c1ccccc1]

115. The method of Embodiment 95, wherein an occurrence of an occurrence of R² is —N(R′)₂.
116. The method of Embodiment 116, wherein an occurrence of R² is —NMe₂.
117. The method of any one of Embodiments 95-116, wherein the other occurrence of R² is —OR.
118. The method of any one of Embodiments 1-51, wherein R′ is —H.
119. The method of any one of the preceding Embodiments, wherein n is 0.
120. The method of any one of the preceding Embodiments, wherein n is 1.
121. The method of any one of the preceding Embodiments, wherein n is 2.
122. The method of any one of the preceding Embodiments, wherein n is 3.
123. The method of any one of the preceding Embodiments, wherein the compound of formula P or a salt thereof is formed with a de of about or at least about 50%, 60%, 70%, 80%, 90%, 965%, 96%, 97%, 98%, or 99%.
124. The method of any one of the preceding Embodiments, wherein the compound of formula P-a has the structure of:

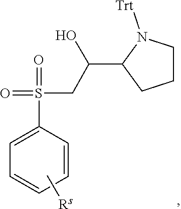

or a salt thereof,
wherein R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon.
   125. The method of Embodiment 124, wherein the compound of formula P-a has the structure of:

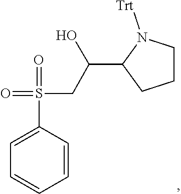

or a salt thereof.
126. The method of any one of Embodiments 1-123, wherein the compound of formula P-a has the structure of:

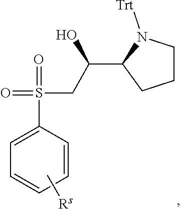

or a salt thereof,
wherein R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon.
   127. The method of Embodiment 126, wherein the compound of formula P-a has the structure of:

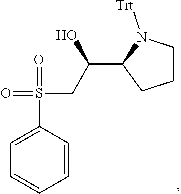

or a salt thereof.
128. The method of any one of Embodiments 1-123, wherein the compound of formula P-a has the structure of:

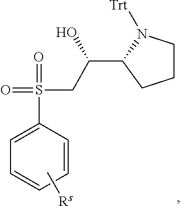

or a salt thereof,
wherein R^{s} is H, Cl, Br, CN, COOMe, COOEt, OMe, NMe₂, Ph, or a 5-20 membered heteroaryl having 1-3 heteroatoms independently selected from oxygen, nitrogen, sulfur, phosphorus and silicon. 129. The method of Embodiment 128, wherein the compound of formula P-a has the structure of:

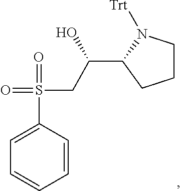

or a salt thereof.
130. The method of any one of Embodiments 1-123, wherein the compound of formula P-a has the structure of:

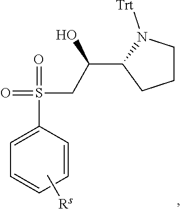

or a salt thereof,
wherein R^{s} is H, Cl, Br, CN, COOMe, COOEt, OMe, NMe₂, Ph, or a 5-20 membered heteroaryl having 1-3 heteroatoms independently selected from oxygen, nitrogen, sulfur, phosphorus and silicon.
   131. The method of Embodiment 130, wherein the compound of formula P-a has the structure of:

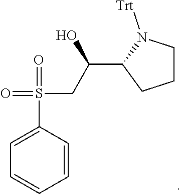

132. The method of any one of Embodiments 1-123, wherein the compound of formula P-a has the structure of:

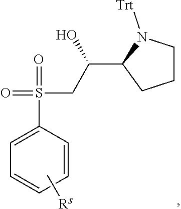

or a salt thereof,
wherein R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon.
   133. The method of Embodiment 132, wherein the compound of formula P-a has the structure of:

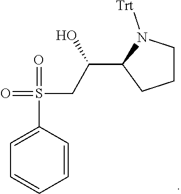

134. A method for preparing a compound of formula INT-1:

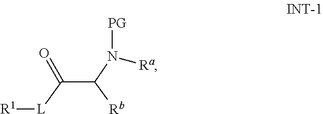

or a salt thereof, comprising reacting a compound of formula INT-2:

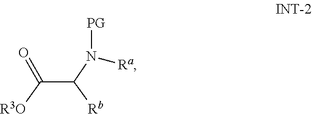

or a salt thereof with a compound of formula INT-3:

R¹-L-H, INT-3

or a salt thereof to provide the compound of formula INT-1 or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or

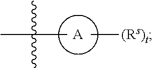

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   135. A method for preparing a compound of formula INT-1-a:

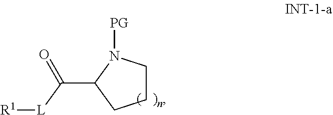

or a salt thereof,
comprising reacting a compound of formula INT-2-a:

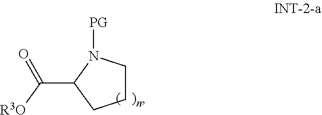

or a salt thereof with a compound of formula INT-3:

R¹-L-H, INT-3

or a salt thereof to provide the compound of formula INT-1 or a salt thereof, wherein:
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

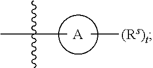

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   136. The method of any one of Embodiments 1-133, further comprising a method of Embodiment 134.
   137. The method of any one of Embodiments 1-133, further comprising a method of Embodiment 135.
   138. The method of any one of Embodiments 134-137, wherein L is —CH₂—.
   139. The method of any one of Embodiments 134-137, wherein L is —CH(CN)—.
   140. The method of any one of Embodiments 134-139, wherein R¹ is —S(O)₂R².
   141. The method of Embodiment 140, wherein R² is

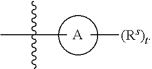

142. The method of Embodiment 141, wherein Ring A is optionally substituted phenyl.
143. The method of any one of Embodiments 141-142, wherein R² is optionally substituted

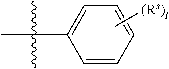

144. The method of any one of any one of Embodiments 141-143, wherein t is 1.
145. The method of any one of any one of Embodiments 141-144, wherein an occurrence of R^{s} is halogen.
146. The method of any one of any one of Embodiments 141-145, wherein an occurrence of R^{s} is Cl.
147. The method of any one of Embodiments 141-145, wherein an occurrence of R^{s} is Br.
148. The method of any one of Embodiments 141-144, wherein an occurrence of R^{s} is CN.
149. The method of any one of Embodiments 141-144, wherein an occurrence of R^{s} is COOR′, wherein R′ is optionally substituted C₁₋₆ aliphatic.
150. The method of Embodiment 149, wherein an occurrence of R^{s} is —COOMe.
151. The method of Embodiment 149, wherein an occurrence of R^{s} is —COOEt.
152. The method of any one of Embodiments 141-144, wherein an occurrence of R^{s} is —OR′.
153. The method of Embodiment 152, wherein an occurrence of R^{s} is —OMe.
154. The method of any one of Embodiments 141-144, wherein an occurrence of R^{s} is N(R′)₂.
155. The method of Embodiment 154, wherein an occurrence of R^{s} is NMe₂.
156. The method of any one of Embodiments 141-144, wherein an occurrence of R^{s} is optionally substituted C₆₋₁₀ aryl.
157. The method of Embodiment 156, wherein an occurrence of R^{s} is phenyl.
158. The method of any one of Embodiments 141-144, wherein an occurrence of R^{s} is 5-20 membered heteroaryl having 1-3 heteroatoms independently selected from oxygen, nitrogen, sulfur, phosphorus and silicon.
159. The method of any one of Embodiments 140-144, wherein R² is optionally substituted phenyl.
160. The method of any one of Embodiments 140-144, wherein R² is phenyl.
161. The method of Embodiment 140, wherein R² is optionally substituted C₁₋₆ aliphatic.
162. The method of Embodiment 161, wherein R² is optionally substituted C₁₋₆ alkyl.
163. The method of Embodiment 162, wherein R² is methyl.
164. The method of Embodiment 162, wherein R² is ethyl.
165. The method of Embodiment 162, wherein R² is propyl.
166. The method of Embodiment 162, wherein R² is isopropyl.
167. The method of Embodiment 162, wherein R² is n-butyl.
168. The method of Embodiment 162, wherein R² is cyclopentyl.
169. The method of Embodiment 162, wherein R² is cyclohexyl.
170. The method of Embodiment 162, wherein R² is cyclobutyl.
171. The method of Embodiment 140, wherein R² is —N(R′)₂.
172. The method of Embodiment 171, wherein R² is —NMe₂.
173. The method of any one of Embodiments 134-139, wherein R¹ is —Si(R)₃.
174. The method of Embodiment 173, wherein each R is independently an optionally substituted C₁₋₃₀ aliphatic group.
175. The method of Embodiment 174, wherein each R is independently a C₁₋₁₀ aliphatic group.
176. The method of Embodiment 175, wherein each R is independently methyl.
177. The method of Embodiment 175, wherein each R is independently ethyl.
178. The method of Embodiment 173, wherein each R is independently an optionally substituted group selected from C₁₋₃₀ aliphatic and C₆₋₃₀ aryl.
179. The method of Embodiment 178, wherein each R is independently an optionally substituted group selected from C₁₋₁₀ aliphatic and phenyl.
180. The method of Embodiment 179, wherein R¹ is —Si(Ph)₂Me.
181. The method of any one of Embodiments 134-139, wherein R¹ is —P(O)(R²)₂.
182. The method of Embodiment 181, wherein an occurrence of R² is

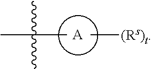

183. The method of any one of Embodiments 181-182, wherein Ring A is an optionally substituted phenyl ring.
184. The method of any one of Embodiments 181-183, wherein R² is optionally substituted

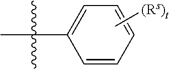

185. The method of any one of any one of Embodiments 181-184, wherein t is 1.
186. The method of any one of any one of Embodiments 181-185, wherein an occurrence of R^{s} is halogen.
187. The method of any one of any one of Embodiments 181-186, wherein an occurrence of R^{s} is Cl.
188. The method of any one of Embodiments 181-186, wherein an occurrence of R^{s} is Br.
189. The method of any one of Embodiments 181-185, wherein an occurrence of R^{s} is CN.
190. The method of any one of Embodiments 181-185, wherein an occurrence of R^{s} is COOR′, wherein R′ is optionally substituted C₁₋₆ aliphatic.
191. The method of Embodiment 190, wherein an occurrence of R^{s} is —COOMe.
192. The method of Embodiment 190, wherein an occurrence of R^{s} is —COOEt.
193. The method of any one of Embodiments 181-185, wherein an occurrence of R^{s} is —OR′.
194. The method of Embodiment 193, wherein an occurrence of R^{s} is —OMe.
195. The method of any one of Embodiments 181-185, wherein an occurrence of R^{s} is N(R′)₂.
196. The method of Embodiment 195, wherein an occurrence of R^{s} is NMe₂.
197. The method of any one of Embodiments 181-185, wherein an occurrence of R^{s} is optionally substituted C₆₋₁₀ aryl.
198. The method of Embodiment 197, wherein an occurrence of R^{s} is phenyl.
199. The method of any one of Embodiments 181-185, wherein an occurrence of R^{s} is 5-20 membered heteroaryl having 1-3 heteroatoms independently selected from oxygen, nitrogen, sulfur, phosphorus and silicon.
200. The method of any one of Embodiments 181-185, wherein an occurrence of R² is

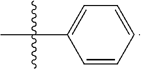 [SMILES: *c1ccccc1]

201. The method of Embodiment 181, wherein an occurrence of R² is —N(R′)₂.
202. The method of Embodiment 201, wherein an occurrence of R² is —NMe₂.
203. The method of any one of Embodiments 181-202, wherein an occurrence of R² is —OR.
204. The method of any one of Embodiments 134-139, wherein R¹ is H.
205. The method of any one of Embodiments 134-204, wherein n is 0.
206. The method of any one of Embodiments 134-204, wherein n is 1.
207. The method of any one of Embodiments 134-204, wherein n is 2.
208. The method of any one of Embodiments 134-204, wherein n is 3.
209. The method of any one of Embodiments 134-208, wherein R³ is optionally substituted C₁₋₁₀ aliphatic.
210. The method of Embodiment 209, wherein R³ is methyl.
211. The method of any one of Embodiments 134-210, wherein the reacting is performed in the presence of a base.
212. The method of Embodiment 211, wherein the base is LiHMDS.
213. The method of any one of Embodiments 134-212, wherein a compound of INT-1 or a salt thereof is a compound of formula INT-1-1, INT-1-a-1 or INT-1-b-1 or a salt thereof.
214. The method of Embodiment 213, wherein a compound of INT-2 or a salt thereof is a compound of formula INT-2-1, INT-2-a-1 or INT-2-b-1 or a salt thereof.
215. The method of any one of Embodiments 134-212, wherein a compound of INT-1 or a salt thereof is a compound of formula INT-1-2, INT-1-a-2 or INT-1-b-2 or a salt thereof.
216. The method of Embodiment 215, wherein a compound of INT-2 or a salt thereof is a compound of formula INT-2-2, INT-2-a-2 or INT-2-b-2 or a salt thereof.
217. The method of any one of the preceding Embodiments, further comprising: providing a compound of formula INT-4:

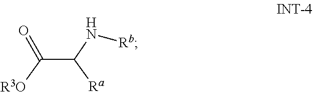

or a salt thereof; and
reacting a compound of formula INT-4 with an amino protecting agent to provide a compound of formula INT-2.
   218. The method of any one of the preceding Embodiments, further comprising: providing a compound of formula INT-4-a:

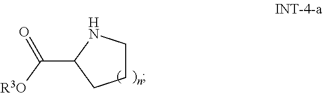

or a salt thereof, and
reacting a compound of formula INT-4-a with an amino protecting agent to provide a compound of formula INT-2-a.
   219. The method of any one of Embodiments 217-218, wherein the amino protecting agent has the structure of PG-LG, wherein LG is a leaving group.
   220. The method of Embodiment 219, wherein LG is —Cl.
   221. The method of any one of Embodiments 217-220, wherein a compound of INT-2 or a salt thereof is a compound of formula INT-2-1, INT-2-a-1 or INT-2-b-1 or a salt thereof.
   222. The method of Embodiment 221, wherein a compound of INT-4 or a salt thereof is a compound of formula INT-4-1, INT-4-a-1 or INT-4-b-1 or a salt thereof.
   223. The method of any one of Embodiments 217-220, wherein a compound of INT-2 or a salt thereof is a compound of formula INT-2-2, INT-2-a-2 or INT-2-b-2 or a salt thereof.
   224. The method of Embodiment 223, wherein a compound of INT-4 or a salt thereof is a compound of formula INT-4-2, INT-4-a-2 or INT-4-b-2 or a salt thereof.
   225. A method for preparing the compound of formula I:

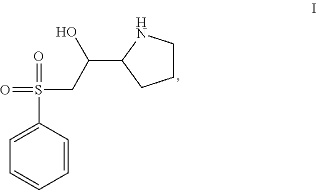 [SMILES: O=S(=O)(CC(O)C1CCCN1)c1ccccc1]

or a salt thereof, comprising:
(a) providing a compound having the structure of:

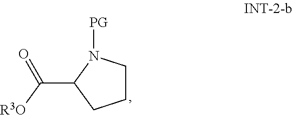

or a salt thereof, wherein:
PG is an amino protecting group;
R³ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; and
(b) reacting a compound of formula INT-2-b with

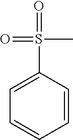

to provide a compound of formula INT-7:

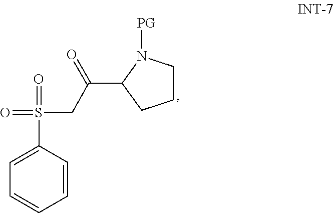

or a salt thereof, and
(c) reducing the compound of formula INT-7 to provide a compound of formula P-I:

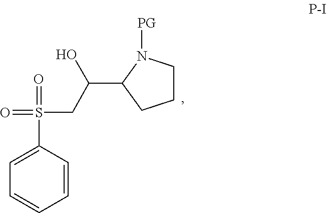

or a salt thereof, wherein the reduction of a compound of formula INT-7 or a salt thereof is carried out in the presence of a reducing agent; and
(e) subject a compound of formula P-I to deprotection to provide a compound of formula I.
   226. The method of Embodiment 225, wherein the reduction of a compound of formula INT-7 or a salt thereof is carried out in the presence of HCOOH or a salt thereof.
   227. The method of Embodiment 225, wherein the reduction of a compound of formula INT-7 or a salt thereof is carried out in the presence of H₂.
   228. The method of any one of Embodiments 225-227, wherein the reduction is carried out in the present of an agent comprising a metal.
   229. The method of any one of Embodiments 225-228, wherein the reduction is carried out in the present of an agent comprising Ru.
   230. The method of any one of Embodiments 225-229, wherein the reduction is carried out in the present of Ru—[(S, S)-Ts-DPEN].
   231. The method of any one of Embodiments 225-229, wherein the reduction is carried out in the present of Ru—[(R, R)-Ts-DPEN].
   232. The method of any one of Embodiments 225-229, wherein the reduction is carried out in the present of RuCl[(S, S)-TsDPEN](mesitylene).
   233. The method of any one of Embodiments 225-229, wherein the reduction is carried out in the present of RuCl[(R, R)-TsDPEN](mesitylene).
   234. The method of any one of Embodiments 225-229, wherein the reduction is carried out in the present of RuCl(p-cymene)[(S, S)-Fsdpen].
   235. The method of any one of Embodiments 225-229, wherein the reduction is carried out in the present of RuCl(p-cymene)[(R, R)-Fsdpen].
   236. The method of Embodiment 225, wherein the reduction of a compound of formula INT-7 or a salt thereof is carried out in the presence of an agent that delivers hydride.
   237. The method of Embodiment 236, wherein the agent that delivers hydride comprises or is NaBH₄.
   238. The method of Embodiment 236, wherein the agent that delivers hydride comprises or is NaBH₃CN.
   239. The method of Embodiment 236, wherein the agent that delivers hydride comprises or is LiAlH₄.
   240. The method of any one of Embodiments 225-239, wherein the reduction of a compound of formula INT-7 or a salt thereof is carried out in the presence of a protic solvent.
   241. The method of Embodiment 240, wherein the protic solvent is methanol.
   242. The method of Embodiment 240, wherein the protic solvent is ethanol.
   243. The method of any one of Embodiments 225-242, further comprising: reacting a compound of formula INT-4-b:

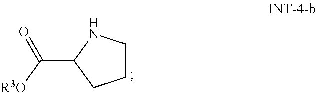

with an amino protecting agent to provide a compound of formula INT-2-b.
244. The method of any one of Embodiments 225-243, further comprising the:
(a) providing a compound of formula INT-5-b:

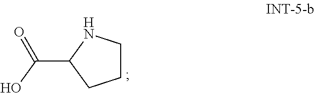

and
(b) reacting a compound of formula INT-5-b with a R³OH to provide a compound of formula INT-4-b.
   245. The method of any one of the preceding Embodiments, wherein the amino protecting agent has the structure of LG-PG or a salt thereof, wherein LG is a leaving group.
   246. The method of Embodiment 245, wherein the leaving group is chloride.
   247. The method of Embodiment 245, wherein the leaving group is tBu-O—COO—.
   248. The method of any one of the preceding Embodiments, wherein PG is —C(R)₃.
   249. The method of Embodiment 248, wherein PG is —C(R)₃, wherein each R is optionally substituted C₆₋₁₀ aryl.
   250. The method of Embodiment 248, wherein PG is —C(R)₃, wherein each R is optionally substituted phenyl.
   251. The method of Embodiment 248, wherein PG is trityl group.
   252. The method of any one of Embodiments 1-245, wherein PG is —C(O)O—C(R)₃.
   253. The method of Embodiment 252, wherein PG is Boc.
   254. The method of any one of Embodiments 1-245, wherein PG is R′.
   255. The method of any one of the preceding Embodiments, further comprising: reacting a compound of formula INT-5:

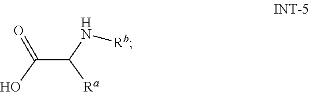

with a compound of R³OH to provide the compound of formula INT-4.
256. The method of any one of the preceding Embodiments, further comprising: reacting a compound of formula INT-5-a:

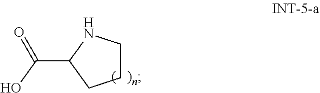

with a compound of R³OH to provide the compound of formula INT-4-a.
257. The method of any one of the preceding Embodiments, wherein R³ is optionally substituted C₁₋₁₀ aliphatic.
258. The method of Embodiment 256, wherein R³ is methyl.
259. The method of any one of the preceding Embodiments, further comprising subjecting the compound of formula P to an amino group deprotection condition to provide a compound of formula DP:

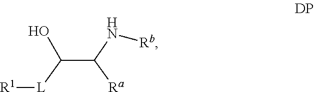

or a salt thereof.
260. The method of any one of the preceding Embodiments, further comprising subjecting the compound of formula P-a to an amino group deprotection condition to provide a compound of formula DP-a:

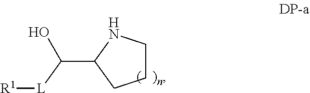

or a salt thereof.
261. The method of Embodiment 259 or 260, therein the deprotection is carried out in the presence of HCl.
262. The method of Embodiment 259 or 260, wherein the compound of formula DP or DP-a has the structure of:

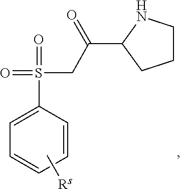

or a salt thereof,
wherein R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon.
   263. The method of Embodiment 259 or 260, wherein the compound of formula DP or DP-a has the structure of:

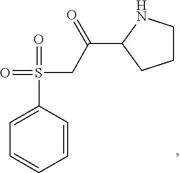 [SMILES: O=C(CS(=O)(=O)c1ccccc1)C1CCCN1]

or a salt thereof.
264. The method of any one of the preceding Embodiments, wherein the agent comprising a metal, if present, is of an amount of about or no more than about 0.05 equivalent.
265. The method of any one of the preceding Embodiments, wherein the agent comprising a metal, if present, is of an amount of about or no more than about 0.025 equivalent.
266. The method of any one of the preceding Embodiments, wherein the agent comprising a metal, if present, is of an amount of about or no more than about 0.01 equivalent.
267. The method of any one of the preceding Embodiments, wherein —OH and —N(PG)- are cis in a reduction product.
268. The method of Embodiment 266, wherein —OH and —N(PG)- are cis in a reduction product, and a cis product is formed with a selectivity of about 80%, 85%, 90%, 91%, 92%, 93%, 94%, 95%, 96%, 97%, 98%, 99% or more.
269. The method of Embodiment 268, wherein the selectivity is about 90% or more.
270. The method of Embodiment 268, wherein the selectivity is about 94% or more.
271. The method of Embodiment 268, wherein the selectivity is about 95% or more.
272. The method of Embodiment 268, wherein the selectivity is about 96% or more.
273. The method of any one of Embodiments 1-263, wherein —OH and —N(PG)- are trans in a reduction product.
274. The method of Embodiment 273, wherein —OH and —N(PG)- are trans in a reduction product, and a trans product is formed with a selectivity of about 80%, 85%, 90%, 91%, 92%, 93%, 94%, 95%, 96%, 97%, 98%, 99% or more.
275. The method of Embodiment 274, wherein the selectivity is about 90% or more.
276. The method of Embodiment 274, wherein the selectivity is about 94% or more.
277. The method of Embodiment 274, wherein the selectivity is about 95% or more.
278. The method of Embodiment 274, wherein the selectivity is about 96% or more.
279. The method of any one of the preceding Embodiments, wherein Ring B is 4-10 membered.
280. The method of any one of the preceding Embodiments, wherein Ring B is 4-membered.
281. The method of any one of the preceding Embodiments, wherein Ring B is 5-membered.
282. The method of any one of the preceding Embodiments, wherein Ring B is 6-membered.
283. The method of any one of the preceding Embodiments, wherein Ring B is 7-membered.
284. The method of any one of the preceding Embodiments, wherein Ring B is 8-membered.
285. The method of any one of the preceding Embodiments, wherein Ring B is monocyclic.
286. The method of any one of the preceding Embodiments, wherein Ring B is bicyclic.
287. The method of any one of the preceding Embodiments, wherein Ring B is polycyclic.
288. The method of any one of the preceding Embodiments, wherein each monocyclic ring unit is independently 3-10 membered having 0-4 heteroatoms.
289. The method of any one of the preceding Embodiments, wherein each monocyclic ring unit is independently 3-10 membered having 1-4 heteroatoms.
290. The method of any one of the preceding Embodiments, wherein Ring B has no heteroatoms in addition to the nitrogen atom.
291. The method of any one of the preceding Embodiments, wherein Ring B is saturated.
292. The method of any one of the preceding Embodiments, wherein Ring B is partially unsaturated.
293. The method of Embodiment 292, wherein each monocyclic ring unit is independently saturated, partially saturated or aromatic.
294. The Embodiments of any one of the preceding Embodiments, wherein Ring B is unsaturated.
295. A compound having the structure of formula INT-1:

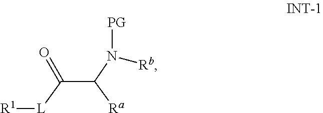

or a salt thereof to provide a compound of formula P or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or

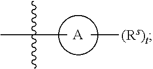

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   296. A compound having the structure of formula INT-1-a:

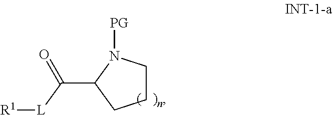

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

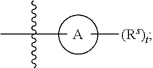

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   297. The compound of Embodiment 295 or 296, wherein the compound is a compound of formula INT-1-1 or a salt there of as described any one of the preceding Embodiments.
   298. The compound of Embodiment 295 or 296, wherein the compound is a compound of formula INT-1-2 or a salt there of as described any one of the preceding Embodiments.
   299. The compound of Embodiment 295 or 296, wherein the compound is a compound of formula INT-1-a-1 or a salt there of as described any one of the preceding Embodiments.
   300. The compound of Embodiment 295 or 296, wherein the compound is a compound of formula INT-1-a-2 or a salt there of as described any one of the preceding Embodiments.
   301. The compound of Embodiment 295 or 296, wherein the compound is a compound of formula INT-1-b-1 or a salt there of as described any one of the preceding Embodiments.
   302. The compound of Embodiment 295 or 296, wherein the compound is a compound of formula INT-1-b-2 or a salt there of as described any one of the preceding Embodiments.
   303. The compound of any one of Embodiments 302, wherein L is —CH₂—.
   304. The compound of any one of Embodiments 302, wherein L is —CH(CN)—.
   305. The compound of any one of Embodiments 295-304, wherein R¹ is —H.
   306. The compound of any one of Embodiments 295-304, wherein R¹ is —S(O)₂R².

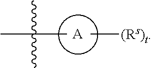

307. The compound of Embodiment 306, wherein R² is.
308. The compound of any one of the Embodiments 295-307, wherein Ring A is an optionally substituted phenyl ring.
309. The compound of any one of Embodiments 295-308, wherein R² is optionally substituted

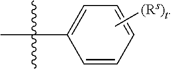

310. The compound of any one of Embodiments 295-309, wherein t is 1.
311. The compound of any one of Embodiments 295-310, wherein an occurrence of R^{s} is halogen.
312. The compound of any one of Embodiments 295-311, wherein an occurrence of R^{s} is Cl.
313. The compound of any one of Embodiments 295-311, wherein an occurrence of R^{s} is Br.
314. The compound of any one of Embodiments 295-310, wherein an occurrence of R^{s} is CN.
315. The compound of any one of Embodiments 295-310, wherein an occurrence of R^{s} is COOR′, wherein R′ is optionally substituted C₁₋₆ aliphatic.
316. The compound of Embodiment 315, wherein an occurrence of R^{s} is —COOMe.
317. The compound of Embodiment 315, wherein an occurrence of R^{s} is —COOEt.
318. The compound of any one of Embodiments 295-310, wherein an occurrence of R^{s} is —OR′.
319. The compound of Embodiment 318, wherein an occurrence of R^{s} is —OMe.
320. The compound of any one of Embodiments 295-310, wherein an occurrence of R^{s} is N(R′)₂.
321. The compound of Embodiment 320, wherein an occurrence of R^{s} is NMe₂.
322. The compound of any one of Embodiments 295-310, wherein an occurrence of R^{s} is optionally substituted C₆₋₁₀ aryl.
323. The compound of Embodiment 322, wherein an occurrence of R^{s} is phenyl.
324. The compound of any one of Embodiments 295-310, wherein an occurrence of R^{s} is 5-20 membered heteroaryl having 1-3 heteroatoms independently selected from oxygen, nitrogen, sulfur, phosphorus and silicon.
325. The compound of any one of Embodiments 295-310, wherein R² is optionally substituted phenyl.
326. The compound of any one of Embodiments 295-310, wherein R² is phenyl.
327. The compound of any one of Embodiments 295-306, wherein R² is optionally substituted C₁₋₆ aliphatic.
328. The compound of Embodiment 327, wherein R² is optionally substituted C₁₋₆ alkyl.
329. The compound of Embodiment 328, wherein R² is methyl.
330. The compound of Embodiment 328, wherein R² is ethyl.
331. The compound of Embodiment 328, wherein R² is propyl.
332. The compound of Embodiment 328, wherein R² is isopropyl.
333. The compound of Embodiment 328, wherein R² is n-butyl.
334. The compound of Embodiment 328, wherein R² is cyclopentyl.
335. The compound of Embodiment 328, wherein R² is cyclohexyl.
336. The compound of Embodiment 328, wherein R² is cyclobutyl.
337. The compound of any one of Embodiments 295-306, wherein R² is —N(R′)₂.
338. The compound of Embodiment 337, wherein R² is NMe₂.
339. The compound of any one of Embodiments 295-304, wherein R¹ is —Si(R)₃.
340. The compound of Embodiment 339, wherein each R is independently an optionally substituted C₁₋₃₀ aliphatic group.
341. The compound of Embodiment 340, wherein each R is independently a C₁₋₁₀ aliphatic group.
342. The compound of Embodiment 341, wherein each R is independently methyl.
343. The compound of Embodiment 341, wherein each R is independently ethyl.
344. The compound of Embodiment 339, wherein each R is independently an optionally substituted group selected from C₃₋₁₀ aliphatic and C₆₋₃₀ aryl.
345. The compound of Embodiment 344, wherein each R is independently an optionally substituted group selected from C₁₋₁₀ aliphatic and phenyl.
346. The compound of Embodiment 345, wherein R¹ is —Si(Ph)₂Me.
347. The compound of any one of Embodiments 295-346, wherein n is 0.
348. The compound of any one of Embodiments 295-346, wherein n is 1.
349. The compound of any one of Embodiments 295-346, wherein n is 2.
350. The compound of any one of Embodiments 295-346, wherein n is 3.
351. The compound of any one of Embodiments 295-350, wherein PG is —C(R′)₃.
352. The compound of Embodiment 351, wherein each R′ in PG is independently —H, or an optionally substituted group selected from C₁₋₃₀ aliphatic, C₃₋₁₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.
353. The compound of Embodiment 351 or 352, wherein each R′ in PG is independently optionally substituted C₆₋₃₀ aryl or 5-20 membered heteroaryl having 1-4 heteroatoms independently selected from oxygen, nitrogen, sulfur, phosphorus and silicon.
354. The compound of Embodiment 353, wherein each R′ in PG is independently optionally substituted phenyl.
355. The compound of Embodiment 354, wherein PG is -Tr.
356. The compound of any one of Embodiments 295-350, wherein PG is —C(O)R.
357. The compound of any one of Embodiments 295-350, wherein PG is —C(O)OR.
358. The compound of any one of Embodiments 295-350, wherein PG is Boc.
359. The compound of any one of the preceding Embodiments, wherein Ring B is 4-10 membered.
360. The compound of any one of the preceding Embodiments, wherein Ring B is 4-membered.
361. The compound of any one of the preceding Embodiments, wherein Ring B is 5-membered.
362. The compound of any one of the preceding Embodiments, wherein Ring B is 6-membered.
363. The compound of any one of the preceding Embodiments, wherein Ring B is 7-membered.
364. The compound of any one of the preceding Embodiments, wherein Ring B is 8-membered.
365. The compound of any one of the preceding Embodiments, wherein Ring B is monocyclic.
366. The compound of any one of the preceding Embodiments, wherein Ring B is bicyclic.
367. The compound of any one of the preceding Embodiments, wherein Ring B is polycyclic.
368. The compound of any one of the preceding Embodiments, wherein each monocyclic ring unit is independently 3-10 membered having 0-4 heteroatoms.
369. The compound of any one of the preceding Embodiments, wherein each monocyclic ring unit is independently 3-10 membered having 1-4 heteroatoms.
370. The compound of any one of the preceding Embodiments, wherein Ring B has no heteroatoms in addition to the nitrogen atom.
371. The compound of any one of the preceding Embodiments, wherein Ring B is saturated.
372. The compound of any one of the preceding Embodiments, wherein Ring B is partially unsaturated.
373. The compound of Embodiment 372, wherein each monocyclic ring unit is independently saturated, partially saturated or aromatic.
374. The compound of any one of the preceding Embodiments, wherein Ring B is unsaturated.
375. The compound of any one of Embodiments 295-306, wherein the compound has the structure of

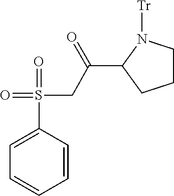

or a salt thereof.
376. The compound of any one of Embodiments 295-306, wherein the compound has the structure of

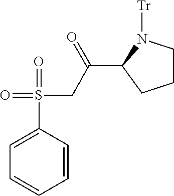

or a salt thereof.
377. The compound of any one of Embodiments 295-306, wherein the compound has the structure of

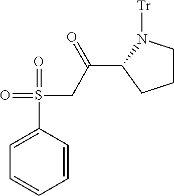

or a salt thereof.
378. A compound having the structure of

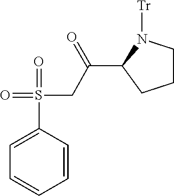

or a salt thereof.
379. A compound having the structure of

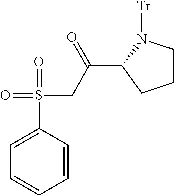

or a salt thereof.
380. The compound of any one of Embodiments 295-306, wherein the compound having the structure of

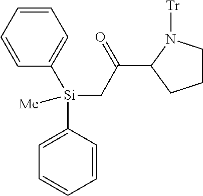

or a salt thereof.
381. The compound of any one of Embodiments 295-306, wherein the compound has the structure of

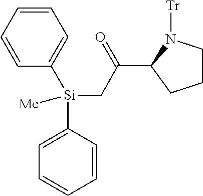

or a salt thereof.
382. The compound of any one of Embodiments 295-306, wherein the compound has the structure of

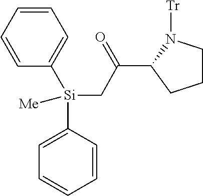

or a salt thereof.

383. A compound having the structure of

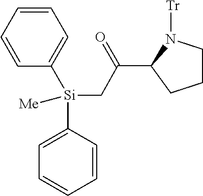

or a salt thereof.
384. A compound having the structure of

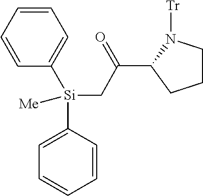

or a salt thereof.
385. The compound of any one of Embodiments 295-384, wherein the purity of the compound is or greater than about 10%, 20%, 30%, 40%, 50%, 60%, 70%, 80%, 85%, 90%, 91%, 92%, 93%, 94%, 95%, 96%, 97%, 98%, 99%, 99.7%, or 99.9%.
386. The compound of any one of Embodiments 295-384, wherein the purity of the compound is or greater than about 85%.
387. The compound of any one of Embodiments 295-384, wherein the purity of the compound is or greater than about 90%.
388. The compound of any one of Embodiments 295-384, wherein the purity of the compound is or greater than about 95%.
389. The compound of any one of Embodiments 295-384, wherein the purity of the compound is or greater than about 96%.
390. The compound of any one of Embodiments 295-384, wherein the purity of the compound is or greater than about 97%.
391. The compound of any one of Embodiments 295-384, wherein the purity of the compound is or greater than about 98%.
392. The compound of any one of Embodiments 295-384, wherein the purity of the compound is or greater than about 99%.
393. The compound of any one of Embodiments 295-384, wherein the purity of the compound is or greater than about 99.7%.
394. The compound of any one of Embodiments 295-384, wherein the purity of the compound is or greater than about 99.9%.
395. A composition comprising:
(1) a compound of formula INT-1

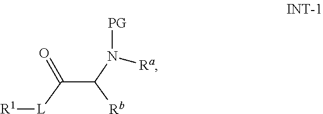

or a salt thereof, and
(2) a compound of formula P:

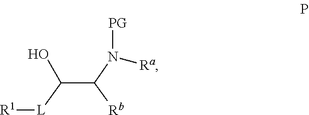

or a salt thereof; wherein
PG is an amino protecting group;
L is optionally substituted —CH₂—;
R¹ is R, —P(O)(R²)₂, —S(O)₂R², or —Si(R)₃;
R² is R, —OR, —N(R′)₂, or

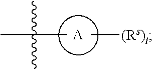

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   396. A composition comprising:
(1) a compound of formula INT-1-a:

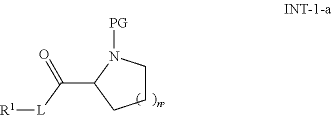

or a salt thereof, and
(2) a compound of formula P-a:

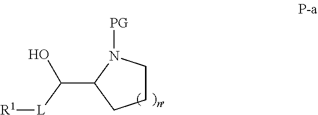

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

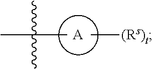

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   397. A composition comprising:
(1) a compound of formula INT-1:

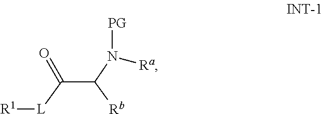

or a salt thereof, and
(2) a compound of formula INT-3:

R¹-L-H, INT-3

or a salt thereof; wherein
PG is an amino protecting group;
L is optionally substituted —CH₂—;
R¹ is R, —P(O)(R²)₂, —S(O)₂R², or —Si(R)₃;
R² is R, —OR, —N(R′)₂, or

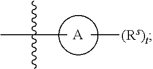

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   398. A composition comprising:
(1) a compound of formula INT-1-a:

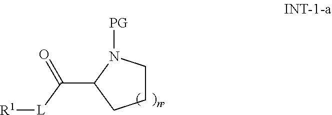

or a salt thereof, and
(2) a compound of formula INT-3:

R¹-L-H, INT-3

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

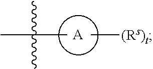

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   399. A composition comprising:
(1) a compound of formula INT-1:

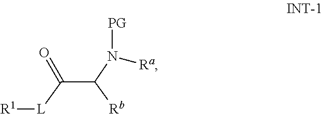

or a salt thereof, and
(2) a compound of formula INT-2:

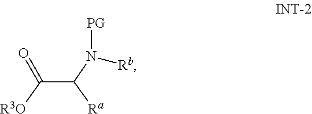

or a salt thereof; wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or

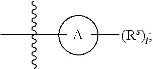

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   400. A composition comprising:
(1) a compound of formula INT-1-a:

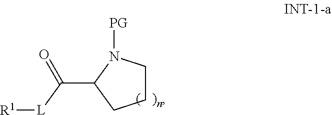

or a salt thereof, and
(2) a compound of formula INT-2-a:

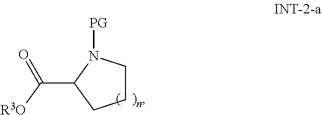

or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or

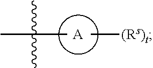

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   401. The composition of any one of Embodiments 395-400, wherein L is —CH₂—.
   402. The composition of any one of Embodiments 395-400, wherein L is —CH(CN)—.
   403. The composition of any one of Embodiments 395-402, wherein R¹ is —H.
   404. The composition of any one of Embodiments 395-402, wherein R¹ is —S(O)₂R².
   405. The composition of Embodiment 404, wherein R² is

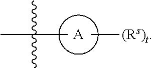

406. The composition of any one of the Embodiments 395-405, wherein Ring A is an optionally substituted phenyl ring.
407. The composition of any one of Embodiments 395-406, wherein R² is optionally substituted

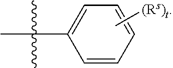

408. The composition of any one of Embodiments 395-407, wherein t is 1.
409. The composition of any one of Embodiments 395-408, wherein an occurrence of R^{s} is halogen.
410. The composition of any one of Embodiments 395-409, wherein an occurrence of R^{s} is Cl.
411. The composition of any one of Embodiments 395-409, wherein an occurrence of R^{s} is Br.
412. The composition of any one of Embodiments 395-408, wherein an occurrence of R^{s} is CN.
413. The composition of any one of Embodiments 395-408, wherein an occurrence of R^{s} is COOR′, wherein R′ is optionally substituted C₁₋₆ aliphatic.
414. The composition of Embodiment 413, wherein an occurrence of R^{s} is —COOMe.
415. The composition of Embodiment 413, wherein an occurrence of R^{s} is —COOEt.
416. The composition of any one of Embodiments 395-408, wherein an occurrence of R^{s} is —OR′.
417. The composition of Embodiment 416, wherein an occurrence of R^{s} is —OMe.
418. The composition of any one of Embodiments 395-408, wherein an occurrence of R^{s} is N(R′)₂.
419. The compound of Embodiment 419, wherein an occurrence of R^{s} is NMe₂.
420. The composition of any one of Embodiments 395-408, wherein an occurrence of R^{s} is optionally substituted C₆₋₁₀ aryl.
421. The composition of Embodiment 420, wherein an occurrence of R^{s} is phenyl.
422. The composition of any one of Embodiments 395-408, wherein an occurrence of R^{s} is 5-20 membered heteroaryl having 1-3 heteroatoms independently selected from oxygen, nitrogen, sulfur, phosphorus and silicon.
423. The composition of any one of Embodiments 395-407, wherein R² is optionally substituted phenyl.
424. The composition of any one of Embodiments 395-407, wherein R² is phenyl.
425. The composition of any one of Embodiments 395-404, wherein R² is optionally substituted C₁₋₆ aliphatic.
426. The composition of Embodiment 425, wherein R² is optionally substituted C₁₋₆ alkyl.
427. The composition of Embodiment 426, wherein R² is methyl.
428. The composition of Embodiment 426, wherein R² is ethyl.
429. The composition of Embodiment 426, wherein R² is propyl.
430. The composition of Embodiment 426, wherein R² is isopropyl.
431. The composition of Embodiment 426, wherein R² is n-butyl.
432. The composition of Embodiment 426, wherein R² is cyclopentyl.
433. The composition of Embodiment 426, wherein R² is cyclohexyl.
434. The composition of Embodiment 426, wherein R² is cyclobutyl.
435. The composition of any one of Embodiments 395-404, wherein R² is —N(R′)₂.
436. The composition of Embodiment 435, wherein R² is NMe₂.
437. The composition of any one of Embodiments 395-402, wherein R¹ is —P(O)(R²)₂.
438. The composition of any one of Embodiments 395-402, wherein R¹ is —Si(R)₃.
439. The composition of Embodiment 438, wherein each R is independently an optionally substituted C₁₋₃₀ aliphatic group.
440. The composition of Embodiment 439, wherein each R is independently a C₁₋₁₀ aliphatic group.
441. The composition of Embodiment 440, wherein each R is independently methyl.
442. The composition of Embodiment 440, wherein each R is independently ethyl.
443. The composition of Embodiment 438, wherein each R is independently an optionally substituted group selected from C₁₋₃₀ aliphatic and C₆₋₃₀ aryl.
444. The composition of Embodiment 443, wherein each R is independently an optionally substituted group selected from C₁₋₁₀ aliphatic and phenyl.
445. The composition of Embodiment 444, wherein R¹ is —Si(Ph)₂Me.
446. The composition of any one of Embodiments 395-445, wherein n is 0.
447. The composition of any one of Embodiments 395-445, wherein n is 1.
448. The composition of any one of Embodiments 395-445, wherein n is 2.
449. The composition of any one of Embodiments 395-445, wherein n is 3.
450. The composition of any one of Embodiments 395-449, wherein R³ is optionally substituted C₁₋₁₀ aliphatic.
451. The composition of Embodiment 450, wherein R³ is methyl.
452. The composition of any one of Embodiments 395-451, wherein PG is —C(O)R.
453. The composition of any one of Embodiments 395-451, wherein PG is —C(O)OR.
454. The composition of any one of Embodiments 395-451, wherein PG is -Boc.
455. The composition of any one of Embodiments 395-451, wherein PG is —C(R′)₃.
456. The composition of Embodiment 455, wherein each R′ in PG is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.
457. The composition of Embodiment 455 or 456, wherein each R′ in PG is independently optionally substituted C₆₋₃₀ aryl or 5-20 membered heteroaryl having 1-3 heteroatoms independently selected from oxygen, nitrogen, sulfur, phosphorus and silicon.
458. The composition of Embodiment 457, wherein each R′ in PG is independently optionally substituted phenyl.
459. The composition of Embodiment 458, wherein PG is -Tr.
460. The composition of any one of the preceding Embodiments, wherein Ring B is 4-10 membered.
461. The composition of any one of the preceding Embodiments, wherein Ring B is 4-membered.
462. The composition of any one of the preceding Embodiments, wherein Ring B is 5-membered.
463. The composition of any one of the preceding Embodiments, wherein Ring B is 6-membered.
464. The composition of any one of the preceding Embodiments, wherein Ring B is 7-membered.
465. The composition of any one of the preceding Embodiments, wherein Ring B is 8-membered.
466. The composition of any one of the preceding Embodiments, wherein Ring B is monocyclic.
467. The composition of any one of the preceding Embodiments, wherein Ring B is bicyclic.
468. The c composition of any one of the preceding Embodiments, wherein Ring B is polycyclic.
469. The composition of any one of the preceding Embodiments, wherein each monocyclic ring unit is independently 3-10 membered having 0-4 heteroatoms.
470. The composition of any one of the preceding Embodiments, wherein each monocyclic ring unit is independently 3-10 membered having 1-4 heteroatoms.
471. The composition of any one of the preceding Embodiments, wherein Ring B has no heteroatoms in addition to the nitrogen atom.
472. The composition of any one of the preceding Embodiments, wherein Ring B is saturated.
473. The composition of any one of the preceding Embodiments, wherein Ring B is partially unsaturated.
474. The composition of Embodiment 473, wherein each monocyclic ring unit is independently saturated, partially saturated or aromatic.
475. The composition of any one of the preceding Embodiments, wherein Ring B is unsaturated.
476. A composition, wherein the composition comprises

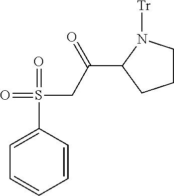

or a salt thereof and

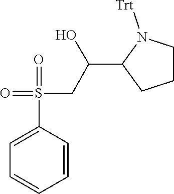

or a salt thereof.
477. A composition, wherein the composition comprises

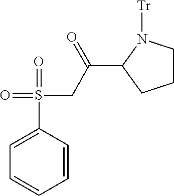

or a salt thereof and

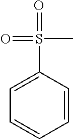

or a salt thereof.
478. A composition, wherein the composition comprises

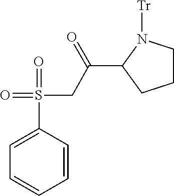

or a salt thereof and

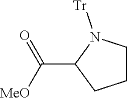

or a salt thereof.
479. The composition of any one of the preceding Embodiments, comprising a compound of formula P-1, P-a-1, P-b-1, or a salt thereof.
480. The composition of 479, wherein the compound of formula P-1, P-a-1, P-b-1, or a salt thereof has a de of about or at least about 50%, 60%, 70%, 80%, 90%, 965%, 96%, 97%, 98%, or 99% independently relative to each of its diastereomers in the composition.
481. The composition of Embodiment 479 or 480, wherein the compound of formula P-1, P-a-1, P-b-1, or a salt thereof has a ee of about or at least about 50%, 60%, 70%, 80%, 90%, 965%, 96%, 97%, 98%, or 99% in the composition.
482. The composition of any one of Embodiments 479-481, wherein the composition comprises a compound of formula INT-1-1, INT-1-a-1 or INT-1-b-1, or a salt thereof.
483. The composition of Embodiment 482, wherein the compound of formula INT-1-1, INT-1-a-1 or INT-1-b-1, or a salt thereof has a ee of about or at least about 50%, 60%, 70%, 80%, 90%, 965%, 96%, 97%, 98%, or 99% in the composition.
484. The composition of any one of Embodiments 479-483, further comprises an agent of any one of Embodiments 4-28 and 33-35.
485. The composition of any one of the preceding Embodiments, comprising a compound of formula P-2, P-a-2, P-b-2, or a salt thereof.
486. The composition of 485, wherein the compound of formula P-2, P-a-2, P-b-2, or a salt thereof has a de of about or at least about 50%, 60%, 70%, 80%, 90%, 965%, 96%, 97%, 98%, or 99% independently relative to each of its diastereomers in the composition.
487. The composition of Embodiment 485 or 486, wherein the compound of formula P-2, P-a-2, P-b-2, or a salt thereof has a ee of about or at least about 50%, 60%, 70%, 80%, 90%, 965%, 96%, 97%, 98%, or 99% in the composition.
488. The composition of any one of Embodiments 485-487, wherein the composition comprises a compound of formula INT-1-2, INT-1-a-2 or INT-1-b-2, or a salt thereof.
489. The composition of Embodiment 482, wherein the compound of formula INT-1-2, INT-1-a-2 or INT-1-b-2, or a salt thereof has a ee of about or at least about 50%, 60%, 70%, 80%, 90%, 965%, 96%, 97%, 98%, or 99% in the composition.
490. The composition of any one of Embodiments 485-489, further comprises an agent of any one of Embodiments 4-28 and 36-38.
491. The composition of any one of Embodiments 479-490, comprising HCOOH or a salt thereof.
492. The composition of any one of Embodiments 479-490, comprising HCOONa.
493. The composition of any one of the preceding Embodiments, comprising a compound of formula P-4, P-a-4, P-b-4, or a salt thereof.
494. The composition of 493, wherein the compound of formula P-4, P-a-4, P-b-4, or a salt thereof has a de of about or at least about 50%, 60%, 70%, 80%, 90%, 965%, 96%, 97%, 98%, or 99% independently relative to each of its diastereomers in the composition.
495. The composition of Embodiment 493 or 494, wherein the compound of formula P-4, P-a-4, P-b-4, or a salt thereof has a ee of about or at least about 50%, 60%, 70%, 80%, 90%, 965%, 96%, 97%, 98%, or 99% in the composition.
496. The composition of any one of Embodiments 493-495, wherein the composition comprises a compound of formula INT-1-1, INT-1-a-1 or INT-1-b-1, or a salt thereof.
497. The composition of Embodiment 496, wherein the compound of formula INT-1-1, INT-1-a-1 or INT-1-b-1, or a salt thereof has a ee of about or at least about 50%, 60%, 70%, 80%, 90%, 965%, 96%, 97%, 98%, or 99% in the composition.
498. The composition of any one of Embodiments 479-483, further comprises an agent of any one of Embodiments 40-42.
499. The composition of any one of the preceding Embodiments, comprising a compound of formula P-3, P-a-3, P-b-3, or a salt thereof.
500. The composition of 499, wherein the compound of formula P-3, P-a-3, P-b-3, or a salt thereof has a de of about or at least about 50%, 60%, 70%, 80%, 90%, 965%, 96%, 97%, 98%, or 99% independently relative to each of its diastereomers in the composition.
501. The composition of Embodiment 499 or 500, wherein the compound of formula P-3, P-a-3, P-b-3, or a salt thereof has a ee of about or at least about 50%, 60%, 70%, 80%, 90%, 965%, 96%, 97%, 98%, or 99% in the composition.
502. The composition of any one of Embodiments 499-501, wherein the composition comprises a compound of formula INT-1-2, INT-1-a-2 or INT-1-b-2, or a salt thereof.
503. The composition of Embodiment 502, wherein the compound of formula INT-1-2, INT-1-a-2 or INT-1-b-2, or a salt thereof has a ee of about or at least about 50%, 60%, 70%, 80%, 90%, 965%, 96%, 97%, 98%, or 99% in the composition.
504. The composition of any one of Embodiments 499-503, further comprises an agent of any one of Embodiments 40-42.
505. The composition of any one of the preceding Embodiments, comprising a compound of formula INT-2-1, INT-2-a-1, INT-2-b-1, or a salt thereof.
506. The composition of Embodiment 505, wherein the compound of formula INT-2-1, INT-2-a-1, INT-2-b-1, or a salt thereof has a ee of about or at least about 50%, 60%, 70%, 80%, 90%, 965%, 96%, 97%, 98%, or 99% in the composition.
507. The composition of any one of Embodiments 1-506, comprising a compound of formula INT-2-2, INT-2-a-2, INT-2-b-2, or a salt thereof.
508. The composition of Embodiment 507, wherein the compound of formula INT-2-2, INT-2-a-2, INT-2-b-2, or a salt thereof has a ee of about or at least about 50%, 60%, 70%, 80%, 90%, 965%, 96%, 97%, 98%, or 99% in the composition.
509. The method, compound or composition of any one of the preceding Embodiments, wherein the de is about or at least about 80%.
510. The method, compound or composition of any one of the preceding Embodiments, wherein the de is about or at least about 85%.
511. The method, compound or composition of any one of the preceding Embodiments, wherein the de is about or at least about 90%.
512. The method, compound or composition of any one of the preceding Embodiments, wherein the de is about or at least about 95%.
513. The method, compound or composition of any one of the preceding Embodiments, wherein the de is about or at least about 98%.
514. The method, compound or composition of any one of the preceding Embodiments, wherein the ee is about or at least about 80%.
515. The method, compound or composition of any one of the preceding Embodiments, wherein the ee is about or at least about 85%.
516. The method, compound or composition of any one of the preceding Embodiments, wherein the ee is about or at least about 90%.
517. The method, compound or composition of any one of the preceding Embodiments, wherein the ee is about or at least about 95%.
518. The method, compound or composition of any one of the preceding Embodiments, wherein the ee is about or at least about 98%.
519. The method, compound or composition of any one of the preceding Embodiments, wherein each R is independently —H, or an optionally substituted group selected from C₁₋₁₀ aliphatic, C₁₋₁₀ heteroaliphatic having 1-5 heteroatoms independently selected from oxygen, nitrogen, sulfur, phosphorus and silicon, C₆₋₁₀ aryl, C₆₋₂₀ arylaliphatic, C₆₋₂₀ arylheteroaliphatic having 1-5 heteroatoms independently selected from oxygen, nitrogen, sulfur, phosphorus and silicon, 5-10 membered heteroaryl having 1-5 heteroatoms independently selected from oxygen, nitrogen, sulfur, phosphorus and silicon, and 3-10 membered heterocyclyl having 1-5 heteroatoms independently selected from oxygen, nitrogen, sulfur, phosphorus and silicon; or:
two R groups are optionally and independently taken together to form a covalent bond; or:
two R groups are optionally and independently taken together with the atom to form an optionally substituted, 3-15 membered, monocyclic, bicyclic or polycyclic ring having, in addition to the atom, 0-5 heteroatoms independently selected from oxygen, nitrogen, sulfur, phosphorus and silicon; or
two or more R groups are optionally and independently taken together with their intervening atoms to form an optionally substituted, 3-15 membered, monocyclic, bicyclic or polycyclic ring having, in addition to the intervening atoms, 0-5 heteroatoms independently selected from oxygen, nitrogen, sulfur, phosphorus and silicon.
   520. A compound, wherein the compound has a structure of PMT-A:

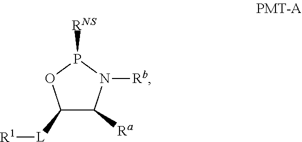

or a salt thereof, wherein:
R^{NS} is a nucleoside;
L is optionally substituted —CH₂—;
R¹ is R, —P(O)(R²)₂, —S(O)₂R², or —Si(R)₃;
R² is R, —OR, —N(R′)₂, or

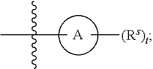

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   521. A compound, wherein the compound has a structure of PMT-B:

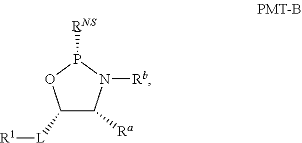

or a salt thereof, wherein:
R^{NS} is a nucleoside;
L is optionally substituted —CH₂—;
R¹ is R, —P(O)(R²)₂, —S(O)₂R², or —Si(R)₃;
R² is R, —OR, —N(R′)₂, or

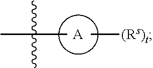

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   522. The compound of Embodiment 520 or 521, wherein R^{NS} is a nucleoside comprising a protecting group.
   523. The compound of any one of Embodiments 520-522, wherein R^{NS} is a nucleoside suitably protected for oligonucleotide synthesis.
   524. The compound of any one of Embodiments 520-523, wherein R^{NS} is —O—SU-BA, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
—O—SU— is —O-L^{s}- or

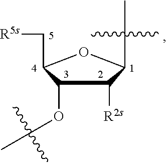

wherein —O—SU— is connected to the phosphorus atom in formula PMT-A or PMT-B through the oxygen atom;
L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon.
   525. The compound of Embodiment 524, wherein —O—SU— is

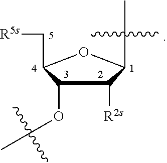

526. A compound, wherein the compound has a structure of PMT-A1:

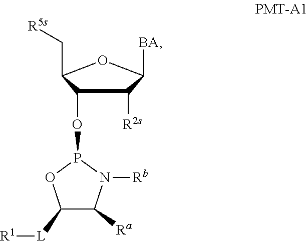

or a salt thereof, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon;
L is optionally substituted —CH₂—;
R¹ is R, —P(O)(R²)₂, —S(O)₂R², or —Si(R)₃;
R² is R, —OR, —N(R′)₂, or

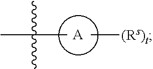

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   527. A compound, wherein the compound has a structure of PMT-B1:

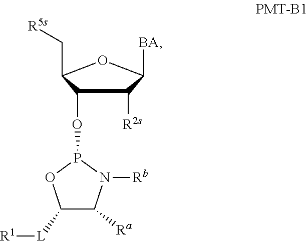

or a salt thereof, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
L^{s} is a covalent bond, or a bivalent, optionally substituted, linear or branched group selected from C₁₋₃₀ aliphatic and C₃₋₁₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon;
L is optionally substituted —CH₂—;
R¹ is R, —P(O)(R²)₂, —S(O)₂R², or —Si(R)₃;
R² is R, —OR, —N(R′)₂, or

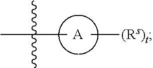

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   528. The compound of any one of Embodiments 524-542, wherein R^{2s} is L^{s} connecting C2 with C₄, wherein L^{s} is (C2)-O-(optionally substituted methylene)-.
   529. The compound of any one of Embodiments 524-542, wherein R^{2s} is —OR′, wherein R′ is optionally substituted C₁₋₆ aliphatic.
   530. The compound of any one of Embodiments 524-542, wherein R^{2s} is selected form —H, —F, —OMe, and —OCH₂CH₂OMe.
   531. The compound of any one of Embodiments 524-542, wherein R^{2s} is —OMe.
   532. The compound of any one of Embodiments 524-542, wherein R^{2s} is —OCH₂CH₂OMe.
   533. The compound of any one of Embodiments 524-542, wherein R^{2s} is —F.
   534. The compound of any one of Embodiments 524-542, wherein R^{2s} is —H.
   535. The compound of any one of Embodiments 524-534, wherein R^{5s} is —OR′.
   536. The compound of any one of Embodiments 524-535, wherein R^{5s} is -ODMTr.
   537. The compound of any one of Embodiments 524-536, wherein BA is a natural nucleobase moiety or a modified nucleobase moiety.
   538. The compound of any one of Embodiments 524-536, wherein BA is an optionally substituted group selected from C₃₋₁₅ cycloaliphatic, C₆₋₁₄ aryl, C₃₋₁₅ heterocyclyl having 1-5 heteroatoms independently selected from oxygen, nitrogen, sulfur, phosphorus and silicon, C₅₋₁₄ heteroaryl having 1-5 heteroatoms independently selected from oxygen, nitrogen, sulfur, phosphorus and silicon.
   539. The compound of any one of Embodiments 524-536, wherein BA is optionally substituted C₅₋₁₄ heteroaryl having 1-5 heteroatoms independently selected from oxygen, nitrogen, and sulfur.
   540. The compound of any one of Embodiments 524-536, wherein BA is optionally substituted A, T, C, G, U or a tautomer thereof.
   541. The compound of any one of Embodiments 524-536, wherein BA is optionally protected A, T, C, G, U or a tautomer thereof.
   542. The compound of any one of Embodiments 524-537, wherein BA is optionally substituted or protected U, or is an optionally substituted or protected tautomer of U, or is optionally substituted or protected C, or is an optionally substituted or protected tautomer of C, or is optionally substituted or protected A, or is an optionally substituted or protected tautomer of A, or is optionally substituted or protected nucleobase of pseudoisocytosine, or is an optionally substituted or protected tautomer of the nucleobase of pseudoisocytosine.
   543. The compound of any one of Embodiments 524-542, wherein BA is an optionally substituted group which group is selected from

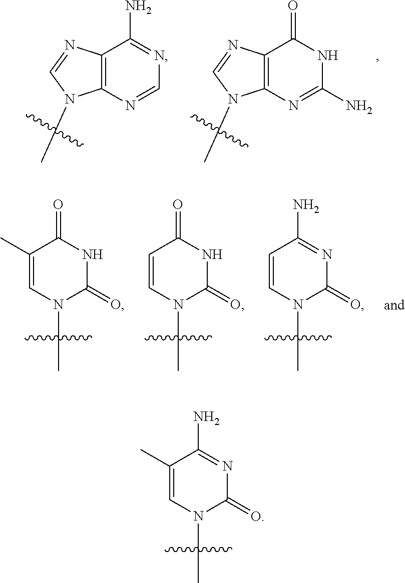

544. The compound of any one of Embodiments 524-542, wherein BA an optionally substituted guanine residue wherein its O⁶ is unprotected.
545. The compound of any one of Embodiments 520-544, wherein R¹ is —S(O)₂R².
546. The compound of any one of Embodiments 520-545, wherein R² is

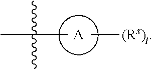

547. The compound of any one of Embodiments 520-546, wherein Ring A is an optionally substituted phenyl ring.
548. The compound of any one of Embodiments 520-547, wherein t is 1.
549. The compound of any one of Embodiments 520-548, wherein an occurrence of R^{s} is halogen.
550. The compound of any one of Embodiments 520-548, wherein an occurrence of R^{s} is Cl.
551. The compound of any one of Embodiments 520-548, wherein an occurrence of R^{s} is Br.
552. The compound of any one of Embodiments 520-548, wherein an occurrence of R^{s} is CN.
553. The compound of any one of Embodiments 520-548, wherein an occurrence of R^{s} is COOR′, wherein R′ is optionally substituted C₁₋₆ aliphatic.
554. The compound of any one of Embodiments 520-548, wherein an occurrence of R^{s} is —COOMe.
555. The compound of any one of Embodiments 520-548, wherein an occurrence of R^{s} is —COOEt.
556. The compound of any one of Embodiments 520-548, wherein an occurrence of R^{s} is —OR′.
557. The compound of any one of Embodiments 520-548, wherein an occurrence of R^{s} is —OR′, wherein R is optionally substituted C₁₋₆ aliphatic.
558. The compound of any one of Embodiments 520-548, wherein an occurrence of R^{s} is —OMe.
559. The compound of any one of Embodiments 520-548, wherein an occurrence of R^{s} is N(R′)₂.
560. The compound of any one of Embodiments 520-548, wherein an occurrence of R^{s} is NMe₂.
561. The compound of any one of Embodiments 520-548, wherein an occurrence of R^{s} is optionally substituted C₆₋₁₀ aryl.
562. The compound of any one of Embodiments 520-548, wherein an occurrence of R^{s} is phenyl.
563. The compound of any one of Embodiments 520-548, wherein an occurrence of R^{s} is 5-20 membered heteroaryl having 1-4 heteroatoms independently selected from oxygen, nitrogen, sulfur, phosphorus and silicon.
564. The compound of any one of Embodiments 520-548, wherein an occurrence of R^{s} is 5-10 membered heteroaryl having 1-4 heteroatoms independently selected from oxygen, nitrogen, sulfur, phosphorus and silicon.
565. The compound of any one of Embodiments 520-545, wherein R² is optionally substituted phenyl.
566. The compound of any one of Embodiments 520-545, wherein R² is phenyl.
567. The compound of any one of Embodiments 520-545, wherein R² is optionally substituted C₁₋₆ aliphatic.
568. The compound of any one of Embodiments 520-545, wherein R² is optionally substituted C₁₋₆ alkyl.
569. The compound of Embodiment 568, wherein R² is methyl.
570. The compound of Embodiment 568, wherein R² is ethyl.
571. The compound of Embodiment 568, wherein R² is propyl.
572. The compound of Embodiment 568, wherein R² is isopropyl.
573. The compound of Embodiment 568, wherein R² is n-butyl.
574. The compound of Embodiment 568, wherein R² is cyclopentyl.
575. The compound of Embodiment 568, wherein R² is cyclohexyl.
576. The compound of Embodiment 568, wherein R² is cyclobutyl.
577. The compound of any one of Embodiments 520-545, wherein R² is —N(R′)₂.
578. The compound of Embodiment 577, wherein R² is —NMe₂.
579. The compound of any one of Embodiments 520-544, wherein R¹ is —Si(R)₃.
580. The compound of Embodiment 579, wherein each R is not —H.
581. The compound of Embodiment 580, wherein each R is independently an optionally substituted C₁₋₃₀ aliphatic group.
582. The compound of Embodiment 580, wherein each R is independently a C₁₋₁₀ aliphatic group.
583. The compound of Embodiment 580, wherein each R is independently methyl.
584. The compound of Embodiment 580, wherein each R is independently ethyl.
585. The compound of Embodiment 579, wherein each R is independently an optionally substituted group selected from C₃₋₁₀ aliphatic and C₆₋₃₀ aryl.
586. The compound of Embodiment 579, wherein each R is independently an optionally substituted group selected from C₁₋₁₀ aliphatic and phenyl.
587. The compound of Embodiment 579, wherein R¹ is —Si(Ph)₂Me.
588. The compound of any one of Embodiments 520-587, wherein L is —CH₂—.
589. The compound of any one of Embodiments 520-588, wherein L is —CH(CN)—.
590. The compound of any one of Embodiments 520-589, wherein R^{a} and R^{b} are taken together with their intervening atoms to form an optionally substituted Ring B, wherein Ring B is 4-10 membered and has 0-4 (e.g., 0, 1-4, 1, 2, 3, 4, etc.) heteroatoms in addition to the nitrogen atom.
591. The compound of any one of Embodiments 520-590, wherein Ring B is 4-membered.
592. The compound of any one of Embodiments 520-590, wherein Ring B is 5-membered.
593. The compound of any one of Embodiments 520-590, wherein Ring B is 6-membered.
594. The compound of any one of Embodiments 520-590, wherein Ring B is monocyclic.
595. The compound of any one of Embodiments 520-590, wherein Ring B is bicyclic.
596. The compound of any one of Embodiments 520-590, wherein Ring B is polycyclic.
597. The compound of any one of Embodiments 520-596, wherein each monocyclic ring unit is independently 3-10 membered having 0-4 heteroatoms.
598. The compound of any one of Embodiments 520-596, wherein each monocyclic ring unit is independently 3-10 membered having 1-4 heteroatoms.
599. The compound of any one of Embodiments 520-598, wherein Ring B has no heteroatoms in addition to the nitrogen atom.
600. The compound of any one of Embodiments 520-589, wherein Ring B is optionally substituted

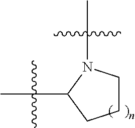

and n is 0, 1, 2, or 3.
601. The compound of any one of Embodiments 520-589, wherein Ring B is optionally substituted

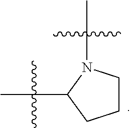 [SMILES: *C1CCCN1*]

602. The compound of any one of Embodiments 520-589, wherein Ring B is

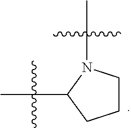 [SMILES: *C1CCCN1*]

603. The compound of any one of Embodiments 520-602, having a diastereomeric purity of about or at least about 10%, 15%, 20%, 25%, 30%, 35%, 40%, 45%, 50%, 55%, 60%, 65%, 70%, 75%, 80%, 85%, 90%, or 100%.
604. The compound of any one of Embodiments 520-603, having a diastereomeric purity of about or at least about 50%.
605. The compound of any one of Embodiments 520-604, having a diastereomeric purity of about or at least about 90%.
606. The compound of any one of Embodiments 520-605, having a purity of about or at least about 10%, 15%, 20%, 25%, 30%, 35%, 40%, 45%, 50%, 55%, 60%, 65%, 70%, 75%, 80%, 85%, 90%, or 100%.
607. The compound of any one of Embodiments 520-606, having a purity of about or at least about 50%.
608. The compound of any one of Embodiments 520-607, having a purity of about or at least about 90%.
609. A composition comprising a compound of any one of Embodiments 520-609, wherein the ratio of the compound and its epimer with respect to the chiral phosphorus is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%.
610. The composition comprising a compound of any one of Embodiments 520-610, wherein the ratio of cis isomer:trans isomer is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%.
611. The composition comprising a compound of any one of Embodiments 520-610, wherein the ratio of trans isomer:cis isomer is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%.
612. The composition comprising a compound of any one of Embodiments 520-611, wherein the ratio is about or at least about 50%:5%.
613. The composition comprising a compound of any one of Embodiments 520-611, wherein the ratio is about or at least about 75%:25%.
614. The composition comprising a compound of any one of Embodiments 520-611, wherein the ratio is about or at least about 80%:20%.
615. The composition comprising a compound of any one of Embodiments 520-611, wherein the ratio is about or at least about 90%:10%.
616. The composition comprising a compound of any one of Embodiments 520-611, wherein the ratio is about or at least about 95%:5%.
617. The composition comprising a compound of any one of Embodiments 520-611, wherein the ratio is about or at least about 99%:10%.
618. A method of preparing a compound or composition of any one of Embodiments 520-617, comprising reacting a compound having the structure of formula CA:

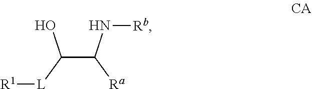

or a salt thereof with a nucleoside, wherein:
L is optionally substituted —CH₂—;
R¹ is R, —P(O)(R²)₂, —S(O)₂R², or —Si(R)₃;
R² is R, —OR, —N(R′)₂, or

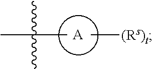

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   619. A method of preparing a compound or composition of any one of Embodiments 520-618, comprising reacting a compound having the structure of formula CA-A:

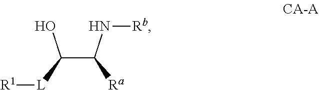

or a salt thereof with a nucleoside, wherein:
L is optionally substituted —CH₂—;
R¹ is R, —P(O)(R²)₂, —S(O)₂R², or —Si(R)₃;
R² is R, —OR, —N(R′)₂, or

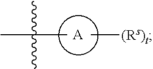

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   620. A method of preparing a compound or composition of any one of Embodiments 520-618, comprising reacting a compound having the structure of formula CA-B:

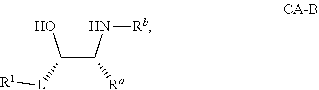

or a salt thereof with a nucleoside, wherein:
L is optionally substituted —CH₂—;
R¹ is R, —P(O)(R²)₂, —S(O)₂R², or —Si(R)₃;
R² is R, —OR, —N(R′)₂, or

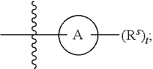

Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   621. The method of preparing a compound or composition of any one of Embodiments 618-620, wherein the nucleoside is R^{NS}—H or a salt thereof, wherein R^{NS} is —O—SU-BA, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
—O—SU— is —O-L^{s}- or

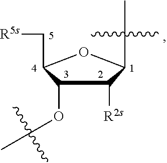

wherein —O—SU— is connected to the phosphorus atom in PMT-A, PMT-B, PMT-A1, or PMT-B1 through the oxygen atom;
L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon.
   622. The method of any one of Embodiments 618-621 wherein R^{NS} is

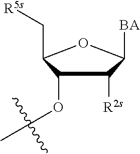

623. The method of any one of Embodiments 621-622, wherein R^{2S} is L^{s} connecting C2 with C₄, wherein L^{s} is (C2)-O-(optionally substituted methylene)-.
624. The method of any one of Embodiments 621-622, wherein R^{2S} is —OR′, wherein R′ is optionally substituted C₁₋₆ aliphatic.
625. The method of any one of Embodiments 621-622, wherein R^{2S} is selected form —H, —F, —OMe, and —OCH₂CH₂OMe.
626. The method of any one of Embodiments 621-622, wherein R^{2S} is —OMe.
627. The method of any one of Embodiments 621-622, wherein R^{2s} is —OCH₂CH₂OMe.
628. The method of any one of Embodiments 621-622, wherein R^{2S} is —F.
629. The method of any one of Embodiments 621-622, wherein R^{2s} is —H.
630. The method of any one of Embodiments 621-629, wherein R^{5s} is —OR′.
631. The method of any one of Embodiments 621-629, wherein R^{5s} is -ODMTr.
632. The method of any one of Embodiments 621-631, wherein BA is a natural nucleobase moiety or a modified nucleobase moiety.
633. The method of any one of Embodiments 621-631, wherein BA is an optionally substituted group selected from C₃₋₁₅ cycloaliphatic, C₆₋₁₄ aryl, C₃₋₁₅ heterocyclyl having 1-5 heteroatoms independently selected from oxygen, nitrogen, sulfur, phosphorus and silicon, C₅₋₁₄ heteroaryl having 1-5 heteroatoms independently selected from oxygen, nitrogen, sulfur, phosphorus and silicon.
634. The method of any one of Embodiments 621-631, wherein BA is optionally substituted C₅₋₁₄ heteroaryl having 1-5 heteroatoms independently selected from oxygen, nitrogen, and sulfur.
635. The method of any one of Embodiments 621-631, wherein BA is optionally substituted A, T, C, G, U or a tautomer thereof.
636. The method of any one of Embodiments 621-631, wherein BA is optionally protected A, T, C, G, U or a tautomer thereof.
637. The method of any one of Embodiments 621-631, wherein BA is nucleobase is optionally substituted or protected U, or is an optionally substituted or protected tautomer of U, or is optionally substituted or protected C, or is an optionally substituted or protected tautomer of C, or is optionally substituted or protected A, or is an optionally substituted or protected tautomer of A, or is optionally substituted or protected nucleobase of pseudoisocytosine, or is an optionally substituted or protected tautomer of the nucleobase of pseudoisocytosine.
638. The method of any one of Embodiments 621-631, wherein BA is an optionally substituted group which group is selected from

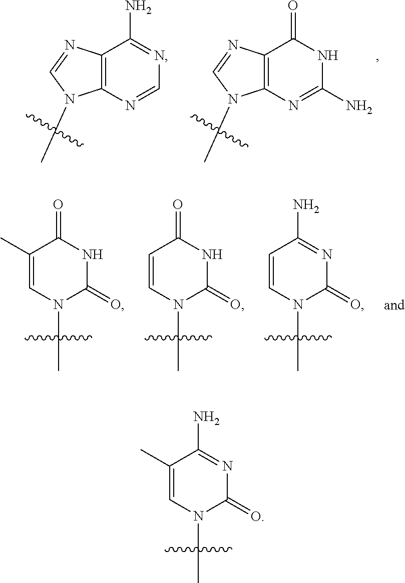

639. The method of any one of Embodiments 621-631, wherein BA an optionally substituted guanine residue wherein its O⁶ is unprotected.
640. The method of any one of Embodiments 621-639, wherein R¹ is —S(O)₂R².
641. The method of any one of Embodiments 621-640, wherein R² is

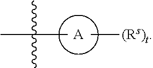

642. The method of any one of Embodiments 621-641, wherein Ring A is an optionally substituted phenyl ring.
643. The method of any one of Embodiments 621-641, wherein t is 1.
644. The method of any one of Embodiments 621-643, wherein an occurrence of R^{s} is halogen.
645. The method of any one of Embodiments 621-643, wherein an occurrence of R^{s} is Cl.
646. The method of any one of Embodiments 621-643, wherein an occurrence of R^{s} is Br.
647. The method of any one of Embodiments 621-643, wherein an occurrence of R^{s} is CN.
648. The method of any one of Embodiments 621-643, wherein an occurrence of R^{s} is COOR′, wherein R′ is optionally substituted C₁₋₆ aliphatic.
649. The method of any one of Embodiments 621-643, wherein an occurrence of R^{s} is —COOMe.
650. The method of any one of Embodiments 621-643, wherein an occurrence of R^{s} is —COOEt.
651. The method of any one of Embodiments 621-643, wherein an occurrence of R^{s} is —OR′.
652. The method of any one of Embodiments 621-643, wherein an occurrence of R^{s} is —OR′, wherein R is optionally substituted C₁₋₆ aliphatic.
653. The method of any one of Embodiments 621-643, wherein an occurrence of R^{s} is —OMe.
654. The method of any one of Embodiments 621-643, wherein an occurrence of R^{s} is N(R′)₂.
655. The method of any one of Embodiments 621-643, wherein an occurrence of R^{s} is NMe₂.
656. The method of any one of Embodiments 621-643, wherein an occurrence of R^{s} is optionally substituted C₆₋₁₀ aryl.
657. The method of any one of Embodiments 621-643, wherein an occurrence of R^{s} is phenyl.
658. The method of any one of Embodiments 621-643, wherein an occurrence of R^{s} is 5-20 membered heteroaryl having 1-4 heteroatoms independently selected from oxygen, nitrogen, sulfur, phosphorus and silicon.
659. The method of any one of Embodiments 621-643, wherein an occurrence of R^{s} is 5-10 membered heteroaryl having 1-4 heteroatoms independently selected from oxygen, nitrogen, sulfur, phosphorus and silicon.
660. The method of any one of Embodiments 621-640, wherein R² is optionally substituted phenyl.
661. The method of any one of Embodiments 621-640, wherein R² is phenyl.
662. The method of any one of Embodiments 621-640, wherein R² is optionally substituted C₁₋₆ aliphatic.
663. The method of any one of Embodiments 621-640, wherein R² is optionally substituted C₁₋₆ alkyl.
664. The method of Embodiment 663, wherein R² is methyl.
665. The method of Embodiment 663, wherein R² is ethyl.
666. The method of Embodiment 663, wherein R² is propyl.
667. The method of Embodiment 663, wherein R² is isopropyl.
668. The method of Embodiment 663, wherein R² is n-butyl.
669. The method of Embodiment 663, wherein R² is cyclopentyl.
670. The method of claim 663, wherein R² is cyclohexyl.
671. The method of Embodiment 663, wherein R² is cyclobutyl.
672. The method of any one of Embodiments 621-640, wherein R² is —N(R′)₂.
673. The method of Embodiment 672, wherein R² is —NMe₂.
674. The method of any one of Embodiments 621-639, wherein R¹ is —Si(R)₃.
675. The method of Embodiment 674, wherein each R is not —H.
676. The method of Embodiment 675, wherein each R is independently an optionally substituted C₁₋₃₀ aliphatic group.
677. The method of Embodiment 675, wherein each R is independently a C₁₋₁₀ aliphatic group.
678. The method of Embodiment 675, wherein each R is independently methyl.
679. The method of Embodiment 675, wherein each R is independently ethyl.
680. The method of Embodiment 675, wherein each R is independently an optionally substituted group selected from C₁₋₃₀ aliphatic and C₆₋₃₀ aryl.
681. The method of Embodiment 675, wherein each R is independently an optionally substituted group selected from C₁₋₁₀ aliphatic and phenyl.
682. The method of Embodiment 675, wherein R¹ is —Si(Ph)₂Me.
683. The method of any one of Embodiments 618-682, wherein L is —CH₂—.
684. The method of any one of Embodiments 618-682, wherein L is —CH(CN)—.
685. The method of any one of Embodiments 618-684, wherein R^{a} and R^{b} are taken together with their intervening atoms to form an optionally substituted Ring B, wherein Ring B is 4-10 membered and has 0-4 (e.g., 0, 1-4, 1, 2, 3, 4, etc.) heteroatoms in addition to the nitrogen atom.
686. The method of any one of Embodiments 618-684, wherein Ring B is 4-membered.
687. The method of any one of Embodiments 618-684, wherein Ring B is 5-membered.
688. The method of any one of Embodiments 618-684, wherein Ring B is 6-membered.
689. The method of any one of Embodiments 618-684, wherein Ring B is monocyclic.
690. The method of any one of Embodiments 618-684, wherein Ring B is bicyclic.
691. The method of any one of Embodiments 618-684, wherein Ring B is polycyclic.
692. The method of any one of Embodiments 618-691, wherein each monocyclic ring unit is independently 3-10 membered having 0-4 heteroatoms.
693. The method of any one of Embodiments 618-691, wherein each monocyclic ring unit is independently 3-10 membered having 1-4 heteroatoms.
694. The method of any one of Embodiments 618-684, wherein Ring B is optionally substituted

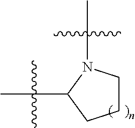

and n is 0, 1, 2, or 3.
695. The method of any one of Embodiments 618-684, wherein Ring B is optionally substituted

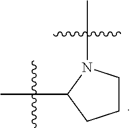 [SMILES: *C1CCCN1*]

696. The method of any one of Embodiments 618-684, wherein Ring B is

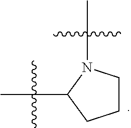 [SMILES: *C1CCCN1*]

697. The method of any one of Embodiments 618-696, wherein the method is for preparing a compound or composition of any of Embodiments 520-617.
698. The method of Embodiment 618, wherein the method is for preparing a compound of

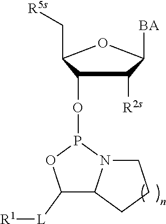

or a salt thereof, and the ratio of trans isomer:cis isomer with respect to the chiral phosphorus is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%.
699. The method of Embodiment 698, wherein the method is for preparing a compound of

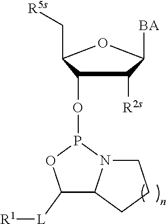

or a salt thereof, and the ratio of trans isomer:cis isomer with respect to the chiral phosphorus is about or at least about 90%:10%.
700. The method of Embodiment 698, wherein the method is for preparing a compound of

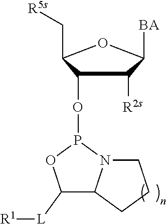

or a salt thereof, and the ratio of trans isomer:cis isomer with respect to the chiral phosphorus is about or at least about 95%:5%.
701. The method of Embodiment 698, wherein the method is for preparing a compound of

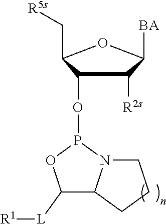

or a salt thereof, and the ratio of trans isomer:cis isomer with respect to the chiral phosphorus is about or at least about 99%:1%.
702. The method of Embodiment 698, wherein the method is for preparing a compound of

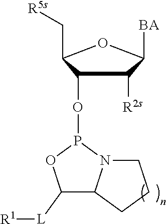

or a salt thereof, and the ratio of trans isomer:cis isomer with respect to the chiral phosphorus is about or at least about 99.5%:0.5%.
703. The method of Embodiment 698, wherein the method is for preparing a compound of

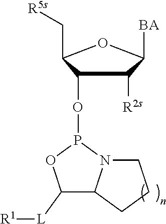

or a salt thereof, and the ratio of trans isomer:cis isomer with respect to the chiral phosphorus is about or at least about 99.9%:0.1%.
704. The method of Embodiment 618, wherein the method is for preparing a compound of

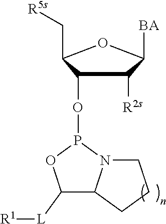

or a salt thereof, and the ratio of trans isomer:cis isomer with respect to the chiral phosphorus is about or less than about 50%:50%, about 40%:60%, about 30%:70%, about 20%:80%, about 10%:90%, about 9%:91%, about 8%:92%, about 7%:93%, about 6%:94%, about 5%:95%, about 4%:96%, about 3%:97%, about 2%:98%, about 1%:99%, about 0.05%:99.5%, or about 0.1%:99.9%.
705. The method of Embodiment 704, wherein the method is for preparing a

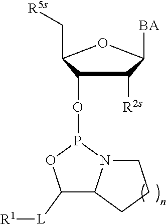

or a salt thereof, and the ratio of trans isomer:cis isomer with respect to the chiral phosphorus is about or less than about 10%:90%.
706. The method of Embodiment 704, wherein the method is for preparing a compound of

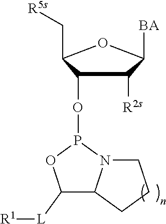

or a salt thereof, and the ratio of trans isomer:cis isomer with respect to the chiral phosphorus is about or less than about 5%:95%.
707. The method of Embodiment 704, wherein the method is for preparing a compound of

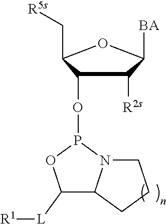

or a salt thereof, and the ratio of trans isomer:cis isomer with respect to the chiral phosphorus is about or less than about 1%:99%.
708. The method of Embodiment 704, wherein the method is for preparing a compound of

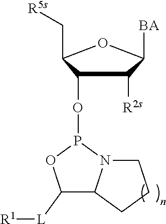

or a salt thereof, and the ratio of trans isomer:cis isomer with respect to the chiral phosphorus is about or less than about 0.5%:99.5%.
709. The method of Embodiment 704, wherein the method is for preparing a compound of

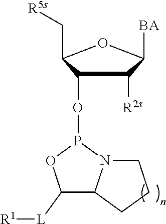

or a salt thereof, and the ratio of trans isomer:cis isomer with respect to the chiral phosphorus is about or less than about 0.1%:99.9%.
710. The method of any one of Embodiments 698-709, wherein the trans isomer and cis isomer are

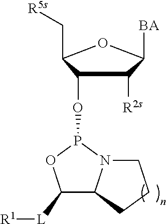

or a salt thereof and

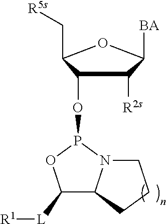

or a salt thereof, respectively.
711. The method of any one of Embodiments 698-709, wherein the trans isomer and cis isomer are

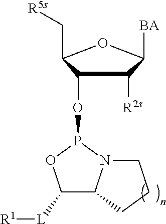

or a salt thereof and

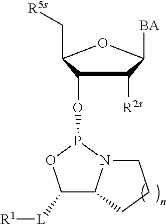

or a salt thereof, respectively.
712. The method of any one of Embodiments 618-696, wherein the reacting is performed in the presence of base.
713. The method of any one of Embodiments 618-712, wherein the base is a sterically hindered base (compared to triethyl amine).
714. The method of any one of Embodiments 618-712, wherein the base is of low nucleophilicity (compared to triethyl amine).
715. The method of any one of Embodiments 618-712, wherein the base is a tertiary amine that has the structure of N(R)₃ wherein the three R groups are taken together with nitrogen to form an optionally substituted 8-20 (e.g., 8-10, 8, 9, 10, 11, 12, 13, 14, 15, 16, 17, 18, 19, 20, etc.) membered bicyclic or polycyclic ring having 0-3 (e.g., 0, 1-3, 1, 2, 3, etc.) heteroatoms in addition to the nitrogen atom.
716. The method of any one of Embodiments 618-712, wherein the base is a tertiary amine that has the structure of N(R)₃ wherein the three R groups are taken together with nitrogen to form an optionally substituted 8-20 (e.g., 8-10, 8, 9, 10, 11, 12, 13, 14, 15, 16, 17, 18, 19, 20, etc.) membered bicyclic or polycyclic ring having 0-3 (e.g., 0, 1-3, 1, 2, 3, etc.) nitrogen atoms in addition to the nitrogen atom.
717. The method of any one of Embodiments 618-712, wherein the base is DBU.
718. The method of any one of Embodiments 618-712, wherein the base is DBN.
719. The method of any one of Embodiments 618-712, wherein the base is DABCO.
720. The method of any one of Embodiments 618-712, wherein the base is N-methylmorpholine (NMM).
721. The method of any one of Embodiments 618-712, wherein the base is N,N-diisopropylethylamine (DIPEA).
722. The method of any one of Embodiments 618-721, wherein the equivalent of the base is about or at least about 1, 1.1, 1.2, 1.3, 1.4, 1.5, 1.6, 1.7, 1.8, 1.9, 2, 2.5, 3, 3.5, 4, 4.5, 5, 6, 7, 8, 9, or 10 relative to the nucleoside.
723. The method of any one of Embodiments 618-722, wherein the reaction is performed in the presence of another base.
724. The method of any one of Embodiments 618-723, wherein the another base is TEA.
725. The method of any one of Embodiments 618-724, wherein the ratio of the base and the another base is about or at least about 3:1.
726. The method of any one of Embodiments 618-724, wherein the ratio of the base and the another base is about or at least about 1:1.
727. The method of any one of Embodiments 618-726, wherein the reacting is performed at a reduced temperature.
728. The method of any one of Embodiments 618-727, comprising increasing the reaction temperature from a reduced temperature to an ambient temperature (about 25° C.).
729. A method for isomerizing a compound of any one of Embodiments 520-617 with respect to its chiral phosphorus, comprising contacting the compound with a phosphoramidite activator for oligonucleotide synthesis.
730. A method for preparing an oligonucleotide, comprising steps of:
1) contacting a phosphoramidite composition comprising a compound of any one of Embodiments 520-617 with a phosphoramidite activator to provide an isomerized phosphoramidite composition, wherein the isomerized phosphoramidite composition comprises a lower level of the compound compared to the phosphoramidite composition; and
2) contacting the isomerized phosphoramidite composition with a coupling partner.
   731. The method of any one of Embodiments 730-731, wherein the coupling partner comprises —OH.
   732. The method of any one of Embodiments 730-731, wherein the coupling partner is or comprise a nucleoside.
   733. The method of any one of Embodiments 730-731, wherein the coupling partner is an oligonucleotide.
   734. The method of any one of Embodiments 730-733, wherein the coupling partner is linked to a solid support.
   735. The method of any one of Embodiments 730-734, wherein the coupling partner is linked to a solid support through a linker.
   736. The method of any one of Embodiments 729-735, wherein the activator is a mildly acidic compound.
   737. A method for isomerizing a compound of any one of Embodiments 520-608 with respect to its chiral phosphorus, comprising contacting the compound with a mildly acidic compound.
   738. The method of any one of Embodiments 736-737, wherein the mildly acidic compound is a salt of N(R)₃, wherein two R groups are taken together with the nitrogen to form an optionally substituted 5-10 membered ring having 0-3 (e.g., 0, 1-3, 1, 2, 3, etc.) heteroatoms in addition to the nitrogen atom.
   739. The method of any one of Embodiments 736-737, wherein the activator is CMPT.
   740. The method of any one of Embodiments 736-737, wherein the mildly acidic compound is a salt of a heteroaryl base comprising a sp2 nitrogen atom.
   741. The method of any one of Embodiments 736-737, wherein the mildly acidic compound is a salt of a heteroaryl base comprising a sp3 nitrogen atom.
   742. The method of any one of Embodiments 729-741, wherein the method comprises contacting a salt of the base.
   743. The method of Embodiment 742, wherein the salt is a triflate.
   744. The method of any one of Embodiments 729-741, wherein the activator is CMIMT.
   745. The method of any one of Embodiments 729-741, wherein the activator is 4-nitrophenol.
   746. A method for assessing level of a compound in a composition, comprising using a compound or composition of any one of Embodiments 520-617 as a reference.
   747. A method, comprising:
1) contacting a P(III) agent with an azide to provide a composition for coupling;
2) contacting the composition for coupling with a coupling partner.
   748. The method of Embodiment 747, wherein the P(III) agent is contacted with an azide in the absence of the coupling partner.
   749. The method of any one of Embodiments 747-748, wherein the ratio of the azide to the coupling partner is about or less than about 2, 1.9, 1.8, 1.7, 1.6, 1.5, 1.4, 1.3, 1.2, or 1.1.
   750. The method of any one of Embodiments 747-749, wherein the ratio of the azide to the coupling partner is about or less than about 1.5.
   751. The method of any one of Embodiments 747-749, wherein the ratio of the azide to the coupling partner is about or less than about 1.5-1.
   752. The method of any one of Embodiments 747-751, wherein the ratio of the azide to the P(III) agent is about or less than about 2, 1.9, 1.8, 1.7, 1.6, 1.5, 1.4, 1.3, 1.2, or 1.1.
   753. The method of any one of Embodiments 747-751, wherein the ratio of the azide to the P(III) agent is about or less than about 1.5.
   754. The method of any one of Embodiments 747-751, wherein the ratio of the azide to the P(III) agent is about or less than about 1.5-1.
   755. The method of any one of Embodiments 747-754, wherein the coupling partner comprises —OH.
   756. The method of any one of Embodiments 747-754, wherein the coupling partner is or comprise a nucleoside.
   757. The method of any one of Embodiments 747-754, wherein the coupling partner is an oligonucleotide.
   758. The method of any one of Embodiments 747-757, wherein the coupling partner is linked to a solid support.
   759. The method of any one of Embodiments 747-758, wherein the coupling partner is linked to a solid support through a linker.
   760. The method of any one of Embodiments 747-759, wherein the P(III) agent is a phosphoramidite.
   761. The method of any one of Embodiments 747-760, wherein the P(III) agent has the structure of formula PIII:

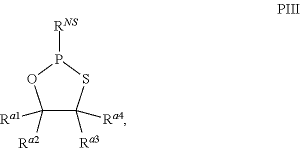

or a salt thereof, wherein R^{NS} is a nucleoside and each of R^{a1}, R^{a2}, R^{a3} and R^{a4} is independently R′ as described herein.
762. The method of any one of Embodiments 747-761, wherein R^{a2} is —H.
763. The method of any one of Embodiments 747-762, wherein R^{a4} is —H.
764. The method of any one of Embodiments 747-763, wherein R^{a1} is optionally substituted C₁₋₆ alkyl.
765. The method of any one of Embodiments 747-764, wherein R^{a1} is methyl.
766. The method of any one of Embodiments 747-765, wherein R^{a3} is optionally substituted C₁₋₆ alkyl.
767. The method of any one of Embodiments 747-766, wherein R^{a3} is methyl.
768. The method of any one of Embodiments 747-763, wherein R^{a1} and R^{a3} are taken together to form an optionally substitute 3-10 (e.g., 3-6, 4-10, 5-6, 3, 4, 5, 6, 7, 8, 9, or 10, etc.) membered ring.
769. The method of any one of Embodiments 747-768, wherein R^{NS} is —O—SU-BA, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
—O—SU— is —O-L^{s}- or

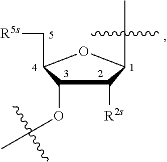

wherein —O—SU— is connected to the phosphorus atom in formula PMT-A or PMT-B through the oxygen atom; L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon.
   770. The method of Embodiment 769, wherein —O—SU— is

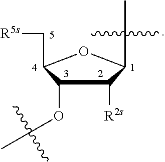

771. The method of any one of Embodiments 769-770, wherein R^{2s} is selected form —H, —F, —OMe, and —OCH₂CH₂OMe.
772. The method of any one of Embodiments 769-771, wherein R^{5s} is -ODMTr.
773. The method of any one of Embodiments 769-772, wherein BA is a natural nucleobase moiety or a modified nucleobase moiety.
774. The method of any one of Embodiments 769-772, wherein BA is an optionally substituted group selected from C₃₋₁₅ cycloaliphatic, C₆₋₁₄ aryl, C₃₋₁₅ heterocyclyl having 1-5 heteroatoms independently selected from oxygen, nitrogen, sulfur, phosphorus and silicon, C₅₋₁₄ heteroaryl having 1-5 heteroatoms independently selected from oxygen, nitrogen, sulfur, phosphorus and silicon.
775. The method of any one of Embodiments 769-772, wherein BA is optionally substituted C₅₋₁₄ heteroaryl having 1-5 heteroatoms independently selected from oxygen, nitrogen, and sulfur.
776. The method of any one of Embodiments 769-772, wherein BA is optionally substituted A, T, C, G, U or a tautomer thereof.
777. The method of any one of Embodiments 769-772, wherein BA is optionally protected A, T, C, G, U or a tautomer thereof.
778. The method of any one of Embodiments 769-772, wherein BA is optionally substituted or protected U, or is an optionally substituted or protected tautomer of U, or is optionally substituted or protected C, or is an optionally substituted or protected tautomer of C, or is optionally substituted or protected A, or is an optionally substituted or protected tautomer of A, or is optionally substituted or protected nucleobase of pseudoisocytosine, or is an optionally substituted or protected tautomer of the nucleobase of pseudoisocytosine.
779. The method of any one of Embodiments 769-772, wherein BA is an optionally substituted group which group is selected from

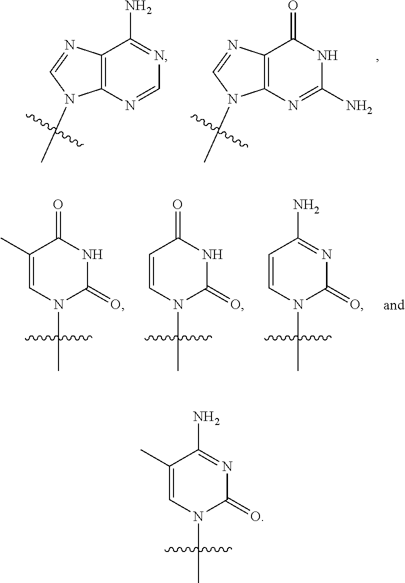

780. The method of any one of Embodiments 769-772, wherein BA an optionally substituted guanine residue wherein its O⁶ is unprotected.
781. The method of any one of Embodiments 769-772, wherein BA is

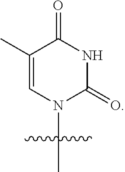 [SMILES: *n1cc(C)c(=O)[nH]c1=O]

782. The method of any one of Embodiments 747-769, wherein the P(III) agent has the structure of

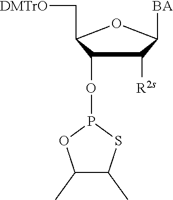

or a salt thereof.
783. The method of any one of Embodiments 747-769, wherein the P(III) agent has the structure of

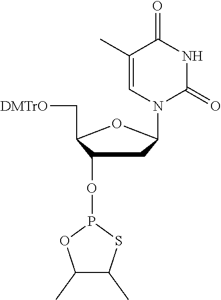

or a salt thereof.

### EXEMPLIFICATION

Non-limiting examples are provided below. A person of ordinary skill in the art appreciates that other technologies, e.g., compounds, compositions, methods, etc., may also be utilized in the present technologies in accordance with the present disclosure.

As described herein, provided technologies can provide a number of advantages, e.g., high synthetic efficiency (e.g., fewer steps, higher yields, etc.), improved manufacturing processes (e.g., avoidance or reduction of low temperature operations, use of more stable intermediates, etc.), high stereoselectivity, high yield, high product purity, etc. Various advantages are demonstrated in Examples below.

### Example 1: Synthesis of WV-CA-108

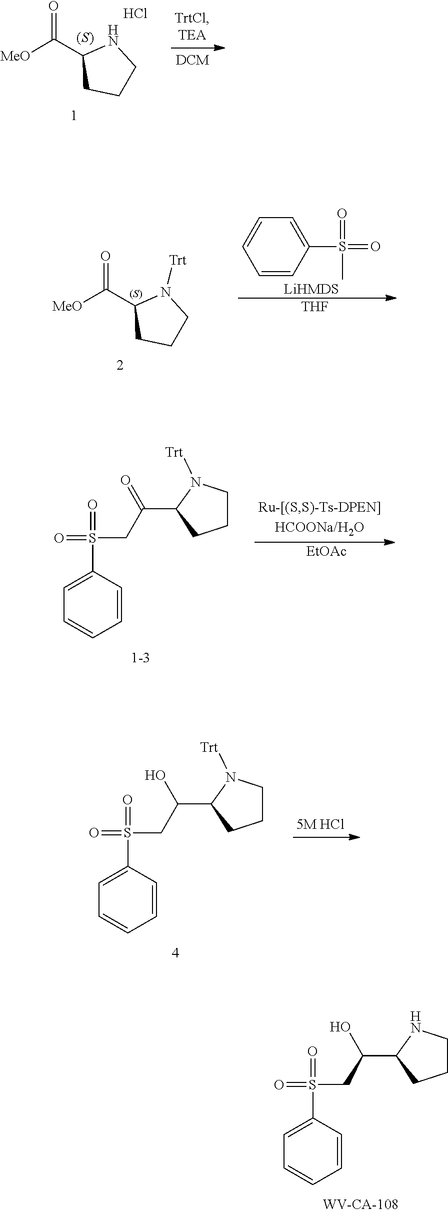

Preparation of compound 2: To a solution of compound 1 (50 g, 301.90 mmol, 1 eq., HCl) in DCM (500 mL) was added TEA (61.10 g, 603.80 mmol, 84.04 mL, 2 eq.) and TrtCl (92.58 g, 332.09 mmol, 1.1 eq.). The mixture was stirred at 15° C. for 12 hr. TLC (Petroleum ether: Ethyl acetate=10:1, Rf=0.5) indicated compound 1 was consumed completely and one new spot formed. The reaction mixtures of four batches were combined, and sat.NaHCO₃ (1500 mL) was added to the reaction mixture, then the reaction mixture was extracted with EtOAc (900 mL, 300 mL×3). The combined organic layers were dried over Na₂SO₄, filtered and concentrated under reduced pressure to give a residue. The residue was purified by column chromatography (SiO₂, Petroleum ether/Ethyl acetate=I/O to 1/1). Compound 2 (424 g, crude) was obtained as a white solid. Enantiomeric ratio (er)=0.44:99.56 as observed from SFC which separated the two enantiomers. TLC (Petroleum ether: Ethyl acetate=10:1, Rf=0.5).

Preparation of compound 1-3: To a solution of compound 2 (25 g, 67.30 mmol, 1 eq.) in THF (250 mL) was added LiHMDS (1 M, 134.60 mL, 2 eq.) and methylsulfonylbenzene (10.51 g, 67.30 mmol, 1 eq.). The mixture was stirred at 0° C. for 2 hr. TLC (Petroleum ether: Ethyl acetate=3:1, Rf=0.65) indicated compound 2 was consumed and one new spot formed. The reaction mixture of two batches was combined, and quenched by addition sat. NH₄Cl (300 mL) at 0° C., then extracted with ethyl acetate (450 mL, 150 mL×3). The combined organic layers were dried over Na₂SO₄, filtered and concentrated under reduced pressure to give a residue. Compound 1-3 (66 g, crude) was obtained as a yellow solid. TLC (Petroleum ether: Ethyl acetate=3:1, Rf=0.65).

Preparation of compound 4: To a solution of compound 1-3 (30 g, 60.53 mmol, 1 eq.) in EtOAc (150 mL) was added HCOONa (192.24 g, 2.83 mol, 152.57 mL, 46.7 eq.) in H₂O (600 mL) then added N-[(1S,2S)-2-amino-1,2-diphenyl-ethyl]-4-methyl-benzenesulfonamide;chlororuthenium;1-isopropyl-4-methyl-benzene (1.93 g, 3.03 mmol, 0.05 eq.) in N₂. The mixture was stirred at 30° C. for 16 hour. TLC (Petroleum ether: Ethyl acetate=3:1, Rf=0.6) indicated compound 1-3 was consumed completely and one main new spot formed. The reaction mixture was concentrated under reduced pressure to remove solvent. The residue was purified by MPLC (SiO₂, Petroleum ether: Ethyl acetate=I/O to 2/1). For compound 4, ¹H NMR-1 showed a dr=18.51: 1. The crude was purified by re-crystallization from THF (30 mL) and methanol (300 mL) at 20° C. ¹HNMR indicated a clean compound 4 product. Compound 4 (15 g, 30.14 mmol, 49.80% yield) was obtained as a white solid. TLC (Petroleum ether: Ethyl acetate=3:1, Rf=0.6).

Preparation of compound WV-CA-108: To a solution of compound 4 (15 g, 30.14 mmol, 1 eq.) in THF (150 mL) was added HCl (5 M, 60.28 mL, 10 eq.). The mixture was stirred at 15° C. for 2 hr. TLC (Petroleum ether: Ethyl acetate=0:1, Rf=0) indicated compound 4 was consumed completely and one new spot formed. The reaction mixture was diluted with EtOAc (20 mL) and extracted with H₂O (20 mL×3). The combined water layers was adjusted to pH 12 with 5M NaOH aq., and extracted with DCM (10 mL×3). The combined organic layers were dried over anhydrous Na₂SO₄, filtered and concentrated to give a residue. TLC (Ethyl acetate: Methanol=5:1, Rf=0.1). Compound WV-CA-108 (7 g, 27.12 mmol, 89.98% yield, 98.93% purity) was obtained as a brown solid. ¹H NMR (400 MHz, CHLOROFORM-d) δ ppm 1.54-1.82 (m, 4H) 2.67-2.95 (m, 4H) 3.16-3.23 (m, 1H) 3.24-3.38 (m, 2H) 4.05 (ddd, J=8.38, 5.25, 2.75 Hz, 1H) 7.53-7.62 (m, 2H) 7.63-7.72 (m, 1H) 7.92-8.00 (m, 2H). LCMS (M+H⁺): 256.1; purity: 98.93%. SFC: de=100%. TLC (Ethyl acetate: Methanol=5:1, R_{f}=0.1). AAS: Ru content is 35 ppm.

Reduction of compound 1-3 to compound 4 were also performed at various other scales and Ru-complex loads (e.g., 0.01, 0.025, 0.05 etc. equivalent). In various instances, about 10:1 or higher diastereomeric ratio were observed. In some embodiments, at lower Ru-complex loading, lower conversion rates were observed compared to higher Ru-complex loading; for example, under one set of conditions with 2 g starting ketone, about ½ and about ⅓ reactant remained for 0.01 (1:0 cis:trans by ¹H NMR) and 0.025 (1:0.11 cis:trans by ¹H NMR)equivalent of Ru-complex, respectively, when all reactant was consumed for 0.05 equivalent (1:0.077 cis:trans by ¹H NMR).

### Example 2: Synthesis of WV-CA-236D

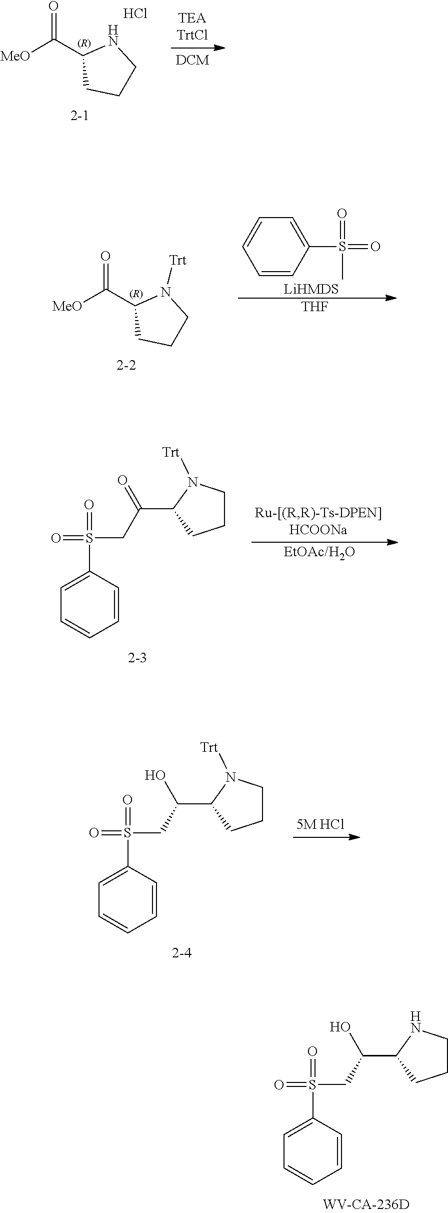

Preparation of compound 2-2: To a solution of compound 2-1 (24 g, 144.91 mmol, HCl salt) in DCM (250 mL) was added TEA (29.33 g, 289.82 mmol) and TrtCl (40.40 g, 144.91 mmol). The mixture was stirred at 15° C. for 3 hr. TLC indicated compound 2-1 was consumed completely and one new spot formed. The reaction was clean according to TLC. The reaction mixture was quenched by addition NH₄Cl (200 mL), and extracted with Ethyl acetate (500 mL). The combine dried over Na₂SO₄, filtered and concentrated under reduced pressure to give a residue. The residue was purified by column chromatography (SiO₂, Petroleum ether/Ethyl acetate=20/1 tol/1). Compound 2-2 (50 g, 96.15% yield) was obtained as a white solid. TLC: Petroleum ether: Ethyl acetate=4: 1, Rf=0.38. Enantiomeric excess (ee %)=96.76%.

Preparation of compound 2-3: To a solution of compound 2-2 (50 g, 134.60 mmol) in THF (500 mL) was added LiHMDS (1 M, 269.20 mL) and methylsulfonylbenzene (21.02 g, 134.60 mmol). The mixture was stirred at 0° C. for 2 hr. TLC indicated compound 2-2 was consumed completely and one new spot formed. The reaction was clean according to TLC. The reaction mixture was quenched by addition NH₄Cl (500 mL), and then extracted with Ethyl acetate 1000 mL (500 mL×2). The combined organic layers were dried over Na₂SO₄, filtered and concentrated under reduced pressure to give a residue. The residue was purified by column chromatography (SiO₂, Petroleum ether/Ethyl acetate=30/1 to 1/1). Compound 2-3 (58 g, 87.88% yield) was obtained as a yellow solid. TLC (Petroleum ether: Ethyl acetate=3: 1), Rf=0.28. ee=100%.

Preparation of compound 2-4: To a solution of compound 2-3 (20 g, 40.35 mmol) in EtOH (200 mL) was added HCOONa (128.16 g, 1.88 mol) in the H₂O (400 mL), then added [[(1R,2R)-2-amino-1,2-diphenyl-ethyl]-(p-tolylsulfonyl)amino]-chloro-ruthenium;1-isopropyl-4-methyl-benzene (1.28 g, 2.02 mmol) in N₂. The mixture was stirred at 30° C. for 16 hour. TLC indicated compound 2-3 was consumed completely, and one new spot formed. The reaction was clean according to TLC. The combined organic layers were washed with saturated brine (200 mL), dried over Na₂SO₄, filtered and concentrated under reduced pressure to give a residue. The crude product was dissolved in THF (20 mL), and then added methanol (200 mL) slowly, stirred and triturated at 15° C. for 5 hr. Compound 2-4 (10.1 g, 50.30% yield) was obtained as a white solid. TLC: Petroleum ether: Ethyl acetate=3/1, Rf=0.39. Crude product: dr=99.68: 0.32. After purification: dr=99.86: 0.14.

Preparation of compound WV-CA-236D: To a solution of compound 2-4 (10 g, 20.09 mmol) in THF (100 mL) was added HCl (5 M, 40.19 mL). The mixture was stirred at 15° C. for 3 hr. LCMS (M+H⁺) showed compound 2-4 was consumed completely and one main peak with desired m/z. The reaction mixture was diluted with EtOAc (100 mL) and extracted with H₂O (30 mL×3). The combined water layers was adjusted to pH 12 with 5M NaOH aq., and extracted with DCM (100 mL×5). The combined organic layers were dried over Na₂SO₄, filtered and concentrated to give a residue. Compound WV-CA-236D (5 g, 97.51% yield) was obtained as a yellow solid. TLC: Ethyl acetate: Methanol=5: 1, Rf=0.22. LCMS (M+H⁺): 256.0; purity: 97.79%. Diastereomeric excess (de)=100%. ¹H NMR (400 MHz, METHANOL-d4) δ ppm 1.53-1.67 (m, 1H), 1.67-1.93 (m, 4H), 2.76-2.84 (m, 1H), 2.91 (dt, J=10.58, 6.44 Hz, 1H), 2.97-3.05 (m, 1H), 3.28-3.39 (m, 2H), 3.46-3.53 (m, 1H), 4.04 (ddd, J=8.77, 5.92, 2.85 Hz, 1H), 7.60-7.76 (m, 3H), and 7.94-8.01 (m, 2H).

### Example 3: Synthesis of WV-CA-235R

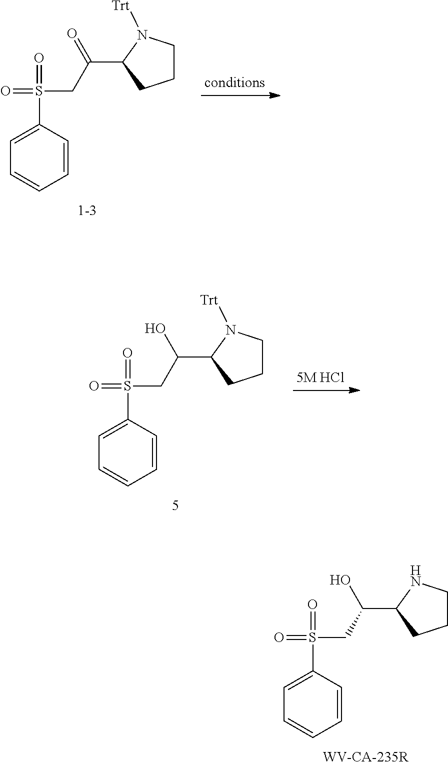

Preparation of compound 5: To a solution of compound 1-3 (0.5 g, 1.01 mmol, 1 eq.) in EtOH (5 mL) was added NaBH₄ (381.63 mg, 10.09 mmol, 10 eq.). The mixture was stirred at 15° C. for 16 hr. TLC (Petroleum ether: Ethyl acetate=3:1, Rf=0.6) indicated compound 1-3 was consumed completely and one main new spot formed. The reaction mixture was concentrated under reduced pressure to remove solvent. The residue was purified by prep-TLC (SiO₂, Petroleum ether: Ethyl acetate=3:1) to get compound 5 (0.2 g, crude) was obtained as a yellow oil. TLC (Petroleum ether: Ethyl acetate=3:1, Rf=0.6).

To a solution of compound 5 (0.1 g, 200.95 μmol, 1 eq.) in DCM (2 mL) was added TFA (22.91 mg, 200.95 μmol, 14.88 μL, 1 eq.). The mixture was stirred at 15° C. for 10 min. TLC (Petroleum ether: Ethyl acetate=3:1, Rf=0) indicated compound 5 was consumed completely and one new spot formed. The reaction mixture was extracted with water (10 mL×3), the combined water layers were added sat. Na₂CO₃ aq. until pH˜10, then extracted with DCM (10 mL×3), the combined water layers were dried over Na₂SO₄, filtered and concentrated under reduced pressure to give a residue. ¹HNMR (400 MHz, CHLOROFORM-d) δ=8.02-7.90 (m, 2H), 7.71-7.62 (m, 1H), 7.62-7.54 (m, 2H), 3.86 (dt, J=2.8, 5.5 Hz, 1H), 3.35-3.13 (m, 2H), 3.00-2.79 (m, 2H), 2.02-1.39 (m, 8H). TLC (Petroleum ether: Ethyl acetate=3:1), Rf=0. ¹H NMR showed only the trans isomer. When LiBH₄ was utilized, similar selectivity were observed.

### Example 4: Provided Technologies can be Utilized to Manufacture Various Compounds

Those skilled in the art appreciate that provided technologies can be utilized to manufacture various compounds. Certain additional examples and results are presented below.

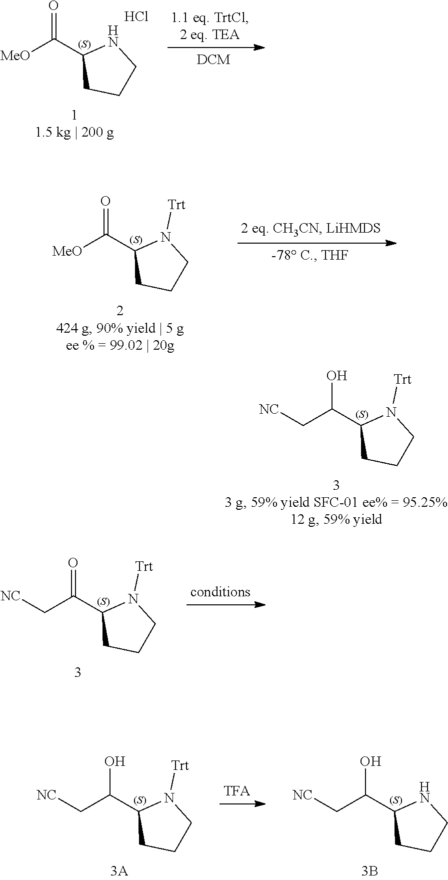

| | Scale of | | Ratio Cis/Trans |
|---|---|---|---|
| Metal Complex | ketone | eq. | of 3A (¹H NMR) |
| RuCl(p-cymene)[(S,S)-Fsdpen]] | 1 g | 0.05 | 1:0.13 |
| RuCl(p-cymene) [(S,S)-Ts-DPEN] | 1 g | 0.025 | 1:0.28 |
| RuCl(p-cymene) [(S,S)-Ts-DPEN] | 1 g | 0.05 | 1:0.31 |

### Example 5: Synthesis of WV-CA-084

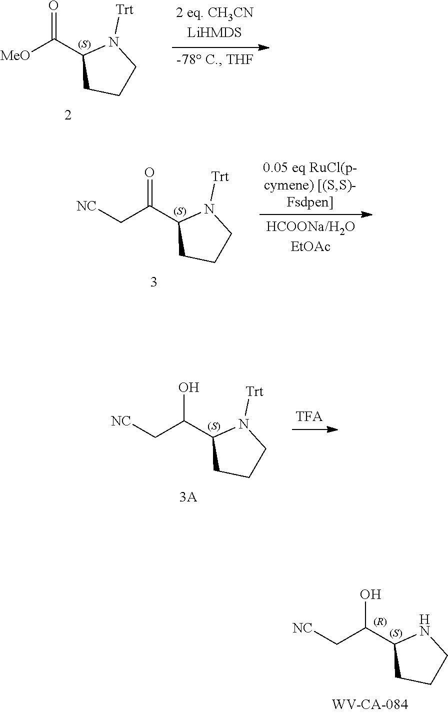

Preparation of Compound 3.

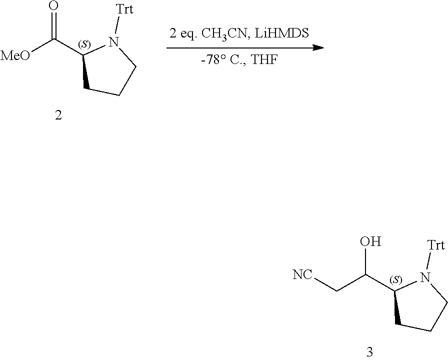

Batches were set up: To a solution of ACN (3.69 g, 89.91 mmol, 4.73 mL, 2.0 eq.) in THF (500 mL) was added LiHMDS (1 M, 89.91 mL, 2 eq.) at −78° C., 0.5 hr later compound 2 (16.7 g, 44.96 mmol, 1 eq.) was added. The mixture was stirred at −78° C. for 2 hr. TLC (Petroleum ether: Ethyl acetate=0:1) indicated compound 2 was consumed completely and many new spots formed. The mixture was quenched by addition sat.NH₄Cl aq. 100.0 mL at 0° C., and extracted with EtOAc 300 mL (100 mL×3), dried over Na₂SO₄, filtered and concentrated under reduced pressure to give a residue. The residue was purified by column chromatography (SiO₂, Petroleum ether/Ethyl acetate=100/1 to 0/1). The compound 3 (33.2 g, 87.26 mmol, 64.70% yield) was obtained as yellow solid. TLC: (Petroleum ether: Ethyl acetate=0:1), Rf=0.69.

Preparation of Compound 3.

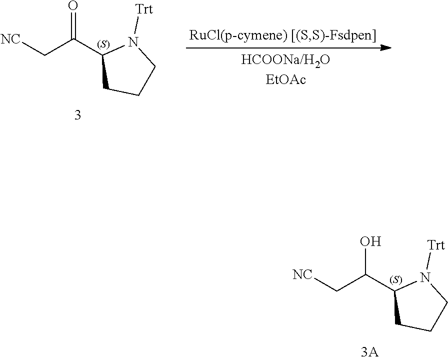

To a solution of compound 3 (15.0 g, 39.42 mmol, 1 eq.) in EtOAc (30 mL) was added RuCl(p-cymene)[(S,S)-Fsdpen](1.40 g, 1.97 mmol, 0.05 eq.) and HCOONa (111.54 g, 1.64 mol, 88.52 mL, 41.6 eq.) in H₂O (120 mL). The mixture was stirred at 30° C. for 60 hrs under N₂. TLC (Plate 1: Petroleum ether: Ethyl acetate=2:1) indicated compound 3 was remained and one new spot formed. The reaction mixture was concentrated under reduced pressure to give a residue. The residue was purified by column chromatography (SiO₂, Petroleum ether/Ethyl acetate=100/1 to 1/1). The compound 3A (12.4 g, 32.42 mmol, 82.23% yield) was obtained was brown solid. ¹HNMR (400 MHz, CHLOROFORM-d) δ=7.57-7.48 (m, 6H), 7.29 (s, 1H), 7.25 (d, J=1.0 Hz, 2H), 7.22-7.16 (m, 3H), 4.21-4.15 (m, 1H), 4.12 (q, J=7.1 Hz, 1H), 3.50 (ddd, J=3.1, 5.7, 8.5 Hz, 1H), 3.20 (s, 1H), 3.21-3.10 (m, 1H), 3.08-3.01 (m, 1H), 2.39 (dd, J=8.0, 16.5 Hz, 1H), 2.20 (dd, J=6.1, 16.6 Hz, 1H), 1.62-1.50 (m, 2H), 1.38-1.29 (m, 1H), 1.23-1.17 (m, 1H). TLC: (Petroleum ether: Ethyl acetate=2:1, Rf=0.30).

Preparation of WV-CA-084.

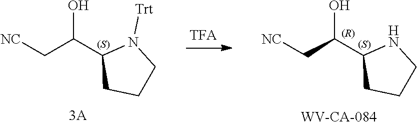

To a solution of compound 3A (7.4 g, 19.35 mmol, 1 eq.) in DCM (90 mL) was added TFA (4.41 g, 38.69 mmol, 2.86 mL, 2.0 eq.) at 0° C. The mixture was stirred at 15° C. for 2 hrs. LCMS (ET32900-198-plal) showed compound 3A was consumed completely and one main peak with desired mass was detected. The mixture was added sat. NaHCO₃ solution to adjust pH for 7 and washed by DCM 50.0 mL and the organic with washed three times by H₂O 10 mL*2. The H₂O phase was dryness by freeze-dryness. The crude product was purified by reversed-phase HPLC (column: C18 20-35 um 100A 20 g; mobile phase: [water-ACN]; B %: 0%-60% @50 mL/min). The WV-CA-084 (1.97 g, 14.00 mmol, 72.35% yield, 99.6% purity) was obtained as yellow solid. ¹HNMR (400 MHz, METHANOL-d₄) δ=4.43-4.34 (m, 1H), 4.08-3.99 (m, 1H), 3.42-3.34 (m, 2H), 3.29-3.21 (m, 1H), 3.16-3.07 (m, 1H), 2.37-2.26 (m, 2H), 2.26-2.19 (m, 1H), 1.70-1.54 (m, 1H). ¹³CNMR (101 MHz, METHANOL-d₄) δ=164.73, 118.32, 115.41, 73.86, 73.14, 72.55, 67.09, 43.23, 42.88, 28.16, 27.56, 27.11, 21.72. LCMS (M+H⁺):141.1.

For additional conditions, see, e.g., Example 1 and Example 2.

### Example 6: Provided Technologies can be Utilized to Manufacture Various Compounds

Those skilled in the art appreciate that provided technologies can be utilized to manufacture various compounds. Certain additional examples and results are presented below.

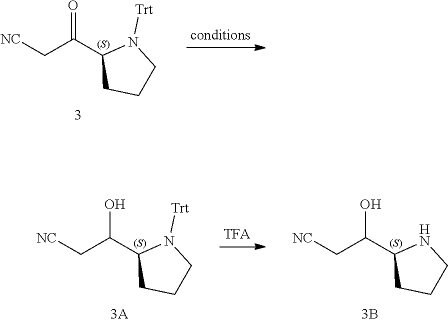

In some embodiments, RuCl(p-cymene)[(S,S)-Fsdpen]was observed to provide high selectivity. Certain results were presented below (80 mg each, metal complex, HCOONa/H₂O, EtOAc):

| | | Ratio Cis/Trans of 3A |
|---|---|---|
| Metal Complex/Catalyst | eq. | (¹H NMR) |
| RuCl(p-cymene)[(S,S)-Fsdpen] | | 1:0.09 |
| Ru-[(S,S)-Ts-DPEN] | 0.01 | ~1:0.31 |
| Ru-[(S,S)-Ts-DPEN] | 0.05 | ~1:0.33 |

### Example 7: Provided Technologies can Provide Various Stereoisomers

As demonstrated herein, provided technologies can provide stereoselective preparation of all stereoisomers of various compounds. An example is described herein.

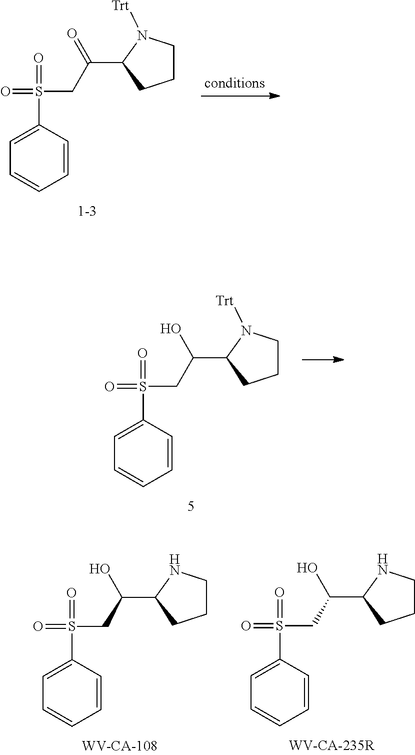

In some embodiments, NaBH₄ was utilized for stereoselective preparation of WV-CA-235R. In some embodiments, 10 equiv. of NaBH₄ was utilized and the reduction was performed at 15° C. in EtOH (0.5 g ketone), and in ¹H NMR only WV-CA-235R was observed. In some embodiments, LiBH₄ was utilized as the reducing agent and similar results to NaBH₄ were observed. In some embodiments, the reduction was performed utilizing Ru—[(S,S)-Ts-DPEN], HCOONa/H₂O in EtOAc (0.5 g ketone) and in ¹H NMR only WV-CA-108 was observed. In some embodiments, the reduction was performed utilizing Ru—[(S,S)-Ts-DPEN], HCOONa/H₂O in EtOAc (3 g ketone) and after deprotection of an amount of compound 5 WV-CA-108 and WV-CA-235R were obtained with a ratio of about 1:0.05 observed by ¹H NMR.

Provided technologies can be utilized to manufacture chirally pure compounds that are useful for various purposes. For example, in some embodiments, compounds described herein, e.g., those of formula DP or salts thereof, were utilized for chirally controlled oligonucleotide synthesis, e.g., as described in WO2019/055951, WO2020/191252, etc.

### Example 8: Technologies for Preparing Phosphoramidites

As demonstrated herein, provided technologies can provide stereoselective preparation of chiral phosphoramidites. Certain examples are described herein.

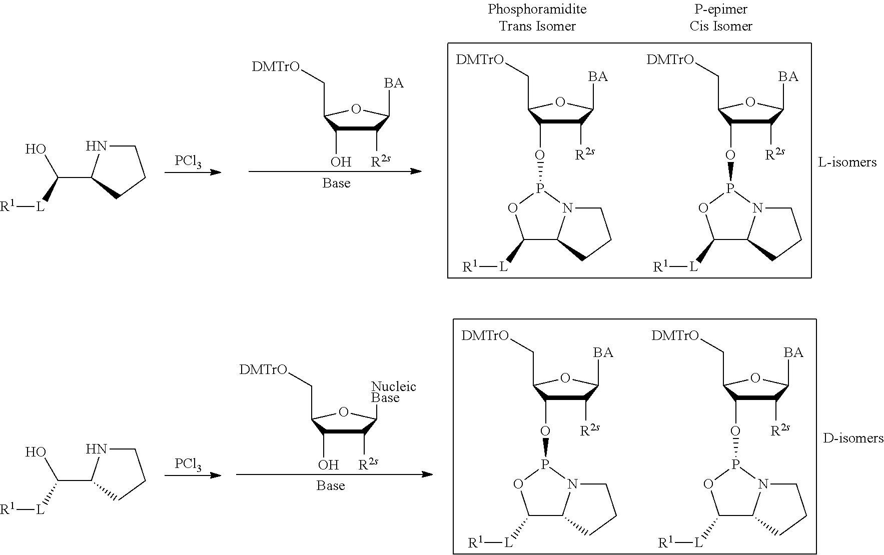

As described herein, in some embodiments, -L-R¹ is —CH₂—SiMePh₂. In some embodiments, -L-R¹ is —CH₂—SO₂Ph. In some embodiments, BA is optionally substituted A, T, C, G or U or a tautomer thereof. In some embodiments, BA is optionally protected (e.g., for oligonucleotide synthesis) A, T, C, G or U or a tautomer thereof. In some embodiments, R^{2s} is —H. In some embodiments, R^{2s} is optionally substituted C₁₋₆ alkyl. In some embodiments, R^{2s} is —OMe. In some embodiments, R^{2s} is -MOE. In some embodiments, R^{2s} is —F.

In some embodiments, a product, e.g., a phosphoramidite such as 8-3 or 8-4, was produced with stereoselectivity. In some embodiments, a trans isomer, e.g., 8-3, was produced stereoselectively over a cis isomer, e.g., 8-4. In some embodiments, ratio of trans:cis isomers is about or at least about 1.1:1, 1.5:1, 2:1, 2.5:1, 3:1, 4:1, 5:1, 6:1, 7:1, 8:1, 9:1, 10:1, 11:1, 12:1, 13:1, 14:1, 15:1, 20:1, 25:1, 30:1, 40:1, 50:1, 60:1, 70:1, 80:1, 90:1, 95:1, 99:1, or 100:1. In some embodiments, a cis isomer, e.g., 8-4 was produced stereoselectively over a trans isomer, e.g., 8-3. In some embodiments, ratio of cis:trans isomers is about or at least about 1.1:1, 1.5:1, 2:1, 2.5:1, 3:1, 4:1, 5:1, 6:1, 7:1, 8:1, 9:1, 10:1, 11:1, 12:1, 13:1, 14:1, 15:1, 20:1, 25:1, 30:1, 40:1, 50:1, 60:1, 70:1, 80:1, 90:1, 95:1, 99:1, or 100:1. In some embodiments, compound 8-1 was converted to phosphoramidites 8-3 and 8-4 at a ratio of about 100%:0. In some embodiments, compound 8-1 is converted to phosphoramidites 8-3 and 8-4 at a ratio of about 0:100%. In some embodiments, cis and trans phosphoramidites were produced, wherein a cis isomer (e.g., 8-4) is more than about 60%. In some embodiments, a cis isomer (e.g., 8-4) is more than about 70%. In some embodiments, a cis isomer (e.g., 8-4) is more than about 80%. In some embodiments, a cis isomer (e.g., 8-4) is more than about 90%. In some embodiments, a cis isomer (e.g., 8-4) is more than about 91%. In some embodiments, a cis isomer (e.g., 8-4) is more than about 92%. In some embodiments, a cis isomer (e.g., 8-4) is more than about 93%. In some embodiments, a cis isomer (e.g., 8-4) is more than about 94%. In some embodiments, a cis isomer (e.g., 8-4) is more than about 95%. In some embodiments, a cis isomer (e.g., 8-4) is more than about 96%. In some embodiments, a cis isomer (e.g., 8-4) is more than about 97%. In some embodiments, a cis isomer (e.g., 8-4) is more than about 98%. In some embodiments, a cis isomer (e.g., 8-4) is more than about 99%. In some embodiments, a cis isomer (e.g., 8-4) is more than about 99.5%. In some embodiments, a cis isomer (e.g., 8-4) is more than about 99.9%.

In some embodiments, as shown in the table below, a cis isomer is formed with higher percentages, in some embodiments, selectively over a trans isomer, compared to a reference condition (e.g., TEA). In some embodiments, certain bases as described herein, e.g., DBU, DBN, etc., provides higher percentages of a cis isomer. In some embodiments, a lower reaction temperature provides a higher percentage of a cis isomer.

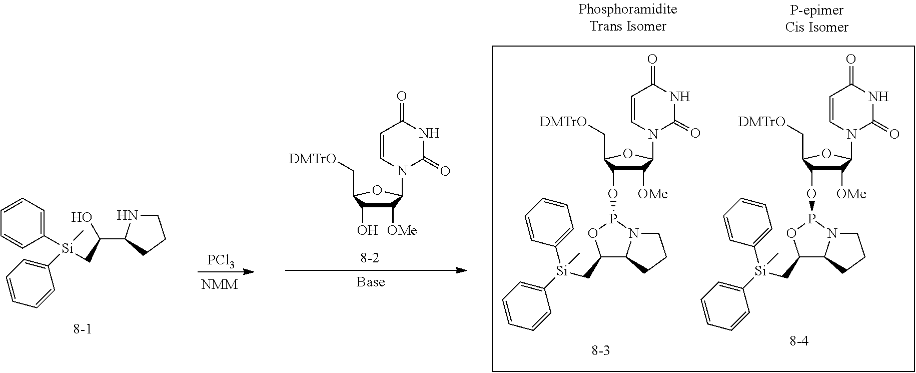

| Base | Temperature (° C.) | Cis Isomer (%) |
|---|---|---|
| TEA | 20 | 0 |
| DBU | 20 | 7.1 |
| DBU | 0 to rt | 10.9 |
| DBU | −15 to rt | 13.0 |
| DBU | −20 to rt | 56.1 |
| DBN | −20 to rt | 91.5 |

Preparation of OMeU-L-DPSE P-epimer Product (8-4, Cis Isomer).

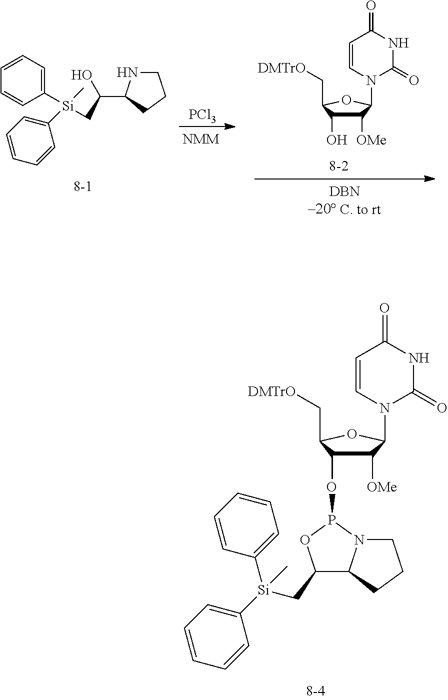

To a solution of OMeU nucleoside (8-2) (5.2 g, 9.28 mmol, 1.0 eq.) in THF (21 mL) was added DBN (5.76 g, 46.38 mmol, 5.7 mL, 5.0 eq.) at 20° C. The mixture was cooled to −20° C. using a dry ice/isopropanol bath. L-DPSE-C₁ (13.6 mL of 0.9574 M solution in THF, 13.03 mmol, 1.4 eq.) was added to the reaction dropwise over 4 min at −20° C. After addition, the reaction was warmed to 20° C. by removing the dry ice/isopropanol bath and stirred at 20° C. for 1 h. LCMS showed compound was consumed completely and one main peak with desired mass was detected. The reaction was quenched with water (80 μL, 0.5 eq.) and stirred at 20° C. for 5 min. 4 Å molecular sieve power (1.29 g) was added and stirred at 20° C. for 10 min. The reaction was filtered using vacuum, then concentrated under reduced pressure at 30° C. to give a white foam. The crude product was purified by column chromatography (SiO₂, Ethyl acetate/hexane=20/1 to 100/1, solvents contained 5% TEA). The compound (1.61 g, 1.79 mmol, 19.3% yield, 91.5% p-epimer, 8-4) was obtained as a white solid. ¹H NMR (600.13 MHz, chloroform-d) δ=9.79 (bs, 1H), 8.02 (d, J=8.1 Hz, 1H), 7.47-7.44 (m, 4H), 7.32 (d, J=7.8 Hz, 2H), 7.27-7.20 (m, 14H), 7.16-7.13 (m, 2H), 6.76 (dd, J=8.9, 2.8 Hz, 4H), 5.82 (s, 1H), 5.19 (d, J=8.1 Hz, 1H), 4.59-4.55 (m, 2H), 4.01-3.99 (m, 1H), 3.68 (d, J=2.3 Hz, 6H), 3.55-3.52 (m, 2H), 3.37 (s, 3H), 3.27 (d, J=10.2 Hz, 2H), 2.79-2.73 (m, 1H), 2.69-2.62 (m, 1H), 1.64 (dd, J=14.6, 8.7 Hz, 1H), 1.58-1.49 (m, 4H), 1.40 (dd, J=14.6, 6.0 Hz, 1H), 1.31-1.27 (m, 1H), 1.22-1.14 (m, 2H), 0.80-0.74 (m, 1H), 0.58 (s, 3H). ¹³C NMR (150.92 MHz, chloroform-d) δ=163.74, 158.83, 158.80, 150.22, 144.10, 140.14, 136.61, 136.30, 135.03, 134.89, 134.67, 134.59, 134.40, 130.36, 130.31, 129.47, 129.44, 128.34, 128.08, 127.97, 127.94, 127.27, 113.36, 102.04, 87.77, 87.15, 83.92, 81.79, 81.77, 80.02, 79.94, 69.97, 69.83, 66.90, 66.85, 60.32, 58.53, 55.30, 43.09, 31.62, 26.82, 26.65, 22.69, 19.16, 14.18, −3.24. ³¹P NMR (242.96 MHz, chloroform-d) δ=146.33 ppm. LCMS (M+K⁺): 938.58.

Preparation of dA(N6-Bz)-L-DPSE P-epimer Product (8-6, Cis Isomer).

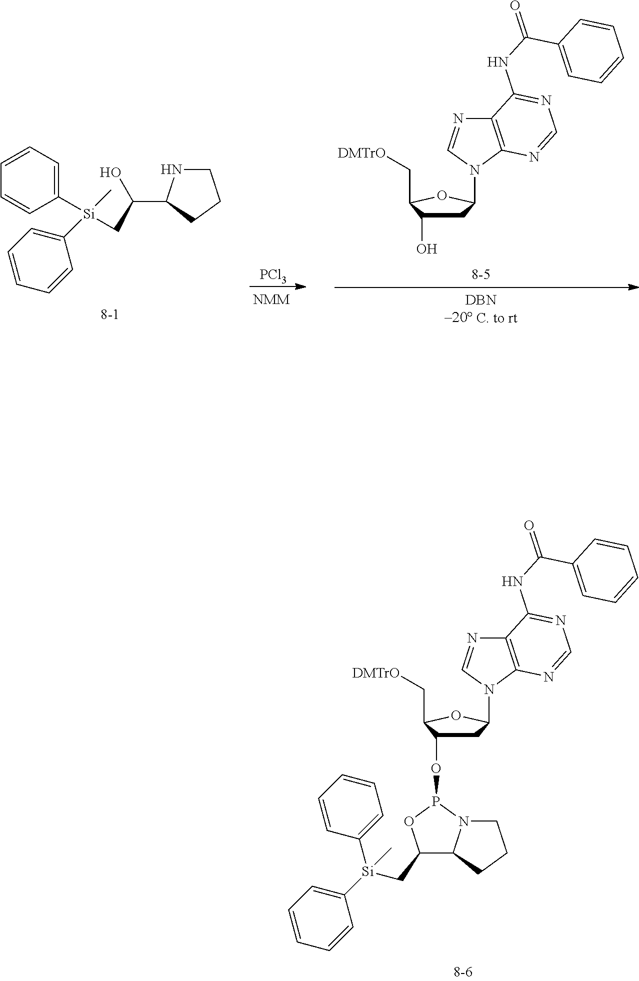

To a solution of dA(N6-Bz) nucleoside (8-5) (5.0 g, 7.60 mmol, 1.0 eq.) in THF (18 mL) was added DBN (4.82 g, 38.84 mmol, 4.8 mL, 5.0 eq.) at 20° C. The mixture was cooled to −20° C. using a dry ice/isopropanol bath. L-DPSE-C₁ (11.4 mL of 0.9574 M solution in THF, 10.64 mmol, 1.4 eq.) was added to the reaction dropwise over 4 min at −20° C. After addition, the reaction was warmed to 20° C. by removing the dry ice/isopropanol bath and stirred at 20° C. for 1 h. LCMS showed compound was consumed completely and one main peak with desired mass was detected. The reaction was quenched with water (80 μL, 0.5 eq.) and stirred at 20° C. for 5 min. 4 Å molecular sieve power (1.24 g) was added and stirred at 20° C. for 10 min. The reaction was filtered using vacuum, then concentrated under reduced pressure at 30° C. to give a yellow oil. The crude product was purified by column chromatography (SiO₂, Ethyl acetate/hexane=20/1 to 100/1, solvents contained 5% TEA). The compound (0.96 g, 0.96 mmol, 12.7% yield, 93.1% p-epimer, 8-6) was obtained as a white solid. ¹H NMR (600.13 MHz, chloroform-d) δ=8.58 (s, 1H), 8.01 (s, 1H), 7.91 (d, J=7.5 Hz, 2H), 7.46-7.40 (m, 6H), 7.35 (t, J=7.6 Hz, 2H), 7.30 (d, J=7.8 Hz, 2H), 7.25-7.19 (m, 8H), 7.16-7.14 (m, 5H), 7.09 (t, J=7.2 Hz, 1H), 6.69 (d, J=8.5 Hz, 4H), 6.22 (t, J=6.6 Hz, 1H), 4.75-4.71 (m, 1H), 4.62-4.57 (m, 1H), 4.01-3.98 (m, 2H), 3.64 (s, 6H), 3.37 (ddt, J=17.4, 10.0, 6.5 Hz, 1H), 3.26 (ddd, J=31.6, 10.5, 4.4 Hz, 2H), 3.04-2.98 (m, 1H), 2.84-2.76 (m, 1H), 2.56 (dt, J=13.4, 6.5 Hz, 1H), 2.13 (ddd, J=13.6, 6.1, 3.4 Hz, 1H), 1.80-1.54 (m, 5H), 1.46-1.42 (m, 1H), 1.37 (dd, J=14.6, 4.6 Hz, 1H), 0.58 (s, 3H). ¹³C NMR (150.92 MHz, chloroform-d) δ=158.65, 144.49, 141.39, 136.79, 136.50, 135.64, 135.63, 134.68, 134.57, 134.54, 133.84, 132.82, 130.12, 130.10, 129.47, 129.39, 128.93, 128.20, 127.98, 127.94, 127.90, 127.04, 123.47, 113.27, 86.65, 86.24, 86.21, 84.47, 79.87, 79.79, 74.10, 73.95, 67.06, 67.01, 63.25, 60.46, 55.31, 43.24, 39.72, 39.71, 27.34, 26.92, 21.13, 19.45, 14.29, −3.32. ³¹P NMR (242.96 MHz, chloroform-d) δ=145.76 ppm. LCMS (M−H⁺): 996.80.

Preparation of dT-D-DPSE P-epimer Product (8-9, Cis Isomer).

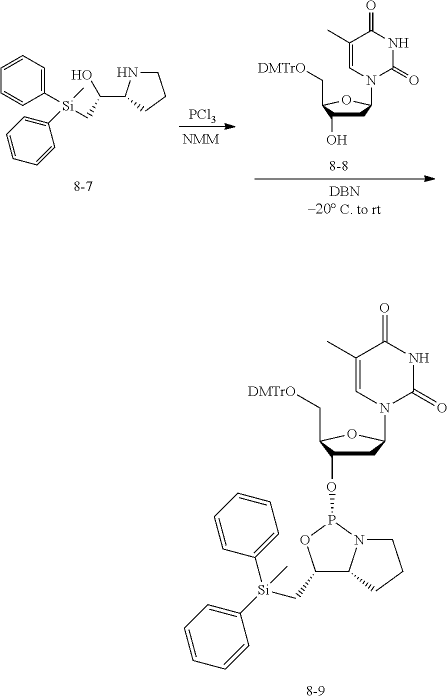

To a solution of dT nucleoside (8-8) (5.1 g, 9.36 mmol, 1.0 eq.) in THF (22 mL) was added DBN (6.03 g, 48.56 mmol, 6.0 mL, 5.0 eq.) at 20° C. The mixture was cooled to −20° C. using a dry ice/isopropanol bath. D-DPSE-C₁ (14.0 mL of 0.9574 M solution in THF, 13.40 mmol, 1.4 eq.) was added to the reaction dropwise over 7 min at −20° C. After addition, the reaction was warmed to 20° C. by removing the dry ice/isopropanol bath and stirred at 20° C. for 1 h. LCMS showed compound was consumed completely and one main peak with desired mass was detected. The reaction was quenched with water (80 μL, 0.5 eq.) and stirred at 20° C. for 5 min. 4 Å molecular sieve power (1.30 g) was added and stirred at 20° C. for 10 min. The reaction was filtered using vacuum, then concentrated under reduced pressure at 30° C. to give a yellow oil. The crude product was purified by column chromatography (SiO₂, Ethyl acetate/hexane=20/1 to 100/1, solvents contained 5% TEA). The compound (1.66 g, 1.88 mmol, 20.1% yield, 81.2% p-epimer, 8-9) was obtained as a white solid. ¹H NMR (600.13 MHz, chloroform-d) δ=9.55 (bs, 1H), 7.53 (s, 2H), 7.46-7.38 (m, 7H), 7.32-7.30 (m, 4H), 7.25-7.17 (m, 18H), 7.13 (dd, J=8.5, 6.1 Hz, 2H), 6.75 (d, J=8.9 Hz, 6H), 6.25 (t, J=6.9 Hz, 1H), 4.73-4.70 (m, 1H), 4.51 (ddd, J=12.1, 9.9, 5.5 Hz, 1H), 3.80 (q, J=2.7 Hz, 1H), 3.67 (s, 9H), 3.37-3.24 (m, 4H), 3.18-3.11 (m, 3H), 2.87 (dddd, J=20.2, 11.0, 9.3, 5.8 Hz, 1H), 2.24 (dd, J=7.0, 4.4 Hz, 2H), 1.93 (s, 3H), 1.85-1.77 (m, 1H), 1.74-1.66 (m, 2H), 1.58-1.48 (m, 3H), 1.14 (t, J=7.2 Hz, 3H), 0.48 (s, 3H). ¹³C NMR (150.92 MHz, chloroform-d) δ=170.07, 163.16, 157.71, 157.69, 149.60, 143.31, 135.51, 135.31, 134.49, 134.36, 134.31, 133.56, 133.54, 133.43, 133.38, 129.13, 129.09, 129.07, 128.31, 128.27, 127.15, 126.97, 126.92, 126.87, 126.84, 126.80, 126.10, 112.27, 112.21, 112.19, 110.23, 85.98, 84.76, 83.54, 78.66, 78.58, 73.46, 73.30, 65.78, 65.74, 62.00, 59.34, 54.19, 42.11, 39.58, 39.55, 26.13, 25.74, 19.99, 18.09, 13.17, 10.76, 10.72, −4.23, −4.32. ³¹P NMR (242.96 MHz, chloroform-d) δ=146.52 ppm. LCMS (M−H⁺): 882.97.

Preparation of additional cis isomers are presented below as examples. In some embodiments, a mixture of bases are utilized. In some embodiments, a base is DBU. In some embodiments, a base is DBN. In some embodiments, a base is TEA. In some embodiments, two bases are utilized, one of which is DBU. In some embodiments, two bases are utilized, one of which is DBN. In some embodiments, two bases are utilized, one of which is TEA. In some embodiments, a mixture of DBU and TEA is utilized. In some embodiments, a mixture of DBN and TEA is utilized. In some embodiments, ratio of a base (e.g., DBU, DBN, etc.) with TEA in a mixture is about or at least 1:1, 2:1, or 3:1. In some embodiments, it is about or at least about 1:1. In some embodiments, it is about or at least about 2:1. In some embodiments, it is about or at least about 3:1. In some embodiments, a higher ratio of DBU or DBN provides higher percentages of a cis isomer. In some embodiments, about 1 equivalent of base is utilized. In some embodiments, more than 1 equivalent of base (either a single base or a mixture of bases) is utilized. In some embodiments, equivalent of base (either a single base or a mixture of bases) is about or at least about 1.5, 2, 2.5, or 3. In some embodiments, it is about 2. In some embodiments, it is about 3. In some embodiments, a higher equivalent of base provides higher percentage of cis isomer. In some embodiments, equivalent of base is higher than equivalent of a phosphorus chloride agent (e.g., PSM-C₁). Certain preparations are presented below as examples.

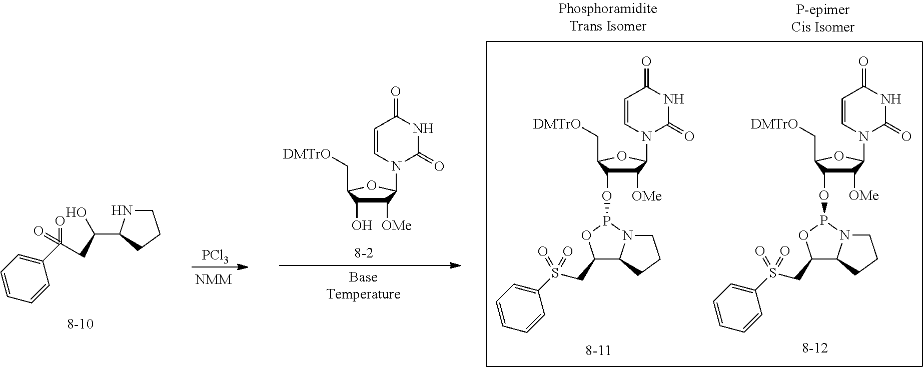

| Base | Base Eq. | Temperature (° C.) | Cis Isomer (%) |
|---|---|---|---|
| DBU | 1.0 | −20° C. to rt | 2.2 |
| DBU:TEA (1:1) | 2.0 | −20° C. to rt | 13.3 |
| DBN:TEA (1:1) | 2.0 | −20° C. to rt | 32.0 |
| DBN:TEA (3:1) | 2.0 | −20° C. to rt | 33.6 |
| DBN:TEA (1:1) | 3.0 | −20° C. to rt | 54.2 |
| DBN:TEA (3:1) | 2.0 | 0° C. to rt | 65.5 |

Preparation of OMeU-L-PSM P-epimer Product (8-12, Cis Isomer).

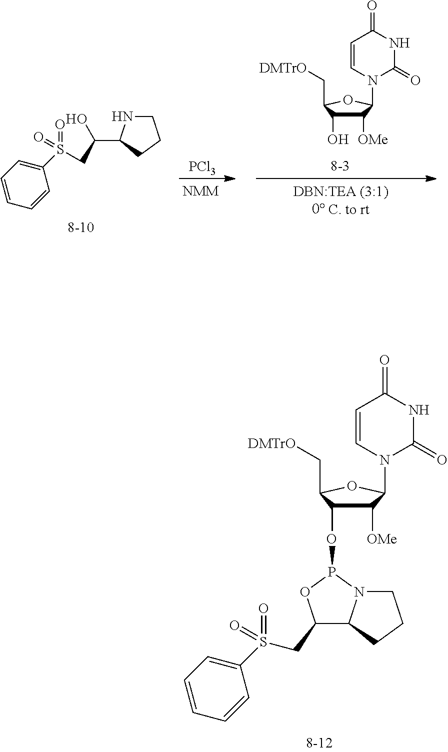

To a solution of OMeU nucleoside (8-3) (1.05 g, 1.87 mmol, 1.0 eq.) in THF (5.3 mL) was added a solution of DBN (0.35 g, 2.83 mmol, 0.35 mL, 1.5 eq.) and TEA (94.38 mg, 0.93 mmol, 0.13 mL, 0.5 eq.) in THF (1.1 mL) at 20° C. The mixture was cooled to 0° C. using a dry ice/isopropanol bath. L-PSM-C₁ (13.6 mL of 0.9 M solution in THF, 12.24 mmol, 1.6 eq.) was added to the reaction dropwise over 4 min at 0° C. After addition, the reaction was warmed to 20° C. by removing the dry ice/isopropanol bath and stirred at 20° C. for 1 h. LCMS showed compound was consumed completely and one main peak with desired mass was detected. The reaction was quenched with water (80 μL, 0.5 eq.) and stirred at 20° C. for 5 min. 4 Å molecular sieve powder (1.29 g) was added and stirred at 20° C. for 10 min. The reaction was filtered using vacuum, then concentrated under reduced pressure at 30° C. to give a white foam. The crude product was purified by column chromatography (SiO₂, Ethyl acetate/hexane=50/1 to 100/1). The compound (383 mg, 0.45 mmol, 24.2% yield, 65.5% p-epimer (8-12), 34.5% trans isomer (8-11)) was obtained as a white solid. ¹H NMR (600.13 MHz, chloroform-d) δ=10.01 (bs, 2H), 8.06 (d, J=8.1 Hz, 1H), 7.98-7.96 (m, 3H), 7.89 (d, J=7.8 Hz, 1H), 7.66-7.63 (m, 1H), 7.61-7.55 (m, 3H), 7.49 (t, J=7.7 Hz, 2H), 7.39 (d, J=7.8 Hz, 4H), 7.32-7.28 (m, 10H), 7.24 (t, J=7.4 Hz, 2H), 6.86 (d, J=8.4 Hz, 7H), 6.02 (d, J=3.0 Hz, 1H), 5.94 (s, 1H), 5.31 (d, J=8.1 Hz, 1H), 5.27 (d, J=8.1 Hz, 1H), 5.06 (q, J=6.1 Hz, 1H), 4.88 (dq, J=7.1, 4.9 Hz, 1H), 4.70-4.65 (m, 2H), 4.17-4.16 (m, 1H), 4.11 (q, J=7.1 Hz, 3H), 4.06-4.04 (m, 1H), 3.92 (dd, J=4.9, 3.1 Hz, 1H), 3.78 (s, 12H), 3.75-3.70 (m, 3H), 3.68-3.63 (m, 1H), 3.61 (dd, J=11.4, 2.3 Hz, 2H), 3.55 (s, 3H), 3.47 (s, 6H), 3.40 (dd, J=14.6, 5.4 Hz, 1H), 3.32 (dd, J=11.4, 2.4 Hz, 1H), 3.13 (qd, J=10.0, 4.1 Hz, 1H), 2.86-2.76 (m, 2H), 2.03 (s, 4H), 1.88-1.69 (m, 5H), 1.66-1.62 (m, 1H), 1.59-1.54 (m, 1H), 1.25 (t, J=7.2 Hz, 4H), 1.15-1.09 (m, 1H). ¹³C NMR (150.92 MHz, chloroform-d) δ=171.14, 163.76, 158.79, 158.77, 158.73, 150.41, 150.31, 144.37, 144.01, 140.17, 139.91, 139.45, 139.43, 135.17, 135.03, 134.91, 134.78, 134.00, 133.96, 130.29, 130.26, 130.22, 129.37, 129.31, 128.26, 128.22, 128.11, 128.06, 128.02, 127.25, 127.19, 113.33, 113.31, 102.21, 87.36, 87.13, 87.12, 86.99, 83.60, 83.35, 83.32, 82.37, 82.35, 81.79, 81.76, 74.97, 74.90, 74.83, 74.76, 69.85, 69.80, 69.73, 66.22, 66.20, 65.74, 65.69, 61.20, 60.38, 60.32, 59.32, 58.59, 58.11, 58.04, 58.02, 55.27, 55.26, 46.49, 46.26, 42.43, 27.66, 27.32, 27.29, 26.02, 26.00, 21.04, 14.21. ³¹P NMR (242.96 MHz, chloroform-d) δ=144.73 ppm. LCMS (M−H⁺): 842.32.

Preparation of OMeC(N4-Ac)-L-PSM P-epimer Product (8-14, Cis Isomer).

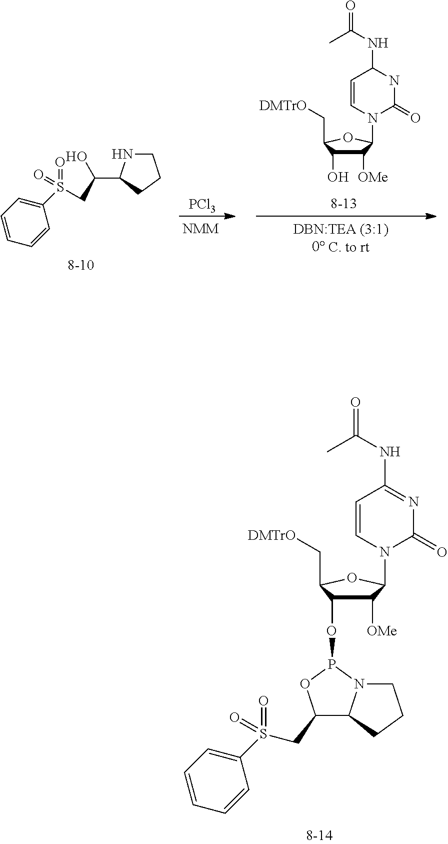

To a solution of OMeC(N4-Ac) nucleoside (8-13) (9.96 g, 16.55 mmol, 1.0 eq.) in THF (47 mL) was added a solution of DBN (3.12 g, 25.09 mmol, 3 mL, 1.5 eq.) and TEA (0.87 g, 8.61 mmol, 1.20 mL, 0.5 eq.) in THF (9.2 mL) at 20° C. The mixture was cooled to 0° C. using a dry ice/isopropanol bath. L-PSM-C₁ (29.0 mL of 0.9 M solution in THF, 26.10 mmol, 1.6 eq.) was added to the reaction dropwise over 4 min at 0° C. After addition, the reaction was warmed to 20° C. by removing the dry ice/isopropanol bath and stirred at 20° C. for 1 h. LCMS showed compound was consumed completely and one main peak with desired mass was detected. The reaction was quenched with water (0.2 mL, 0.5 eq.) and stirred at 20° C. for 5 min. 4 Å molecular sieve power (2.54 g) was added and stirred at 20° C. for 10 min. The reaction was filtered using vacuum, then concentrated under reduced pressure at 30° C. to give a white foam. The crude product was purified by column chromatography (SiO₂, Ethyl acetate/hexane=60/1 to 100/1). The compound (2.61 g, 2.95 mmol, 17.8% yield, 91.2% p-epimer (8-14)) was obtained as a white solid. ¹H NMR (600.13 MHz, chloroform-d) δ=10.41 (bs, 2H), 8.59 (d, J=7.5 Hz, 1H), 7.95 (d, J=7.5 Hz, 2H), 7.63 (t, J=7.4 Hz, 1H), 7.56 (t, J=7.7 Hz, 2H), 7.42 (d, J=7.4 Hz, 2H), 7.35-7.31 (m, 7H), 7.27 (t, J=7.2 Hz, 1H), 7.14 (d, J=7.5 Hz, 1H), 6.87 (d, J=8.0 Hz, 4H), 5.99 (s, 1H), 4.83 (p, J=6.7 Hz, 1H), 4.63-4.59 (m, 1H), 4.11 (q, J=7.0 Hz, 2H), 3.81 (d, J=1.8 Hz, 6H), 3.70-3.63 (m, 3H), 3.53 (s, 3H), 3.49 (dd, J=14.7, 5.5 Hz, 1H), 3.30 (dd, J=11.5, 2.3 Hz, 1H), 2.76-2.73 (m, 2H), 2.29 (s, 3H), 1.81-1.75 (m, 1H), 1.73-1.66 (m, 2H), 1.60-1.53 (m, 1H). ¹³C NMR (150.92 MHz, chloroform-d) δ=171.13, 171.02, 163.24, 158.79, 144.67, 143.86, 139.38, 135.01, 134.90, 134.01, 130.24, 130.22, 129.40, 128.27, 128.26, 128.15, 128.10, 128.06, 127.26, 113.38, 113.37, 96.95, 88.35, 87.12, 83.64, 81.35, 81.32, 75.01, 74.93, 69.17, 69.04, 65.68, 65.63, 60.38, 59.80, 59.38, 58.04, 55.28, 42.43, 27.68, 27.23, 24.88, 21.06, 14.22. ³¹P NMR (242.96 MHz, chloroform-d) δ=144.38 ppm. LCMS (M+H⁺): 885.30.

Preparation of OMOE-T-D-PSM P-epimer Product (8-17, Cis Isomer).

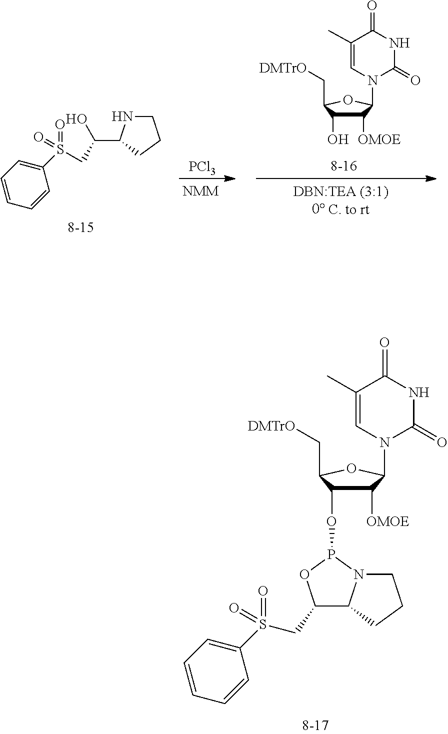

To a solution of OMOE-T nucleoside (8-16) (25.32 g, 40.78 mmol, 1.0 eq.) in THF (114 mL) was added a solution of DBN (7.61 g, 61.25 mmol, 7.3 mL, 1.5 eq.) and TEA (2.11 g, 20.81 mmol, 2.9 mL, 0.5 eq.) in THF (23 mL) at 20° C. The mixture was cooled to 0° C. using a dry ice/isopropanol bath. D-PSM-C₁ (82 mL of 0.8 M solution in THF, 65.6 mmol, 1.6 eq.) was added to the reaction dropwise over 15 min at 0° C. After addition, the reaction was warmed to 20° C. by removing the dry ice/isopropanol bath and stirred at 20° C. for 3 h. LCMS showed compound was consumed completely and one main peak with desired mass was detected. The reaction was quenched with water (0.36 mL, 0.5 eq.) and stirred at 20° C. for 5 min. 4 Å molecular sieve power (6.08 g) was added and stirred at 20° C. for 10 min. The reaction was filtered using vacuum, then concentrated under reduced pressure at 30° C. to give a yellow oil. The crude product was purified by column chromatography (SiO₂, Ethyl acetate/hexane=60/1 to 100/1). The compound (19.2 g, 21.29 mmol, 52.2% yield, 49.1% p-epimer (8-17), 50.9% trans isomer) was obtained as a white solid. ¹H NMR (600.13 MHz, chloroform-d) δ=9.52 (bs, 2H), 7.84 (d, J=7.8 Hz, 2H), 7.79 (d, J=7.6 Hz, 2H), 7.64 (s, 1H), 7.56 (t, J=7.2 Hz, 1H), 7.48 (dd, J=16.2, 8.6 Hz, 3H), 7.38 (t, J=7.2 Hz, 7H), 7.35-7.31 (m, 6H), 7.27-7.14 (m, 13H), 6.78-6.75 (m, 7H), 5.94 (t, J=5.8 Hz, 2H), 4.97 (q, J=6.2 Hz, 1H), 4.74 (qd, J=7.6, 4.0 Hz, 1H), 4.62 (dt, J=10.5, 5.5 Hz, 1H), 4.44 (ddd, J=8.2, 4.6, 2.6 Hz, 1H), 4.25 (t, J=4.8 Hz, 1H), 4.15-4.14 (m, 1H), 4.09 (t, J=4.3 Hz, 1H), 3.85-3.81 (m, 2H), 3.76-3.65 (m, 15H), 3.57-3.40 (m, 9H), 3.35-3.31 (m, 2H), 3.29-3.27 (m, 1H), 3.25 (s, 3H), 3.23 (d, J=4.1 Hz, 1H), 3.20 (s, 3H), 3.15-3.12 (m, 1H), 3.01 (qd, J=9.6, 3.9 Hz, 1H), 2.89-2.82 (m, 1H), 1.92-1.88 (m, 1H), 1.86-1.74 (m, 2H), 1.72-1.61 (m, 2H), 1.57-1.53 (m, 1H), 1.40 (dq, J=11.7, 9.0 Hz, 1H), 1.26 (s, 3H), 1.24 (s, 3H), 1.04 (p, J=9.6 Hz, 1H). ¹³C NMR (150.92 MHz, chloroform-d) δ=171.18, 164.16, 164.03, 158.88, 158.87, 158.79, 158.77, 150.85, 150.42, 144.21, 144.16, 139.89, 139.51, 135.72, 135.71, 135.28, 135.24, 135.13, 134.12, 133.74, 130.31, 130.24, 129.44, 129.13, 128.35, 128.33, 128.31, 128.20, 128.10, 128.04, 127.32, 127.20, 113.39, 113.32, 113.29, 111.29, 110.80, 87.67, 87.36, 86.98, 85.67, 83.49, 83.47, 82.37, 82.34, 81.92, 81.72, 81.71, 74.36, 74.28, 74.12, 74.06, 72.68, 72.56, 72.50, 72.37, 70.42, 70.40, 70.29, 70.20, 66.14, 66.12, 65.69, 65.63, 63.15, 61.52, 60.43, 59.54, 59.11, 59.05, 58.13, 58.11, 55.34, 55.30, 46.62, 46.39, 42.94, 27.92, 27.36, 26.09, 26.06, 21.09, 14.25, 11.73, 11.62. ³¹P NMR (242.96 MHz, chloroform-d) δ=145.55 ppm. LCMS (M+K⁺): 940.33.

Additional preparations of cis isomers of various phosphoramidites are presented below as examples.

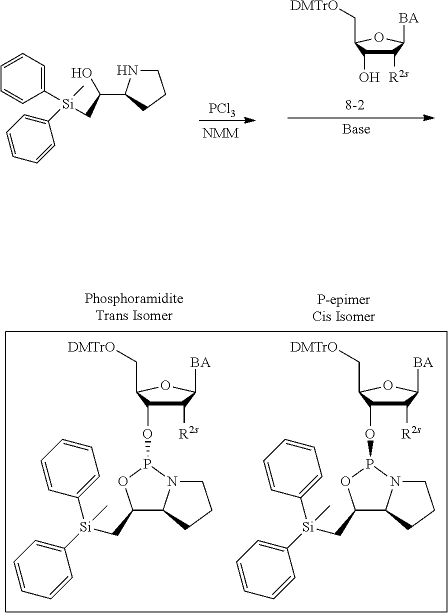

| | % P-epimer | P-epimer 31P |
|---|---|---|
| Amidite | (cis isomer) | Chemical Shift (ppm) |
| dT-L-DPSE | 93.9% | 145.34 |
| dC(N4-Ac)-L-DPSE | 99.1% | 145.22 |
| dC(N4-Ac)-D-DPSE | 87.1% | 146.82 |
| OMeA(N6-Bz)-L-DPSE | 92.0% | 147.26 |
| dG(N2-iBu)-L-DPSE | 19.7% | 145.75 |
| dG(N2-iBu)-D-DPSE | 23.3% | 145.86 |
| OMeG(N2-iBu)-L-DPSE | 51.0% | 147.06 |
| MOE-5MeC(N4-Bz)-L-DPSE | 94.2% | 147.25 |
| 5Me-dC(N4-Bz)-L-DPSE | 83.3% | 145.28 |
| FdA(N6-Bz)-L-DPSE | 92.2% | 145.94 |
| FdC(N4-Ac)-L-DPSE | 93.2% | 146.01 |
| FdU-L-DPSE | 93.3% | 145.96 |
| FdG(N2-iBu)-L-DPSE | 84.0% | 145.76 |

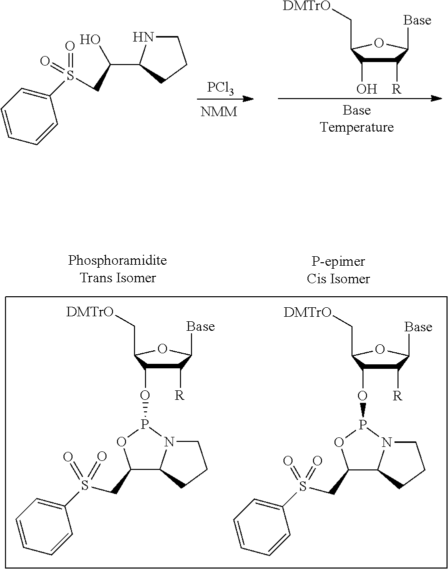

| | % | P-epimer 31P |
|---|---|---|
| Amidite | P-epimer | Chemical Shift (ppm) |
| FdC(N4-Ac)-L-PSM | 86.0% | 144.75 |
| FdG(N2-iBu)-L-PSM | 69.8% | 144.09 |
| MOE-5MeC(N4-Bz)-L-PSM | 99.8% | 145.83 |
| OMeG(N2-iBu)-L-PSM | 75.4% | 142.91 |
| MOE-G(N2-iBu)-L-PSM | 75.4% | 142.33 |

### Example 9: Technologies for Converting Chiral Phosphorus Configurations

In some embodiments, the present disclosure provides technologies for converting configurations of chiral phosphorus in a compound, e.g., a cis phosphoramidite as described herein. As demonstrated herein, in some embodiments, certain agents, e.g., weak acids (e.g., certain salts of amines), phosphoramidite activators, etc., can convert a cis cyclic phosphoramidite into a trans cyclic phosphoramidite. Certain examples are presented below.

Isomerization of OMeU-L-DPSE cis isomer (8-3).

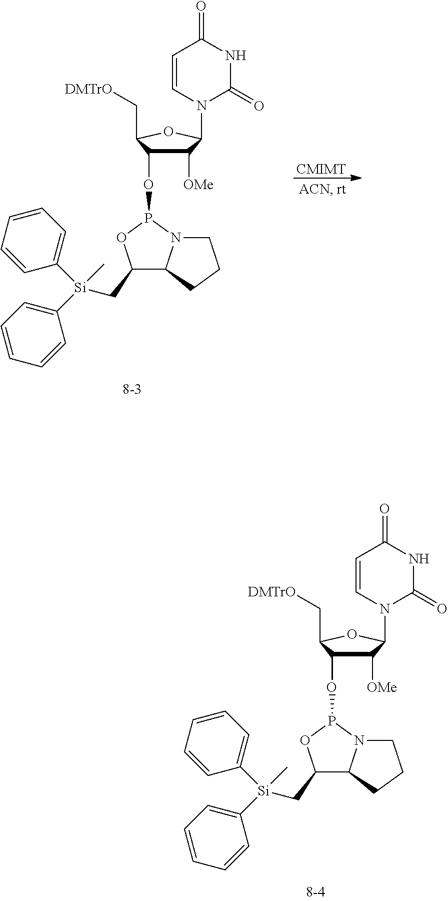

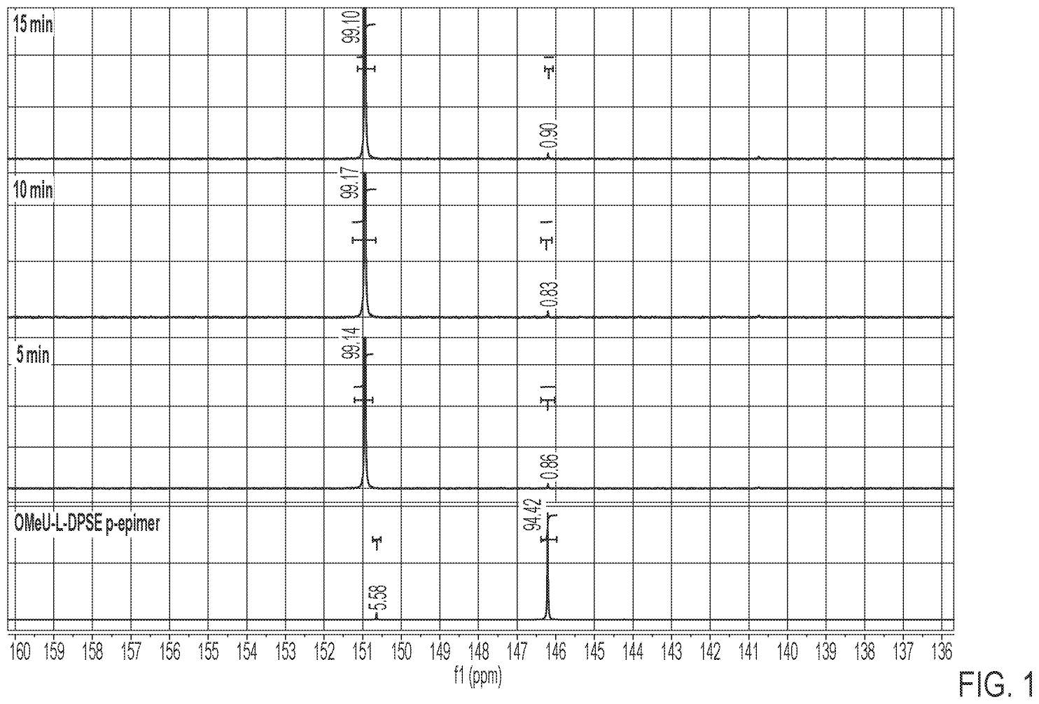

In one example, CMIMT was used to isomerize cis isomer 8-3 to trans isomer 8-4. 0.5M CMIMT stock solution in ACN was prepared. A mixture of cis and trans isomers of OMeU-L-DPSE (100 mg, 0.11 mmol, 1.0 eq.) in ACN (0.5 mL) was prepared in an NMR tube. CMIMT (10.3 mg, 0.2 mL of 0.5M solution, 0.1 mmol, 0.9 eq.) were added to the NMR tube at room temperature. ³¹P NMR was taken immediately after addition three times in a row. The change of NMR spectrum over a course of 15 min under above conditions was recorded and is shown in FIG. 1.

Isomerization of OMeU-L-PSM cis isomer (8-11).

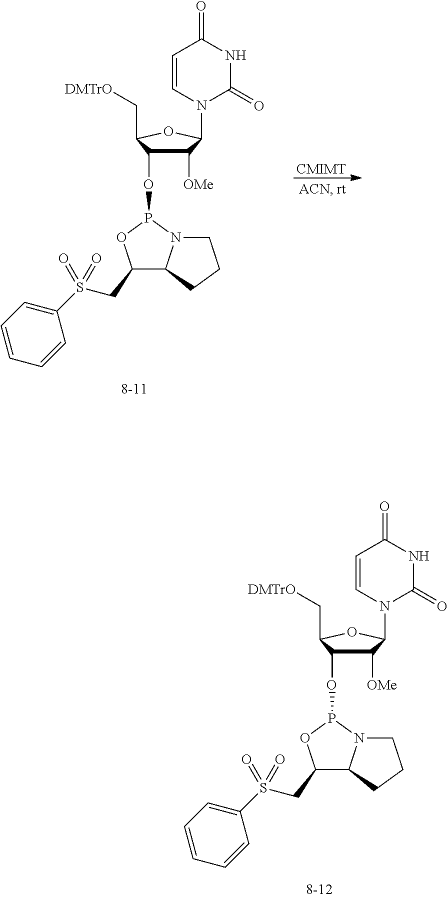

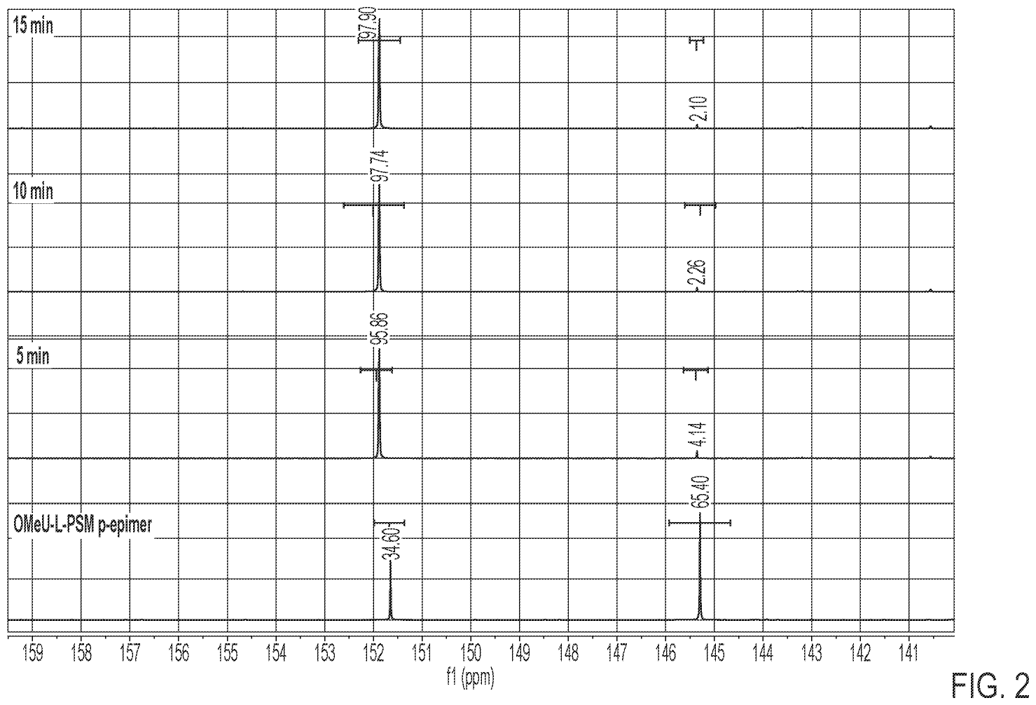

In one example, CMIMT was used to isomerize cis isomer 8-11 to trans isomer 8-12. 0.5M CMIMT stock solution in ACN was made. A mixture of cis and trans isomers of OMeU-L-PSM (100 mg, 0.12 mmol, 1.0 eq.) in ACN (0.5 mL) was prepared in an NMR tube. CMIMT (10.3 mg, 0.2 mL of 0.5M solution, 0.1 mmol, 0.8 eq.) were added to the NMR tube at room temperature. ³¹P NMR was taken immediately after addition three times in a row. The change of NMR spectrum over a course of 15 min under above conditions was recorded and is shown in FIG. 2.

### Example 10: Technologies for Oligonucleotide Synthesis

In some embodiments, the present disclosure provides technologies for manufacturing oligonucleotides. In some embodiments, the present disclosure provides technologies for constructing sulfonyl PN linkages, e.g., those having or comprising the structure of —OP(O)(NHSO₂R^{s})O— wherein R^{s} is as described herein (e.g., in some embodiments, R^{s} is R as described herein; in some embodiments, R is optionally substituted C₁₋₆ aliphatic; in some embodiments, R is optionally substituted aryl or heteroaryl; etc.). In some embodiments, reduced amounts and/or equivalents (e.g., relative to phosphoramidites and/or nucleosides/oligonucleotides to which new units are added, etc.) of azide agents are utilized. In some embodiments, equivalent of an azide agent is about or no more than about 2.0 equivalent. In some embodiments, it is about or no more than 1.5 equivalent. In some embodiments, it is about 1 equivalent. In some embodiments, an equivalent is relative to a phosphoramidite. As those skilled in the art appreciate, reduced amounts of azide agents can provide safer manufacturing processes and/or can reduce manufacturing cost. In some embodiments, provided technologies comprise reacting a P(III) agent (e.g., a phosphoramidite) with an azide agent (e.g., R²—SO₂N₃) to provide a composition, and then contact the composition with a coupling partner which is or comprises a nucleotide or oligonucleotide, which may be linked to a solid support optionally via a linker (e.g., as in solid-support based oligonucleotide synthesis). Certain examples are presented below as examples.

A useful experimental procedure for forming sulfonyl PN linkages:

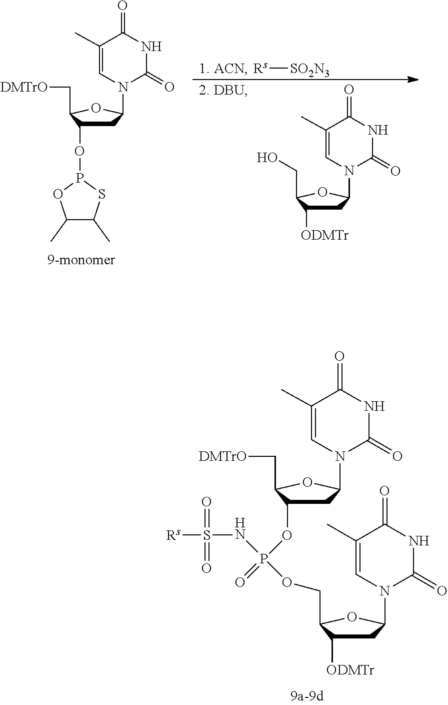

9a: R^{s}=—CH₃; 9b: R^{s}=—C₆H₁₃; 9c: R^{s}=—C₁₂H₂₅; 9d: R^{s}=

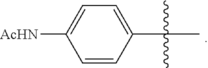 [SMILES: *c1ccc(NC(C)=O)cc1]

To a stirred solution of 9-monomer (0.091 mmol, 2 eq., pre-dried by co-evaporation with dry acetonitrile followed by under vacuum for minimum 12 h) in dry acetonitrile (0.5 mL) was added a solution of sulfonyl azide (0.18 mmol, 4 eq., in various cases crude) in acetonitrile (0.3 mL) under argon atmosphere at room temperature. Resulting reaction mixture was stirred for 30 min. then DMTr protected alcohol (0.046 mmol, pre-dried by co-evaporation with dry acetonitrile and dried under vacuum for minimum 12 h) in dry acetonitrile (0.2 mL) and 1,8-Diazabicyclo [5.4.0]undec-7-ene (0.22 mmol, 5 eq., 1 M solution in dry acetonitrile) were added. After the reaction was completed, 10-20 mins, it was analyzed by LCMS.

Data for 9a (R: Me): MS (ES) m/z calculated molecular weight for C₆₃H₆₆N₅O₁₇PS 1228.27, Observed mass [M−H]⁻: 1227.01.

Data for 9b (R: C₆H₁₃): MS (ES) m/z calculated molecular weight for C₆₈H₇₆N₅O₁₇PS: 1298.41, Observed mass [M−H]⁻: 1296.95.

Data for 9c (R: C₁₂H₂₅): MS (ES) m/z calculated molecular weight for C₇₄H₈₈N₅O₁₇PS: 1382.57, Observed mass [M−H]⁻: 1380.92.

Data for 9d (R: NHAc-Ph): MS (ES) m/z calculated molecular weight for C₇₀H₇₁N₆O₁₈PS: 1347.40, Observed mass [M−H]⁻: 1345.85.

A useful process for preparing oligonucleotides is described below as an example. Certain abbreviations: DS1 reagent: TEA-3HF: TEA: H₂O: DMSO=5.0: 7.0: 14.7: 73.3 (v/v/v/v); ETT: 5-(Ethylthio)-1H-tetrazole; CMIMT: N-cyanomethylimidazolium triflate; CPG: controlled pore glass; DBU: 1,8-diazabicyclo[5.4.0]undec-7-ene; DCM: dichloromethane, CH₂Cl₂; DMTr: 4,4′-dimethoxytrityl; IBN: isobutyronitrile; MeIm: N-methylimidazole; TCA: trichloroacetic acid; TEA: triethylamine; TEA-3HF: triethylamine trihydrofluoride; XH: xanthane hydride.

In an example, automated solid-phase synthesis of oligonucleotides was performed according to the cycles shown below: regular amidite cycle (e.g., for natural phosphate linkages, stereorandom phosphorothioate internucleotidic linkages, etc.), DPSE amidite cycle (e.g., using 2′-deoxynucleoside phosphoramidites, for chiral PS linkages such as phosphorothioate internucleotidic linkages, etc.), PSM amidite cycle (e.g., using 2′-O-methyl and 2′-O-methoxyethyl nucleoside phosphoramidites, for chiral PS linkages such as phosphorothioate internucleotidic linkages, etc.), MBR amidite cycle (e.g., for stereo-random PN linkages such as sulfonyl PN linkages). In some embodiments, an amidite in a MBR amidite cyce has the structure of PIII or a salt thereof, e.g. 9-monomer. For stereorandom PN cycles using MBR amidites, sulfonyl azides are dissolved into the amidite solution, dried at least for 2 h under 4 Å molecular sieves and the resulting mixture was used in the synthetic cycle.

### Regular Amidite Cycle

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M ETT/MeCN | 1.0 mL | |
| 3 | oxidation | 50 mM 12/pyridine-H₂O (9:1, v/v) | 2.0 mL | 1 min |
| 4 | cap-2 | 20% Ac₂O, 30% 2,6-lutidine/MeCN | 1.0 mL | 45 s |
| | | 20% MeIm/MeCN | 1.0 mL | |

### DPSE Amidite Cycle

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | cap-1 | 20% Ac₂O, 30% 2,6-lutidine/MeCN | 2.0 mL | 2 min |
| 4 | sulfurization | 0.2M XH / pyridine | 2.0 mL | 6 min |
| 5 | cap-2 | 20% Ac₂O, 30% 2,6-lutidine/MeCN | 1.0 mL | 45 s |
| | | 20% MeIm/MeCN | 1.0 mL | |

### PSM Amidite Cycle

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 8 min |
| | | 0.5M CMIMT/MeCN | 1.0 mL | |
| 3 | cap-1 | 20% Ac₂O, 30% 2,6-lutidine/MeCN | 2.0 mL | 2 min |
| 4 | sulfurization | 0.2M XH/pyridine | 2.0 mL | 6 min |
| 5 | cap-2 | 20% Ac₂O, 30% 2,6-lutidine/MeCN | 1.0 mL | 45 s |
| | | 20% MeIm/MeCN | 1.0 mL | |

### MBR Amidite Cycle

| | | | | waiting |
|---|---|---|---|---|
| step | operation | reagents and solvent | volume | time |
| 1 | detritylation | 3% TCA/DCM | 10 mL | 65 s |
| 2 | coupling | 0.2M monomer/20% IBN-MeCN | 0.5 mL | 10 min |
| | | 1.0M DBU/MeCN | 1.0 mL | |
| 3 | cap-2 | 20% Ac₂O, 30% 2,6-lutidine/MeCN | 1.0 mL | 45 s |
| | | 20% MeIm/MeCN | 1.0 mL | |

A useful procedure for cleavage and deprotection is described below.

After completion of the synthesis, the CPG solid support was dried and transferred into 50 mL plastic tube. The CPG was treated with DS1 reagent (2.4 mL; 100 uL/umol) for 3 h at 27° C., then added conc. NH₃ (4.8 mL; 200 umol/umol) for 16 h at 45° C. The reaction mixture was cooled to room temperature and the CPG was separated by membrane filtration, washed with 15 mL of H₂O. The crude material (filtrate) was analyzed by LTQ and RP-UPLC. Results from certain preparations are presented below. In some embodiments, an oligonucleotide comprises a base sequence (or a portion thereof), one or more nucleobase modifications, a pattern of nucleobase modification (or a portion thereof), one or more sugar modifications, a pattern of sugar modification (or a portion thereof), one or more internucleotidic linkages, a pattern of internucleotidic linkage modification (or a portion thereof), a pattern of linkage phosphorus stereochemistry (or a portion thereof) of an oligonucleotide described below.

| | | calcd. | found | UPLC |
|---|---|---|---|---|
| ID | Crude ODs | [M] | [M] | purity |
| 0140094 | 2283.3 OD (97.16 OD/umol) | 7386.1 | 7385.0 | 19.81% |
| 0140095 | 1064.58 OD (45.30 OD/umol) | 7028.8 | 7027.3 | 36.60% |
| 0140096 | 1733.85 OD (73.78 OD/umol) | 7491.7 | 7491.2 | 22.64% |
| 0140097 | 1959.75 OD (83.39 OD/umol) | 7239.2 | 7238.3 | 28.64% |

| ID | Description | Base Sequence |
|---|---|---|
| 0140094 | m(U)[n006].[moe](G)p.[moe]([m5C])[Ssp]. | UGCCAGGC |
| | [moe]([m5C])[n006].m(A)[Ssp].d(G)[Ssp]. | TGGTTATGA |
| | d(G)[Rsp].d(C)[Ssp].d(T)[Ssp].d(G)[Rsp]. | CUC |
| | d(G)[Ssp].d(T)[Ssp].d(T)[Rsp].d(A)[Ssp]. | |
| | d(T)[Ssp].m(G)[Ssp].m(A)[n006].m(C)[Ssp]. | |
| | m(U)[Ssp].m(C) | |
| 0140095 | m(U)[n064].[moe](G)p.[moe]([m5C])[Ssp]. | UGCCAGGC |
| | [moe]([m5C])[n064].m(A)[Ssp].d(G)[Ssp]. | TGGTTATGA |
| | d(G)[Rsp].d(C)[Ssp].d(T)[Ssp].d(G)[Rsp]. | CUC |
| | d(G)[Ssp].d(T)[Sspl.d(T)[Rsp].d(A)[Ssp]. | |
| | d(T)[Ssp].m(G)[Ssp].m(A)[n064].m(C)[Ssp]. | |
| | m(U)[Ssp].m(C) | |
| 0140096 | m(U)[n074].[moe](G)p.[moe]([m5C])[Ssp]. | UGCCAGGC |
| | [moe]([m5C])[n074].m(A)[Ssp].d(G)[Ssp]. | TGGTTATGA |
| | d(G)[Rsp].d(C)[Ssp].d(T)[Ssp].d(G)[Rsp]. | CUC |
| | d(G)[Ssp].d(T)[Ssp].d(T)[Rsp].d(A)[Ssp]. | |
| | d(T)[Ssp].m(G)[Ssp].m(A)[n074].m(C)[Ssp]. | |
| | m(U)[Ssp].m(C) | |
| 0140097 | m(U)[n075].[moe](G)p.[moe]([m5C])[Ssp]. | UGCCAGGC |
| | [moe]([m5C])[n075].m(A)[Ssp].d(G)[Ssp]. | TGGTTATGA |
| | d(G)[Rsp].d(C)[Ssp].d(T)[Ssp].d(G)[Rsp]. | CUC |
| | d(G)[Ssp].d(T)[Ssp].d(T)[Rsp].d(A)[Ssp]. | |
| | d(T)[Ssp].m(G)[Ssp].m(A)[n075].m(C)[Ssp]. | |
| | m(U)[Ssp].m(C) | |

### Notes:

Description and Base Sequence, due to their length, may be divided into multiple lines. Unless otherwise specified, all oligonucleotides in the table above are single-stranded. As appreciated by those skilled in the art, nucleoside units are unmodified and contain unmodified nucleobases and 2′-deoxy sugars unless otherwise indicated (e.g., with m, m5, meo, etc.); linkages, unless otherwise indicated, are natural phosphate linkages; and acidic/basic groups may independently exist in their salt forms. If a sugar is not specified, the sugar is a natural DNA sugar; and if an intemucleotidic linkage is not specified, the internucleotidic linkage is a natural phosphate linkage. A natural DNA sugar may also be indicated with “d” as in d(G), d(A), d(C), d(T), etc., and a natural phosphate linkage may be indicated with “p” in the table above.

### Moieties and Modifications:

m: 2′-OMe;
m5: methyl at 5-position of C (nucleobase is 5-methylcytosine);
moe: 2′-MOE (2′-OCH₂CH₂OCH₃);
Rsp: Phosphorothioate in the Rp configuration;
Ssp: Phosphorothioate in the Sp configuration;

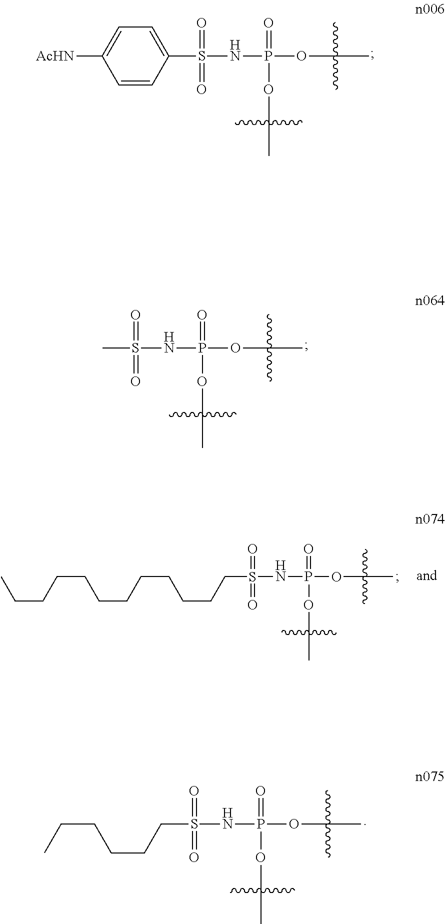 [SMILES: *OP(=O)(NS(=O)(=O)c1ccc(NC(C)=O)cc1)O* ; *OP(=O)(NS(C)(=O)=O)O* ; *OP(=O)(NS(=O)(=O)CCCCCCCCCCCC)O* ; *OP(=O)(NS(=O)(=O)CCCCCC)O*]

### EQUIVALENTS

Having described some illustrative embodiments of the disclosure, it should be apparent to those skilled in the art that the foregoing is merely illustrative and not limiting, having been presented by way of example only. Numerous modifications and other illustrative embodiments are within the scope of one of ordinary skill in the art in accordance with the present disclosure and are within the scope of the disclosure and claims. In particular, although many of the examples presented herein involve specific combinations of method acts or system elements, it should be understood that those acts and those elements may be combined in other ways to accomplish the same objectives. Acts, elements, and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is sufficient to enable one skilled in the art to practice technologies of the present disclosure. The present disclosure is not to be limited in scope by examples provided. Examples are intended as illustrations of one or more aspects of technologies of the present disclosure and other functionally equivalent embodiments are within the scope of the technologies of the present disclosure or claims. Various modifications of the disclosure in addition to those shown and described herein may become apparent to those skilled in the art from the foregoing description and fall within the scope of the appended claims. The advantages and objects of the disclosure are not necessarily encompassed by each embodiment of the disclosure.

1. A method for preparing a compound of formula P: 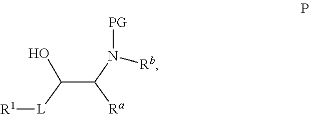
or a salt thereof,
comprising reducing a compound of formula INT-1: 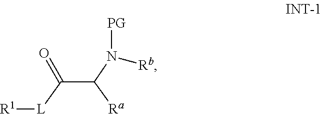
or a salt thereof to provide a compound of formula P or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or 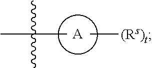
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

2. A method for preparing a compound of formula P-a: 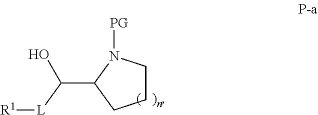
or a salt thereof,
comprising reducing a compound of formula INT-1-a: 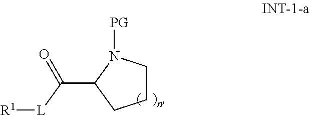
or a salt thereof to provide a compound of formula P-a or a salt thereof, wherein:
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or 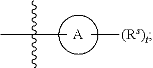
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

3. A method for preparing a compound of formula INT-1: 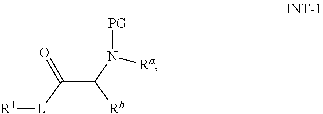
or a salt thereof,
comprising reacting a compound of formula INT-2: 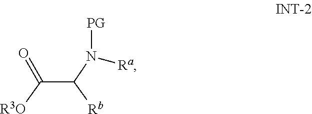
or a salt thereof with a compound of formula INT-3:
R¹-L-H, INT-3
or a salt thereof to provide the compound of formula INT-1 or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or 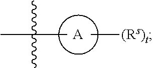
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

4. A method for preparing a compound of formula INT-1-a: 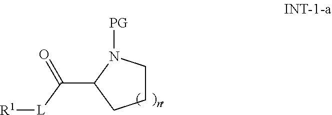
or a salt thereof,
comprising reacting a compound of formula INT-2-a: 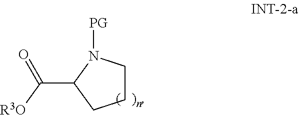
or a salt thereof with a compound of formula INT-3:
R¹-L-H, INT-3
or a salt thereof to provide the compound of formula INT-1 or a salt thereof, wherein:
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or 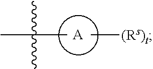
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

5. The method of any one of the preceding claims, wherein R¹ is —S(O)₂R².

6. The method of claim 5, wherein R² is

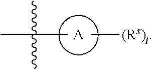

7. The method of claim 6, wherein Ring A is an optionally substituted phenyl ring.

8. The method of any one of claims 5-7, wherein t is 1.

9. The method of claim 5, wherein R² is optionally substituted C₁₋₆ alkyl.

10. The method of any one of claims 1-4, wherein R¹ is —Si(R)₃.

11. The method of claim 10, wherein each R is independently an optionally substituted group selected from C₁₋₃₀ aliphatic and C₆₋₃₀ aryl.

12. The method of claim 10 wherein R¹ is —Si(Ph)₂Me.

13. The method of any one claims 1-2 and 5-12, wherein the compound of formula P-a has the structure of: 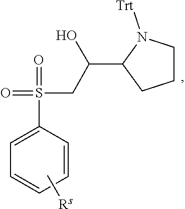
or a salt thereof,
wherein R^{s} is H, Cl, Br, CN, COOMe, COOEt, OMe, NMe₂, or an optionally substituted group selected from phenyl and a 5-20 membered heteroaryl having 1-3 heteroatoms independently selected from oxygen, nitrogen, sulfur, phosphorus and silicon.

14. The method of claim 13, wherein the compound of formula P-a has the structure of: 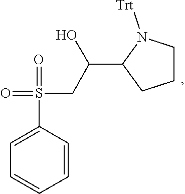
or a salt thereof.

15. The method of any one of the preceding claims, further comprising:
providing a compound of formula INT-4: 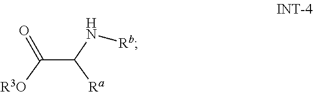
or a salt thereof; and
reacting a compound of formula INT-4 with an amino protecting agent to provide a compound of formula INT-2.

16. The method of any one of the preceding claims, further comprising:
providing a compound of formula INT-4-a: 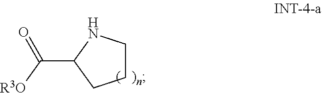
or a salt thereof, and
reacting a compound of formula INT-4-a with an amino protecting agent to provide a compound of formula INT-2-a.

17. The method of any one of claims 15-16, wherein the amino protecting agent has the structure of PG-LG, wherein LG is a leaving group.

18. The method of claim 17, wherein LG is —Cl.

19. A method for preparing the compound of formula I: 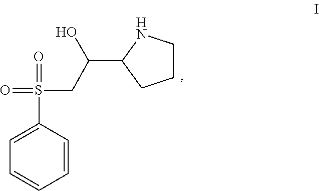 [SMILES: O=S(=O)(CC(O)C1CCCN1)c1ccccc1]
or a salt thereof, comprising:
(a) providing a compound having the structure of: 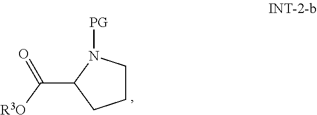
or a salt thereof, wherein:
PG is an amino protecting group;
R³ is —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; and
(b) reacting a compound of formula INT-2-b with 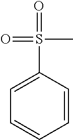
to provide a compound of formula INT-7: 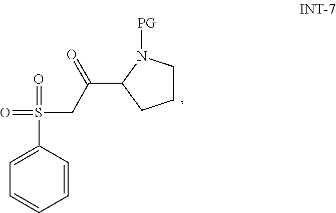
or a salt thereof, and
(c) reducing the compound of formula INT-7 to provide a compound of formula P-I: 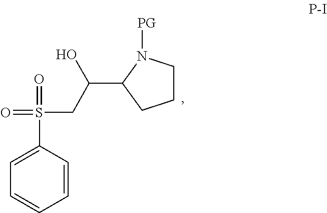
or a salt thereof, wherein the reduction of a compound of formula INT-7 or a salt thereof is carried out in the presence of a reducing agent; and
(e) subject a compound of formula P-I to deprotection to provide a compound of formula I.

20. The method of claim 19, further comprising:
reacting a compound of formula INT-4-b: 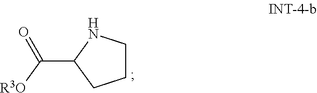
with an amino protecting agent to provide a compound of formula INT-2-b.

21. The method of any one of the preceding claims, further comprising:
reacting a compound of formula INT-5: 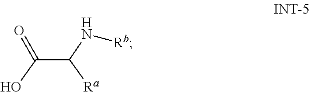
with a compound of R³OH to provide the compound of formula INT-4.

22. The method of any one of the preceding claims, further comprising:
reacting a compound of formula INT-5-a: 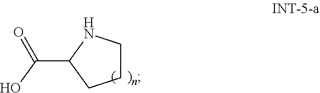
with a compound of R³OH to provide the compound of formula INT-4-a.

23. The method of any one of the claims 20-22, wherein the amino protecting agent has the structure of LG-PG or a salt thereof, wherein LG is a leaving group.

24. A compound having the structure of formula INT-1: 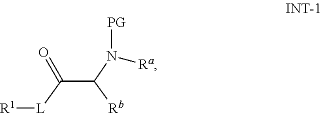
or a salt thereof to provide a compound of formula P or a salt thereof, wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or 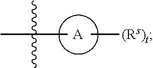
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

25. A compound having the structure of formula INT-1-a: 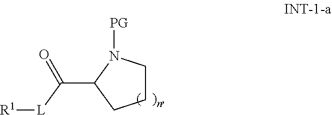
or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or 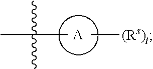
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

26. The compound of any one of claims 24-25, wherein R¹ is —S(O)₂R².

27. The compound of claim 26, wherein R² is

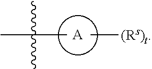

28. The compound of claim 27, wherein Ring A is an optionally substituted phenyl ring.

29. The compound of any one of claims 27-28, wherein t is 1.

30. The compound of claim 26, wherein R² is optionally substituted C₁₋₆ alkyl.

31. The compound of any one of claims 24-25, wherein R¹ is —Si(R)₃.

32. The compound of claim 31, wherein each R is independently an optionally substituted group selected from C₁₋₃₀ aliphatic and C₆₋₃₀ aryl.

33. The compound of any one of claims 31-32, wherein R¹ is —Si(Ph)₂Me.

34. A compound having the structure of 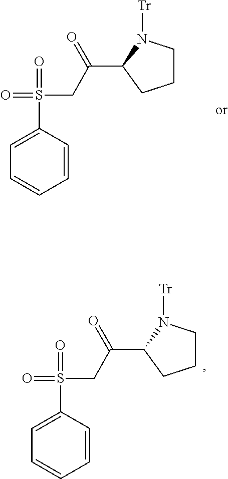
or a salt thereof.

35. A compound having the structure of 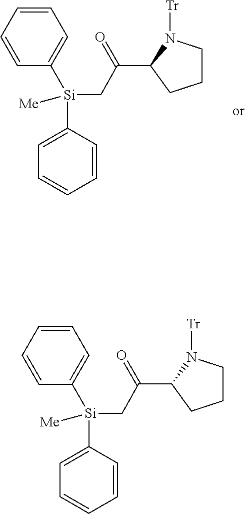
or a salt thereof.

36. A composition comprising:
(1) a compound of formula INT-1 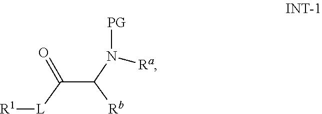
or a salt thereof, and
(2) a compound of formula P: 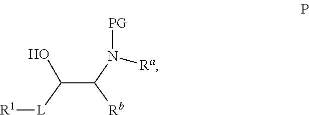
or a salt thereof; wherein
PG is an amino protecting group;
L is optionally substituted —CH₂—;
R¹ is R, —P(O)(R²)₂, —S(O)₂R², or —Si(R)₃;
R² is R, —OR, —N(R′)₂, or 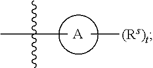
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

37. A composition comprising:
(1) a compound of formula INT-1-a: 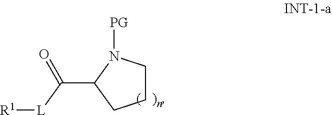
or a salt thereof, and
(2) a compound of formula P-a: 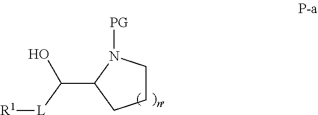
or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or 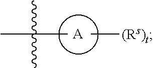
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

38. A composition comprising:
(1) a compound of formula INT-1: 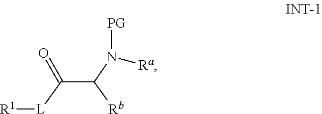
or a salt thereof, and
(2) a compound of formula INT-3:
R¹-L-H, INT-3
or a salt thereof; wherein
PG is an amino protecting group;
L is optionally substituted —CH₂—;
R¹ is R, —P(O)(R²)₂, —S(O)₂R², or —Si(R)₃;
R² is R, —OR, —N(R′)₂, or 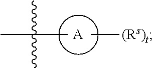
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

39. A composition comprising:
(1) a compound of formula INT-1-a: 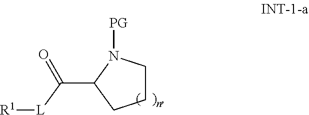
or a salt thereof, and
(2) a compound of formula INT-3:
R¹-L-H, INT-3
or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or 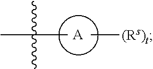
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

40. A composition comprising:
(1) a compound of formula INT-1: 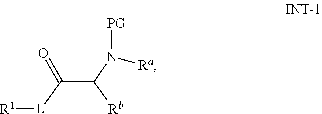
or a salt thereof, and
(2) a compound of formula INT-2: 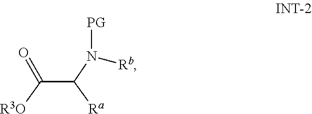
or a salt thereof; wherein:
PG is an amino protecting group;
L is optionally substituted —CH₂—;
R¹ is R, —P(O)(R²)₂, —S(O)₂R², or —Si(R)₃;
R² is R, —OR, —N(R′)₂, or 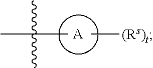
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R^{b} is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

41. A composition comprising:
(1) a compound of formula INT-1-a: 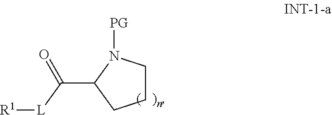
or a salt thereof, and
(2) a compound of formula INT-2-a: 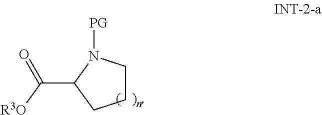
or a salt thereof; wherein
n is 0, 1, 2, or 3;
PG is an amino protecting group;
L is optionally substituted —CH₂—;
R¹ is R, —P(O)(R²)₂, —S(O)₂R², or —Si(R)₃;
R² is R, —OR, —N(R′)₂, or 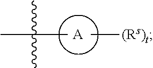
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

42. The composition of any one of claims 36-41, wherein R¹ is —S(O)₂R².

43. The composition of claim 42, wherein R² is 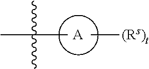
or optionally substituted C₁₋₆ alkyl.

44. The composition of any one of claims 36-41, wherein R¹ is —Si(R)₃, wherein each R is independently an optionally substituted group selected from C₁₋₃₀ aliphatic and C₆₋₃₀ aryl.

45. The composition of claim 44, wherein R¹ is —Si(Ph)₂Me.

46. A compound, wherein the compound has a structure of PMT-A: 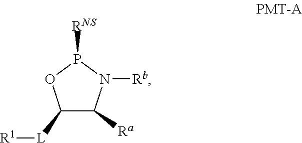
or a salt thereof, wherein:
R^{NS} is a nucleoside;
L is optionally substituted —CH₂—;
R¹ is R, —P(O)(R²)₂, —S(O)₂R², or —Si(R)₃;
R² is R, —OR, —N(R′)₂, or 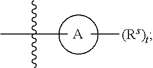
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

47. A compound, wherein the compound has a structure of PMT-B: 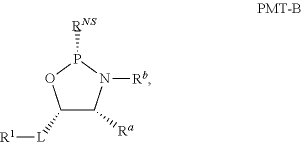
or a salt thereof, wherein:
R^{NS} is a nucleoside;
L is optionally substituted —CH₂—;
R¹ is R, —P(O)(R²)₂, —S(O)₂R², or —Si(R)₃;
R² is R, —OR, —N(R′)₂, or 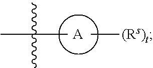
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

48. A compound, wherein the compound has a structure of PMT-A1: 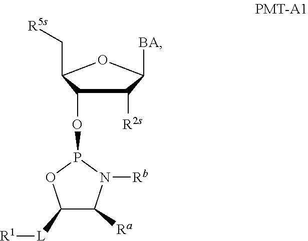
or a salt thereof, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon;
L is optionally substituted —CH₂—;
R¹ is R, —P(O)(R²)₂, —S(O)₂R², or —Si(R)₃;
R² is R, —OR, —N(R′)₂, or 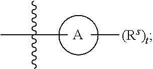
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

49. A compound, wherein the compound has a structure of PMT-B1: 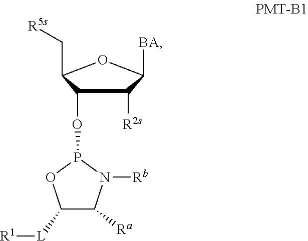
or a salt thereof, wherein:
BA is an optionally substituted group selected from C₃₋₃₀ cycloaliphatic, C₆₋₃₀ aryl, C₃₋₃₀ heterocyclyl having 1-10 heteroatoms independently selected from oxygen, nitrogen, sulfur, phosphorus and silicon, C₅₋₃₀ heteroaryl having 1-10 heteroatoms independently selected from oxygen, nitrogen, sulfur, phosphorus and silicon, a natural nucleobase moiety, and a modified nucleobase moiety;
R^{5s} is R′ or —OR′;
R^{2s} is —F, —CN, —N₃, —NO, —NO₂, —R′ —OR′, —SR′, —N(R′)₂, —O-L^{s}-OR′, —O-L^{s}-SR′, or —O-L^{s}-N(R′)₂, or R^{2s} is L^{s} connecting C2 with C1, C2, C3, C4 or C5; and
L^{s} is a covalent bond, or a bivalent, optionally substituted, linear or branched group selected from C₁₋₃₀ aliphatic and C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted C₁₋₆ alkylene, C₁₋₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
-Cy- is an optionally substituted bivalent ring selected from 3-30 membered carbocyclylene, 6-30 membered arylene, 5-30 membered heteroarylene having 1-10 heteroatoms independently selected from oxygen, nitrogen and sulfur, and 3-30 membered heterocyclylene having 1-10 heteroatoms independently selected from oxygen, nitrogen, sulfur, phosphorus and silicon;
L is optionally substituted —CH₂—;
R¹ is R, —P(O)(R²)₂, —S(O)₂R², or —Si(R)₃;
R² is R, —OR, —N(R′)₂, or 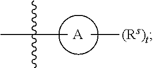
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

50. The compound of any one of claims 46-49, wherein R^{2s} is L^{s} connecting C2 with C₄, wherein L^{s} is (C2)-O-(optionally substituted methylene)-.

51. The compound of any one of claims 46-49, wherein R^{2s} is —OR′, wherein R′ is optionally substituted C₁₋₆ aliphatic, or R^{2s} is selected form —H, —F, —OMe, and —OCH₂CH₂OMe.

52. The compound of any one of claims 46-51, wherein R^{5s} is —OR′.

53. The compound of any one of claims 46-52, wherein R^{5s} is -ODMTr.

54. The compound of any one of claims 46-53, wherein BA is an optionally substituted group which group is selected from

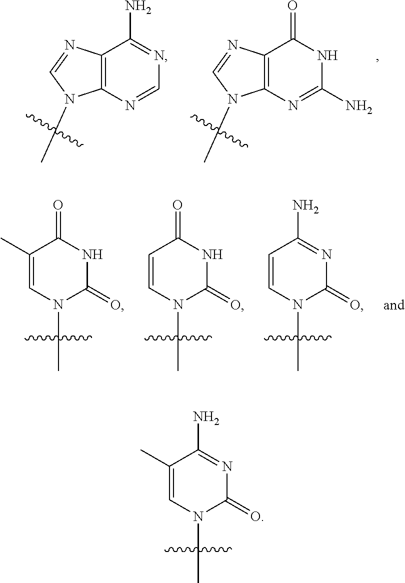

55. The compound of any one of claims 46-54, wherein R¹ is —S(O)₂R².

56. The compound of claim 55, wherein R² is 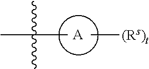
or optionally substituted C₁₋₆ alkyl.

57. The compound of any one of claims 46-54, wherein R¹ is —Si(R)₃, wherein each R is independently an optionally substituted group selected from C₁₋₃₀ aliphatic and C₆₋₃₀ aryl.

58. The compound of claim 57, wherein R¹ is —Si(Ph)₂Me.

59. The compound of any one of claims 46-58, wherein R^{a} and R^{b} are taken together with their intervening atoms to form an optionally substituted Ring B, wherein Ring B is 4-10 membered and has 0-4 (e.g., 0, 1-4, 1, 2, 3, 4, etc.) heteroatoms in addition to the nitrogen atom.

60. The compound of any one of claims 46-59, wherein Ring B is

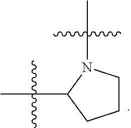 [SMILES: *C1CCCN1*]

61. A composition comprising a compound of any one of claims 46-60, wherein the ratio of the compound and its epimer with respect to the chiral phosphorus is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%, and/or wherein the ratio of cis isomer:trans isomer is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1^{%}.

62. A method of preparing a compound or composition of any one of claims 46-61, comprising reacting a compound having the structure of formula CA: 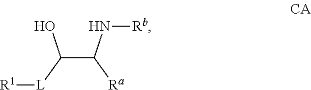
or a salt thereof with a nucleoside, wherein:
L is optionally substituted —CH₂—;
R¹ is R, —P(O)(R²)₂, —S(O)₂R², or —Si(R)₃;
R² is R, —OR, —N(R′)₂, or 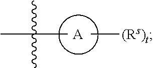
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

63. A method of preparing a compound or composition of any one of claims 46-61, comprising reacting a compound having the structure of formula CA-A: 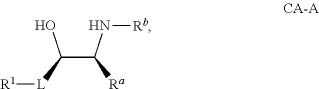
or a salt thereof with a nucleoside, wherein:
L is optionally substituted —CH₂—;
R¹ is R, —P(O)(R²)₂, —S(O)₂R², or —Si(R)₃;
R² is R, —OR, —N(R′)₂, or 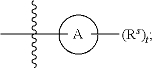
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

64. A method of preparing a compound or composition of any one of claims 46-61, comprising reacting a compound having the structure of formula CA-B: 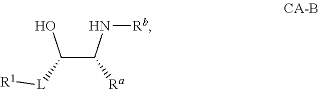
or a salt thereof with a nucleoside, wherein:
L is optionally substituted —CH₂—;
R¹ is R, —P(O)(R²)₂, —S(O)₂R², or —Si(R)₃;
R² is R, —OR, —N(R′)₂, or 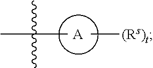
Ring A is an optionally substituted 5-10 membered aromatic ring having 0-5 heteroatoms independently selected from oxygen, nitrogen, sulfur, phosphorus and silicon;
each R^{s} is independently R′, halogen, —CN, —C(O)OR′, —OR′, —N(R′)₂;
t is 0, 1, 2, 3, 4 or 5;
R^{a} and R^{b} are taken together with their intervening atoms to form Ring B, wherein Ring B is an optionally substituted 4-15 membered ring having, in addition to the nitrogen atom to which R is attached, 0-4 heteroatoms;
each R′ is independently R, —C(O)R, —C(O)OR, —C(O)N(R)₂, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon; or
two R groups are optionally and independently taken together to form a covalent bond; or:
two R groups are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

65. The method of preparing a compound or composition of any one of claims 62-64, wherein the nucleoside is R^{NS}—H or a salt thereof and R^{NS} is

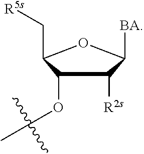

66. The method of any one of claims 62-65, wherein R^{2s} is L^{s} connecting C2 with C₄, wherein L^{s} is (C2)-O-(optionally substituted methylene)-.

67. The method of any one of claims 62-65, wherein R^{2s} is —OR′, wherein R′ is optionally substituted C₁₋₆ aliphatic, or wherein R^{2s} is selected form —H, —F, —OMe, and —OCH₂CH₂OMe.

68. The method of any one of claims 62-67, wherein R^{5s} is —OR′.

69. The method of any one of claims 62-67, wherein R^{5s} is -ODMTr.

70. The method of any one of claims 62-69, wherein BA is an optionally substituted group which group is selected from

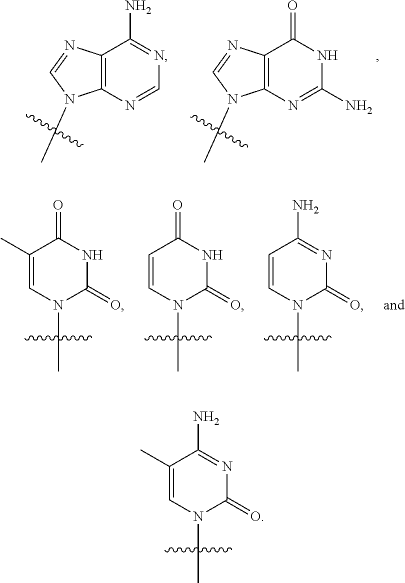

71. The method of any one of claims 62-70, wherein R¹ is —S(O)₂R².

72. The method of claim 71, wherein R² is 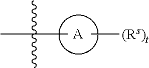
or optionally substituted C₁₋₆ alkyl.

73. The method of any one of claims 62-70, wherein R¹ is —Si(R)₃, wherein each R is independently an optionally substituted group selected from C₁₋₃₀ aliphatic and C₆₋₃₀ aryl.

74. The method of claim 73, wherein R¹ is —Si(Ph)₂Me.

75. The method of any one of claims 62-74, wherein R^{a} and R^{b} are taken together with their intervening atoms to form an optionally substituted Ring B, wherein Ring B is 4-10 membered and has 0-4 (e.g., 0, 1-4, 1, 2, 3, 4, etc.) heteroatoms in addition to the nitrogen atom.

76. The method of any one of claims 62-75, wherein Ring B is

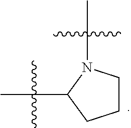 [SMILES: *C1CCCN1*]

77. The method of any one of claims 62-75, wherein the method is for preparing a compound of 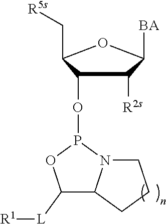
or a salt thereof, and the ratio of trans isomer:cis isomer is about or at least about 50%:50%, about 60%:40%, about 70%:30%, about 80%:20%, about 90%:10%, about 91%:9%, about 92%:8%, about 93%:7%, about 94%:6%, about 95%:5%, about 96%:4%, about 97%:3%, about 98%:2%, about 99%:1%, about 99.5%:0.5%, or about 99.9%:0.1%.

78. The method of any one of claims 62-75, wherein the method is for preparing a compound of 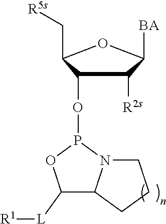
or a salt thereof, and the ratio of trans isomer:cis isomer is about or less than about 50%:5%, about 40%:60%, about 30%:70%, about 20%:80%, about 10%:90%, about 9%:91%, about 8%:92%, about 7%:93%, about 6%:94%, about 5%:95%, about 4%:96%, about 3%:97%, about 2%:98%, about 1%:99%, about 0.05%:99.5%, or about 0.1%:99.9%.

79. The method of any one of claims 77-78, wherein the trans isomer and cis isomer are 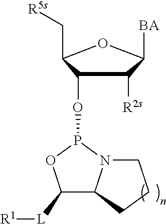
or a salt thereof and 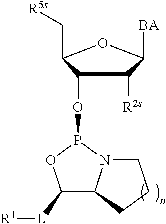
or a salt thereof, respectively, or wherein the trans isomer and cis isomer are 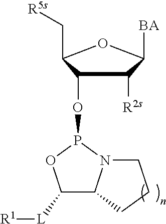
or a salt thereof and 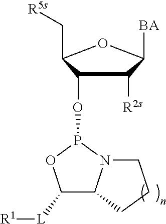
or a salt thereof, respectively.

80. The method of any one of claims 62-79, wherein the reacting is performed in the presence of base.

81. The method of claim 80, wherein the base is DBU or DBN.

82. The method of any one of claims 62-81, wherein the reaction is performed in the presence of another base.

83. The method of claim 82, wherein the another base is TEA.

84. A method for isomerizing a compound of any one of claims 45-60 with respect to its chiral phosphorus, comprising contacting the compound with a phosphoramidite activator for oligonucleotide synthesis.

85. A method for preparing an oligonucleotide, comprising steps of:
1) contacting a phosphoramidite composition comprising a compound of any one of claims 45-60 with a phosphoramidite activator to provide an isomerized phosphoramidite composition, wherein the isomerized phosphoramidite composition comprises a lower level of the compound compared to the phosphoramidite composition; and
2) contacting the isomerized phosphoramidite composition with a coupling partner.

86. The method of any one of claims 84-85, wherein the activator is CMIMT.

87. A method for isomerizing a compound of any one of claims 45-60 with respect to its chiral phosphorus, comprising contacting the compound with a mildly acidic compound.

88. A method for assessing level of a compound in a composition, comprising using a compound or composition of any one of claims 45-60 as a reference.

89. A method, comprising:
1) contacting a P(III) agent with an azide to provide a composition for coupling;
2) contacting the composition for coupling with a coupling partner.

90. The method of claim 89, wherein the ratio of the azide to the coupling partner is about or less than about 2, 1.9, 1.8, 1.7, 1.6, 1.5, 1.4, 1.3, 1.2, or 1.1.

91. The method of any one of claims 89-90, wherein the ratio of the azide to the P(III) agent is about or less than about 2, 1.9, 1.8, 1.7, 1.6, 1.5, 1.4, 1.3, 1.2, or 1.1.

92. The method of any one of claims 89-91, wherein the P(III) agent has the structure of formula PIII: 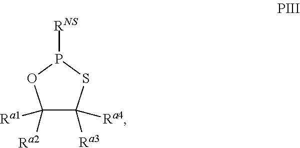
or a salt thereof, wherein R^{NS} is a nucleoside and each of R^{a1}, R^{a2}, R^{a3} and R^{a4} is independently R′ as described herein.

93. The method of any one of claims 89-91, wherein the P(III) agent has the structure of 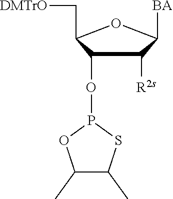
or a salt thereof.

94. The composition, compound, or method described in the Specification or of Embodiments 1-783.
